(19) United States
(12) Patent Application Publication
(10) Pub. No.: US 2026/0102395 A1
(43) Pub. Date: Apr. 16, 2026

(54) INHIBITING HISTONE DEACETYLASE 6

(71) Applicant: Eikonizo Therapeutics, Inc., Cambridge, MA (US)

(72) Inventors: Tonya Marie Gilbert, Somerville, MA (US); Thomas Edward Richardson, Cary, NC (US); Lauren Taylor Evans, Buffalo, NY (US); Rebecca Ellen James, Severna Park, MD (US); Frederick Albert Schroeder, Weston, MA (US); Jianhong Wang, Foster City, CA (US); Florence Fevrier Wagner, Waltham, MA (US)

(73) Assignee: Eikonizo Therapeutics, Inc., Cambridge, MA (US)

(21) Appl. No.: 19/357,695

(22) Filed: Oct. 14, 2025

Related U.S. Application Data
(60) Provisional application No. 63/706,232, filed on Oct. 11, 2024.

(51) Int. Cl. A61K 31/4985; A61K 31/4439; A61K 31/444; A61K 31/4709; A61K 31/4725; A61K 31/497; A61K 31/498; A61K 31/501; A61K 31/506; A61K 31/517; A61K 31/519; C07D 413/04; C07D 413/14; C07D 471/04; C07D 487/04

(52) U.S. Cl. CPC A61K 31/4985; A61K 31/4439; A61K 31/444; A61K 31/4709; A61K 31/4725; A61K 31/497; A61K 31/498; A61K 31/501; A61K 31/506; A61K 31/517; A61K 31/519; C07D 413/04; C07D 413/14; C07D 471/04; C07D 487/04

(57) ABSTRACT

Provided herein are methods of treating a subject with compounds that inhibit HDAC6, including methods of treating a variety of diseases including neurological disorders.

### RELATED PATENT APPLICATIONS

This patent application claims priority under 35 U.S.C. § 119(e) to U.S. Provisional Patent Application Ser. No. 63/706,232, filed Oct. 11, 2024, incorporated by reference herein in its entirety. This patent application is filed on (Tuesday) Oct. 14, 2025, the next succeeding federal business day after (Saturday) Oct. 11, 2025.

### BACKGROUND

Histone deacetylase 6 (HDAC6), a class IIb HDAC, is a cytoplasmic, microtubule-associated enzyme. HDAC6 has unique features among the HDAC paralogs. Unlike other HDACs, HDAC6 contains two deacetylase domains and a ubiquitin binding domain allowing HDAC6 to function in distinct cell signaling systems involving protein acetylation and ubiquitination, respectively. Importantly, it does not deacetylate histones.

Histone deacetylase 6 (HDAC6) is a unique member of the HDAC family that predominantly resides in the cytoplasm and targets non-histone proteins, including α-tubulin, for deacetylation. Tubulin acetylation, particularly on lysine 40 of α-tubulin, is a post-translational modification that stabilizes microtubules and promotes intracellular transport, cellular morphology, and response to stress. HDAC6 counteracts this by deacetylating tubulin, thereby modulating microtubule dynamics and influencing processes such as autophagy, cell motility, and protein aggregate clearance via the aggresome pathway. Unlike nuclear HDACs involved in gene transcription, HDAC6's functions are largely cytoplasmic and structural, making it an appealing therapeutic target for diseases involving disrupted protein homeostasis and transport.

Inhibitors of HDAC6 that promote tubulin acetylation have shown promise in treating several diseases. In neurodegenerative disorders like Alzheimer's, Parkinson's, and Huntington's disease, impaired axonal transport and protein aggregation are key pathological features. Enhancing tubulin acetylation through HDAC6 inhibition can restore axonal trafficking and support the clearance of toxic aggregates. Additionally, HDAC6 inhibitors have shown potential in cancer by disrupting cancer cell migration and inducing apoptosis. They are also being explored for treating inflammatory and autoimmune diseases, as HDAC6 modulates immune signaling and stress responses. Thus, a compound that inhibits HDAC6 and acetylates tubulin holds multi-faceted therapeutic potential.

Developing an HDAC6 inhibitor that also acetylates tubulin and effectively penetrates the brain is especially desirable for treating central nervous system (CNS) disorders. The blood-brain barrier (BBB) presents a major challenge for drug delivery to the brain, so compounds with both efficacy and BBB permeability are valuable. In diseases like Alzheimer's and Huntington's, where HDAC6 activity is upregulated and neuronal transport is compromised, a brain-penetrant inhibitor could restore proper neuronal function and reduce neurodegeneration. Furthermore, such compounds may offer disease-modifying effects rather than merely symptomatic relief, making them especially attractive candidates in CNS drug development.

People with amyotrophic lateral sclerosis (pALS) exhibit defects in proteostasis and axonal transport that are linked to motor dysfunction. Neither pathway is adequately addressed by current standards of care. HDAC6 is a cytoplasmic enzyme with dual functions in axonal transport and proteostasis. HDAC6 deactylates non-histone substrates associated with intracellular transport and delivers ubiquitinated protein aggregates into the autophagy-lysosome pathway for degradation.

### SUMMARY

Compounds and methods for inhibiting histone deacetylase 6 (HDAC6) and increasing levels of acetylated tubulin in cells are provided herein. The compounds and methods are useful in stabilizing microtubules and enhancing intracellular transport. HDAC6 is a cytoplasmic deacetylase that specifically targets non-histone proteins like α-tubulin, and its overactivity can lead to hypoacetylation of tubulin, resulting in impaired axonal transport indicative of many neurodegenerative diseases. Compounds and methods are provided herein for inhibiting HDAC6 and increasing the amount of tubulin that remains acetylated, supporting microtubule function and promoting the transport of essential organelles, such as mitochondria, along axons. This mechanism is particularly relevant in neurodegenerative conditions like Alzheimer's, Parkinson's, and Huntington's disease, where disrupted cellular transport contributes to neuronal dysfunction and degeneration. Therefore, the HDAC6 inhibitor compounds and related methods are useful as therapeutic agents to restore cellular homeostasis and mitigate neurodegenerative progression, for the treatment of various neurodegenerative conditions.

HDAC6 is a cytoplasmic enzyme that specifically deacetylates α-tubulin (K40). The invention is based in part on the discovery that certain compounds provided herein unexpectedly increased acetylated alpha-tubulin (K40) relative to total alpha-tubulin in the homogeneous time resolved fluorescence (HTRF) analysis of Example 5. Acetylation at lysine 40 (K40) on α-tubulin is a post-translational modification associated with microtubule stability and certain cellular functions including intracellular transport and signaling. HTRF measures levels of acetylated α-tubulin (K40) relative to total α-tubulin. An increase in acetyl-K40 signal following HDAC6 inhibitor treatment indicates that the inhibitor is biologically active and successfully blocking HDAC6 function. In neuronal cells like SH-SY5Y, acetylated α-tubulin promotes axonal transport, which is impaired in neurodegenerative diseases. In some embodiments, the disclosed compounds increase acetyl-K40 from HDAC6 inhibition, useful in methods for treating and/or restoring neuronal function and/or reducing protein aggregation in the CNS.

In some embodiments, methods of treating a neurodegenerative condition, comprise administering to a subject in need thereof, a therapeutically effective amount of a compound of Formula (I),

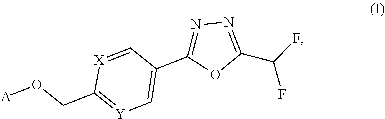

or a pharmaceutically acceptable salt thereof,
wherein:
   X is N or CR¹, and Y is CR¹ or N, provided that at least one of X and Y is N;
R¹ is hydrogen or halogen;
   A is a 6,6-bicyclic aryl ring system, a 5,6-bicyclic heteroaryl ring system or a 6,6-bicyclic heteroaryl ring system, wherein each heteroaryl ring system comprising one or more nitrogen or oxygen heteroatoms, and A is optionally substituted with one or more independent substituents R²; and
   each occurrence of R² is independently halogen, amido, cyano, substituted or unsubstituted acyl, substituted or unsubstituted C₁₋₄ alkyl, substituted or unsubstituted C₁₋₄ alkenyl, substituted or unsubstituted C₁₋₄ alkynyl, substituted or unsubstituted 3-6 member heteroaliphatic, substituted or unsubstituted C₃₋₆ carbocyclyl, substituted or unsubstituted 3-6-member heterocyclyl, substituted or unsubstituted C₆₋₁₄ aryl, or substituted or unsubstituted 6-10 member heteroaryl; or two occurrences of R² are joined with their intervening atoms to form substituted or unsubstituted 3-6 member carbocyclyl, substituted or unsubstituted 3-6 member heterocyclyl, substituted or unsubstituted C₆₋₁₄ aryl, or substituted or unsubstituted 6-10 member heteroaryl.

In some embodiments, methods treating a neurodegenerative disease by selectively inhibiting HDAC6 over HDAC8 while inhibiting alpha tubulin deacetylation (K40), comprise administering to a subject in need thereof, a therapeutically effective amount of a compound of Formula (I).

In some embodiments, R¹ is hydrogen or F. In some embodiments, X is N, and Y is CH or CF.

In some embodiments, A is a 5,6-bicyclic heteroaryl group or a 6,6-bicyclic heteroaryl group comprising one or more nitrogen atoms. In some embodiments, A is an unsubstituted 5,6-bicyclic heteroaryl group or an unsubstituted 6,6-bicyclic heteroaryl group comprising one or more nitrogen atoms. In some embodiments, A is a 5,6-bicyclic heteroaryl group or a 6,6-bicyclic heteroaryl group comprising one or more nitrogen atoms, wherein A is substituted with halogen, pyridine, pyrimidine, or phenyl optionally substituted with one or more halogen. In some embodiments, A is,

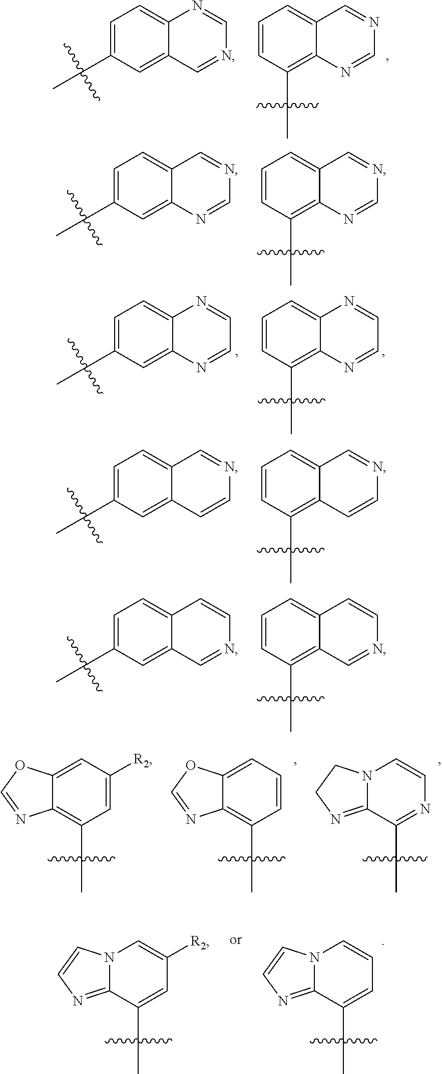

In some embodiments, A is unsubstituted, or A is substituted with one R².

In some embodiments, each R² is independently halogen, —CN, —CH₂CN, C₁₋₄ alkyl, C₃₋₆ cycloalkyl, C₁₋₄ haloalkyl, acyl, or amide. In some embodiments, A is quinazoline, quinoxaline, isoquinoline, 2,3-dihydroimidazo[1,2-a]pyrazine, imidazo[1,2-a]pyridine, or benzo[d]oxazole. In some embodiments, R² is halogen, C₁₋₄ alkyl, phenyl, or 5-6 member heteroaryl, wherein the phenyl or 6-member heteroaryl is optionally substituted with halogen, —CN, —CH₂CN, C₁₋₄ alkyl, C₃₋₆ cycloalkyl, C₁₋₄ haloalkyl, acyl, or amide. In some embodiments, R² is halogen, C₁₋₄ alkyl, phenyl optionally substituted with halogen or 6-member heteroaryl comprising one or more nitrogen heteroatom. In some embodiments, R² is halogen, phenyl optionally substituted with fluoro, or 6-member heteroaryl comprising one or more nitrogen heteroatoms.

In some embodiments, the compound is selected from the group consisting of:

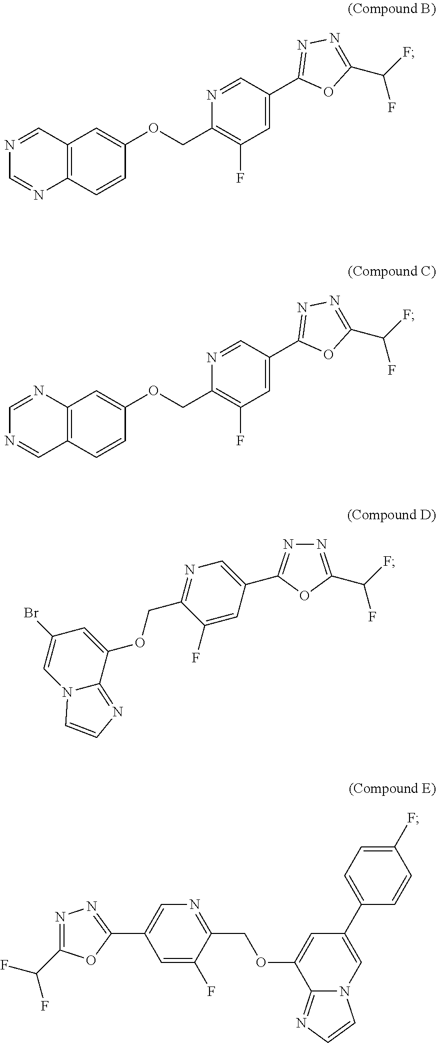 [SMILES: Fc1cc(-c2nnc(C(F)F)o2)cnc1COc1ccc2ncncc2c1 ; Fc1cc(-c2nnc(C(F)F)o2)cnc1COc1ccc2cncnc2c1 ; Fc1cc(-c2nnc(C(F)F)o2)cnc1COc1cc(Br)cn2ccnc12 ; Fc1ccc(-c2cc(OCc3ncc(-c4nnc(C(F)F)o4)cc3F)c3nccn3c2)cc1]

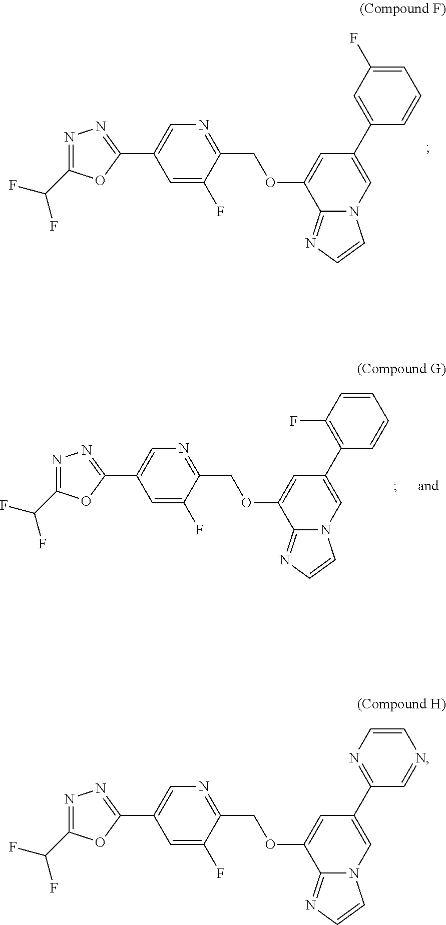 [SMILES: Fc1cccc(-c2cc(OCc3ncc(-c4nnc(C(F)F)o4)cc3F)c3nccn3c2)c1 ; Fc1ccccc1-c1cc(OCc2ncc(-c3nnc(C(F)F)o3)cc2F)c2nccn2c1 ; Fc1cc(-c2nnc(C(F)F)o2)cnc1COc1cc(-c2cnccn2)cn2ccnc12]

or a pharmaceutically acceptable salt thereof.

In one aspect, provided is the compound (A)

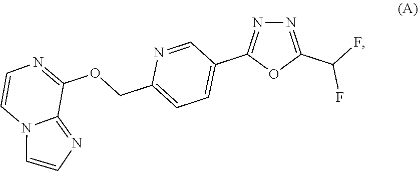 [SMILES: FC(F)c1nnc(-c2ccc(COc3nccn4ccnc34)nc2)o1]

and pharmaceutically acceptable salts thereof. In some embodiments, methods comprising the use or administration of Compound A,

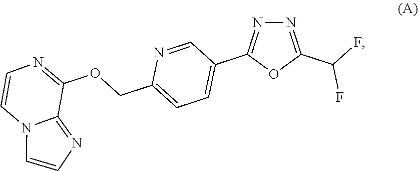 [SMILES: FC(F)c1nnc(-c2ccc(COc3nccn4ccnc34)nc2)o1]

or a pharmaceutically acceptable salt thereof, are provided. In some embodiments, methods comprising the use of 2-(difluoromethyl)-5-(6-((imidazo[1,2-a]pyrazin-8-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(6-((imidazo[1,2-a]pyrazin-8-yloxy)methyl)pyridin-3-yl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(difluoromethyl)-5-(6-((imidazo[1,2-a]pyrazin-8-yloxy)methyl)pyridin-3-yl)-1,3,4-oxadiazole (Compound A), or a pharmaceutically acceptable salt thereof.

In one aspect, provided is the compound (D)

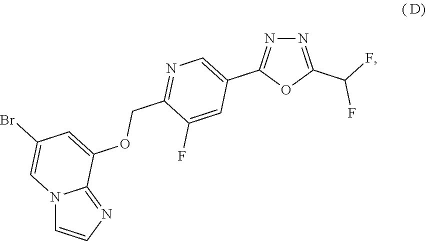 [SMILES: Fc1cc(-c2nnc(C(F)F)o2)cnc1COc1cc(Br)cn2ccnc12]

and pharmaceutically acceptable salts thereof. In some embodiments, methods comprising the use or administration of Compound D,

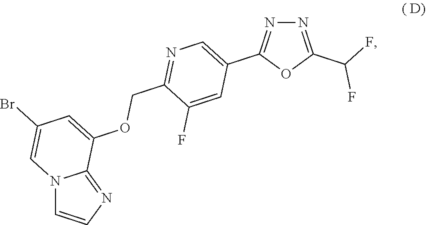 [SMILES: Fc1cc(-c2nnc(C(F)F)o2)cnc1COc1cc(Br)cn2ccnc12]

or a pharmaceutically acceptable salt thereof, are provided. In some embodiments, methods comprising the use of 2-(6-(((6-bromoimidazo[1,2-a]pyridin-8-yl)oxy)methyl)-5-fluoropyridin-3-yl)-5-(difluoromethyl)-1,3,4-oxadiazole, or a pharmaceutically acceptable salt thereof for the inhibition of HDAC6 are provided. In some embodiments, methods comprising the administration of a therapeutically acceptable amount of 2-(6-(((6-bromoimidazo[1,2-a]pyridin-8-yl)oxy)methyl)-5-fluoropyridin-3-yl)-5-(difluoromethyl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(6-(((6-bromoimidazo[1,2-a]pyridin-8-yl)oxy)methyl)-5-fluoropyridin-3-yl)-5-(difluoromethyl)-1,3,4-oxadiazole (Compound D), or a pharmaceutically acceptable salt thereof.

In one aspect, provided is the compound (36)

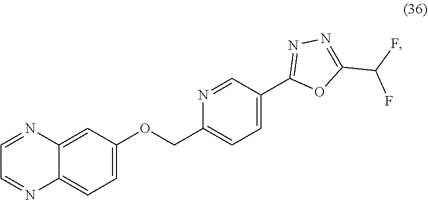 [SMILES: FC(F)c1nnc(-c2ccc(COc3ccc4nccnc4c3)nc2)o1]

and pharmaceutically acceptable salts thereof. In some embodiments, methods comprising the use or administration of Compound 36,

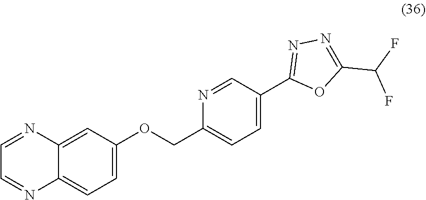 [SMILES: FC(F)c1nnc(-c2ccc(COc3ccc4nccnc4c3)nc2)o1]

or a pharmaceutically acceptable salt thereof, are provided. In some embodiments, methods comprising the use of 2-(difluoromethyl)-5-(6-((quinoxalin-6-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(6-((quinoxalin-6-yloxy)methyl)pyridin-3-yl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(difluoromethyl)-5-(6-((quinoxalin-6-yloxy)methyl)pyridin-3-yl)-1,3,4-oxadiazole (Compound 36), or a pharmaceutically acceptable salt thereof.

In one aspect, provided is the compound (151)

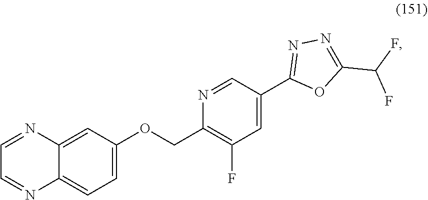 [SMILES: Fc1cc(-c2nnc(C(F)F)o2)cnc1COc1ccc2nccnc2c1]

and pharmaceutically acceptable salts thereof. In some embodiments, methods comprising the use or administration of Compound 151,

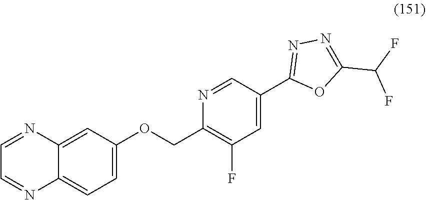 [SMILES: Fc1cc(-c2nnc(C(F)F)o2)cnc1COc1ccc2nccnc2c1]

or a pharmaceutically acceptable salt thereof, are provided. In some embodiments, methods comprising the use of 2-(difluoromethyl)-5-(5-fluoro-6-((quinoxalin-6-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(5-fluoro-6-((quinoxalin-6-yloxy)methyl)pyridin-3-yl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(difluoromethyl)-5-(5-fluoro-6-((quinoxalin-6-yloxy)methyl)pyridin-3-yl)-1,3,4-oxadiazole (Compound 151), or a pharmaceutically acceptable salt thereof.

In one aspect, provided is the compound (155)

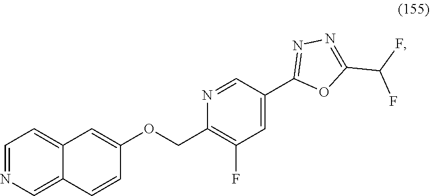 [SMILES: Fc1cc(-c2nnc(C(F)F)o2)cnc1COc1ccc2cnccc2c1]

and pharmaceutically acceptable salts thereof. In some embodiments, methods comprising the use or administration of Compound 155,

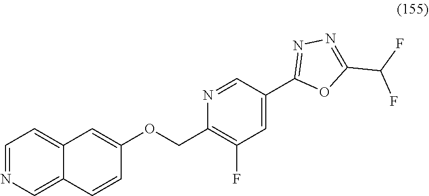 [SMILES: Fc1cc(-c2nnc(C(F)F)o2)cnc1COc1ccc2cnccc2c1]

or a pharmaceutically acceptable salt thereof, are provided. In some embodiments, methods comprising the use of 2-(difluoromethyl)-5-(5-fluoro-6-((isoquinolin-6-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(5-fluoro-6-((isoquinolin-6-yloxy)methyl)pyridin-3-yl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(difluoromethyl)-5-(5-fluoro-6-((isoquinolin-6-yloxy)methyl)pyridin-3-yl)-1,3,4-oxadiazole (Compound 155), or a pharmaceutically acceptable salt thereof.

In one aspect, provided is the compound (157)

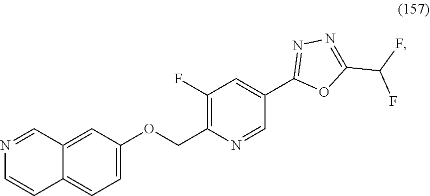 [SMILES: Fc1cc(-c2nnc(C(F)F)o2)cnc1COc1ccc2ccncc2c1]

and pharmaceutically acceptable salts thereof. In some embodiments, methods comprising the use or administration of Compound 157,

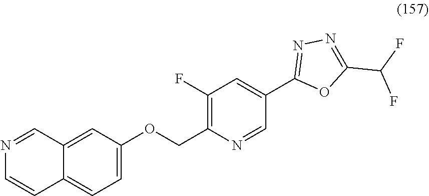 [SMILES: Fc1cc(-c2nnc(C(F)F)o2)cnc1COc1ccc2ccncc2c1]

or a pharmaceutically acceptable salt thereof, are provided. In some embodiments, methods comprising the use of 2-(difluoromethyl)-5-(5-fluoro-6-((isoquinolin-7-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(5-fluoro-6-((isoquinolin-7-yloxy)methyl)pyridin-3-yl)-1,3,4-oxadiazole, or a pharmaceutically acceptable salt thereof, to a subject in need thereof are provided. In some embodiments, a method of treatment comprising the oral administration to a subject in need thereof a pharmaceutical composition comprising 2-(difluoromethyl)-5-(5-fluoro-6-((isoquinolin-7-yloxy)methyl)pyridin-3-yl)-1,3,4-oxadiazole (Compound 157), or a pharmaceutically acceptable salt thereof.

In some embodiments, the subject can be an adult human diagnosed with advanced ovarian carcinoma, optionally in combination with other approved chemotherapeutic agents. One established combination regimen consists of carboplatin and cyclophosphamide.

### BRIEF DESCRIPTION OF THE DRAWINGS

FIG. 1A is a graph showing the plasma, brain and spinal cord exposure (concentration ng/mL) of Compound A for 8 hours after oral administration of a single dose of 3 mg/kg to mice.

FIG. 1B is a graph showing the plasma, brain and spinal cord exposure (concentration ng/mL) of Compound A for 8 hours after oral administration of a single dose of 10 mg/kg to mice.

FIG. 1C is a graph showing the brain and spinal cord exposure (concentration ng/mL) of Compound A for 8 hours, and the plasma exposure (concentration ng/mL) of Compound A for 24 hours, after oral administration of a single dose of 30 mg/kg to mice.

FIG. 2 is a graph showing target engagement data obtained for the HDAC6 substrate acetyl alpha tubulin relative to total alpha tubulin (Ac-Tubulin/Tubulin) normalized to the 0.1% DMSO condition vs concentration (micromolar) of Compound A in human SH-SY5Y cells.

FIG. 3A is a bar graph comparing the specific target engagement via the HDAC6 substrate acetyl alpha tubulin relative to total alpha tubulin (Ac-Tub/Total Tub) normalized to the 0.1% DMSO condition for Compound A and SAHA at 10 micromolar and 30 micromolar in human SH-SY5Y cells.

FIG. 3B is a bar graph comparing off-target engagement via the class I HDAC substrate acetyl histone H3 lysine 9 relative to total histone H3 (Ac-H3K9/Total H3) normalized to the 0.1% DMSO condition for Compound A and SAHA at 10 micromolar and 30 micromolar in human SH-SY5Y cells.

FIG. 4A is a linear plot of the plasma, brain, and spinal cord concentrations-time profiles (mean+SD) of Compound A following a single oral administration in male C57BL/6 mice (Dose: 3 mg/kg).

FIG. 4B is a semi-log plot of the plasma, brain, and spinal cord concentrations-time profiles (mean+SD) of Compound A following a single oral administration in male C57BL/6 mice (Dose: 3 mg/kg).

FIG. 5A is a linear plot of the plasma, brain, and spinal cord concentrations-time profiles (mean+SD) of Compound A following a single oral administration in male C57BL/6 mice (Dose: 10 mg/kg).

FIG. 5B is a semi-log plot of the plasma, brain, and spinal cord concentrations-time profiles (mean+SD) of Compound A following a single oral administration in male C57BL/6 mice (Dose: 10 mg/kg).

FIG. 6A is a linear plot of the plasma, brain, and spinal cord concentrations-time profiles (mean+SD) of Compound A following a single oral administration in male C57BL/6 mice (Dose: 30 mg/kg).

FIG. 6B is a Semi-log plot of the plasma, brain, and spinal cord concentrations-time profiles (mean+SD) of Compound A following a single oral administration in male C57BL/6 mice (Dose: 30 mg/kg).

FIG. 7 is a dose-response curve for acetyl-a-tubulin (K40)/total a-tubulin in SH-SY % Y cells following treatment with Vorinostat (suberoylanilide hydroxamic acid, SAHA).

### DETAILED DESCRIPTION

In some embodiments, a method of treating a neurodegenerative condition by inhibiting HDAC6 and increasing acetylated tubulin in cells, comprises administering to a subject in need thereof, a therapeutically effective amount of a compound of Formula (I),

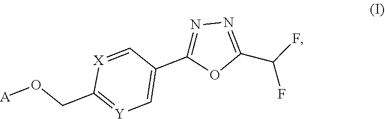

or a pharmaceutically acceptable salt thereof,
wherein:
   X is N or CR¹, and Y is CR¹ or N, provided that at least one of X and Y is N;
R¹ is hydrogen or halogen;
   A is heteroaryl, aryl, carbocyclyl, or heterocyclyl, wherein A is optionally substituted with one or more substituents R²; and
   each occurrence of R² is independently halogen, amido, cyano, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, or substituted or unsubstituted heteroaryl; or two occurrences of R² are joined with their intervening atoms to form substituted or unsubstituted carbocyclyl, substituted or unsubstituted heterocyclyl, substituted or unsubstituted aryl, or substituted or unsubstituted heteroaryl.

### X and Y

As described herein, X is CR¹ or N, and Y is CR¹ or N, provided that at least one of X and Y is N; and R¹ is hydrogen or halogen.

In certain embodiments, X is N. In certain embodiments, X is CR¹. In certain embodiments, X is CR¹; and R¹ is hydrogen or halogen. In certain embodiments, X is CR¹; and R¹ is hydrogen or fluoro. In certain embodiments, X is CH or CF. In certain embodiments, X is CH. In certain embodiments, X is CF.

In certain embodiments, Y is N. In certain embodiments, Y is CR¹. In certain embodiments, Y is CR¹; and R¹ is hydrogen or halogen. In certain embodiments, Y is CR¹; and R¹ is hydrogen or fluoro. In certain embodiments, Y is CH or CF. In certain embodiments, Y is CH. In certain embodiments, Y is CF.

In certain embodiments, at least one of X and Y is N. In certain embodiments, one of X and Y is N. In certain embodiments, X is N, and Y is CR¹. In certain embodiments, X is N, and Y is CH. In certain embodiments, X is N, and Y is CF. In certain embodiments, X is N, and Y is N.

In certain embodiments, Y is N, and X is CR¹. In certain embodiments, Y is N, and X is CH. In certain embodiments, Y is N, and X is CF. In certain embodiments, Y is N, and X is N.

### A

As described herein, A is aryl, heteroaryl, carbocyclyl, or heterocyclyl, wherein A is optionally substituted with one or more substituents R².

In certain embodiments, A is C₆₋₁₄ aryl, 5-11 membered heteroaryl, C₃₋₁₀ cycloalkyl, or 4-11 membered heterocyclyl, wherein A is optionally substituted with 1-3 independent substituents R². In certain embodiments, A is aryl optionally substituted with 1-3 independent substituents R². In certain embodiments, A is C₆₋₁₄ aryl optionally substituted with 1-3 independent substituents R². In certain embodiments, A is phenyl or C₉₋₁₄ fused bicyclic aryl, wherein A is optionally substituted with 1-3 independent substituents R².

In certain embodiments, A is heteroaryl optionally substituted with 1-3 independent substituents R². In certain embodiments, A is 5-11 membered heteroaryl optionally substituted with 1-3 independent substituents R². In certain embodiments, A is a bicyclic heteroaryl, wherein A is optionally substituted with 1-3 independent substituents R². In certain embodiments, A is bicyclic heteroaryl optionally substituted with 1-3 independent substituents R². In certain embodiments, A is 5-membered monocyclic heteroaryl optionally substituted with 1-3 independent substituents R² fused with another 5-membered monocyclic heteroaryl optionally substituted with 1-3 independent substituents R². In certain embodiments, A is 5-membered monocyclic heteroaryl optionally substituted with 1-3 independent substituents R² fused with 6-membered monocyclic heteroaryl optionally substituted with 1-3 independent substituents R², wherein the attachment point is on the 5-membered monocyclic heteroaryl. In certain embodiments, A is 6-membered monocyclic heteroaryl optionally substituted with 1-3 independent substituents R² fused with 5-membered monocyclic heteroaryl optionally substituted with 1-3 independent substituents R², wherein the attachment point is on the 6-membered monocyclic heteroaryl. In certain embodiments, A is 6-membered monocyclic heteroaryl optionally substituted with 1-3 independent substituents R² fused with another 6-membered monocyclic heteroaryl optionally substituted with 1-3 independent substituents R².

In some embodiments, the compound is a compound of Formula (I), wherein A is 5-11 membered heteroaryl, C₆₋₁₄ aryl, C₃₋₁₀ cycloalkyl, or 4-11 membered heterocyclyl, wherein A is optionally substituted with 1-3 independent substituents R²; and each occurrence of R² is independently halogen, cyano, C₁₋₆ alkyl, C₃₋₆ cycloalkyl, C₂₋₆ alkenyl, or C₂₋₆ alkynyl, wherein each C₁₋₆ alkyl, C₃₋₆ cycloalkyl, C₂₋₆ alkenyl, or C₂₋₆ alkynyl is optionally substituted with one or more halogen or cyano. In some embodiments, the compound is a compound of Formula (I), wherein A is a 5,6-bicyclic heteroaryl optionally substituted with 1-3 independent substituents R²; and each R² is independently chlorine, fluorine, —CN, —CH₂CN, C₁₋₄ alkyl, C₃₋₆ cycloalkyl, C₁₋₄ haloalkyl, acyl, amide, or 5-6 membered heterocyclyl comprising one or more nitrogen, oxygen, or sulfur heteroatom. In some embodiments, the compound is a compound of Formula (I), wherein A is a 5,6-bicyclic heteroaryl ring system comprising one or more nitrogen or oxygen heteroatoms. In some embodiments, the compound is a compound of Formula (I), wherein A is a quinoline, isoquinoline, quinoxaline, quinazoline, cinnoline, phthalazine, phenazine, phenathridine or acridine. In some embodiments, the compound is a compound of Formula (I), wherein A is benzo-1,2,3-triazine, benzo-1,2,4-triazine or benzo-1,3,5-triazine. In some embodiments, the compound is a compound of Formula (I), wherein A is pteridine, triazino[3,4-b][1,4]oxazine or a diazino-diazine. In some embodiments, the compound is a compound of Formula (I), wherein A is a furo[3,2-b]pyridine, a thieno[3,2-b]pyridine or a benzo[c][1,2,5]thiadiazole. In some embodiments, the compound is a compound of Formula (I), wherein A is an imidazopyrazine, an imidazopyridine, a quinazoline, a quinoxaline, a quinoline, or a benzoxazole.

In certain embodiments, A is 9-14 membered fused bicyclic heteroaryl optionally substituted with 1-3 independent substituents R².

In certain embodiments, A is phenyl fused to a 5-6 membered heteroaryl, a 4-6 membered heterocyclyl, or a C₄₋₆ carbocyclyl ring, or A is a 5-6 membered heteroaryl fused to a phenyl, a 5-6 membered heteroaryl, a 4-6 membered heterocyclyl, or a C₄₋₆ carbocyclyl ring, wherein A is optionally substituted with 1-3 independent substituents R². In certain embodiments, A is phenyl fused to a 5-6 membered heteroaryl, a 4-6 membered heterocyclyl, or a C₄₋₆ carbocyclyl ring, or A is a 5-6 membered heteroaryl fused to a phenyl, a 4-6 membered heterocyclyl, or a C₄₋₆ carbocyclyl ring, wherein A is optionally substituted with 1-3 independent substituents R². In certain embodiments, A is phenyl fused to a a 4-6 membered heterocyclyl or a C₄₋₆ carbocyclyl ring, or A is a 5-6 membered heteroaryl fused to a 4-6 membered heterocyclyl or a C₄₋₆ carbocyclic ring, wherein A is optionally substituted with 1-3 independent substituents R². In certain embodiments, A is phenyl fused to a 5-6 membered heteroaryl, wherein A is optionally substituted with 1-3 independent substituents R². In certain embodiments, A is phenyl fused to a 4-6 membered heterocyclyl, wherein A is optionally substituted with 1-3 independent substituents R². In certain embodiments, A is phenyl fused to a C₄₋₆ carbocyclic ring, wherein A is optionally substituted with 1-3 independent substituents R². In certain embodiments, A is a 5-6 membered heteroaryl fused to a phenyl, wherein A is optionally substituted with 1-3 independent substituents R². In certain embodiments, A is a 5-6 membered heteroaryl fused to a 4-6 membered heterocyclyl, wherein A is optionally substituted with 1-3 independent substituents R². In certain embodiments, A is a 5-6 membered heteroaryl fused to a C₄₋₆ carbocyclic ring, wherein A is optionally substituted with 1-3 independent substituents R².

In certain embodiments, A is benzoxazolyl, indazolyl, isoquinolinyl, tetrahydroisoquinolinyl, tetrahydroisoxazolo[4,5-c]pyridinyl, tetrahydroisoxazolo[5,4-c]pyridinyl, quinazolinyl, triazolo[4,3-a]pyridinyl, imidazo[1,2-a]pyrazinyl, imidazo[1,2-a]pyridinyl, pyrazolo[1,5-a]pyrimidinyl, [1,2,4]triazolo[4,3-a]pyridinyl, quinolinyl, benzoisoxazolyl, benzoimidazolyl, benzopyrazolyl, benzotriazolyl, indolyl, or quinoxalinyl, wherein A is optionally substituted with 1-3 independent substituents R². In certain embodiments, A is benzoxazolyl, indazolyl, isoquinolinyl, tetrahydroisoquinolinyl, quinazolinyl, triazolo[4,3-a]pyridinyl, imidazo[1,2-a]pyrazinyl, imidazo[1,2-a]pyridinyl, quinolinyl, benzoisoxazolyl, benzoimidazolyl, or quinoxalinyl, wherein A is optionally substituted with 1-3 independent substituents R². In certain embodiments, A is quinazolinyl, imidazo[1,2-a]pyrazinyl, benzoisoxazolyl, or benzopyrazolyl, wherein A is optionally substituted with 1-3 independent substituents R². In certain embodiments, A is phenyl, quinazolinyl, imidazo[1,2-a]pyrazinyl, benzoisoxazolyl, or benzopyrazolyl, wherein A is optionally substituted with 1-3 independent substituents R². In certain embodiments, A is quinazolinyl, imidazo[1,2-a]pyrazinyl, benzoisoxazolyl, or benzopyrazolyl, wherein A is optionally substituted with 1-3 independent substituents R². In certain embodiments, A is quinazolinyl, wherein A is optionally substituted with 1-3 independent substituents R². In certain embodiments, A is imidazo[1,2-a]pyrazinyl, wherein A is optionally substituted with 1-3 independent substituents R². In certain embodiments, A is benzoisoxazolyl, wherein A is optionally substituted with 1-3 independent substituents R². In certain embodiments, A is benzopyrazolyl, wherein A is optionally substituted with 1-3 independent substituents R².

In certain embodiments, A is

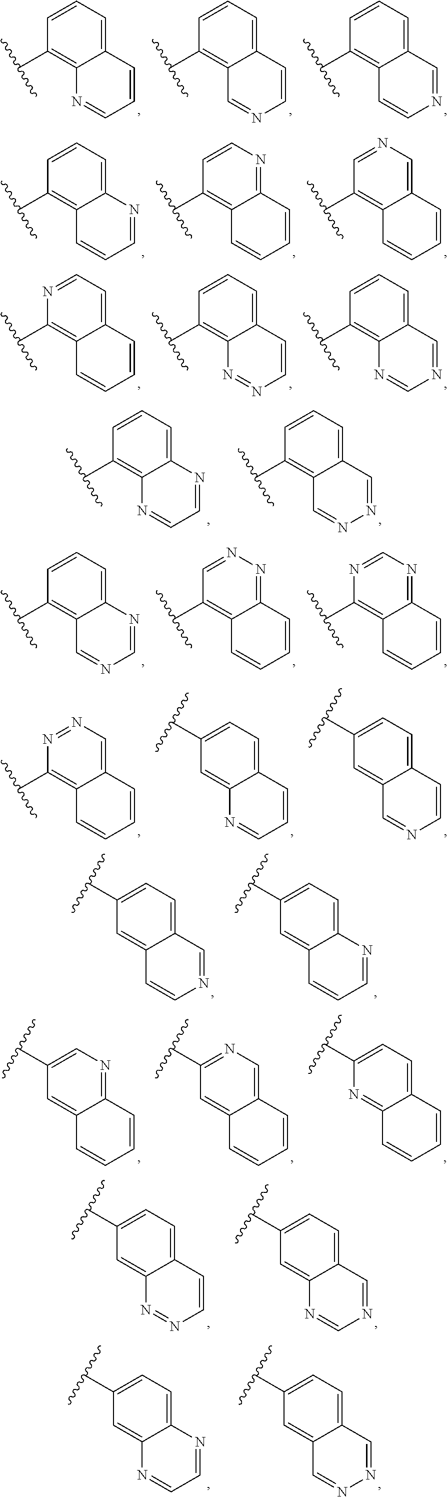

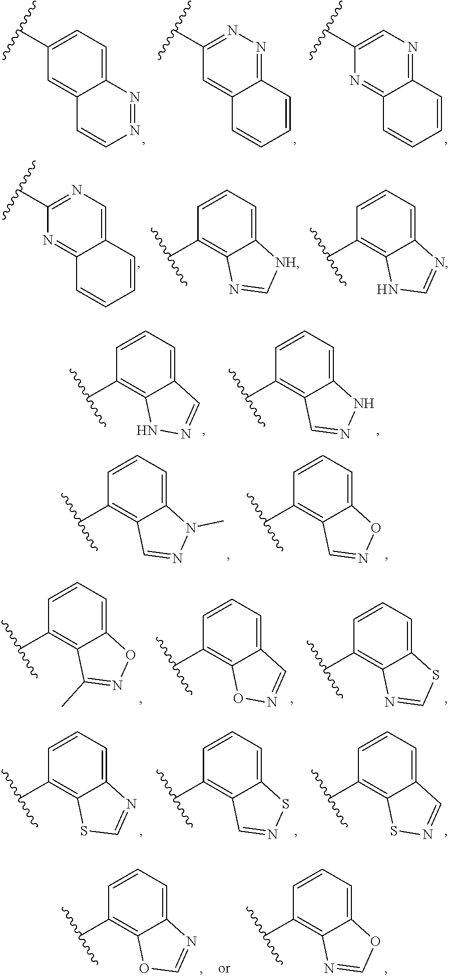

wherein A is optionally substituted with 1-3 independent substituents R².

In certain embodiments, A is

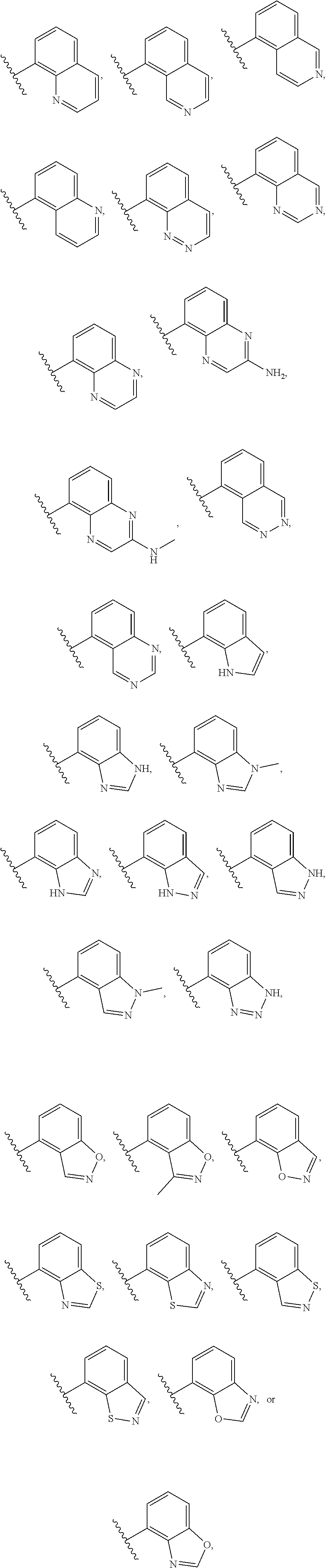

wherein A is optionally substituted with 1-3 independent substituents R². In certain embodiments, A is

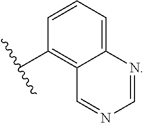 [SMILES: *c1cccc2ncncc12]

In certain embodiments, A is

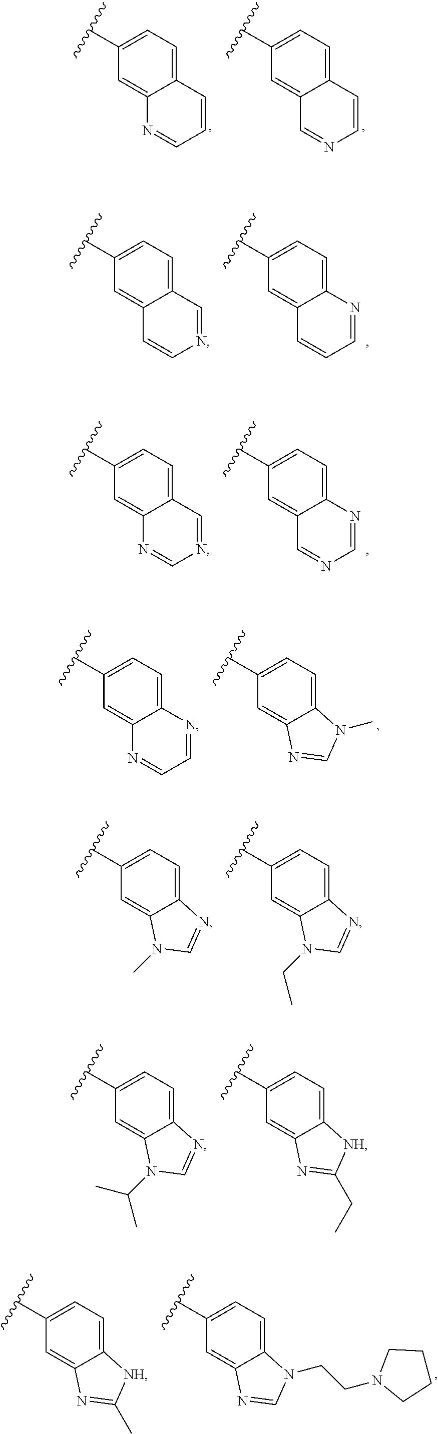

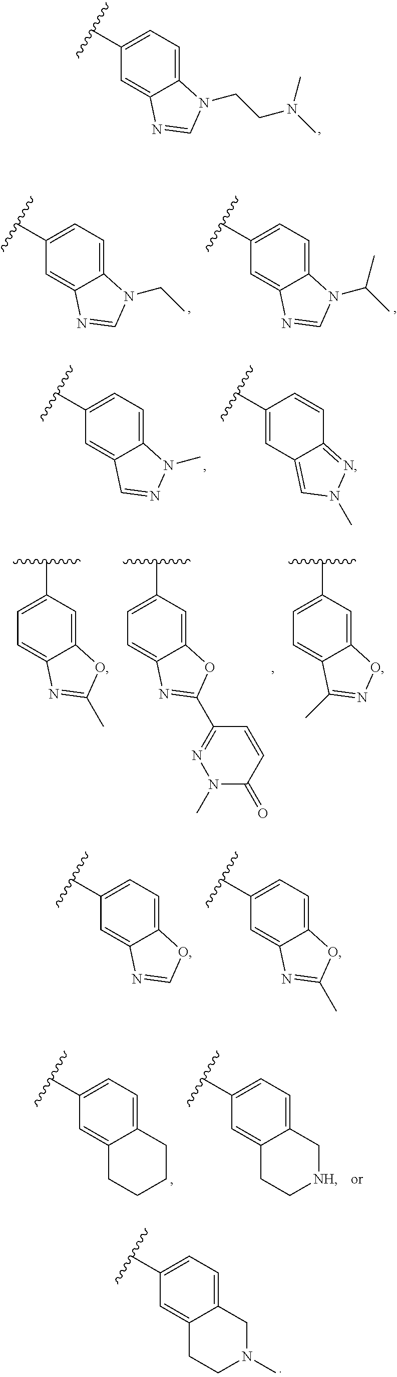

In certain embodiments, A is

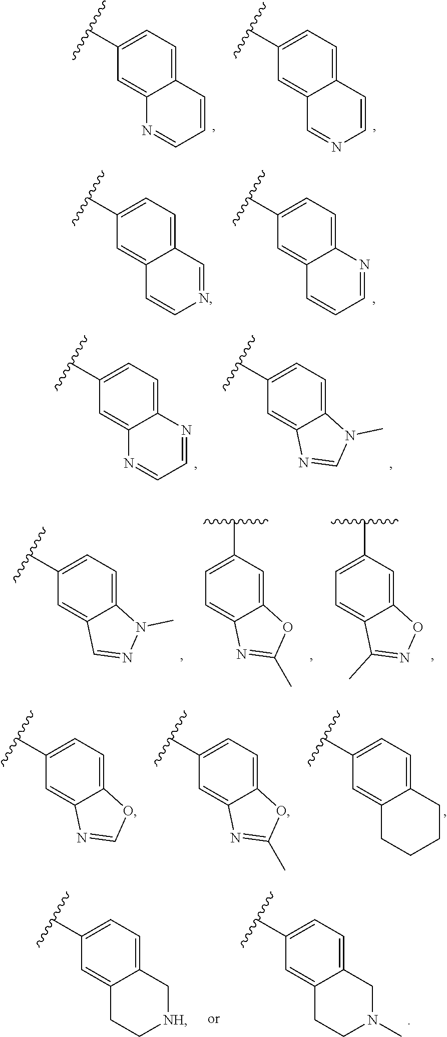

In certain embodiments, A is

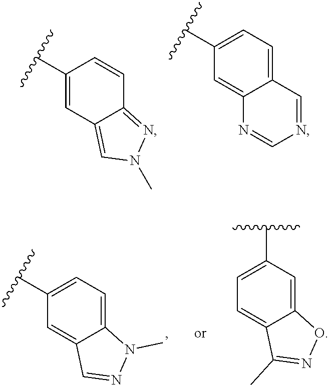 [SMILES: *c1ccc2nn(C)cc2c1 ; *c1ccc2cncnc2c1 ; *c1ccc2c(cnn2C)c1 ; *c1ccc2c(C)noc2c1]

In certain embodiments, A is

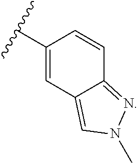 [SMILES: *c1ccc2nn(C)cc2c1]

In certain embodiments, A is

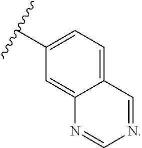 [SMILES: *c1ccc2cncnc2c1]

In certain embodiments, A is

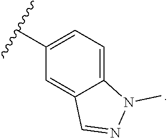 [SMILES: *c1ccc2c(cnn2C)c1]

In certain embodiments, A is

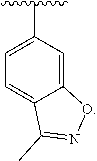 [SMILES: *c1ccc2c(C)noc2c1]

In certain embodiments, A is

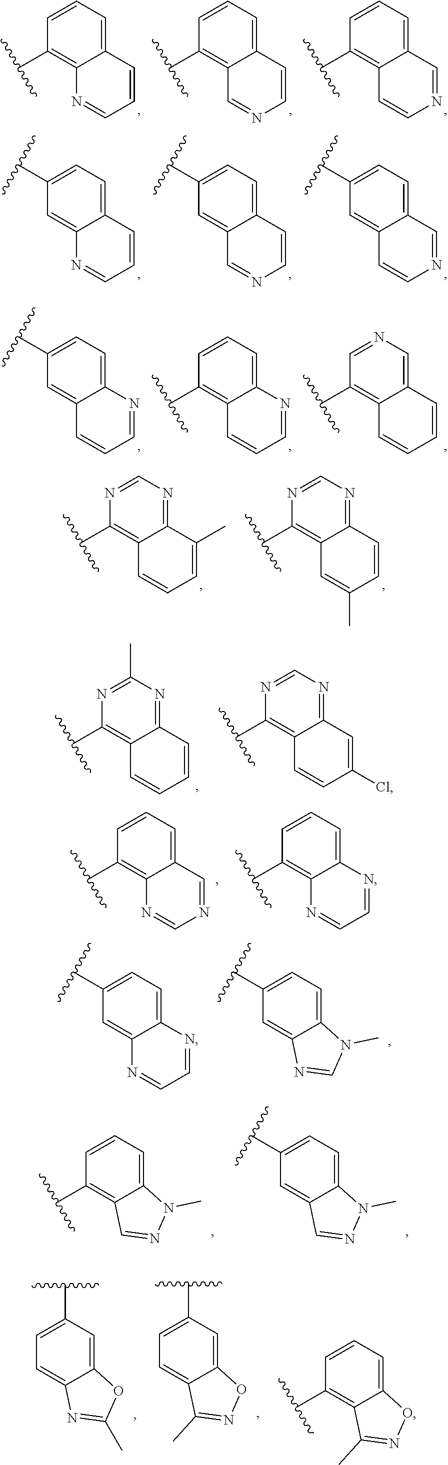

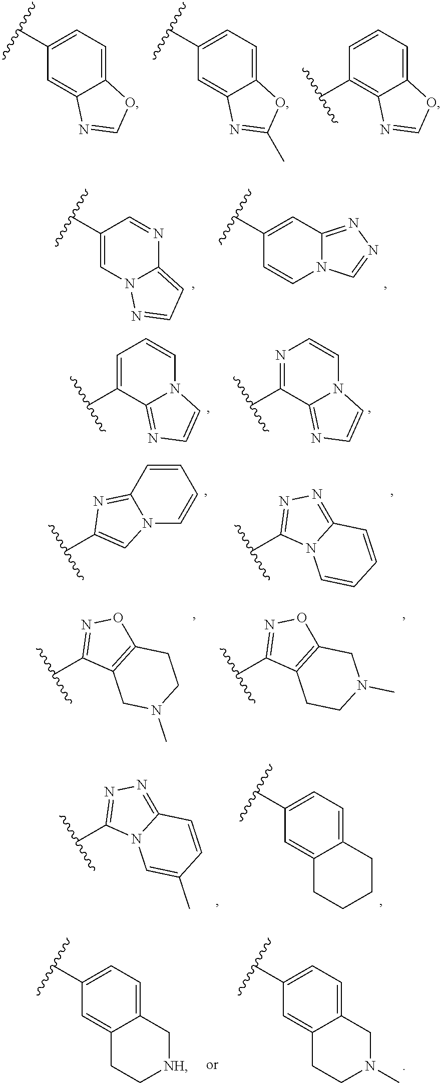

In certain embodiments, A is

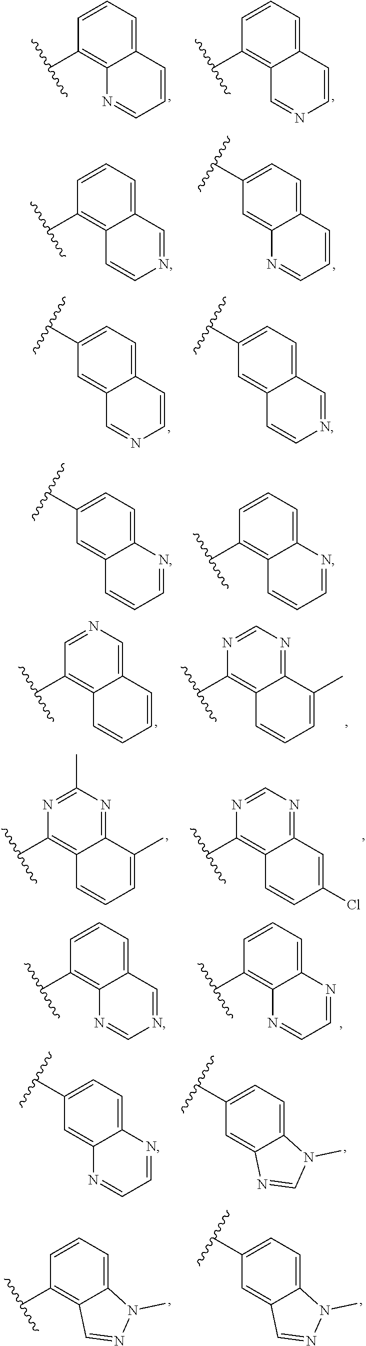

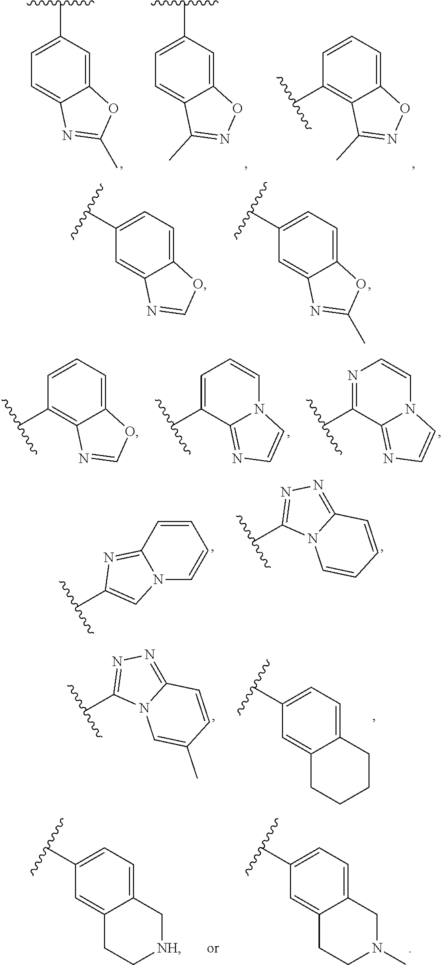

In certain embodiments, A is

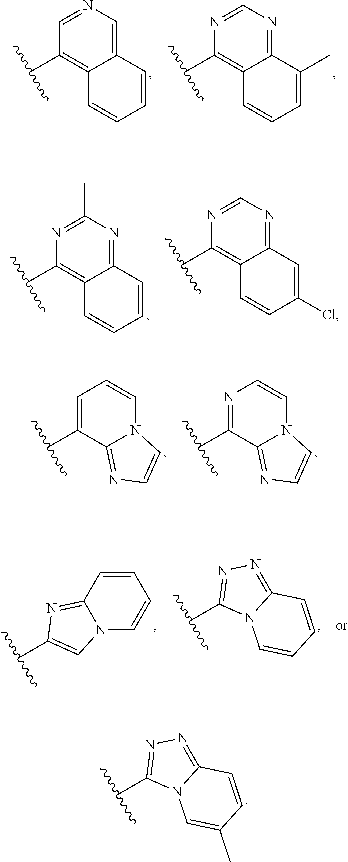

In certain embodiments, A is

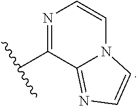 [SMILES: *c1nccn2ccnc12]

In certain embodiments, A is

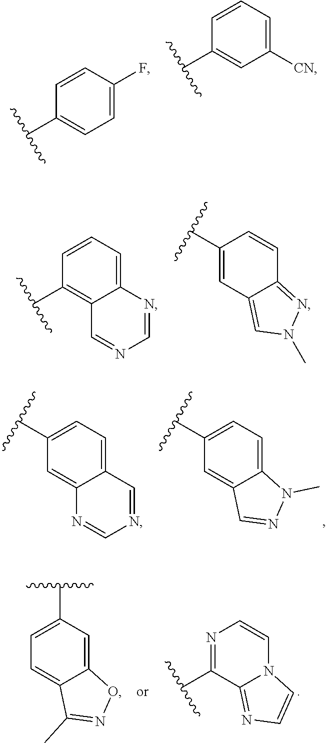

In certain embodiments, A is carbocyclyl or heterocyclyl, wherein A is optionally substituted with one or more substituents R². In certain embodiments, A is C₃₋₁₀ cycloalkyl or 4-11 membered heterocyclyl, wherein A is optionally substituted with 1-3 independent substituents R².

In some embodiments, A is

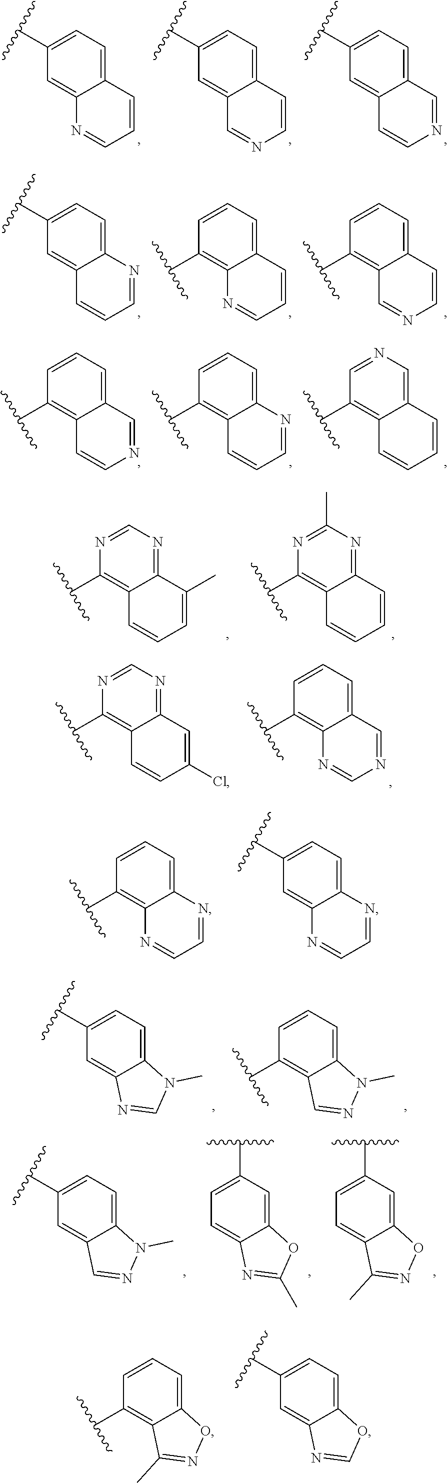

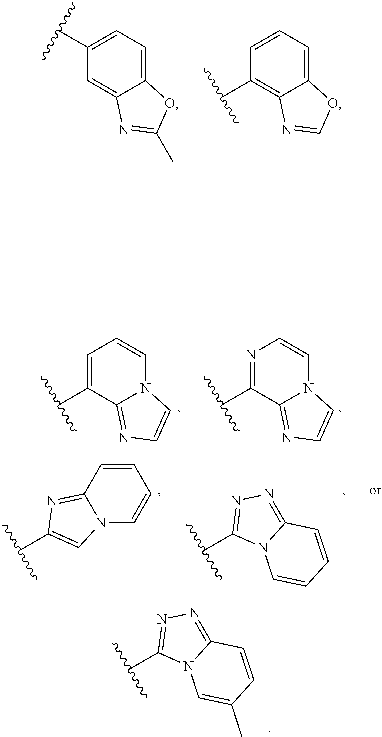

In some embodiments, A is

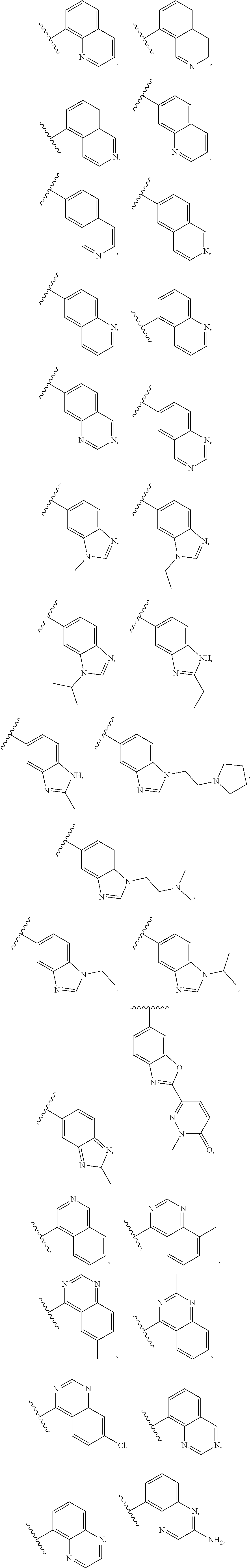

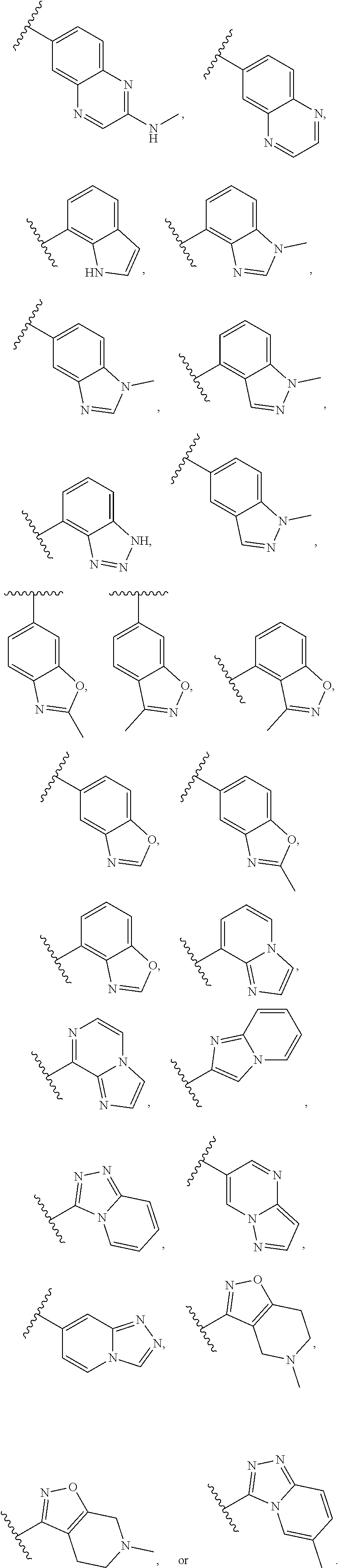

### R²

As described herein, each occurrence of R² is independently halogen, amino, amido, cyano, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, or substituted or unsubstituted heteroaryl; or two occurrences of R² are joined with their intervening atoms to form substituted or unsubstituted carbocyclyl, substituted or unsubstituted heterocyclyl, substituted or unsubstituted aryl, or substituted or unsubstituted heteroaryl. In certain embodiments, each occurrence of R² is independently halogen, amido, cyano, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, or substituted or unsubstituted heteroaryl; or two occurrences of R² are joined with their intervening atoms to form substituted or unsubstituted carbocyclyl, substituted or unsubstituted heterocyclyl, substituted or unsubstituted aryl, or substituted or unsubstituted heteroaryl. In certain embodiments, each occurrence of R² is independently halogen, amido, cyano, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, or substituted or unsubstituted heteroaryl. In certain embodiments, each occurrence of R² is independently halogen, amido, cyano, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, or substituted or unsubstituted heterocyclyl.

In certain embodiments, each occurrence of R² is independently halogen, cyano, —NH₂, —NHCH₃, —(C═O)OH, —(C═O)NH₂, —(C═O)NHCH₃, —(C═O)NHPh, substituted or unsubstituted C₁₋₆ alkyl, substituted or unsubstituted aminoalkyl, 4-6 membered substituted or unsubstituted heterocyclylalkyl, substituted or unsubstituted phenyl, substituted or unsubstituted C₃₋₆ cycloalkyl, substituted or unsubstituted 4-6 membered heterocyclyl, substituted or unsubstituted 5-6 membered heteroaryl, substituted or unsubstituted C₂₋₆ alkenyl, or substituted or unsubstituted C₂₋₆ alkynyl. In certain embodiments, each occurrence of R² is independently halogen, cyano, —NH₂, —NHCH₃, —(C═O)OH, —(C═O)NH₂, —(C═O)NHCH₃, —(C═O)NHPh, C₁₋₆ alkyl, aminoalkyl, 4-6 membered heterocyclylalkyl, substituted or unsubstituted phenyl, C₃₋₆ cycloalkyl, 4-6 membered heterocyclyl, 5-6 membered substituted or unsubstituted heteroaryl, C₂₋₆ alkenyl, or C₂₋₆ alkynyl, wherein each C₁₋₆ alkyl, C₃₋₆ cycloalkyl, C₂₋₆ alkenyl, or C₂₋₆ alkynyl is optionally substituted with one or more halogen or cyano. In certain embodiments, each occurrence of R² is independently halogen, cyano, —(C═O)NH₂, C₁₋₆ alkyl, C₃₋₆ cycloalkyl, 4-6 membered heterocyclyl, C₂₋₆ alkenyl, or C₂₋₆ alkynyl, wherein each C₁₋₆ alkyl, C₃₋₆ cycloalkyl, C₂₋₆ alkenyl, or C₂₋₆ alkynyl is optionally substituted with one or more halogen or cyano.

In certain embodiments, each occurrence of R² is independently fluoro, chloro, methyl, ethyl, isopropyl, trifluoromethyl, cyclopropyl, —(C═O)NH₂, cyano, cyanomethyl, ethynyl, or morpholinyl. In certain embodiments, each occurrence of R² is independently fluoro. In certain embodiments, each occurrence of R² is independently chloro. In certain embodiments, each occurrence of R² is independently methyl. In certain embodiments, each occurrence of R² is independently ethyl. In certain embodiments, each occurrence of R² is independently isopropyl. In certain embodiments, each occurrence of R² is independently trifluoromethyl. In certain embodiments, each occurrence of R² is independently cyclopropyl. In certain embodiments, each occurrence of R² is independently —(C═O)NH₂. In certain embodiments, each occurrence of R² is independently cyano. In certain embodiments, each occurrence of R² is independently cyanomethyl. In certain embodiments, each occurrence of R² is independently ethynyl. In certain embodiments, each occurrence of R² is independently morpholinyl.

In certain embodiments, each occurrence of R² is independently halogen. In certain embodiments, each occurrence of R² is independently fluoro or chloro. In certain embodiments, each occurrence of R² is fluoro.

In certain embodiments, each occurrence of R² is independently halogen, cyano, or substituted or unsubstituted C₁₋₆ alkyl. In certain embodiments, each occurrence of R² is independently halogen, cyano, or unsubstituted C₁₋₆ alkyl. In certain embodiments, each occurrence of R² is independently fluoro, cyano, or methyl.

In certain embodiments, A is substituted with 1 or 2 R² groups, wherein each occurrence of R² is independently halogen, cyano, —(C═O)NH₂, C₁₋₆ alkyl, C₃₋₆ cycloalkyl, 4-6 membered heterocyclyl, C₂₋₆ alkenyl, or C₂₋₆ alkynyl, wherein each C₁₋₆ alkyl, C₃₋₆ cycloalkyl, C₂₋₆ alkenyl, or C₂₋₆ alkynyl is optionally substituted with one or more halogen or cyano.

In certain embodiments, A is substituted with 1 or 2 R² groups, wherein each occurrence of R² is independently fluoro, chloro, methyl, ethyl, isopropyl, trifluoromethyl, cyclopropyl, —(C═O)NH₂, cyano, cyanomethyl, ethynyl, or morpholinyl.

In certain embodiments, A is substituted with 1 or 2 R² groups, wherein each occurrence of R² is independently fluoro, chloro, cyano, cyclopropyl, C₂₋₄ alkynyl, or C₁₋₄ alkyl optionally substituted with one or more fluoro, cyano, or alkynyl.

In certain embodiments, A is substituted with 1 or 2 R² groups, wherein each occurrence of R² is independently halogen. In certain embodiments, A is substituted with 1 or 2 R² groups, wherein each occurrence of R² is independently fluoro or chloro. In certain embodiments, A is substituted with 1 or 2 R² groups, wherein each occurrence of R² is fluoro.

In certain embodiments, A is C₆₋₁₄ aryl, 5-11 membered heteroaryl, C₃₋₁₀ cycloalkyl, or 4-11 membered heterocyclyl, wherein A is optionally substituted with 1-3 independent substituents R²; each occurrence of R¹ is independently hydrogen, halogen, or C₁₋₆ alkyl optionally substituted with one or more halogen or alkoxy; and each occurrence of R² is independently halogen, cyano, C₁₋₆ alkyl, C₃₋₆ cycloalkyl, C₂₋₆ alkenyl, or C₂₋₆ alkynyl, wherein each C₁₋₆ alkyl, C₃₋₆ cycloalkyl, C₂₋₆ alkenyl, or C₂₋₆ alkynyl is optionally substituted with one or more halogen or cyano.

In certain embodiments, the compound of Formula (I) is of Formula (I-a):

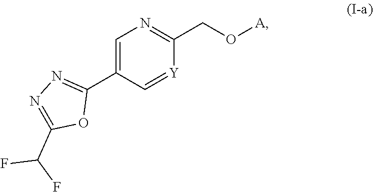

or a pharmaceutically acceptable salt thereof; wherein A, and Y are as defined herein.

In certain embodiments of Formula (I-a), Y is CH, CF or N. In certain embodiments of Formula (I-a), Y is CH. In certain embodiments of Formula (I-a), Y is CF. In certain embodiments of Formula (I-a), Y is N.

In certain embodiments of Formula (I-a), A is a bicyclic fused heteroaryl comprising at least two heteroatoms (e.g., S, N, O), and A is optionally substituted with 1 or 2 R² groups, wherein each occurrence of R² is independently fluoro, cyano, or substituted or unsubstituted C₁₋₆ alkyl. In certain embodiments of Formula (I-e), A is a 5,6 bicyclic fused heteroaryl comprising at least two heteroatoms (e.g., S, N, O), and A is optionally substituted with 1 or 2 R² groups, wherein each occurrence of R² is independently fluoro, cyano, or substituted or unsubstituted C₁-C₆ alkyl. In certain embodiments of Formula (I-a), A is a 5,6 bicyclic fused heteroaryl comprising at least two heteroatoms (e.g., S, N, O), and A is optionally substituted with 1 or 2 R² groups, wherein each occurrence of R² is independently fluoro, cyano, or substituted or unsubstituted C₁₋₆ alkyl. In certain embodiments of Formula (I-a), A is quinazolinyl, imidazo[1,2-a]pyrazinyl, benzoisoxazolyl, or benzopyrazolyl, and A is optionally substituted with 1 or 2 R² groups, wherein each occurrence of R² is independently fluoro, cyano, or substituted or unsubstituted C₁₋₆ alkyl. In certain embodiments of Formula (I-a), A is imidazo[1,2-a]pyrazinyl, and A is optionally substituted with 1 or 2 R² groups, wherein each occurrence of R² is independently fluoro, cyano, or substituted or unsubstituted C₁₋₆ alkyl. In certain embodiments of Formula (I-a), A is an unsubstituted 5,6 bicyclic heteroaryl. In certain embodiments of Formula (I-a), A is unsubstituted imidazo[1,2-a]pyrazinyl.

In certain embodiments, the compound of Formula (I) is one of the following compounds, or a pharmaceutically acceptable salt thereof:

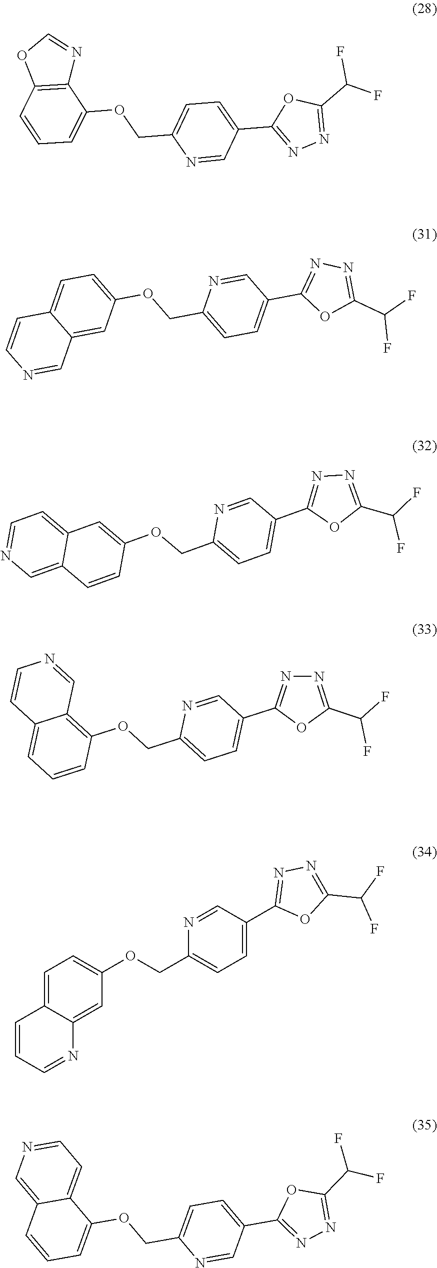 [SMILES: FC(F)c1nnc(-c2ccc(COc3cccc4ocnc34)nc2)o1 ; FC(F)c1nnc(-c2ccc(COc3ccc4ccncc4c3)nc2)o1 ; FC(F)c1nnc(-c2ccc(COc3ccc4cnccc4c3)nc2)o1 ; FC(F)c1nnc(-c2ccc(COc3cccc4ccncc34)nc2)o1 ; FC(F)c1nnc(-c2ccc(COc3ccc4cccnc4c3)nc2)o1 ; FC(F)c1nnc(-c2ccc(COc3cccc4cnccc34)nc2)o1]

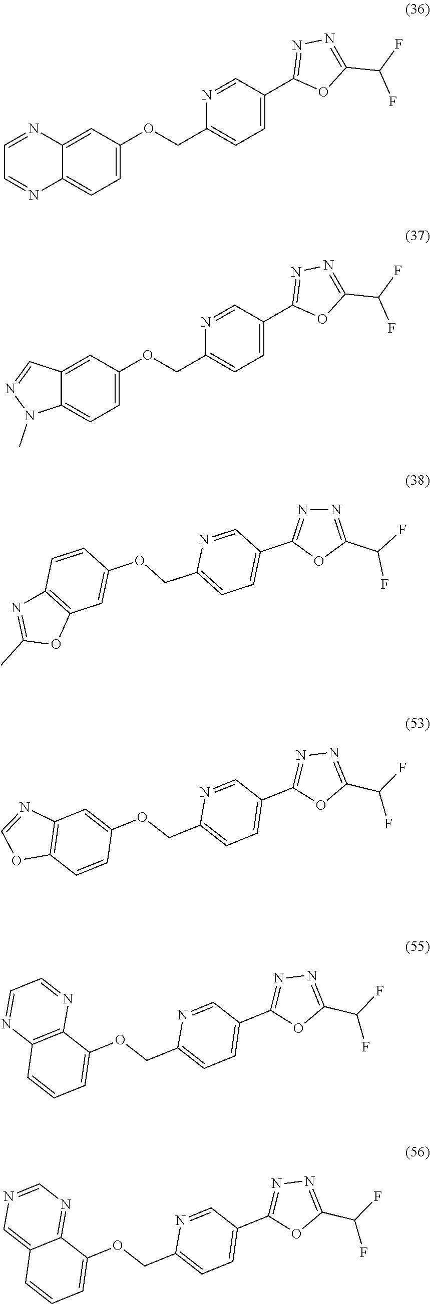 [SMILES: FC(F)c1nnc(-c2ccc(COc3ccc4nccnc4c3)nc2)o1 ; Cn1ncc2cc(OCc3ccc(-c4nnc(C(F)F)o4)cn3)ccc21 ; Cc1nc2ccc(OCc3ccc(-c4nnc(C(F)F)o4)cn3)cc2o1 ; FC(F)c1nnc(-c2ccc(COc3ccc4ocnc4c3)nc2)o1 ; FC(F)c1nnc(-c2ccc(COc3cccc4nccnc34)nc2)o1 ; FC(F)c1nnc(-c2ccc(COc3cccc4cncnc34)nc2)o1]

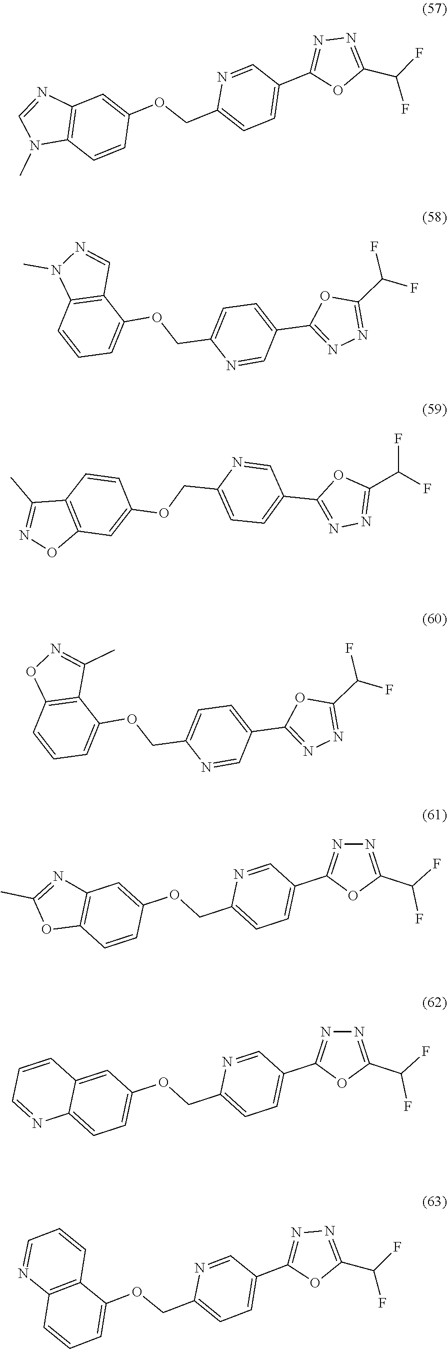

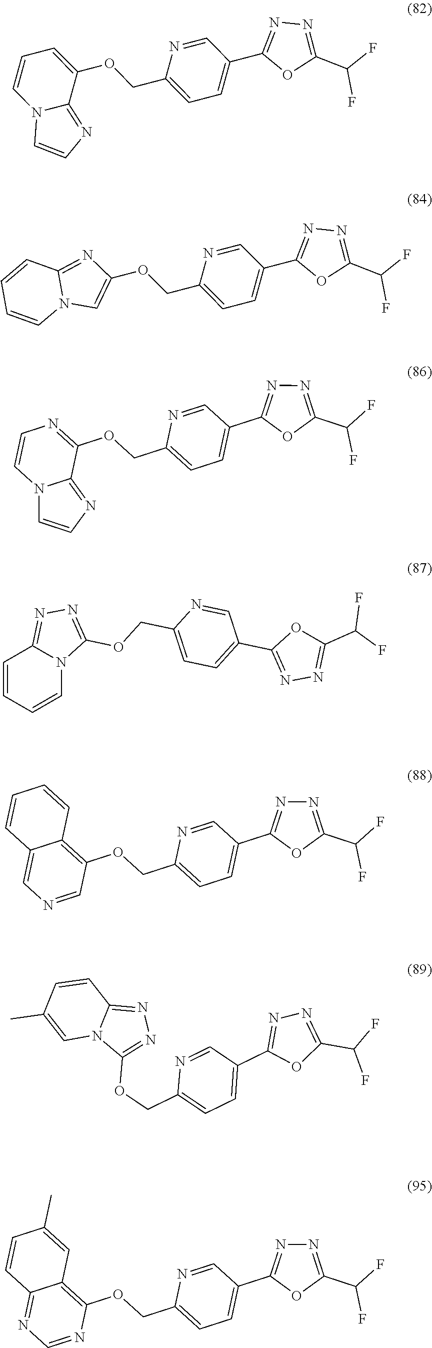

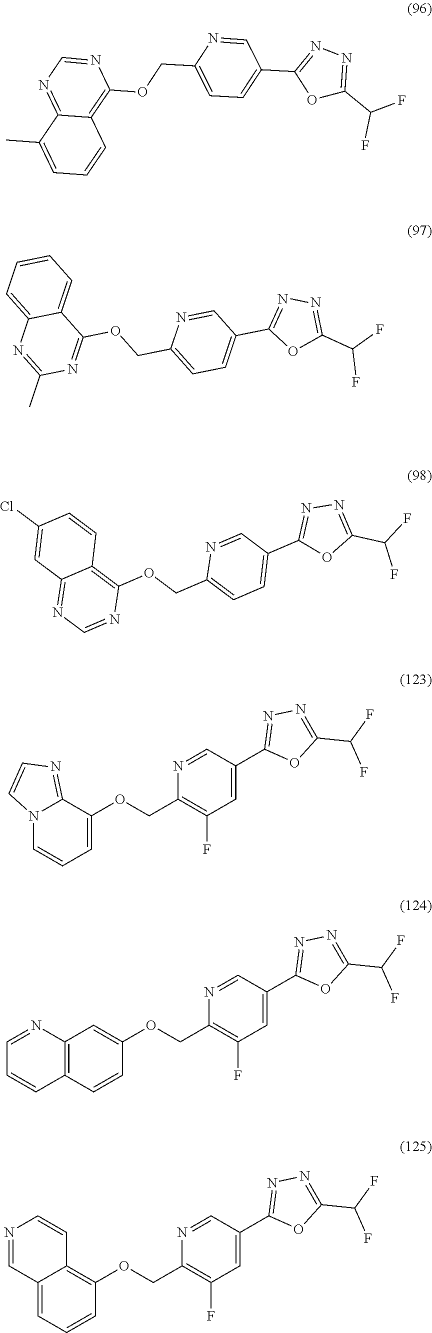 [SMILES: Cc1cccc2c(OCc3ccc(-c4nnc(C(F)F)o4)cn3)ncnc12 ; Cc1nc(OCc2ccc(-c3nnc(C(F)F)o3)cn2)c2ccccc2n1 ; FC(F)c1nnc(-c2ccc(COc3ncnc4cc(Cl)ccc34)nc2)o1 ; Fc1cc(-c2nnc(C(F)F)o2)cnc1COc1cccn2ccnc12 ; Fc1cc(-c2nnc(C(F)F)o2)cnc1COc1ccc2cccnc2c1 ; Fc1cc(-c2nnc(C(F)F)o2)cnc1COc1cccc2cnccc12]

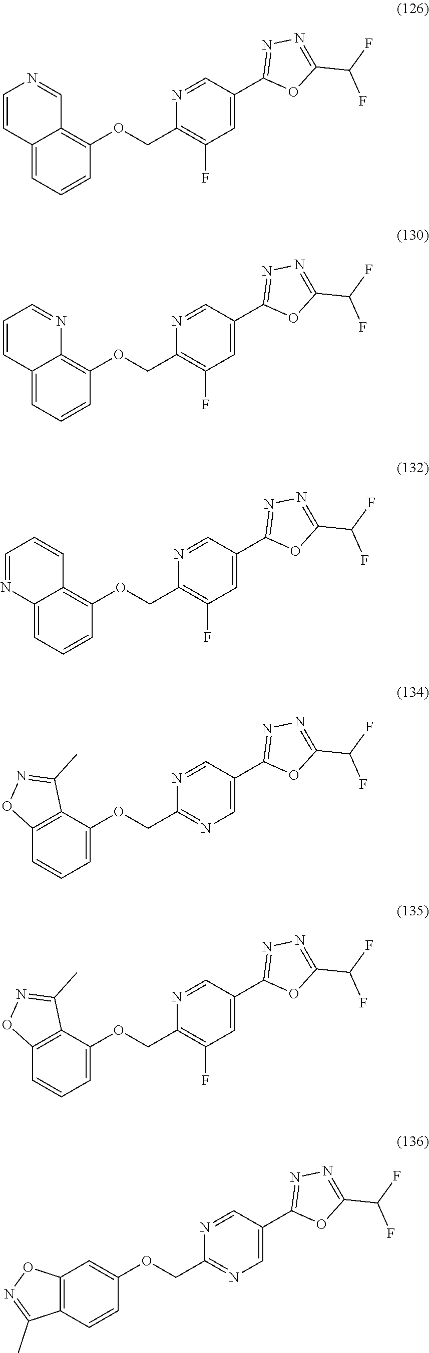 [SMILES: Fc1cc(-c2nnc(C(F)F)o2)cnc1COc1cccc2ccncc12 ; Fc1cc(-c2nnc(C(F)F)o2)cnc1COc1cccc2cccnc12 ; Fc1cc(-c2nnc(C(F)F)o2)cnc1COc1cccc2ncccc12 ; Cc1noc2cccc(OCc3ncc(-c4nnc(C(F)F)o4)cn3)c12 ; Cc1noc2cccc(OCc3ncc(-c4nnc(C(F)F)o4)cc3F)c12 ; Cc1noc2cc(OCc3ncc(-c4nnc(C(F)F)o4)cn3)ccc12]

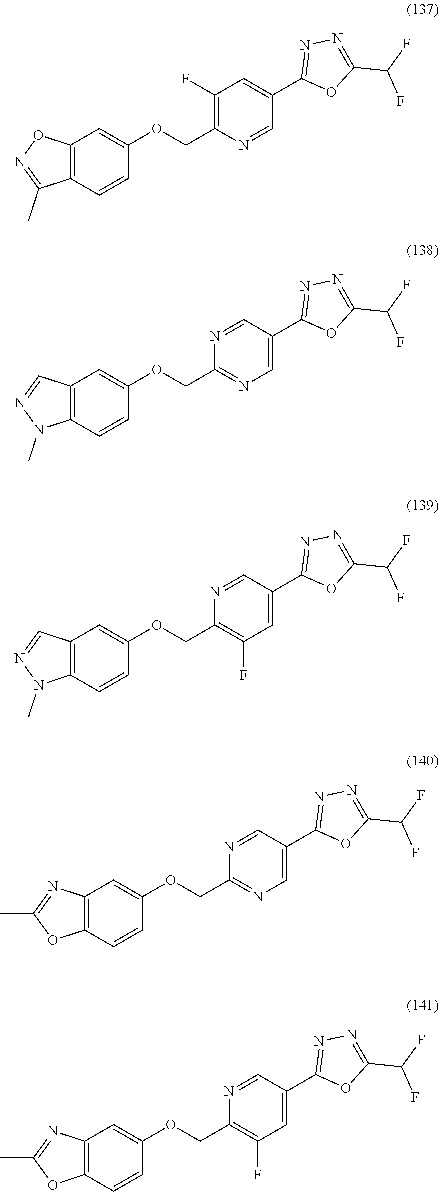 [SMILES: Cc1noc2cc(OCc3ncc(-c4nnc(C(F)F)o4)cc3F)ccc12 ; Cn1ncc2cc(OCc3ncc(-c4nnc(C(F)F)o4)cn3)ccc21 ; Cn1ncc2cc(OCc3ncc(-c4nnc(C(F)F)o4)cc3F)ccc21 ; Cc1nc2cc(OCc3ncc(-c4nnc(C(F)F)o4)cn3)ccc2o1 ; Cc1nc2cc(OCc3ncc(-c4nnc(C(F)F)o4)cc3F)ccc2o1]

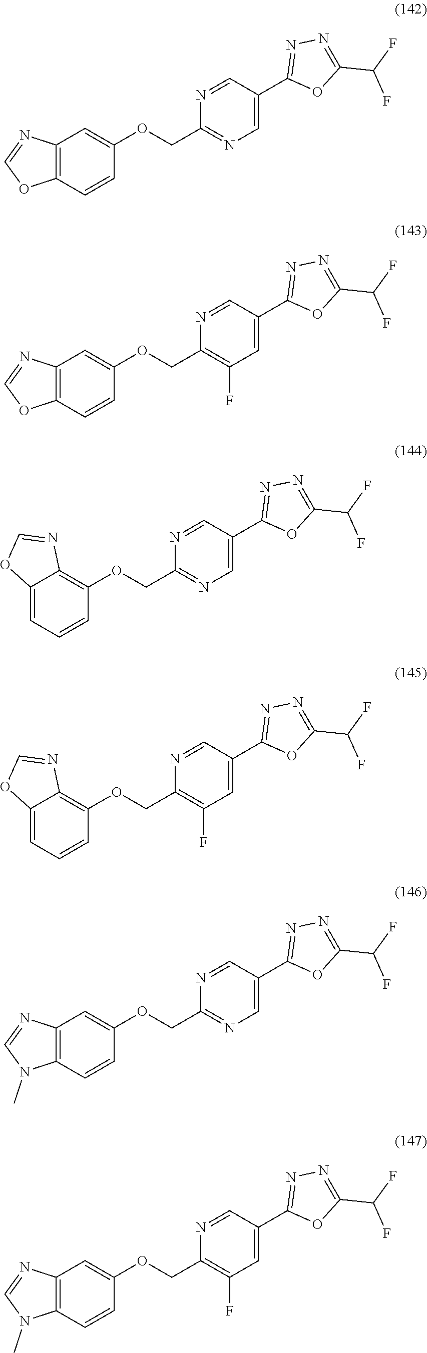 [SMILES: FC(F)c1nnc(-c2cnc(COc3ccc4ocnc4c3)nc2)o1 ; Fc1cc(-c2nnc(C(F)F)o2)cnc1COc1ccc2ocnc2c1 ; FC(F)c1nnc(-c2cnc(COc3cccc4ocnc34)nc2)o1 ; Fc1cc(-c2nnc(C(F)F)o2)cnc1COc1cccc2ocnc12 ; Cn1cnc2cc(OCc3ncc(-c4nnc(C(F)F)o4)cn3)ccc21 ; Cn1cnc2cc(OCc3ncc(-c4nnc(C(F)F)o4)cc3F)ccc21]

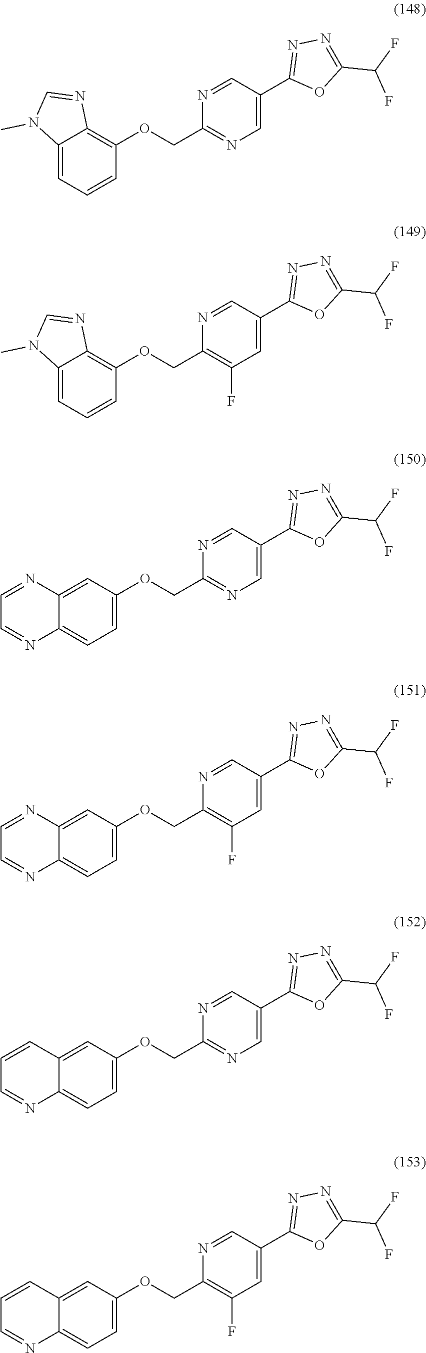 [SMILES: Cn1cnc2c(OCc3ncc(-c4nnc(C(F)F)o4)cn3)cccc21 ; Cn1cnc2c(OCc3ncc(-c4nnc(C(F)F)o4)cc3F)cccc21 ; FC(F)c1nnc(-c2cnc(COc3ccc4nccnc4c3)nc2)o1 ; Fc1cc(-c2nnc(C(F)F)o2)cnc1COc1ccc2nccnc2c1 ; FC(F)c1nnc(-c2cnc(COc3ccc4ncccc4c3)nc2)o1 ; Fc1cc(-c2nnc(C(F)F)o2)cnc1COc1ccc2ncccc2c1]

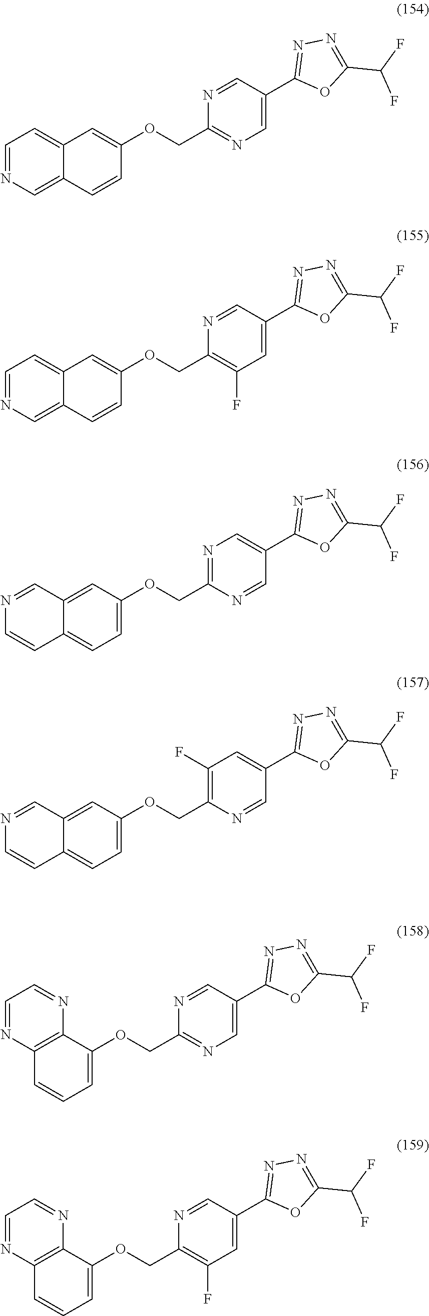 [SMILES: FC(F)c1nnc(-c2cnc(COc3ccc4cnccc4c3)nc2)o1 ; Fc1cc(-c2nnc(C(F)F)o2)cnc1COc1ccc2cnccc2c1 ; FC(F)c1nnc(-c2cnc(COc3ccc4ccncc4c3)nc2)o1 ; Fc1cc(-c2nnc(C(F)F)o2)cnc1COc1ccc2ccncc2c1 ; FC(F)c1nnc(-c2cnc(COc3cccc4nccnc34)nc2)o1 ; Fc1cc(-c2nnc(C(F)F)o2)cnc1COc1cccc2nccnc12]

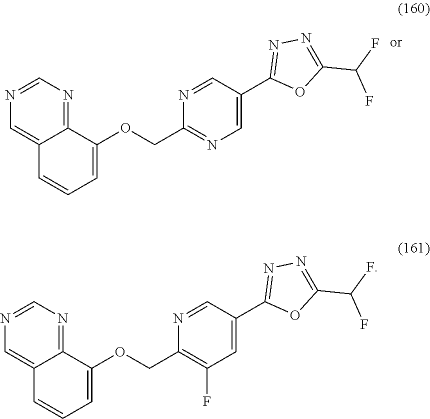 [SMILES: FC(F)c1nnc(-c2cnc(COc3cccc4cncnc34)nc2)o1 ; "or" ; Fc1cc(-c2nnc(C(F)F)o2)cnc1COc1cccc2cncnc12]

In certain embodiments, the compound of Formula (I) is one of the following compounds, or a pharmaceutically acceptable salt thereof:

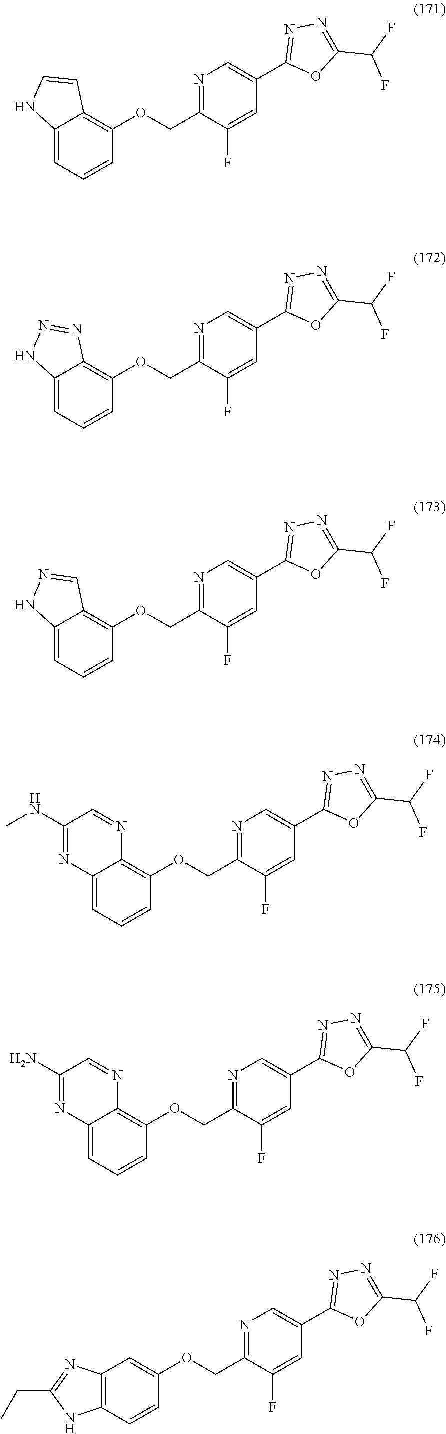 [SMILES: Fc1cc(-c2nnc(C(F)F)o2)cnc1COc1cccc2[nH]ccc12 ; Fc1cc(-c2nnc(C(F)F)o2)cnc1COc1cccc2[nH]nnc12 ; Fc1cc(-c2nnc(C(F)F)o2)cnc1COc1cccc2[nH]ncc12 ; CNc1cnc2c(OCc3ncc(-c4nnc(C(F)F)o4)cc3F)cccc2n1 ; Nc1cnc2c(OCc3ncc(-c4nnc(C(F)F)o4)cc3F)cccc2n1 ; CCc1nc2cc(OCc3ncc(-c4nnc(C(F)F)o4)cc3F)ccc2[nH]1]

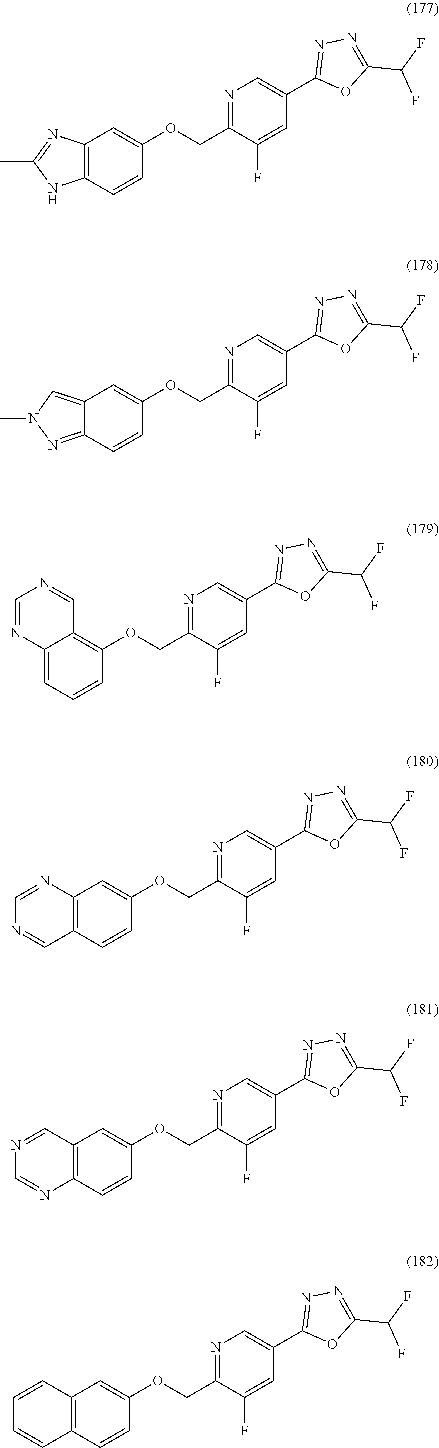 [SMILES: Cc1nc2cc(OCc3ncc(-c4nnc(C(F)F)o4)cc3F)ccc2[nH]1 ; Cn1cc2cc(OCc3ncc(-c4nnc(C(F)F)o4)cc3F)ccc2n1 ; Fc1cc(-c2nnc(C(F)F)o2)cnc1COc1cccc2ncncc12 ; Fc1cc(-c2nnc(C(F)F)o2)cnc1COc1ccc2cncnc2c1 ; Fc1cc(-c2nnc(C(F)F)o2)cnc1COc1ccc2ncncc2c1 ; Fc1cc(-c2nnc(C(F)F)o2)cnc1COc1ccc2ccccc2c1]

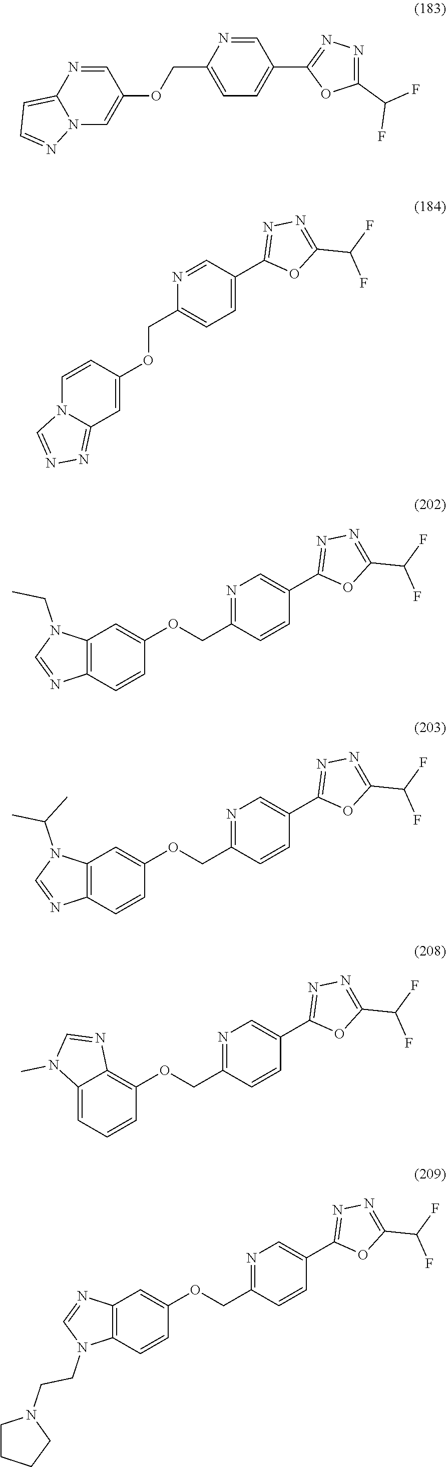 [SMILES: FC(F)c1nnc(-c2ccc(COc3cnc4ccnn4c3)nc2)o1 ; FC(F)c1nnc(-c2ccc(COc3ccn4cnnc4c3)nc2)o1 ; CCn1cnc2ccc(OCc3ccc(-c4nnc(C(F)F)o4)cn3)cc21 ; CC(C)n1cnc2ccc(OCc3ccc(-c4nnc(C(F)F)o4)cn3)cc21 ; Cn1cnc2c(OCc3ccc(-c4nnc(C(F)F)o4)cn3)cccc21 ; FC(F)c1nnc(-c2ccc(COc3ccc4c(c3)ncn4CCN3CCCC3)nc2)o1]

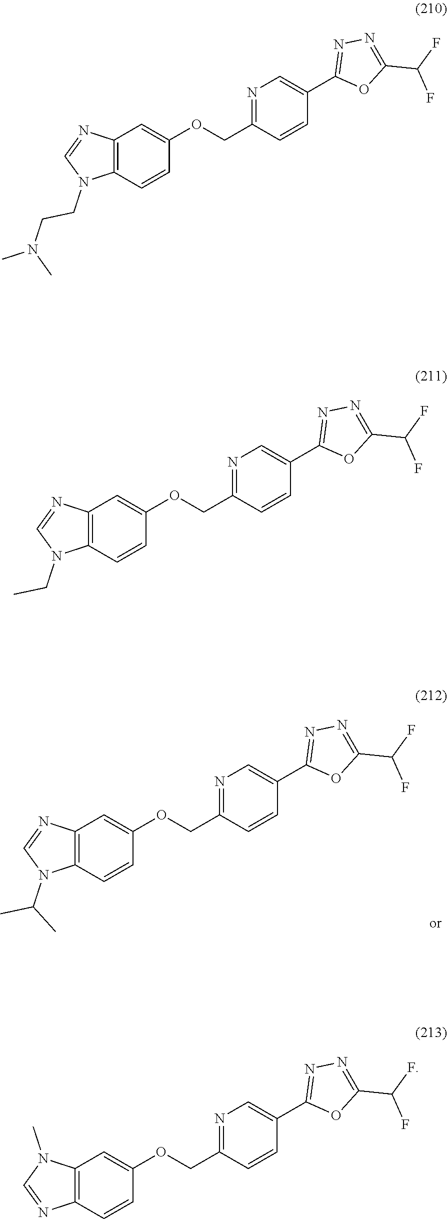 [SMILES: CN(C)CCn1cnc2cc(OCc3ccc(-c4nnc(C(F)F)o4)cn3)ccc21 ; CCn1cnc2cc(OCc3ccc(-c4nnc(C(F)F)o4)cn3)ccc21 ; CC(C)n1cnc2cc(OCc3ccc(-c4nnc(C(F)F)o4)cn3)ccc21 ; Cn1cnc2ccc(OCc3ccc(-c4nnc(C(F)F)o4)cn3)cc21]

In some embodiments, methods comprising the use or administration of Compound A,

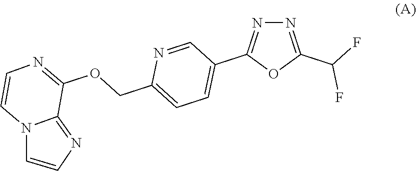 [SMILES: FC(F)c1nnc(-c2ccc(COc3nccn4ccnc34)nc2)o1]

or a pharmaceutically acceptable salt thereof, are provided.

In some embodiments, methods comprising the use of 2-(difluoromethyl)-5-(6-((imidazo[1,2-a]pyrazin-8-yloxy)methyl)pyridin-3-yl)-1,3,4-oxadiazole, or a pharmaceutically acceptable salt thereof for the inhibition of HDAC6 are provided. In some embodiments, methods comprising the administration of a therapeutically acceptable amount of 2-(difluoromethyl)-5-(6-((imidazo[1,2-a]pyrazin-8-yloxy)methyl)pyridin-3-yl)-1,3,4-oxadiazole, or a pharmaceutically acceptable salt thereof, to a subject in need thereof are provided.

Example 1 details a chemical synthesis of Compounds of Formula (I).

Example 2 details experiments performed to determine the potency of Compound A to inhibit recombinant human HDAC6 enzyme from deacetylating a synthetic acetylated peptide substrate. The data shows that Compound A has low nanomolar potency (24 nM) to inhibit HDAC6. This data demonstrates that Compound A was able to block the deacetylase function of HDAC6 at low nanomolar levels. In addition, experiments were performed to determine the selectivity of Compound A for human HDAC6 enzyme compared to all other human HDAC enzymes with Zn²⁺ dependent binding, specifically those paralogs comprising Class I, II and IV (HDACs 1,2,3,4,5,7,8,9,10,11). The data show that Compound A is >1699 fold selective for HDAC6 over all other HDAC paralogs (recombinant human HDAC enzymes tested against individually optimized acetylated peptide substrates). This data demonstrates that Compound A has high selectivity for HDAC6. The level of selectivity is superior to hydroxamic acid comparators. Compound A does not inhibit class I HDAC paralogs (HDACs 1, 2, 3, 8). Inhibition of class I HDACs is linked to dose-limiting toxicity in the clinic and the high selectivity of Compound A for HDAC6 supports a good safety profile.

Example 4 describes experiments performed to determine the pharmacokinetic profile of Compound A in mouse across multiple doses (3, 10, 30 mg/kg) after a single oral administration. The data show that Compound A has a Cmax (ng/mL) of 1,469 at 3 mg/kg, 5,002 at 10 mg/kg, and 11,335 at 30 mg/kg. Compound A has a T max (h) of 0.5 at 3 mg/kg, 0.5 at 10 mg/kg, and 2.0 at 30 mg/kg. Compound A has an AUClast (ng*h/mL) of 4,571 at 3 mg/kg, 16,939 at 10 mg/kg, and 80,797 at 30 mg/kg. This data demonstrates that the exposure of Compound A increases with increasing dose in a more-than dose proportional manner and exhibits sustained target coverage relative to the biochemical human HDAC6 IC50 for at least 8 h following a single oral dose at 3, 10, or 30 mg/kg in mouse. Example 4A also describes experiments performed to determine the pharmacokinetic profile of Compound A in rat across multiple doses (10, 1000 mg/kg) after a single oral administration. The data show that Compound A has a Cₘₐₓ (ng/mL) of 4,508-7,571 at 10 mg/kg and 83,022 at 1000 mg/kg. Compound A has a T max (h) of 0.5-1.2 at 10 mg/kg and 8.0 at 1000 mg/kg. Compound A has an AUCₗₐₛₜ (ng*h/mL) of 23,168-23,689 at 10 mg/kg and 1,532,158 at 1000 mg/kg. This data demonstrates that the exposure of Compound A increases with increasing dose and exhibits sustained target coverage relative to the biochemical human HDAC6 IC50 for at least 8 h following a single oral dose at 10 or 1000 mg/kg in rat.

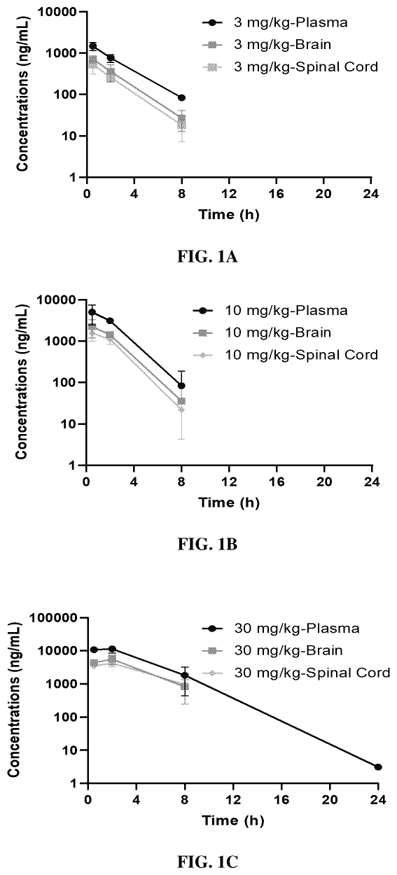

FIG. 1A shows the plasma, brain and spinal cord exposure (concentration ng/mL) of Compound A for 8 hours after oral administration of a single dose of 3 mg/kg to mice. FIG. 1B shows the plasma, brain and spinal cord exposure (concentration ng/mL) of Compound A for 8 hours after oral administration of a single dose of 10 mg/kg to mice. FIG. 1C shows the brain and spinal cord exposure (concentration ng/mL) of Compound A for 8 hours, and the plasma exposure (concentration ng/mL) of Compound A for 24 hours, after oral administration of a single dose of 30 mg/kg to mice. The exposure of Compound A increased with increasing doses in a more than dose proportional manner following single oral doses at 3, 10 and 30 mg/kg in mice and exhibited sustained target coverage.

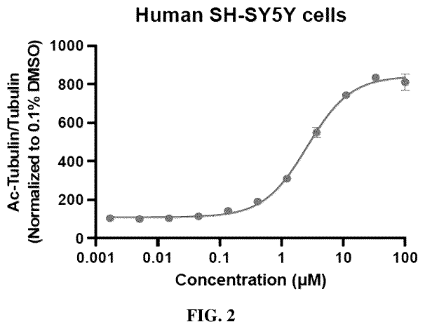

FIG. 2A shows the plasma exposure (concentration ng/mL) of Compound A for 8 hours (IV) or 24 hours (oral NMP and CMC formulations) after oral administration of a single dose of 1 mg/kg (IV) or 10 mg/kg (oral) to rats. FIG. 2B shows the plasma exposure (concentration ng/mL) of Compound A for 24 hours after oral administration of a single dose of 1,000 mg/kg to rats. The NMP formulation was 7.5% NMP, 5% solutol HS-15, 30% PEG-400 and 57.5% citric acid (10 mM). The CMC formulation was 1% carboxymethyl cellulose in water.

Example 4B describes experiments performed to determine the brain to plasma ratio (Kp) and brain to plasma ratio corrected for unbound compound concentrations (Kpuu) across multiple doses (3, 10, 30 mg/kg) in mouse. The data show that Compound A has a Kp range of 0.34-0.47 and a Kpuu range of 0.34-0.47 in mouse. This data demonstrates that Compound A penetrates the brain with a high unbound concentration across doses. The comparator ricolinostat (ACY-1215) does not get into the brain. Ricolinostat Kp could not be quantified as compound concentration in mouse brain was below level of quantification after a single oral administration at 30 mg/kg. Extrapolation from literature suggests that ricolinostat has a Kp of 0.03 in mouse brain at Cmax (Ma et al, Acta Neuropathol Commun 2018). Example 4B also describes experiments performed to determine the brain to plasma ratio (Kp) at 1,000 mg/kg in rat. The data shows that Compound A has a Kp of 0.45 in rat. This data demonstrates that Compound A penetrates the brain.

Additional experiments were performed to confirm Compound A target engagement in vitro, as measured by an increase in alpha-tubulin acetylation levels, which is the dominant substrate for HDAC6. The data shows a dose-dependent increase in alpha-tubulin acetylation levels beginning after ˜0.1 micromolar with a 310% increase observed at 1 micromolar and a >600% increase observed at 10 micromolar and 30 micromolar in human SH-SY5Y cells. This data demonstrates that Compound A is cell permeable and engages the HDAC6 target.

Additional experiments were performed to confirm that Compound A does not engage the off-target class I HDACs in vitro, as measured by an increase in histone H3 lysine 9 acetylation levels. The data shows that at 10 micromolar and 30 micromolar Compound A does not increase histone acetylation in human SH-SY5Y cells, while the broad-acting HDAC inhibitor SAHA increases histone acetylation by ˜600%. This data demonstrates that Compound A is selective for HDAC6.

FIG. 2 shows target engagement data obtained for the HDAC6 substrate acetyl alpha tubulin relative to total alpha tubulin (Ac-Tubulin/Tubulin) normalized to the 0.1% DMSO condition vs concentration (micromolar) of Compound A in human SH-SY5Y cells.

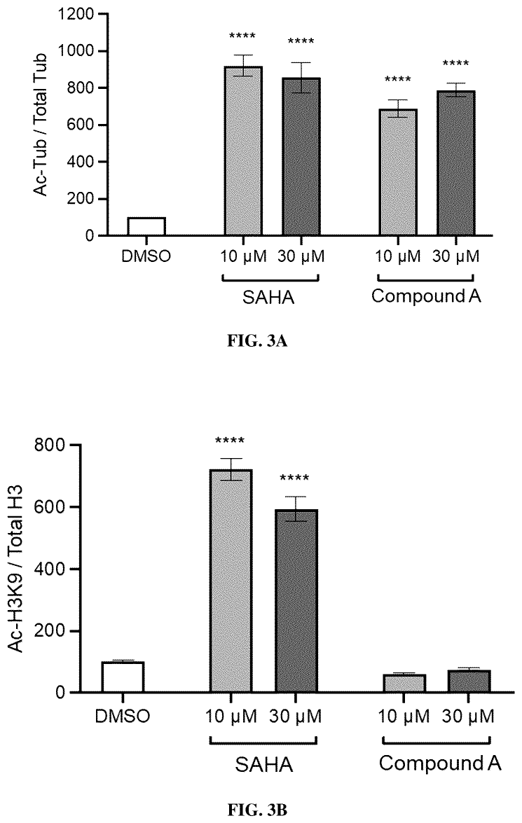

FIG. 3A shows target engagement via the HDAC6 substrate acetyl alpha tubulin relative to total alpha tubulin (Ac-Tub/Total Tub) normalized to the 0.1% DMSO condition for Compound A and SAHA at 10 micromolar and 30 micromolar in human SH-SY5Y cells.

FIG. 3B shows off-target engagement via the class I HDAC substrate acetyl histone H3 lysine 9 relative to total histone H3 (Ac-H3K9/Total H3) normalized to the 0.1% DMSO condition for Compound A and SAHA at 10 micromolar and 30 micromolar in human SH-SY5Y cells.

### Pharmaceutical Compositions and Kits

The present disclosure provides pharmaceutical compositions comprising a disclosed compound, or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of the disclosure, or a pharmaceutically acceptable salt thereof, and a pharmaceutically acceptable excipient.

The present disclosure provides pharmaceutical compositions comprising a compound that interacts with (e.g., inhibits) HDAC6 for use in treating a HDAC6-related disease or disorder in a subject in need thereof. The present disclosure provides pharmaceutical compositions comprising a compound that interacts with (e.g., inhibits) HDAC6 for use in treating a disease or disorder associated with aberrant activity of HDAC6 in a subject in need thereof. The present disclosure provides pharmaceutical compositions comprising a compound that interacts with (e.g., inhibits) HDAC6 for use in treating a disease or disorder associated with increased activity of HDAC6 in a subject in need thereof.

In certain embodiments, the composition is for use in treating a neurological or peripheral disorder. In certain embodiments, the composition is for use in treating a neurodegenerative, neurodevelopmental, neuropsychiatric, or neuropathy disease or disorder.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for subcutaneous injection.

In certain embodiments, the pharmaceutical composition is a liquid (e.g., aqueous solution) for intravenous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

Pharmaceutical compositions described herein can be prepared by any method known in the art of pharmacology. In general, such preparatory methods include the steps of bringing the composition comprising a compound of the disclosure into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., amyotrophic lateral sclerosis (ALS), Parkinson's Disease (PD), a neuropathy, a tauopathy or a neuroinflammation-related condition) in a subject in need thereof. In certain embodiments, the kits are useful for preventing a disease (e.g., amyotrophic lateral sclerosis (ALS), Parkinson's Disease (PD), a neuropathy, a tauopathy or a neuroinflammation-related condition) in a subject in need thereof. In certain embodiments, the kits are useful for reducing the risk of developing a disease (e.g., amyotrophic lateral sclerosis (ALS), Parkinson's Disease (PD), a neuropathy, a tauopathy or a neuroinflammation-related condition) in a subject in need thereof. In certain embodiments, the kits are useful for inhibiting the activity (e.g., aberrant activity, such as increased activity) of HDAC6 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., amyotrophic lateral sclerosis (ALS), Parkinson's Disease (PD), a neuropathy, a tauopathy or a neuroinflammation-related condition) in a subject in need thereof. In certain embodiments, the kits and instructions provide for preventing a disease (e.g., amyotrophic lateral sclerosis (ALS), Parkinson's Disease (PD), a neuropathy, a tauopathy or a neuroinflammation-related condition) in a subject in need thereof. In certain embodiments, the kits and instructions provide for reducing the risk of developing a disease (e.g., amyotrophic lateral sclerosis (ALS), Parkinson's Disease (PD), a neuropathy, a tauopathy or a neuroinflammation-related condition) in a subject in need thereof. In certain embodiments, the kits and instructions provide for inhibiting the activity (e.g., aberrant activity, such as increased activity) of HDAC6 in a subject or cell. A kit described herein may include one or more additional pharmaceutical agents described herein as a separate composition.

### Methods of Treatment

HDAC6 is unique in structure and function among all HDAC paralogs. In particular, it possesses two catalytic (deacetylase) domains and a zinc finger ubiquitin-binding domain. HDAC6 does not deacetylate histones, yet interacts with multiple substrates that affect disease-relevant pathways including microtubule stability, axonal and mitochondrial transport, protein aggregation, and autophagy. For example, HDAC6's direct substrates (e.g., tau, tubulin, and HSP90) engage key mechanisms in Alzheimer's disease. As a result of its unique structure and function, selectively targeting and inhibiting HDAC6 activity may avoid the side effects that are typical of existing FDA-approved HDAC inhibitors that result in clinical toxicity due to broad inhibition of multiple HDAC paralogs an/or inhibition of HDACs 1 and/or 2 (which has been shown to cause thrombocytopenia, a dose-limiting toxicity of most FDA-approved pan-HDAC inhibitors). Thus, treatment of HDAC6-related diseases with HDAC6-selective inhibitors may be particularly effective.

In some embodiments, methods of treating a neurodegenerative condition is provided, wherein the method comprises the administration of a therapeutically effective amount of Compound A or a pharmaceutically acceptable salt form thereof.

Cytotoxic protein aggregates that underlie many neurodegenerative disorders are cleared by the autophagy-lysosome pathway. HDAC6 is positioned to influence autophagic cytotoxic protein aggregate clearance in several ways. The zinc-finger motif (BUZ domain) of HDAC6 directly interacts with ubiquitinated proteins, including mitochondria, targeted for autophagic degradation and facilitates their delivery and concentration within aggresomes, a process dependent on the dynein binding domain of HDAC6 (Bánréti, Á., Sass, M., and Graba, Y. The emerging role of acetylation in the regulation of autophagy. Autophagy. 9:6, 819-829. 2013. DOI: 10.4161/auto.23908; and Richter-Landsberg, C. The α-tubulin deacetylase HDAC6 in aggresome formation and autophagy: implications for neurodegeneration. In: Autophagy: Cancer, Other Pathologies, Inflammation, Immunity, Infection, and Aging. Hayat M A, editor. Academic Press. 2015. 5:273-282. DOI: http://dx.doi.org/10.1016/B978-0-12-

Tubulin acetylation is not required for the initial formation of autophagosomes, but acetylated microtubules are important for the downstream fusion of autophagosomes and lysosomes (Xie, R., Nguyen, S., McKeehan, W., and Liu, L. Acetylated microtubules are required for fusion of autophagosomes with lysosomes. BMC Cell Biol. 11, 89. 2010. https://doi.org/10.1186/1471-2121-11-89).

More directly, HDAC6-mediated deacetylation of cortactin initiates F-actin polymerization to promote the fusion of autophagosomes and lysosomes and subsequent protein degradation (Lee, J., Koga, H., Kawaguchi, Y., Tang, W., Wong, E., Gao, Y., Pandey, U., Kaushik, S., Tresse, E., Lu, J., Taylor, J., Cuervo, A., and Yao, T. HDAC6 controls autophagosome maturation essential for ubiquitin-selective quality-control autophagy. EMBO J. 29(5):969-80. 2010. DOI: 10.1038/emboj.2009.405; and Lee, J. and Yao, T. Quality control autophagy: A joint effort of ubiquitin, protein deacetylase and actin cytoskeleton. Autophagy, 6:4, 555-557. 2010. DOI: 10.4161/auto.6.4.11812). Thus, the coordinated actions of HDAC6 protein aggregation, mediated by the HDAC6 BUZ and dynein binding domains, and catalytic activity are crucial for proper cytotoxic protein degradation.

People living with amyotrophic lateral sclerosis (pALS) and other neurodegenerative diseases exhibit defects in axonal transport and proteostasis, which in ALS are linked to motor neuron death and ultimately loss of motor function. Importantly, neither pathway is adequately addressed by the current standards of care (SoC). Histone deacetylase 6 (HDAC6) is a cytoplasmic enzyme with dual functions in axonal transport and proteostasis pathways. HDAC6 deacetylates non-histone substrates associated with intracellular transport, including α-tubulin, through its catalytic deacetylase domain 2 (CD2) and binds to ubiquitinated proteins to promote autophagy-mediated protein degradation through its ubiquitin-binding zinc finger (ZNF-UBP) domain.

Small molecule inhibition of the HDAC6 CD2 increases axonal transport and concomitantly decreases TDP-43 pathology in human induced pluripotent stem cell (hiPSC)-derived motor neurons (MNs) bearing ALS mutations. Hdac6 knockout increases compound muscle action potential and neuromuscular junction innervation, supporting improved axon health, in SOD1^{G93A} mice.

Historically, advancement of HDAC6 inhibitor therapeutics has been hindered by a lack of CNS penetrating candidates with drug-like properties that avoid off-target class I HDAC activity. Compound A is a CNS-penetrant, oxadiazole small molecule HDAC6 inhibitor compound.

Compound A is potent (<100 nM) and selective (>1000-fold) for HDAC6 over all other HDACs. Compound A demonstrates target engagement in the CNS and has favorable bioavailability. Compound A shows no dose-limiting toxicities or histological findings in a 14-day non-glp exploratory rat toxicology study.

In certain embodiments, Compound A may be administered in combination with at least a second therapeutic agent to an individual suffering from ALS or other neurodegenerative disease, where Compound A and the second therapeutic agent can be administered substantially simultaneously (e.g., within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more.

In certain embodiments, the second therapeutic agent is, e.g., Qalsody (Tofersen), Relyviro (AMX0035), Radicava (Edaravone), Rilutek (Riluzole), Tiglutik (Thickened Riluzole), Exservan (Riluzole Oral Film), or Nuedexta (dextromethorphan HBr and quinidine sulfate).

In some embodiments, methods of treating amyotrophic lateral sclerosis (ALS) are provided, wherein the methods comprise the administration of a therapeutically effective amount of Compound A or a pharmaceutically acceptable salt form thereof. In some embodiments, the ALS can be sporadic or familial ALS cases.

In some embodiments, methods of treating amyotrophic lateral sclerosis (ALS), wherein the ALS is sporadic and ALS with familial mutations including but not limited to TDP-43, SOD1, C9orf72, and FUS, wherein the method comprises the administration of a therapeutically effective amount of Compound A or a pharmaceutically acceptable salt form thereof.

In some embodiments, methods of treating amyotrophic lateral sclerosis (ALS), comprise the administration of Compound A, or a pharmaceutically acceptable salt thereof, to a patient in need thereof, wherein the ALS is characterized by one or more mutations.

In some embodiments, a method of treating ALS with Compound A or a pharmaceutically acceptable salt thereof is provided in a patient diagnosed with a mutation in a gene involved in RNA processing, such as TARDBP, FUS, ANG, SETX, MATR3, ATXN2, TAF15, EWSR1, hnRNPa1, or hnRNPA2B1. In some embodiments, a method of treating ALS is provided in a patient diagnosed with ALS characterized by a diagnostically or therapeutically relevant level of a protein selected from TAR DNA-Binding Protein 43 Kd, fused in sarcoma, angiogenin, senataxin, Matrin 3, Ataxin 2, TATA-binding associated factor 2N, EWS RNA Binding Protein 1, Heterogeneous nuclear ribonucleoprotein A1, heterogeneous nuclear ribonucleoproteins A2/B1, or elongator complex protein 3.

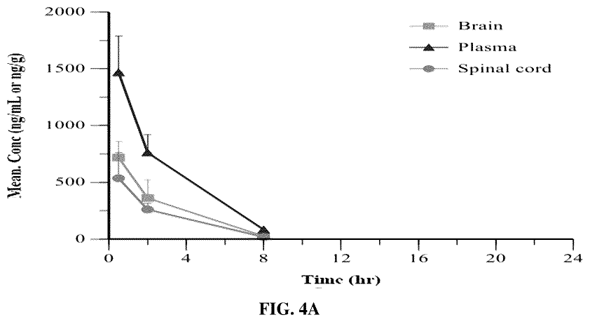

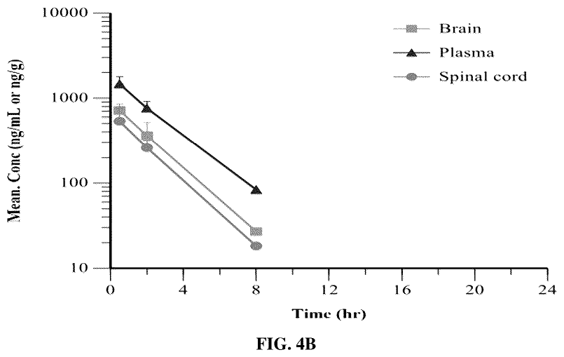

In some embodiments, a method of treating ALS with Compound A or a pharmaceutically acceptable salt thereof is provided in a patient diagnosed with a mutation in a gene involved in protein trafficking and degradation, such as C9ORF72, ALS2, VAPB, CHMP2B, FIG. 4, UBQLN2, SQSTM1, OPTN, VCP, or TBK1. In some embodiments, a method of treating ALS is provided in a patient diagnosed with ALS characterized by a diagnostically or therapeutically relevant level of a protein selected from guanine nucleotide exchange C9orf72, alsin, vesicle-associated membrane protein-associated protein B/C, charged multivesicular body protein 2b, polyphosphoinositide phosphatase, ubiquilin-2, sequestosome-1 (p62), sigma non-opioid intracellular receptor 1, optineurin, valosin containing protein or tank binding kinase 1.

In some embodiments, a method of treating ALS with Compound A or a pharmaceutically acceptable salt thereof is provided in a patient diagnosed with a mutation in a gene involved in cytoskeletal and axonal dynamics, such as DCTN1, PFN1, SPG11, TUBA4A, NEFH, PRPH, or NEK1. In some embodiments, a method of treating ALS is provided in a patient diagnosed with ALS characterized by a diagnostically or therapeutically relevant level of a protein selected from dynactin subunit 1, profilin 1, spatacsin, tubulin alpha-4a chain, neurofilament heavy polypeptide, peripherin, and NIMA (never in mitosis gene A)-related kinase-1.

In some embodiments, a method of treating ALS with Compound A or a pharmaceutically acceptable salt thereof is provided in a patient diagnosed with a mutation in a gene involved in mitochondrial function, such as CHCHD10 or C19ORF12. In some embodiments, a method of treating ALS is provided in a patient diagnosed with ALS characterized by a diagnostically or therapeutically relevant level of a protein selected from coiled-coil-helix-coiled-coil-helix-domain containing 10, or Protein C19orf12.

In some embodiments, a method of treating ALS Compound A or a pharmaceutically acceptable salt thereof is provided in a patient diagnosed with a mutation in a gene such as SOD1, ERBB4, SS18L1, PON1-3, DAO, CHRNA3, 4 and B4, ALS3, ALS7, ALS6-21, and ALS-FTD.

In some embodiments, a method of treating ALS with Compound A or a pharmaceutically acceptable salt thereof is provided in a patient diagnosed with ALS characterized by a diagnostically or therapeutically relevant level of a protein selected from superoxidase dismutase [Cu—Zn], receptor tyrosine-protein kinase erbB-4, calcium-responsive transactivator, neuropathy target esterase, paraoxonase 1-3, D-amino acid oxidase, neuronal acetylcholine receptor subunit alpha-3, alpha-4 or beta-4, or ALS3.

In some embodiments, methods of treating a condition are provided, wherein the method comprises the administration of a therapeutically effective amount of Compound A or a pharmaceutically acceptable salt form thereof, wherein the indication treated is selected from the group consisting of: amyotrophic lateral sclerosis (ALS) and Alzheimer's Disease (AD) and may prevent tau aggregation and stop or slow the progression of AD. HDAC6 expression was shown to be elevated in postmortem brain samples from Alzheimer's disease patients. Small molecule compounds inhibiting HDAC6 are being developed as a potential therapy for neurodegenerative disorders including Alzheimer's disease (AD), amyotrophic lateral sclerosis (ALS), frontotemporal dementia (FTD), progressive supranuclear palsy (PSP), Huntington's disease (HD), Parkinson's disease (PD), and multiple sclerosis. Small molecule compounds inhibiting HDAC6 are also being developed as a potential therapy for peripheral neuropathies including chemotherapy-induced peripheral neuropathy (CIPN) and Charcot-Marie-Tooth disease (CMT), as well as brain cancers such as glioblastoma.

In some embodiments, methods of treating neuropathy are provided, wherein the method comprises the administration of a therapeutically effective amount of Compound A or a pharmaceutically acceptable salt form thereof. In some embodiments, methods of treating chemotherapy induced neuropathy are provided, wherein the method comprises the administration of a therapeutically effective amount of Compound A or a pharmaceutically acceptable salt form thereof. Neuropathy is defined by the International Association for the Study of Pain (IASP) as a disturbance of function or pathological change in one or several nerves (mononeuropathy or polyneuropathy respectively). Neuropathy can be associated or not with pain (painless and painful neuropathy respectively). Neuropathy is induced by several factors such as, but not limited to, metabolic disorders (diabetic neuropathy), injury or disease affecting the nervous system (nerve trauma, stroke), genetic (Guillan-Barre syndrome, Charcot-Marie-Tooth disease), virus infection and antiretroviral drugs (HIV, HSV), and cancer chemotherapy. The latter is called Chemotherapy-Induced Peripheral Neuropathy (CIPN). CIPN can limit the dosage and selection of cytostatic drugs, delay further treatment cycles, or lead to discontinuation of cytostatic therapy. Therefore, altering the optimal chemotherapeutic treatment and reducing the chance of survival for cancer patients. The symptoms range from early post-treatment pain, such as pain from paclitaxel, to paraesthesias, sensory ataxia, numbness (loss of sensation), tingling, and mechanical and cold allodynia. The extremities of the body are predominantly affected, but neuropathy can proceed along the limbs during repetitive treatment. Substances that cause CIPN include the commonly used taxanes, platinum derivatives and vinca alkaloids, as well as the more rarely used ixabepilone, bortezomib and thalidomide. CIPN can be induced by agents such as: cisplatin, dicarbazine, streptozocin, cyclophosphamide, carmustine, lomustine, procarbazine, mitomycin, cytarabine, methotrexate, 5-fluorouracil, vinblastine, vincristine, bleomycin, paclitaxel, asparaginase, busulfan, carboplatin, dacarbazine, fludarabine, hydroxyurea, ifosfamide, mercaptopurine, mitotane, streptozocin, taxol and or a mixture of two or more agents thereof and by vincristine.

Platinum-based compounds such as cisplatin, carboplatin, and oxaliplatin bind and damage DNA triggering cell death in rapidly dividing cells (e.g. cancer cells). These drugs are used in many types of solid tumors. Though all three are known to cause classic symptoms of CIPN, higher incidences are seen with cisplatin and oxaliplatin. CIPN due to cisplatin is more often irreversible than in cases with oxaliplatin. CIPN is a dose-limiting toxicity with both cisplatin and oxaliplatin. While numerous potentially neuroprotective agents have been tested to reduce CIPN induced by cisplatin, none of these agents have proven the ability to prevent or limit the neurotoxicity of cisplatin.

Cisplatin is a potent antineoplastic agent widely used in the treatment of various solid cancers such as lung, ovarian or testicular cancers as well as certain forms of lymphomas. The anti-tumor action of cisplatin requires its intracellular bioactivation by the replacement of chlorides by water molecules, forming a highly reactive molecule that binds to DNA and induces cytotoxic lesions in tumors. However, the unwanted accumulation of cisplatin in healthy cells can also trigger genotoxicity. Indeed, the clinical use of cisplatin is restricted by various severe adverse effects, including chemotherapy-induced peripheral neuropathy (CIPN).

In clinical practice, CIPN is often considered as a frequent but unavoidable adverse effect of cisplatin chemotherapy that should be accepted by patients. Therefore, there is an urgent medical need for strategies that alleviate peripheral neuropathy, without interfering with the efficiency of cisplatin to control tumor growth.

In some embodiments, the present disclosure also relates to a method for the treatment of neuropathy in a subject in need thereof comprising administering to the subject a therapeutically effective amount of a selective HDAC6 inhibitor such as Compound A, or a therapeutically acceptable salt thereof. In some embodiments, the present disclosure also relates to a method for the treatment of chemotherapy induced peripheral neuropathy (CIPN) in a subject in need thereof comprising administering to the subject a therapeutically effective amount of a selective HDAC6 inhibitor such as Compound A, or a therapeutically acceptable salt thereof. In some embodiments, the CIPN occurs after treatment of the subject with a platinum-based neoplastic agent (e.g., cisplatin, carboplatin and oxaliplatin), a vinca alkaloid (e.g., vinblastine, vincristine, vinorelbine or vindesine), an epothilone (e.g., ixabepilone), a taxane (e.g., paclitaxel, docetaxel, cabazitaxel, nab-paclitaxel), a proteasome inhibitor (e.g., brotezomib, carfilzomib or ixazomib) or an immunomodulatory drug (e.g., checkpoint inhibitor, cytokine, adjuvant or other immunomodulator therapy), or any combination thereof. In some embodiments, the CIPN occurs after treatment of the subject with a platinum-based neoplastic agent (e.g., cisplatin, carboplatin and oxaliplatin). In some embodiments, the CIPN occurs after treatment of the subject with a vinca alkaloid (e.g., vinblastine, vincristine, vinorelbine or vindesine). In some embodiments, the CIPN occurs after treatment of the subject with an epothilone (e.g., ixabepilone). In some embodiments, the CIPN occurs after treatment of the subject with a taxane (e.g., paclitaxel, docetaxel, cabazitaxel, nab-paclitaxel). In some embodiments, the CIPN occurs after treatment of the subject with a proteasome inhibitor (e.g., brotezomib, carfilzomib or ixazomib). In some embodiments, the CIPN occurs after treatment of the subject with an immunomodulatory drug (e.g., checkpoint inhibitor, cytokine, adjuvant or other immunomodulator therapy).

In some embodiments, methods of treating a condition are provided, wherein the method comprises the administration of a therapeutically effective amount of Compound A or a pharmaceutically acceptable salt form thereof, during or after administration of a cancer therapy that can also induce neuropathy. In some embodiments, methods of treating a condition are provided, wherein the method comprises the intravenous administration of a therapeutically effective amount of Compound A or a pharmaceutically acceptable salt form thereof, during or after administration of a cancer therapy that can also induce neuropathy.

In some embodiments, methods of treating a condition are provided, wherein the method comprises the administration of a therapeutically effective amount of Compound A or a pharmaceutically acceptable salt form thereof, wherein the indication treated is selected from the group consisting of: a kidney disease (e.g., autosomal dominant polycystic kidney disease (ADPKD, diabetic nephropathy (DN), chronic kidney disease (CKD), acute kidney injury (AKI)) and cardiac disorders (e.g., dilated cardiomyopathy, HF-rEF, HF-pEF, RBM20 mutant, and the like).

The present disclosure provides methods for treating HDAC6-related diseases and disorders. In certain embodiments, the application provides a method of treating a proliferative disease, inflammatory disease, infectious disease, autoimmune disease, heteroimmune disease, neurological disorder, peripheral disease or disorder, metabolic disease, cystic fibrosis, polycystic kidney disease, pulmonary hypertension, cardiac dysfunction, or disease or disorder mediated by or linked to T-cell dysregulation. In certain embodiments, the application provides a method of treating a proliferative disease. In certain embodiments, the application provides a method of treating cancer. In certain embodiments, the application provides a method of treating a hematological cancer. In certain embodiments, the application provides a method of treating leukemia, T-cell lymphoma, Hodgkin's Disease, non-Hodgkin's lymphoma, or multiple myeloma. In certain embodiments, the application provides a method of treating a cancer comprising a solid tumor. In certain embodiments, the application provides a method of treating glioma, glioblastoma, non-small cell lung cancer, brain tumor, neuroblastoma, bone tumor, soft-tissue sarcoma, head and neck cancer, genitourinary cancer, lung cancer, breast cancer, pancreatic cancer, melanoma, stomach cancer, brain cancer, liver cancer, thyroid cancer, clear cell carcinoma, uterine cancer, or ovarian cancer.

In certain embodiments, the application provides a method of treating a hereditary sensory and autonomic neuropathies (HSAN, also known as hereditary sensory neuropathy, HSN).

In certain embodiments, the application provides a method of treating an inflammatory disease. In certain embodiments, the application provides a method of treating osteoarthritis, rheumatoid arthritis, lupus, inflammatory bowel disease, Crohn's Disease, ulcerative colitis, anemia, leukocytosis, asthma, chronic obstructive pulmonary disease, appendicitis, bronchitis, bursitis, conjunctivitis, dermatitis, encephalitis, myelitis myocarditis, sinusitis, dermatitis, psoriasis, eczema, or acne. In certain embodiments, the composition is for use in treating a disease or disorder mediated by or linked to T-cell dysregulation. In certain embodiments, the composition is for use in treating arthritis, colitis, allograft rejection, lupus, asthma, psoriasis, inflammation, allergy, allergic encephalomyelitis, autoimmune lymphoproliferative disorder, autoimmune polyglandular syndrome type II, type I diabetes, lymphoma, Wiskott-Aldrich syndrome, or myasthenia gravis.

In certain embodiments, the application provides a method of treating an infectious disease. In certain embodiments, the application provides a method of treating bacterial, fungal, or protozoal infections.

In certain embodiments, the application provides a method of treating an autoimmune disease. In certain embodiments, the application provides a method of treating diabetes, thyroiditis, Graves' disease, Guillain-Barre syndrome, Addison's disease, scleroderma, primary biliary cirrhosis, Reiter's syndrome, psoriasis, chronic fatigue, or endometriosis.

In certain embodiments, the application provides a method of treating a heteroimmune disease. In certain embodiments, the application provides a method of treating graft versus host disease, transplantation, transfusion, anaphylaxis, allergic conjunctivitis, or allergic rhinitis.

In certain embodiments, the application provides a method of treating a neurological disease or disorder. In certain embodiments, the application provides a method of treating a neurodegenerative, neurodevelopmental, neuropsychiatric, or neuropathy disease or disorder. In certain embodiments, the application provides a method of treating Fragile-X syndrome, Charcot-Marie-Tooth disease, Alzheimer's disease, Parkinson's diseases, Huntington's disease, multiple sclerosis, amyotrophic lateral sclerosis, Creutzfeldt-Jakob disease, Lewy body dementia, vascular dementia, muscular atrophy, seizure induced memory loss, schizophrenia, Rubinstein Taybi syndrome, Rett Syndrome, attention deficit hyperactivity disorder, dyslexia, bipolar disorder, social, cognitive and learning disorders associated with autism, attention deficit disorder, schizophrenia, major depressive disorder, peripheral neuropathy, diabetic retinopathy, diabetic peripheral neuropathy, chemotherapy-induced peripheral neuropathy, chemotherapy-induced cognitive dysfunction, traumatic brain injury (TBI), chronic traumatic encephalopathy (CTE), or a tauopathy. In certain embodiments, the application provides a method of treating primary age-related tauopathy (PART)/neurofibrillary tangle-predominant senile dementia, chronic traumatic encephalopathy, dementia pugilistica, progressive supranuclear palsy, corticobasal degeneration, Pick's disease, frontotemporal dementia, frontotemporal dementia and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, lipofuscinosis, Alzheimer's disease, or argyrophilic grain disease. In certain embodiments, the application provides a method of treating Alzheimer's disease. In certain embodiments, the composition is for use in treating Alzheimer's disease, Fragile-X syndrome, Charcot-Marie-Tooth disease, Parkinson's disease, Huntington's disease, multiple sclerosis, amyotrophic lateral sclerosis, Rett Syndrome, major depressive disorder, chemotherapy-induced cognitive dysfunction, traumatic brain injury (TBI), chronic traumatic encephalopathy (CTE), brain cancer, or a tauopathy such as frontotemporal dementia, progressive supranuclear palsy, or corticobasal degeneration.

In certain embodiments, the application provides a method of treating a Cancer-Related Cognitive Impairment (CRCI). CRCI is a known occurrence in patients who have cancer and/or are receiving cancer therapy. It can manifest across various domains of cognition, including working memory, attention, concentration, processing speed, and executive functioning. CRCI is most common during and immediately after cancer therapy, with most individuals showing recovery over time. Some individuals have a persistent, but stable cognitive impairment, and a small number show, increasing decline over time. Studies have focused especially on breast cancer patients, but CRCI has been reported in multiple types of cancer, including colorectal, lung, ovarian, prostate, testicular cancer and hematological malignancies. While CRCI has been associated with various treatment modalities, including radiotherapy, chemotherapy, hormone therapy and novel systemic therapies, it has been also detected prior to cancer treatment. CRCI is a term used to describe the decline in patients cognitive functions, such as perception, attention, language, thinking, learning and memory, action planning, understanding, reasoning and problem solving. Cognitive changes in cancer patients may be induced by cancer treatment or by the presence of cancer itself.

Formal neuropsychological tests have been established as standard methods for detection of CRCI, because they provide objective assessments of various domains of cognition. The International Cognition and Cancer Task Force (ICCTF) recommends to evaluate learning, memory, processing speed and executive functions (Wefel et al., 2011; Joly et al., 2015). ICCTF suggests the use of validated neuropsychological tests, namely Hopkins Verbal Learning Test-Revised (HVLT-R) (Benedict et al., 1998), Controlled Oral Word Association Test (COWAT) of the Multilingual Aphasia Examination (Benton et al., 1994) and Trail Making Test (TMT) (Reitan, 1992). Investigators are then encouraged to supplement additional executive function tests, based on their own preferences. Cognitive impairment can be defined relative to population norms, to a control group or to an individual pretreatment functioning (a drop from baseline performance) (Horowitz et al., 2018). ICCTF recommends the following criteria for determining cognitive impairment: ≥2 test scores at or below −1.5 standard deviations (SDs) from the normative mean (or the mean score of an appropriate control group) or 1 test score at or below −2.0 SDs from the mean, or both (Wefel et al., 2011).

In certain embodiments, the application provides a method of treating a neurological or peripheral disease or disorder.

In certain embodiments, the application provides a method of treating a neurodegenerative, neurodevelopmental, neuropsychiatric, or neuropathy disease or disorder.

In certain embodiments, the application provides a method of treating cystic fibrosis. In certain embodiments, the application provides a method of treating polycystic kidney disease. In certain embodiments, the application provides a method of treating pulmonary hypertension. In certain embodiments, the application provides a method of treating cardiac dysfunction.

The present disclosure provides methods of inhibiting the activity of HDAC. In certain embodiments, the application provides a method of inhibiting the activity of HDAC6. In certain embodiments, the application provides a method of inhibiting the activity of HDAC6 in vitro. In certain embodiments, the application provides a method of inhibiting the activity of HDAC6 in vivo. In certain embodiments, the application provides a method of inhibiting the activity of HDAC6 in a cell. In certain embodiments, the application provides a method of inhibiting the activity of HDAC6 in a human cell.

In certain embodiments, the methods comprise administering to a subject in need thereof (e.g., a subject with a neurological disorder) a compound that interacts with HDAC6, for example, a compound that is an inhibitor of HDAC6, a modulator of HDAC6, a binder of HDAC6, or a compound that modifies HDAC6. In certain embodiments, the methods comprise administering a compound of the disclosure, or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure, or a pharmaceutically acceptable salt, co-crystal, tautomer, stereoisomer, solvate, hydrate, polymorph, isotopically enriched derivative, or prodrug, or composition thereof, to a subject in need thereof.

In certain embodiments, a compound of the disclosure is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for treating a hematological cancer in a subject in need thereof. In certain embodiments, the effective amount is an amount effective for treating a cancer comprising a solid tumor in a subject in need thereof. In certain embodiments, the effective amount is an amount effective for treating inflammatory disease in a subject in need thereof. In certain embodiments, the effective amount is an amount effective for preventing inflammatory disease in a subject in need thereof. In certain embodiments, the effective amount is an amount effective for treating an infectious disease in a subject in need thereof. In certain embodiments, the effective amount is an amount effective for preventing an infectious disease in a subject in need thereof. In certain embodiments, the effective amount is an amount effective for treating a cardiovascular disease in a subject in need thereof. In certain embodiments, the effective amount is an amount effective for treating a neurological disorder in a subject in need thereof. In certain embodiments, the effective amount is an amount effective for preventing a neurological disorder in a subject in need thereof. In certain embodiments, the effective amount is an amount effective for treating a neurodegenerative, neurodevelopmental, neuropsychiatric, or neuropathy disease in a subject in need thereof. In certain embodiments, the effective amount is an amount effective for preventing a neurodegenerative, neurodevelopmental, neuropsychiatric, or neuropathy disease in a subject in need thereof.

In some embodiments, the subject can be a human and preferably an adult human diagnosed with chemotherapy induced neuropathy (CIPN), such as CIPN resulting from treatment with platinum drugs, such as oxaliplatin; taxanes, such as docetaxel; vinca alkaloids, such as vincristine; and myeloma treatments, such as bortezomib. In some embodiments, the subject can be a human and preferably an adult human diagnosed with chemotherapy induced neuropathy (CIPN), such as CIPN resulting from treatment with cisplatin, or other agents such as paclitaxel or paclitaxel and carboplatin.

In some embodiments, the subject can be an adult human diagnosed with a form of cancer and being treated with a microtubule stabilizing agent such as paclitaxel.

In some embodiments, the subject can be an adult human diagnosed with metastatic breast cancer being treated with paclitaxel, after failure of combination chemotherapy for metastatic disease or relapse within six months of adjuvant chemotherapy. In some embodiments, the subject can be an adult human diagnosed with metastatic breast cancer being treated with paclitaxel in a dose of 260 mg/m² administered intravenously over 30 minutes every three weeks after failure of combination chemotherapy for metastatic breast cancer or relapse within six months of adjuvant chemotherapy.

In some embodiments, the subject can be an adult human diagnosed with locally advanced or metastatic non-small cell lung cancer (NSCLC) being treated with paclitaxel, as first-line treatment in combination with carboplatin, in patients who are not candidates for curative surgery or radiation therapy. In some embodiments, the subject can be an adult human diagnosed with locally advanced or metastatic non-small cell lung cancer (NSCLC) being treated with paclitaxel, as first-line treatment in combination with carboplatin, in patients who are not candidates for curative surgery or radiation therapy, wherein the paclitaxel is administered at a dose of 100 mg/m² administered as an intravenous infusion over 30 minutes on Days 1, 8, and 15 of each 21-day cycle, and wherein carboplatin is administered on Day 1 of each 21-day cycle immediately after the paclitaxel.

In some embodiments, the subject can be an adult human diagnosed with metastatic adenocarcinoma of the pancreas as first-line treatment being treated with paclitaxel, in combination with gemcitabine.

In some embodiments, the subject can be an adult human diagnosed with metastatic adenocarcinoma of the pancreas as first-line treatment being treated with paclitaxel, in combination with gemcitabine, wherein the paclitaxel is administered at a dose of 125 mg/m² administered as an intravenous infusion over 30-40 minutes on Days 1, 8, and 15 of each 28-day cycle, and the gemcitabine is administered immediately after the paclitaxel on Days 1, 8, and 15 of each 28-day cycle.

In certain embodiments, the effective amount is an amount effective for reducing the risk of developing a disease (e.g., proliferative disease, inflammatory disease, infectious disease, a neurological disorder, peripheral disease, or cardiovascular disease) in a subject in need thereof.

In certain embodiments, the effective amount is an amount effective for inhibiting the activity (e.g., aberrant activity, such as increased activity) of HDAC6 in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inhibiting the activity of HDAC6 by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting the activity of HDAC6 by a range between a percentage described in this paragraph and another percentage described in this paragraph, inclusive.

### Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. For example, in treating cancer, an effective amount of an inventive composition may prevent tumor regrowth, reduce the tumor burden, or stop the growth or spread of a tumor. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for HDAC6 inhibition (e.g., at least 5%, at least 10%, at least 20%, at least 30%, at least 40%, at least 50%, at least 60%, at least 70%, at least 80%, at least 90%, at least 95%, or at least 99% inhibition of the activity of HDAC6). In certain embodiments, a therapeutically effective amount is an amount sufficient for treating a disease or disorder (e.g., neurological disorder, cancer). In certain embodiments, a therapeutically effective amount is an amount sufficient for HDAC6 inhibition and treating a disease or disorder (e.g., neurological disorder, cancer).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for HDAC6 inhibition. In certain embodiments, a prophylactically effective amount is an amount sufficient for treating a disease or disorder (e.g., neurological disorder, cancer). In certain embodiments, a prophylactically effective amount is an amount sufficient for HDAC6 inhibition and treating a disease or disorder (e.g., neurological disorder, cancer).

As used herein, the term “inhibit” or “inhibition” in the context of enzymes, for example, in the context of HDAC6, refers to a reduction in the activity of the enzyme. In some embodiments, the term refers to a reduction of the level of enzyme activity, e.g., HDAC6 activity, to a level that is statistically significantly lower than an initial level, which may, for example, be a baseline level of enzyme activity. In some embodiments, the term refers to a reduction of the level of enzyme activity, e.g., HDAC6 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematological cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Pagive the's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Pagive the's disease of the vulva).

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

The term “chemotherapeutic agent” refers to a therapeutic agent known to be of use in chemotherapy for cancer.

A “hematological cancer” includes a cancer which affects a hematopoietic cell or tissue. Hematological cancers include cancers associated with aberrant hematological content and/or function. Examples of hematological cancers include, but are nor limited to, leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), lymphoma such as Hodgkin's lymphoma (HL) (e.g., B-cell HL, T-cell HL), non-Hodgkin's lymphoma (NHL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma, primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma), a mixture of one or more leukemia/lymphoma as described above, multiple myeloma, heavy chain disease (e.g., alpha chain disease, gamma chain disease, mu chain disease) acute non-lymphocytic leukemia (ANLL), acute promyelocytic leukemia (APL), acute myelomonocytic leukemia (AMMoL), polycythemia vera, Wilm's tumor, and Ewing's sarcoma.

The term “heteroimmune disease” refers to a state in which an immune response to an exogenous antigen (e.g., drug, pathogen) results in immunopathological changes. The immune response is triggered by an antigen from a different species (heteroimmune), thus it differs from an infectious disease because the emphasis is on the immune response, not the foreign species (infectious pathogen) causing the disease.

As used herein the term “neuropathy”, also known as “peripheral neuropathy” refers to disease affecting the peripheral nerves, meaning nerves beyond the brain and spinal cord. Damage to peripheral nerves may induce pain, impair sensation, movement, gland or organ function depending on which nerves are affected; in other words, neuropathy affecting motor, sensory, or autonomic nerves result in different symptoms. More than one type of nerve may be affected simultaneously. Peripheral neuropathy may be acute (with sudden onset, rapid progress) or chronic (symptoms begin subtly and progress slowly), and may be reversible or permanent. Common causes include systemic diseases (such as diabetes or leprosy), hyperglycemia-induced glycation, vitamin deficiency, medication (e.g., chemotherapy, or commonly prescribed antibiotics including metronidazole and the fluoroquinolone class of antibiotics (such as ciprofloxacin, levofloxacin, moxifloxacin)), traumatic injury, including ischemia, radiation therapy, excessive alcohol consumption, immune system disease, celiac disease, non-celiac gluten sensitivity, or viral infection. It can also be genetic (present from birth) or idiopathic (no known cause).

In a particular embodiment, the neuropathy is a neuropathic pain or a painful neuropathy.

Neuropathic pain, also known as painful neuropathy, is currently defined as pain initiated or caused by a primary lesion or dysfunction in the nervous system. Nerve damage can be caused by trauma and disease and thus the term “neuropathic pain” encompasses many disorders with diverse aetiologies. These include, but are not limited to, peripheral neuropathy, diabetic neuropathy, CIPN, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S 1 1-S 147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

Neuropathy affecting just one nerve is called “mononeuropathy” and neuropathy involving nerves in roughly the same areas on both sides of the body is called “symmetrical polyneuropathy” or simply “polyneuropathy”. When two or more (typically just a few, but sometimes many) separate nerves in disparate areas of the body are affected it is called “mononeuritis multiplex”, “multifocal mononeuropathy”, or “multiple mononeuropathy”.

Neuropathy may cause painful cramps, fasciculations (fine muscle twitching), muscle loss, bone degeneration, and changes in the skin, hair, and nails. Additionally, motor neuropathy may cause impaired balance and coordination or, most commonly, muscle weakness; sensory neuropathy may cause numbness to touch and vibration, reduced position sense causing poorer coordination and balance, reduced sensitivity to temperature change and pain, spontaneous tingling or burning pain, or skin allodynia (severe pain from normally nonpainful stimuli, such as light touch); and autonomic neuropathy may produce diverse symptoms, depending on the affected glands and organs, but common symptoms are poor bladder control, abnormal blood pressure or heart rate, and reduced ability to sweat normally.

Treatment may be for any purpose, including the therapeutic treatment of subjects suffering from neuropathy (e.g. chemotherapeutic-induced pain neuropathy), as well as the prophylactic treatment of subjects who do not suffer from pain (e.g., subjects identified as being at high risk pain).

In some embodiment the subject of the present disclosure suffers from chemotherapy-induced peripheral neuropathy.

In a particular embodiment, the method of the present disclosure is suitable for the treatment of chemotherapeutic-induced peripheral neuropathy (CIPN).

In a particular embodiment, the method of the present disclosure is suitable for the treatment of a painful neuropathy associated with a chemotherapeutic-induced peripheral neuropathy (CIPN).

As used herein the term “chemotherapy-induced peripheral neuropathy” (CIPN) refers peripheral neuropathy induced by antineoplastic drugs often called cancer chemotherapy. The International Association for the Study of Pain defines neuropathy as “a disturbance of function or pathological change in one of several nerves and may diffuse bilaterally. CIPN is a predominantly sensory neuropathy that may be accompanied by motor and autonomic changes. CIPN symptoms usually emerge late, that is, weeks or months after the completion of chemotherapy, with their severity being usually proportional to the cumulative dose of the drug. Some patients experience paradoxical worsening and/or intensification of symptoms after the cessation of treatment, as well as a phenomenon known as “coasting”, where either mild neuropathy worsens or new CIPN develops. Pain and sensory abnormalities may persist for months or even years after the cessation of chemotherapy.

Clinically, CIPN manifests itself as deficits in sensory, motor and/or autonomic functions of a varying intensity. Sensory symptoms usually develop first, involve the feet and hands and commonly present as a typical “glove and stocking” neuropathy with the most distal parts of the limbs exhibiting the greatest deficits. The symptoms comprise numbness, tingling, altered touch sensation, impaired vibration, paresthesias and dysesthesias induced by touch and warm or cool temperatures. Moreover, painful sensations, including spontaneous burning, shooting or electric shock-like pain as well as mechanical or thermal allodynia or hyperalgesia frequently occur. In severe cases, these symptoms can progress to a loss of sensory perception. Motor symptoms occur less frequently than sensory symptoms and, as a rule, assume the form of distal weakness, gait and balance disturbances, and impaired movements. These symptoms have a marked and often underappreciated impact on quality of life and safety, e.g., cancer patients who develop CIPN are three times more likely to fall. In severe cases, CIPN can lead to paresis, complete patient immobilization and severe disability. Sensory disorders occur more frequently than autonomic symptoms, which usually involve orthostatic hypotension, constipation and altered sexual or urinary function.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### Synthetic Methods

Compounds are prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art.

General details. All oxygen and/or moisture-sensitive reactions were carried out under nitrogen (N₂) atmosphere in glassware that had been flame-dried under vacuum (approximately 0.5 mm Hg) and purged with N₂ prior to use. All reagents and solvents were purchased from commercial vendors and used as received or synthesized according to methods already reported. NMR spectra were recorded on a Bruker 300 (300 MHz ¹H, 75 MHz ¹³C) or Varian UNITY INOVA 500 (500 MHz ¹H, 125 MHz ¹³C) spectrometer. Proton and carbon chemical shifts are reported in ppm (δ) referenced to the NMR solvent. Data are reported as follows: chemical shifts, multiplicity (br=broad, s=singlet, d=doublet, t=triplet, q=quartet, m=multiplet; coupling constant(s) in Hz). Unless otherwise indicated, NMR data were collected at 25° C. Flash chromatography was performed using 40-60 μm Silica Gel (60 Å mesh) on a Teledyne Isco Combiflash R_{f}. Tandem Liquid Chromatography/Mass Spectrometry (LC/MS) was performed on a Waters 2795 separations module and 3100 mass detector. Analytical thin layer chromatography (TLC) was performed on EM Reagent 0.25 mm silica gel 60-F plates.

LCMS_Condition_01: Column: X-Bridge BEH C-18 (3.0×50 mm, 2.5 μm), Mobile Phase: A-0.025% FA in Water, Mobile Phase: B-ACN, Flow Rate: 1.2 mL/min (Gradient).

LCMS Condition_02: Column: X-Select CSH C-18 (150×4.6 mm, 3.5 μm), Mobile Phase: A-0.025% Aq FORMIC ACID, Mobile Phase: B-ACN, Flow Rate: 1.0 mL/min (Gradient).

HPCL_Condition_01: Column: XSELECT CSH C18 (150×4.6 mm, 3.5μ), Mobile Phase-A: 0.05% TFA: ACETONITRILE (95:05), Mobile Phase-B: ACETONITRILE: 0.05% TFA (95:05), Flow: 1.0 mL/min, Diluent: ACN: Water.

HPLC_Condition_02: Column: XSELECT CSH C18 (150×4.6 mm, 3.5μ), Mobile Phase-A: 5 mM Ammonium acetate, Mobile Phase-ACN, Flow: 1.0 mL/min, Diluent: ACN: Water.

Reverse phase PREP Purification methods: Preparative Column X-SELECT (250*30 mm), 5u Mobile Phase A 10 mm ABC in Water Mobile Phase B ACN Flow rate 25 mL, Instrument ID Prep-14 Gradient (Time/% B) 0/10, 3/10, 10/25, 20/40, 30/55, 40/60, 50/75, 60/95.

Compounds of Formula (I) were prepared following the synthetic schemes and procedures are described in detail below.

### Example 1A. Synthesis of Compounds of Formula (I)

### 2-(difluoromethyl)-5-(6-((imidazo[1,2-a]pyrazin-8-yloxy)methyl)pyridin-3-yl)-1,3,4-oxadiazole (Compound A)

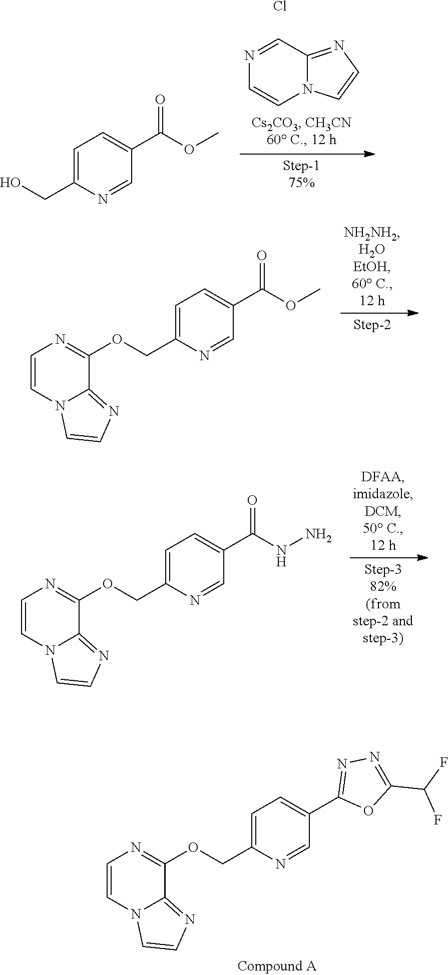 [SMILES: Cl ; COC(=O)c1ccc(CO)nc1 ; c1cn2ccnc2cn1 ; COC(=O)c1ccc(COc2nccn3ccnc23)nc1 ; NNC(=O)c1ccc(COc2nccn3ccnc23)nc1 ; FC(F)c1nnc(-c2ccc(COc3nccn4ccnc34)nc2)o1]

Step-1: Synthesis of methyl 6-((imidazo[1,2-a]pyrazin-8-yloxy)methyl)nicotinate: To a solution of (methyl 6-(hydroxymethyl)pyridine-3-carboxylate (5.0 g, 29.91 mmol) in acetonitrile (100 mL) in were added Cs₂CO₃ (17.0 g, 52.17 mmol) and 8-chloroimidazo[1,2-a]pyrazine (4.1 g, 27 mmol) at room temperature and stirred for 5 min. The reaction mixture was heated at 60° C. temperature and stirred for 12 h. Progress of reaction was monitored by TLC. After complete consumption of the starting material, the reaction mixture was cooled to room temperature and concentrated under reduced pressure. The residue obtained was dissolved in water (100 mL) and extracted with ethyl acetate (2×250 mL). Organic layer was washed with brine (100 mL), dried over anhydrous Na₂SO₄ and concentrated under reduced pressure to obtain crude product. The crude product was purified by Combi Flash column chromatography using methanol in DCM (0-10%) to afford methyl 5-fluoro-6-methylnicotinate (2.70 g, 75%) as an off-white solid. LC-MS: m/z 285.4 [M+H].

Step-2: Synthesis of 6-((imidazo[1,2-a]pyrazin-8-yloxy)methyl)nicotinohydrazide: To a solution of methyl 6-(imidazo[1,2-a]pyrazin-8-yloxymethyl)pyridine-3-carboxylate (4.2 g, 15 mmol) in ethanol (100 mL) were added hydrazine hydride (3.5 g, 110 mmol) at room temperature and stirred for 5 min. The reaction mixture was heated at 70° C. temperature and stirred for 12 h. Progress of reaction was monitored by TLC. After complete consumption of the starting material, the reaction mixture was cooled to room temperature and concentrated under reduced pressure to afford 6-((imidazo[1,2-a]pyrazin-8-yloxy)methyl)nicotinohydrazide (3.6 g, crude). The crude was as such used for next reaction without carried out further purification. LC-MS: m/z 286.0 [M+H].

Step-3: Synthesis of 2-(difluoromethyl)-5-(6-((imidazo[1,2-a]pyrazin-8-yloxy)methyl)pyridin-3-yl)-1,3,4-oxadiazole (A): To a solution of (6-(imidazo[1,2-a]pyrazin-8-yloxymethyl)pyridine-3-carbohydrazide (6.05 g, 21.3 mmol) in DCM (200 mL) were added imidazole (4.34 g, 62.5 mmol) and difluoroacetic anhydride (11.12 g, 54.31 mmol) at 0° C. temperature and stirred for 5 min. The resulting reaction mixture was heated at 50° C. temperature and stirred for 4 h. Progress of reaction was monitored by TLC. After complete consumption of the starting material, the reaction mixture was cooled to room temperature and concentrated under reduced pressure. The residue obtained was dissolved in water (100 mL) and extracted with DCM (2×250 mL). Organic layer was washed with brine (100 mL), dried over anhydrous Na₂SO₄ and concentrated under reduced pressure to obtain crude product. The crude product was purified by Combi Flash column chromatography using methanol in DCM (0-5%) to afford methyl 5-fluoro-6-methylnicotinate (A, 6.0 g, 82%, From 2 steps) as an off-white solid. LC-MS: m/z 344.93 [M+H]. ¹H NMR (400 MHz, DMSO-d₆) δ ppm 9.22 (d, J=1.47 Hz, 1H), 8.48 (dd, J=8.25, 2.14 Hz, 1H), 8.27 (d, J=4.65 Hz, 1H), 8.12 (d, J=0.98 Hz, 1H), 7.69-7.77 (m, 2H), 7.37-7.59 (m, 2H), 5.74 (s, 2H). HPLC=97.54%, RT=6.815 min.

### 2-(Difluoromethyl)-5-(6-(((5-fluoropyridin-3-yl)oxy)methyl)pyridin-3-yl)-1,3,4-oxadiazole (19)

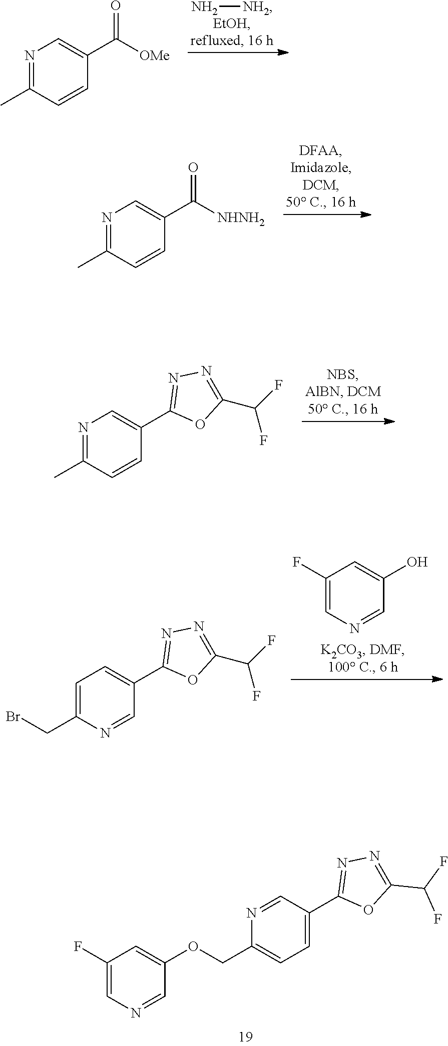

Step-1: Synthesis of 6-methylnicotinohydrazide: To a stirred solution of methyl 6-methylnicotinate (3.0 g, 19.867 mmol, 1.0 equiv.) in EtOH (15 mL) were added hydrazine hydride (4.0 g, 79.47 mmol, 4.0 equiv.) at room temperature and stirred for 5 min. The reaction mixture was refluxed for 16 h and progress of reaction was monitored by TLC. After completion of reaction, reaction mixture was cooled to room temperature and concentrated under reduced pressure to obtain crude product. The obtained crude product was triturated with diethyl ether (50 mL) to afford desired product (2.0 g, 93%) as a white solid. LC-MS: m/z 151.00 (M+1).

Step-2: Synthesis of 2-(difluoromethyl)-5-(6-methylpyridin-3-yl)-1,3,4-oxadiazole: To a solution of 6-methylnicotinohydrazide (2, 2.8 g, 18.543 mmol, 1.0 equiv.) in DCM (20 mL) were added imidazole (3.70 g, 55.62 mmol, 3.0 equiv.), and DFAA (9.64 g, 55.62 mmol, 3.0 equiv.) at room temperature and stirred for 5 min. The resulting reaction mixture was heated at 50° C. and stirred for 16 h. The progress of reaction was monitored by TLC. After completion of the reaction, the reaction mixture was quenched with sat. aq. NaHCO₃ (10 mL) and aq. layer was extracted with DCM (30 mL×2). The organic layer was washed with brine, dried over anhydrous Na₂SO₄ and concentrated under reduced pressure to obtain the crude product (2.40 g) as a sticky solid. The crude product was purified by CombiFlash column chromatography using an eluent ethyl acetate: n-heptane (15%) to afford desired product (1.80 g, 47%) as a white solid. LC-MS: m/z 212.00 (M+1).

Step-3: Synthesis of 2-(6-(bromomethyl)pyridin-3-yl)-5-(difluoromethyl)-1,3,4-oxadiazole: To a solution of 2-(difluoromethyl)-5-(6-methylpyridin-3-yl)-1,3,4-oxadiazole (1.80 g, 8.53 mmol, 1.0 equiv.) in DCM (15 mL) were added NBS (2.30 g, 12.79 mmol, 1.5 equiv.) followed by AIBN (420 mg, 2.55 mmol, 0.3 equiv.) at RT. Reaction mixture was stirred at 50° C. for 16 h. The progress of reaction was monitored by TLC and TLC showed new spot of product along with unreacted starting material. After completion of reaction, the reaction mixture was cooled to RT and concentrated under reduced pressure to obtain crude product. The crude product was purified by CombiFlash column chromatography using an eluent ethyl acetate: n-heptane (10%) to provide the product (950 mg, 39%). LCMS: m/z 292.00 (M+1).

Step-4: Synthesis of 2-(difluoromethyl)-5-(6-(((5-fluoropyridin-3-yl)oxy)methyl)pyridin-3-yl)-1,3,4-oxadiazole (19): To a stirred solution of 5-fluoropyridin-3-ol (46.0 mg, 0.4136 mmol, 1.2 equiv.) in DMF (2 mL) was added K₂CO₃ (142 mg, 1.034 mmol, 3.0 equiv) at room temperature and stirred for 15 min. To the resulting reaction mixture was added 2-(6-(bromomethyl)pyridin-3-yl)-5-(difluoromethyl)-1,3,4-oxadiazole (100 mg, 0.3447 mmol, 1.0 equiv.) at room temperature. The reaction mixture was heated at 100° C. for further 6 h. The progress of reaction was monitored by TLC. After completion of reaction, reaction mixture was quenched with saturated NH₄Cl solution (5 mL) and aq. layer was extracted with ethyl acetate (10 mL×2). The organic layer was washed with water (10 mL) followed by brine (10 mL), dried over anhydrous Na₂SO₄ and concentrated under reduced pressure to obtain crude product. The crude product was purified by CombiFlash chromatography eluted with ethyl acetate: n-heptane (40%) to afford desired product (19, 30 mg, 27%) as a pale yellow solid. ¹H NMR (400 MHz, DMSO-d₆) δ ppm 5.43 (s, 2H) 7.45-7.71 (m, 2H) 7.83 (d, J=8.4 Hz, 1H) 8.14-8.24 (m, 1H), 8.34 (br. s, 1H), 8.52 (dd, J=2.4 Hz, J=10.4 Hz, 1H), 9.20-9.30 (m, 1H). ¹⁹F-NMR (400 MHz, DMSO-d₆): δ −120.70, 126.28. LC-MS: m/z 322.85 [M+H].

The following compounds were prepared in a manner analogous to that used for preparing compound (19).

| Compound | Structure | Characterization |
|---|---|---|
| 213 | 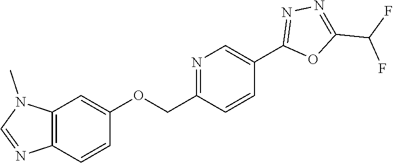 [SMILES: Cn1cnc2ccc(OCc3ccc(-c4nnc(C(F)F)o4)cn3)cc21] | ¹HNMR (400 MHz, METHANOL-d4) δ ppm 9.32 (s, 1H) 9.20 (s, 1H) 8.58 (dd, J = 8.26, 2.00 Hz, 1H), 7.94 (d, J = 8.13 Hz, 1 H) 7.79 (d, J = 9.01 Hz, 1H) 7.60 (d, J = 2.00 Hz, 1H) 7.41-7.48 (m, 1H), 7.13-7.40 (m, 1H) 5.48 (s, 2H) 4.12 (s, 3 H); MS (ESI): 358 [M + H]⁺ |
| 208 | 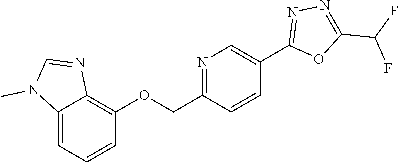 [SMILES: Cn1cnc2c(OCc3ccc(-c4nnc(C(F)F)o4)cn3)cccc21] | ¹HNMR (400 MHz, METHANOL-d4) δ ppm 9.28 (d, J = 1.75 Hz, 1H) 8.54 (dd, J = 8.32, 2.19 Hz, 1H) 8.11 (t, J = 3.94 Hz, 2H) 7.13-7.43 (m, 3 H) 6.89 (d, J = 7.13 Hz, 1H) 5.53 (s, 2H) 3.92 (s, 3 H); MS (ESI): 358.2 [M + H]⁺ |
| 204 | 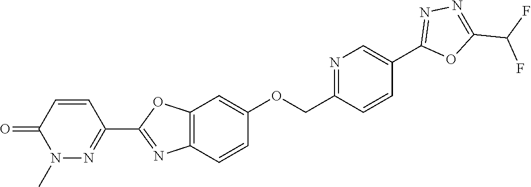 [SMILES: Cn1nc(-c2nc3ccc(OCc4ccc(-c5nnc(C(F)F)o5)cn4)cc3o2)ccc1=O] | ¹HNMR (400 MHz, DMSO-d6) δ ppm 9.25 (d, J = 1.00 Hz, 1H) 8.52 (dd, J = 8.07, 1.94 Hz, 1H) 8.13 (d, J = 9.63 Hz, 1H) 7.85 (d, J = 8.00 Hz, 1H) 7.80 (d, J = 8.76 Hz, 1H) 7.46-7.72 (m, 2H) 7.22 (dd, J = 8.82, 2.19 Hz, 1H) 7.14 (d, J = 9.76 Hz, 1H) 5.44 (s, 2H) 3.80 (s, 3 H) MS (ESI): 453.2 [M + H]⁺ |
| 150 | 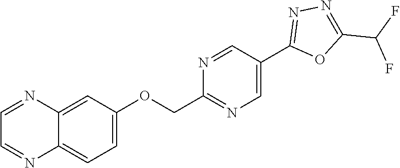 [SMILES: FC(F)c1nnc(-c2cnc(COc3ccc4nccnc4c3)nc2)o1] | ¹HNMR (400 MHz, METHANOL-d4) δ = 9.49 (s, 2H), 8.80 (d, J = 1.9 Hz, 1H), 8.75 (d, J = 2.0 Hz, 1H), 8.06 (d, J = 9.3 Hz, 1H), 7.71-7.68 (m, 1H), 7.50 (d, J = 2.9 Hz, 1H), 7.42-7.15 (m, 1H), 5.67 (s, 2H) ¹⁹F NMR (377 MHz, METHANOL- d4) δ = −122.69 (s, 2F) MS (ESI): 357.0 [M + H]⁺ |
| 156 | 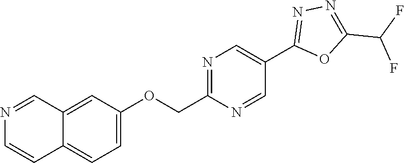 [SMILES: FC(F)c1nnc(-c2cnc(COc3ccc4ccncc4c3)nc2)o1] | MS (ESI): 356.0 [M + H]⁺ ¹HNMR (400 MHz, DMSO-d6) δ = 9.47 (s, 2H), 9.15 (s, 1H), 8.38 (d, J = 5.6 Hz, 1H), 7.96-7.93 (m, 1H), 7.78- 7.75 (m, 1H), 7.62-7.56 (m, 3H), 5.64 (s, 2H) ¹⁹F NMR (377 MHz, DMSO-d6) δ = −120.69 (s, 2F) |
| 158 | 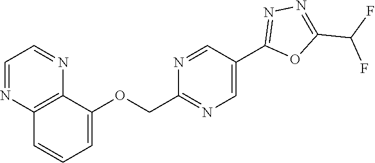 [SMILES: FC(F)c1nnc(-c2cnc(COc3cccc4nccnc34)nc2)o1] | MS (ESI): 357.0 [M + H]⁺ ¹HNMR (400 MHz, METHANOL-d4) δ = 9.46 (s, 2H), 8.95 (d, J = 1.3 Hz, 1H), 8.89 (s, 1H), 7.76-7.71 (m, 2H), 7.41-7.15 (m, 2H), 5.76 (s, 2H) ¹⁹F NMR (377 MHz, METHANOL- d4) δ = −122.37-−122.52 (m, 2F) |
| 154 | 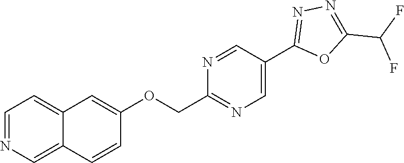 [SMILES: FC(F)c1nnc(-c2cnc(COc3ccc4cnccc4c3)nc2)o1] | MS (ESI): 356.0 [M + H]⁺ ¹HNMR (400 MHz, DMSO-d6) δ = 9.47 (s, 2H), 9.17 (s, 1H), 8.39 (d, J = 5.8 Hz, 1H), 8.07 (d, J = 8.9 Hz, 1H), 7.74-7.61 (m, 2H), 7.45-7.39 (m, 2H), 5.64 (s, 2H) ¹⁹F NMR (377 MHz, DMSO-d6) δ = −119.99-−121.36 (m, 2F) |
| 142 | 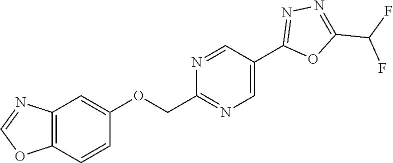 [SMILES: FC(F)c1nnc(-c2cnc(COc3ccc4ocnc4c3)nc2)o1] | MS (ESI): 346.0 [M + H]⁺ ¹HNMR (400 MHz, DMSO-d6) δ = 9.46 (s, 2H), 8.69 (s, 1H), 7.69 (d, J = 8.9 Hz, 1H), 7.63-7.45 (m, 1H), 7.41 (d, J = 2.6 Hz, 1H), 7.14 (dd, J = 2.6, 8.9 Hz, 1H), 5.51 (s, 2H) ¹⁹F NMR (377 MHz, DMSO-d6) δ = −120.68 (s, 2F) |
| 146 | 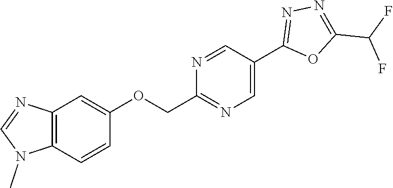 [SMILES: Cn1cnc2cc(OCc3ncc(-c4nnc(C(F)F)o4)cn3)ccc21] | MS (ESI): 359.0 [M + H]⁺ ¹HNMR (400 MHz, DMSO-d6) δ = 9.45 (s, 2H), 8.10 (br s, 1H), 7.84- 7.57 (m, 1H), 7.50-7.45 (m, 1H), 7.21 (br s, 1H), 7.02 (dd, J = 1.6, 8.7 Hz, 1H), 5.45 (s, 2H), 3.80 (s, 3H) ¹⁹F NMR (377 MHz, DMSO-d6) δ = −120.52-−120.89 (m, 2F) |
| 203 | 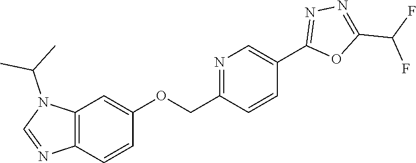 [SMILES: CC(C)n1cnc2ccc(OCc3ccc(-c4nnc(C(F)F)o4)cn3)cc21] | ¹HNMR (400 MHz, DMSO-d6) δ = 9.24 (d, J = 1.5 Hz, 1H), 8.50 (dd, J = 2.2, 8.2 Hz, 1H), 8.22 (s, 1H), 7.85 (d, J = 8.1 Hz, 1H), 7.71-7.48 (m, 2H), 7.33 (d, J = 2.3 Hz, 1H), 6.97 (dd, J = 2.4, 8.8 Hz, 1H), 5.39 (s, 2H), 4.76-4.67 (m, 1H), 1.51 (d, J = 6.8 Hz, 6H) ¹⁹F NMR (377 MHz, DMSO-d6) δ = −120.08-−121.05 (m, 2F) |
| 212 | 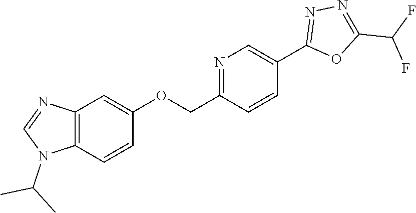 [SMILES: CC(C)n1cnc2cc(OCc3ccc(-c4nnc(C(F)F)o4)cn3)ccc21] | MS (ESI): 386.0 [M + H]⁺ ¹HNMR (400 MHz, DMSO-d6) δ = 9.23 (s, 1H), 8.48 (dd, J = 1.9, 8.2 Hz, 1H), 8.26 (s, 1H), 7.81 (d, J = 8.3 Hz, 1H), 7.65-7.44 (m, 2H), 7.28 (d, J = 2.0 Hz, 1H), 7.04 (dd, J = 2.1, 8.8 Hz, 1H), 5.36 (s, 2H), 4.71 (td, J = 6.6, 13.4 Hz, 1H), 1.52 (d, J = 6.8 Hz, 6H) ¹⁹F NMR (377 MHz, DMSO-d6) δ = −120.02-−121.17 (m, 2F) |
| 209 | 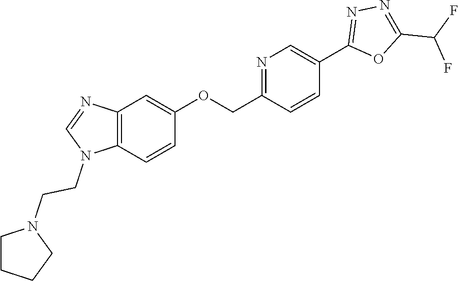 [SMILES: FC(F)c1nnc(-c2ccc(COc3ccc4c(c3)ncn4CCN3CCCC3)nc2)o1] | MS (ESI): 441.0 [M + H]⁺ ¹HNMR (400 MHz, METHANOL-d4) δ = 9.46-9.36 (m, 1H), 9.27 (br d, J = 1.3 Hz, 1H), 8.61 (dd, J = 2.2, 8.1 Hz, 1H), 7.89 (br d, J = 8.1 Hz, 2H), 7.44-7.14 (m, 3H), 5.31 (br t, J = 7.4 Hz, 2H), 4.97 (s, 2H), 4.19-4.12 (m, 2H), 4.01-3.93 (m, 2H), 3.85-3.76 (m, 2H), 2.44-2.35 (m, 4H) ¹⁹F NMR (376 MHz, METHANOL- d4) δ = −76.92 (br s, 3F), −122.37 (s, 2F) |
| 152 | 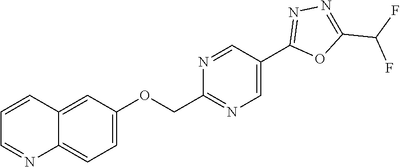 [SMILES: FC(F)c1nnc(-c2cnc(COc3ccc4ncccc4c3)nc2)o1] | MS (ESI): 356.2 [M + H]⁺ ¹HNMR (400 MHz, DMSO-d6) δ = 9.47 (s, 2H), 8.75 (dd, J = 1.6, 4.2 Hz, 1H), 8.21 (d, J = 8.5 Hz, 1H), 7.96 (d, J = 9.1 Hz, 1H), 7.75-7.52 (m, 2H), 7.49- 7.45 (m, 1H), 7.43 (d, J = 2.9 Hz, 1H), 5.61 (s, 2H) ¹⁹F NMR (377 MHz, DMSO-d6) δ = −120.69 (s, 2F) |
| 144 | 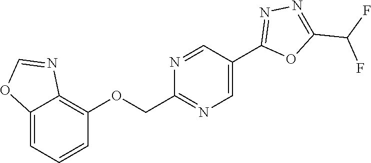 [SMILES: FC(F)c1nnc(-c2cnc(COc3cccc4ocnc34)nc2)o1] | MS (ESI): 346.2 [M + H]⁺ ¹HNMR (400 MHz, METHANOL-d4) δ = 9.45 (s, 2H), 8.36 (s, 1H), 7.41- 7.15 (m, 3H), 7.00 (d, J = 7.9 Hz, 1H), 5.78 (s, 2H) ¹⁹F NMR (376 MHz, METHANOL- d4) δ = −122.44 (s, 2F) |
| 134 | 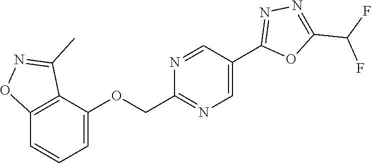 [SMILES: Cc1noc2cccc(OCc3ncc(-c4nnc(C(F)F)o4)cn3)c12] | MS (ESI): 360.2 [M + H]⁺ ¹HNMR (400 MHz, METHANOL-d4) δ = 9.48 (s, 2H), 7.47 (t, J = 8.3 Hz, 1H), 7.42-7.15 (m, 2H), 6.81 (d, J = 8.1 Hz, 1H), 5.63 (s, 2H), 2.73 (s, 3H) ¹⁹F NMR (376 MHz, METHANOL- d4) δ = −122.43 (s, 2F) |
| 160 | 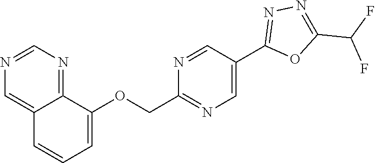 [SMILES: FC(F)c1nnc(-c2cnc(COc3cccc4cncnc34)nc2)o1] | MS (ESI): 357.2 [M + H]⁺ ¹HNMR (400 MHz, METHANOL-d4) δ = 9.55 (s, 1H), 9.45 (s, 2H), 9.27 (s, 1H), 7.75-7.72 (m, 1H), 7.69-7.64 (m, 1H), 7.52 (d, J = 7.8 Hz, 1H), 7.28 (t, J = 51.6 Hz, 1H), 5.77 (s, 2H) ¹⁹F NMR (376 MHz, METHANOL- d4) δ = −122.44 (s, 2F) |
| 210 | 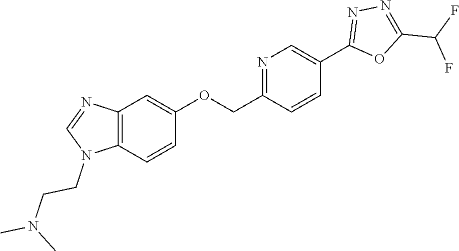 [SMILES: CN(C)CCn1cnc2cc(OCc3ccc(-c4nnc(C(F)F)o4)cn3)ccc21] | MS (ESI): 415.3 [M + H]⁺ ¹HNMR (400 MHz, DMSO-d6) δ = 9.36 (d, J = 2.0 Hz, 1H), 9.04 (s, 1H), 8.63 (dd, J = 2.3, 8.0 Hz, 1H), 7.94 (d, J = 8.1 Hz, 1H), 7.80 (d, J = 8.9 Hz, 1H), 7.62 (t, J = 51.3 Hz, 1H), 7.12 (d, J = 2.0 Hz, 1H), 7.05 (dd, J = 2.0, 8.9 Hz, 1H), 5.05 (br t, J = 7.4 Hz, 2H), 4.92 (s, 2H), 3.96-3.91 (m, 2H), 3.28 (s, 6H) ¹⁹F NMR (376 MHz, DMSO-d6) δ = −73.92 (s, 3F), −120.79 (s, 2F) |
| 202 | 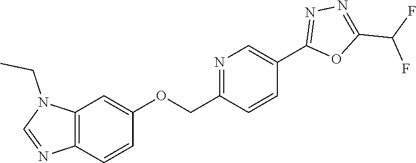 [SMILES: CCn1cnc2ccc(OCc3ccc(-c4nnc(C(F)F)o4)cn3)cc21] | MS (ESI): 372.2 [M + H]⁺ ¹HNMR (400 MHz, DMSO-d6) δ = 9.24 (d, J = 2.0 Hz, 1H), 8.50 (dd, J = 2.3, 8.3 Hz, 1H), 8.13 (s, 1H), 7.84 (d, J = 8.3 Hz, 1H), 7.72-7.46 (m, 2H), 7.33 (d, J = 2.3 Hz, 1H), 6.97 (dd, J = 2.3, 8.8 Hz, 1H), 5.39 (s, 2H), 4.23 (q, J = 7.2 Hz, 2H), 1.39 (t, J = 7.3 Hz, 3H) ¹⁹F NMR (376 MHz, DMSO-d6) δ = −120.70 (s, 2F) |
| 211 | 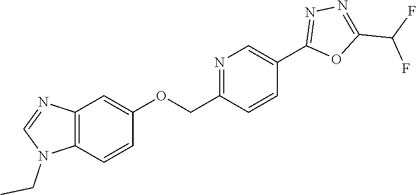 [SMILES: CCn1cnc2cc(OCc3ccc(-c4nnc(C(F)F)o4)cn3)ccc21] | MS (ESI): 372.2 [M + H]⁺ ¹HNMR (400 MHz, DMSO-d6) δ = 9.24 (d, J = 1.5 Hz, 1H), 8.48 (dd, J = 2.1, 8.2 Hz, 1H), 8.18 (s, 1H), 7.81 (d, J = 8.3 Hz, 1H), 7.71-7.45 (m, 2H), 7.28 (d, J = 2.1 Hz, 1H), 7.05 (dd, J = 2.1, 8.8 Hz, 1H), 5.36 (s, 2H), 4.24 (q, J = 7.2 Hz, 2H), 1.40 (t, J = 7.3 Hz, 3H) ¹⁹F NMR (376 MHz, DMSO-d6) δ = −120.70 (s, 2F) |
| 136 | 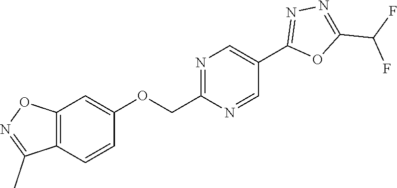 [SMILES: Cc1noc2cc(OCc3ncc(-c4nnc(C(F)F)o4)cn3)ccc12] | ¹HNMR (400 MHz, CDCl3) δ ppm 9.46 (s, 2H), 7.52 (d, J = 8.68 Hz, 1H), 6.83-7.10 (m, 3H), 5.50 (s, 2H), 2.53 (s, 3H). LC-MS: m/z 360.1 [M + H]. |
| 138 | 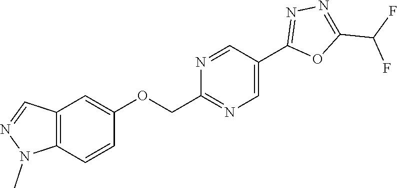 [SMILES: Cn1ncc2cc(OCc3ncc(-c4nnc(C(F)F)o4)cn3)ccc21] | ¹HNMR (400 MHz, DMSO-d6) δ ppm 9.45 (s, 2H), 7.87 (s, 1H), 7.47-7.74 (m, 2H), 7.21 (d, J = 2.25 Hz, 1H), 7.17 (dd, J = 8.94, 2.31 Hz, 1H), 5.45 (s, 2H), 4.00 (s, 3H). LC-MS: m/z 359.0 [M + H]. |
| 140 | 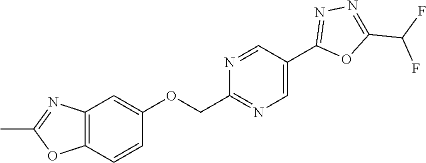 [SMILES: Cc1nc2cc(OCc3ncc(-c4nnc(C(F)F)o4)cn3)ccc2o1] | ¹HNMR (400 MHz, DMSO-d6) δ ppm 9.45 (s, 2H), 7.45-7.76 (m, 2H), 7.27 (d, J = 2.63 Hz, 1H), 7.02 (dd, J = 8.82, 2.56 Hz, 1H), 5.48 (s, 2H), 2.57 (s, 3H). LC-MS: m/z 359.92 [M + H]. |
| 141 | 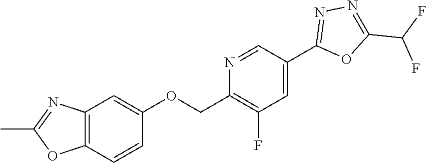 [SMILES: Cc1nc2cc(OCc3ncc(-c4nnc(C(F)F)o4)cc3F)ccc2o1] | ¹HNMR (400 MHz, DMSO-d6) δ ppm 9.10 (d, J = 1.00 Hz, 1H), 8.43-8.48 (m, 1H), 7.44-7.74 (m, 2H), 7.37 (d, J = 2.50 Hz, 1H), 6.97-7.04 (m, 1H), 5.39 (d, J = 1.63 Hz, 2H), 2.40-2.50 (m, 3H). LC-MS: m/z 377.2 [M + H]. |
| 147 | 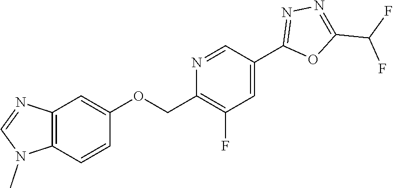 [SMILES: Cn1cnc2cc(OCc3ncc(-c4nnc(C(F)F)o4)cc3F)ccc21] | ¹HNMR (400 MHz, DMSO-d6) δ ppm 9.08-9.13 (m, 1H), 8.42-8.48 (m, 1H), 8.09-8.12 (m, 1H), 7.43-7.74 (m, 2H), 7.34 (d, J = 2.25 Hz, 1H), 6.96-7.02 (m, 1H), 5.38 (d, J = 1.38 Hz, 2H), 3.81 (s, 3H). LC-MS: m/z 376.2 [M + H]. |
| 151 | 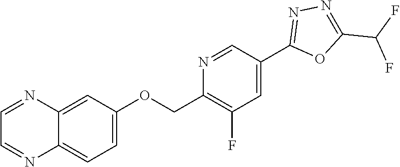 [SMILES: Fc1cc(-c2nnc(C(F)F)o2)cnc1COc1ccc2nccnc2c1] | ¹HNMR (400 MHz, DMSO-d6) δ ppm 9.12 (s, 1H), 8.95 (d, J = 1.75 Hz, 1H), 8.89 (d, J = 1.75 Hz, 1H), 8.50 (dd, J = 9.76, 1.63 Hz, 1H), 7.76-7.82 (m, 1H), 7.67-7.74 (m, 1H), 7.60 (s, 1H), 7.46-7.52 (m, 1H), 5.60 (d, J = 1.50 Hz, 2H). LC-MS: m/z 373.91 [M + H]. |
| 153 | 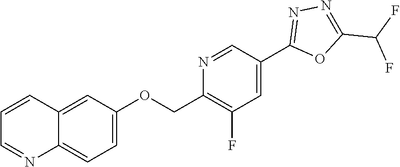 [SMILES: Fc1cc(-c2nnc(C(F)F)o2)cnc1COc1ccc2ncccc2c1] | ¹HNMR (400 MHz, DMSO-d6) δ ppm 9.58 (br. s, 1H), 9.25 (s, 1H), 9.11 (s, 1H), 8.51 (dd, J = 1.6 Hz, J = 11.2 Hz, 1H), 8.26 (d, J = 7.2 Hz, 1H), 7.47- 7.75 (m, 4H), 5.60 (br. s, 2H). LC-MS: m/z 373.2 [M + H]. |
| 155 | 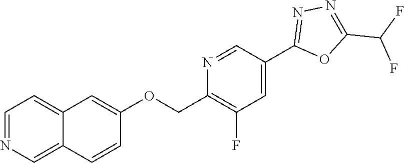 [SMILES: Fc1cc(-c2nnc(C(F)F)o2)cnc1COc1ccc2cnccc2c1] | ¹HNMR (400 MHz, DMSO-d6) δ ppm 9.08-9.21 (m, 2H), 8.36-8.53 (m, 2H), 8.06 (d, J = 9.01 Hz, 1H), 7.68- 7.75 (m, 1H), 7.50-7.61 (m, 2H), 7.31- 7.47 (m, 1H), 5.49-5.59 (m, 2H). LC-MS: m/z 373.2 [M + H]. |
| 157 | 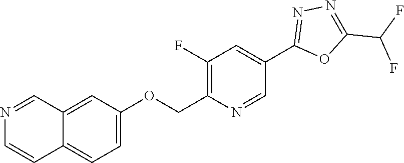 [SMILES: Fc1cc(-c2nnc(C(F)F)o2)cnc1COc1ccc2ccncc2c1] | ¹HNMR (400 MHz, DMSO-d6) δ ppm 9.21-9.21 (m, 1H), 9.12 (s, 1H), 8.49 (dd, J = 9.82, 1.69 Hz, 1H), 8.39 (d, J = 5.63 Hz, 1H), 7.93 (d, J = 9.01 Hz, 1H), 7.77 (d, J = 5.63 Hz, 1H), 7.69- 7.72 (m, 1H), 7.60 (s, 1H), 7.46-7.54 (m, 1H), 5.54 (d, J = 1.50 Hz, 2H). LC-MS: m/z: 373.2 [M + H]⁺ |
| 159 | 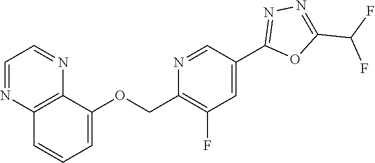 [SMILES: Fc1cc(-c2nnc(C(F)F)o2)cnc1COc1cccc2nccnc12] | ¹HNMR (400 MHz, DMSO-d6) δ ppm 9.12 (s, 1H), 8.95 (d, J = 1.75 Hz, 1H), 8.89 (d, J = 1.75 Hz, 1H), 8.50 (dd, J = 9.76, 1.75 Hz, 1H), 7.76-7.82 (m, 1H), 7.67-7.74 (m, 1H), 7.43-7.62 (m, 2H), 5.60 (d, J = 1.63 Hz, 2H). LC-MS: m/z 374.2 [M + H]. |
| 161 | 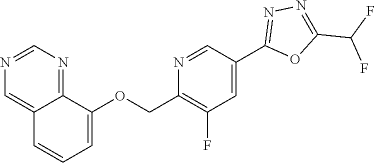 [SMILES: Fc1cc(-c2nnc(C(F)F)o2)cnc1COc1cccc2cncnc12] | ¹HNMR (400 MHz, DMSO-d6) δ ppm 9.59 (s, 1H), 9.25 (s, 1H), 9.12 (s, 1H), 8.50 (dd, J = 9.76, 1.63 Hz, 1H), 7.65- 7.76 (m, 4H), 5.57-5.64 (m, 2H). LC-MS: m/z 373.88 [M + H]. |
| 171 | 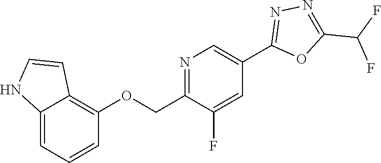 [SMILES: Fc1cc(-c2nnc(C(F)F)o2)cnc1COc1cccc2[nH]ccc12] | ¹HNMR (400 MHz, DMSO-d6) δ ppm 11.10 (br. s, 1H), 9.08-9.12 (m, 1H), 8.46 (dd, J = 9.78, 1.71 Hz, 1H), 7.74 (t, J = 51.2 Hz, 1H), 7.18-7.24 (m, 1H), 7.02-7.05 (m, 1H), 6.95-7.01 (m, 1H), 6.64 (dd, J = 7.46, 0.73 Hz, 1H), 6.37-6.41 (m, 1H), 5.41-5.46 (m, 2H). LC-MS: m/z 361.35 [M + H]. |
| 177 | 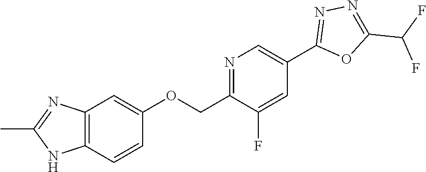 [SMILES: Cc1nc2cc(OCc3ncc(-c4nnc(C(F)F)o4)cc3F)ccc2[nH]1] | ¹HNMR (400 MHz, DMSO-d6) δ ppm 14.26 (bs, 1H), 9.10 (d, J = 0.98 Hz, 1H), 8.48 (dd, J = 9.78, 1.71 Hz, 1H), 7.57-7.74 (m, 2H), 7.34-7.49 (m, 1H), 7.13-7.19 (m, 1H), 5.46 (d, J = 1.47 Hz, 2H) 2.70 (s, 3H). LC-MS: m/z 376.3 [M + H]. |

### 2-(Difluoromethyl)-5-[6-(8-quinolyloxymethyl)-3-pyridyl]-1,3,4-oxadiazole (10)

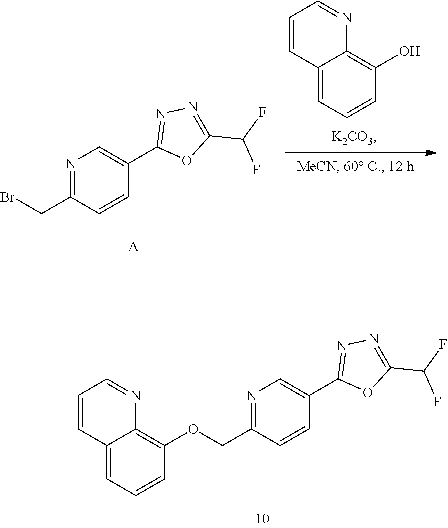

To a solution of 2-[6-(bromomethyl)-3-pyridyl]-5-(difluoromethyl)-1,3,4-oxadiazole (150 mg, 517.13 umol, 1 eq) and quinolin-8-ol (75.07 mg, 517.13 umol, 89.36 μL, 1 eq) in MeCN (4 mL) was added K₂CO₃ (142.94 mg, 1.03 mmol, 2 eq) at 25° C. Then the mixture was stirred at 60° C. for 12 hr. The mixture was concentrated under reduced pressure. The residue was purified by prep-HPLC (neutral) to obtain 2-(difluoromethyl)-5-[6-(8-quinolyloxymethyl)-3-pyridyl]-1,3,4-oxadiazole (18.0 mg, 50.24 umol, 9.72% yield, 98.9% purity) as a white solid. ¹H NMR (400 MHz, METHANOL-d₄) δ=9.31 (d, J=1.6 Hz, 1H), 8.90 (dd, J=1.6, 4.3 Hz, 1H), 8.52 (dd, J=2.1, 8.3 Hz, 1H), 8.38 (dd, J=1.6, 8.3 Hz, 1H), 8.04 (d, J=8.3 Hz, 1H), 7.62 (dd, J=4.3, 8.4 Hz, 1H), 7.58-7.55 (m, 1H), 7.54-7.50 (m, 1H), 7.40-7.13 (m, 2H), 5.62 (s, 2H). MS(ESI): 355.0 [M+H]+

### 2-(6-(difluoro(naphthalen-1-yloxy)methyl)pyridin-3-yl)-5-(difluoromethyl)-1,3,4-oxadiazole (14)

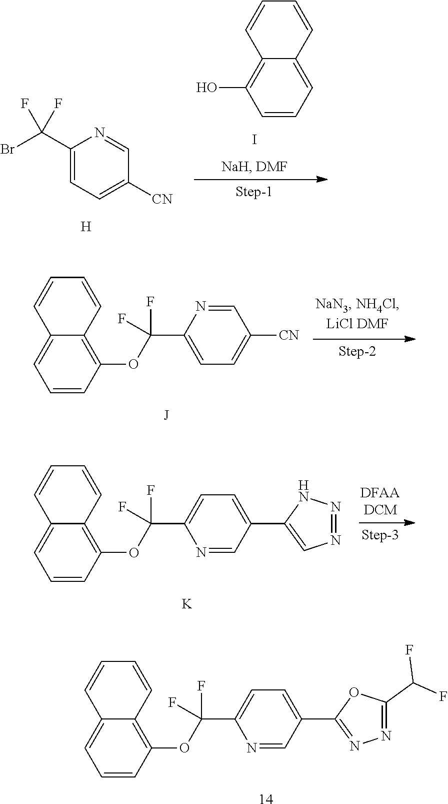

Step-1: 6-(difluoro(naphthalen-1-yloxy)methyl)nicotinonitrile (J): To a stirred solution of compound I (0.19 g, 1.30 mmol) in DMF (2 mL) was added NaH (60%, 0.08 g, 2.00 mmol) at 0° C. and the reaction mixture was stirred for 20 min at the same temperature. To the resulting reaction mixture, Compound H (0.3 g, 1.30 mmol) dissolved in DMF (1 mL) was slowly added at 0° C. and the reaction mixture was stirred at room temperature for 12 h. After completion of reaction (monitored by TLC), the reaction mixture was quenched with cold water (50 mL) and extracted with ethyl acetate (100 mL). The organic layer was dried over sodium sulphate and concentrated under reduced pressure to give the crude product. The crude product was purified by 100-200 mesh silica gel column chromatography by eluting with 0-10% ethyl acetate in hexane to give the compound J (0.32 g, 84.0%) as brown liquid. LC-MS: m/z 296.9 [M+H]⁺.

Step-2: 2-(difluoro(naphthalen-1-yloxy)methyl)-5-(1H-tetrazol-5-yl)pyridine (K): To a stirred solution of compound J (0.32 g, 1.00 mmol) in DMF (5 mL), sodium azide (0.35 g, 5.40 mmol) was added followed by NH₄Cl (0.27 g, 5.40 mmol) and LiCl (0.03 g) and the reaction mixture was stirred at 100° C. for 5 h. After completion of the reaction (monitored by TLC), the reaction mixture was concentrated under reduced pressure and was acidified with 6N HCl. Product was extracted with ethyl acetate (2×50 mL). The organic layer was dried over anhydrous sodium sulphate and concentrated under reduced pressure to afford compound K (0.30 g, 82.0%) as light brown thick liquid which was used as such for the next reaction. LC-MS: m/z 339.9 [M+H]⁺.

Step-3: 2-(6-(difluoro(naphthalen-1-yloxy)methyl)pyridin-3-yl)-5-(difluoromethyl)-1,3,4-oxadiazole (14): To a stirred solution of compound K (0.30 g, 0.88 mmol) in DCM (6 mL), difluoroacetic anhydride (0.5 mL, 4.40 mmol) was added at 0° C. and the reaction mixture was stirred at RT for 16 h. After completion of reaction (monitored by TLC), the reaction mixture was diluted with cold water (20 mL) and extracted with DCM (50 mL). The organic layer was dried over anhydrous sodium sulphate and concentrated under reduced pressure. The crude compound was purified by 100-200 mesh silica gel column chromatography by eluting with 0-7% ethyl acetate in hexane to give (14) (0.10 g, 29.0% yield) as an off-white solid. ¹H NMR (400 MHz, DMSO-d₆) δ ppm 9.43 (d, J=1.48 Hz, 1H), 8.72 (dd, J=8.37, 1.97 Hz, 1H), 8.30 (s, 1H), 8.20 (d, J=7.88 Hz, 1H), 8.00-8.05 (m, 1H), 7.90-7.95 (m, 1H), 7.46-7.79 (m, 5H). ¹⁹F NMR (400 MHz, DMSO-d₆) δ ppm −68 (s, 2F), −122 (s, 2F). LC-MS: m/z 390.15 [M+H]⁺.

The following compounds were prepared in a manner analogous to that used for preparing compounds of Formula (I), above.

| Compound | Structure/Name | Characterization |
|---|---|---|
| 59 | 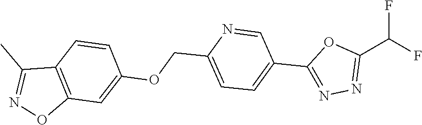 [SMILES: Cc1noc2cc(OCc3ccc(-c4nnc(C(F)F)o4)cn3)ccc12] | ¹H NMR (400 MHz, METHANOL- d₄) δ ppm 9.29 (d, J = 1.63 Hz, 1 H) 8.55 (dd, J = 8.25, 2.13 Hz, 1 H) 7.89 (d, J = 8.63 Hz, 1 H) 7.54 (d, J = 8.76 Hz, 1 H) 7.32 (d, J = 2.38 Hz, 1 H) 7.10-7.29 (m, 2 H) 5.38 (s, 2 H) 2.61 (s, 3 H). MS (ESI): 359.2 [M + H]⁺ |
| | 6-[[5-[5-(difluoromethyl)-1,3,4-oxadiazol-2-yl]-2- | |
| | pyridyl]methoxy]-3-methyl-1,2-benzoxazole | |
| 97 | 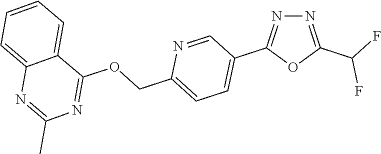 [SMILES: Cc1nc(OCc2ccc(-c3nnc(C(F)F)o3)cn2)c2ccccc2n1] | ¹H NMR (400 MHz, METHANOL- d₄) δ = 9.30 (d, J = 1.6 Hz, 1H), 8.56 (dd, J = 2.3, 8.3 Hz, 1H), 8.39-8.31 (m, 1H), 7.97-7.93 (m, 1H), 7.90 (d, J = 8.3 Hz, 1H), 7.88-7.83 (m, 1H), 7.70-7.65 (m, 1H), 7.40-7.14 (t, J = 51.6 Hz, 1H), 5.90 (s, 2H), 2.69 (s, 3H). MS (ESI): 370.0 [M + H]+ |
| | 2-(difluoromethyl)-5-[6-[(2-methylquinazolin-4- | |
| | yl)oxymethyl]-3-pyridyl]-1,3,4-oxadiazole | |
| 88 | 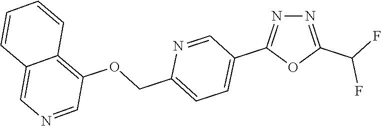 [SMILES: FC(F)c1nnc(-c2ccc(COc3cncc4ccccc34)nc2)o1] 2-(difluoromethyl)-5-[6-(4-isoquinolyloxymethyl)-3- pyridyl]-1,3,4-oxadiazole | ¹H NMR (400 MHz, METHANOL- d₄) δ = 9.33 (d, J = 1.8 Hz, 1H), 8.93 (s, 1H), 8.59 (dd, J = 2.1, 8.1 Hz, 1H), 8.41 (d, J = 8.3 Hz, 1H), 8.17 (s, 1H), 8.13 (d, J = 8.4 Hz, 1H), 8.01 (d, J = 8.1 Hz, 1H), 7.89-7.85 (m, 1H), 7.79-7.75 (m, 1H), 7.41-7.15 (m, 1H), 5.62 (s, 2H). MS (ESI): 355.0 [M + H]+ |
| 82 | 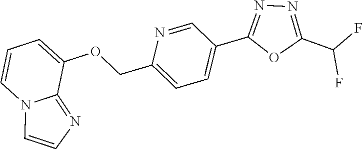 [SMILES: FC(F)c1nnc(-c2ccc(COc3cccn4ccnc34)nc2)o1] | ¹H NMR (400 MHz, METHANOL- d₄) δ = 9.30 (d, J = 1.6 Hz, 1H), 8.57 (dd, J = 2.2, 8.2 Hz, 1H), 8.13-8.11 (m, 2H), 7.88 (d, J = 1.3 Hz, 1H), 7.59 (d, J = 1.1 Hz, 1H), 7.41-7.14 (m, 1H), 6.87-6.81 (m, 2H), 5.53 (s, 2H). MS (ESI): 344.0 [M + H]+ |
| | 2-(difluoromethyl)-5-[6-(imidazo[1,2-a]pyridin-8- | |
| | yloxymethyl)-3-pyridyl]-1,3,4-oxadiazole | |
| 63 | 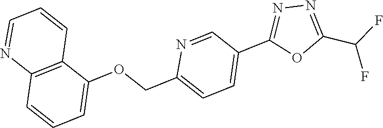 [SMILES: FC(F)c1nnc(-c2ccc(COc3cccc4ncccc34)nc2)o1] | ¹H NMR (400 MHz, METHANOL- d₄) δ = 9.32 (d, J = 1.8 Hz, 1H), 8.90- 8.85 (m, 2H), 8.58 (dd, J = 2.3, 8.3 Hz, 1H), 7.98 (d, J = 8.3 Hz, 1H), 7.73-7.67 (m, 2H), 7.60 (d, J = 4.4, 8.4 Hz, 1H), 7.41-7.14 (m, 2H), 5.57 (s, 2H). MS (ESI): 355.1 [M + H]⁺ |
| | 2-(difluoromethyl)-5-[6-(5-quinolyloxymethyl)-3- | |
| | pyridyl]-1,3,4-oxadiazole | |
| 62 | 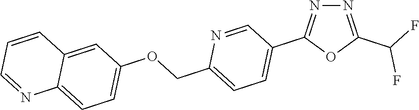 [SMILES: FC(F)c1nnc(-c2ccc(COc3ccc4ncccc4c3)nc2)o1] 2-(difluoromethyl)-5-[6-(6-quinolyloxymethyl)-3- pyridyl]-1,3,4-oxadiazole | ¹H NMR (400 MHz, METHANOL- d₄) δ = 9.32 (d, J = 1.5 Hz, 1H), 8.73 (dd, J = 1.6, 4.3 Hz, 1H), 8.57 (dd, J = 2.3, 8.3 Hz, 1H), 8.30 (d, J = 8.6 Hz, 1H), 8.01 (d, J = 9.3 Hz, 1H), 7.93 (d, J = 8.3 Hz, 1H), 7.62 (dd, J = 2.8, 9.2 Hz, 1H), 7.51 (dd, J = 4.3, 8.3 Hz, 1H), 7.46 (d, J = 2.8 Hz, 1H), 7.40-7.14 (m, 1H), 5.49 (s, 2H). MS (ESI): 355.0 [M + H]⁺ |
| 61 | 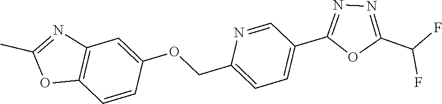 [SMILES: Cc1nc2cc(OCc3ccc(-c4nnc(C(F)F)o4)cn3)ccc2o1] | ¹H NMR (400 MHz, METHANOL- d₄) δ = 9.29 (d, J = 1.9 Hz, 1H), 8.55 (dd, J = 2.1, 8.3 Hz, 1H), 7.89 (d, J = 8.4 Hz, 1H), 7.51 (d, J = 8.9 Hz, 1H), 7.40-7.27 (m, 2H), 7.14-7.11 (m, 1H), 5.37 (s, 2H), 2.63 (s, 3H). MS (ESI): 359.0 [M + H]+ |
| | 5-[[5-[5-(difluoromethyl)-1,3,4-oxadiazol-2-yl]-2- | |
| | pyridyl]methoxy]-2-methyl-1,3-benzoxazole | |
| 58 | 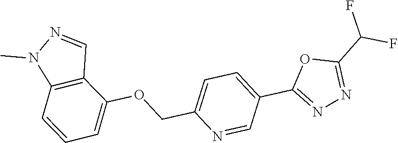 [SMILES: Cn1ncc2c(OCc3ccc(-c4nnc(C(F)F)o4)cn3)cccc21] | ¹H NMR (400 MHz, METHANOL- d₄) δ = 9.30 (d, J = 1.8 Hz, 1H), 8.60- 8.52 (m, 1H), 8.20-8.12 (m, 1H), 7.94 (d, J = 8.3 Hz, 1H), 7.40-7.14 (m, 3H), 6.66 (d, J = 7.6 Hz, 1H), 5.49 (s, 2H), 4.07 (s, 3H). MS (ESI): 358.0 [M + H]+ |
| | 2-(difluoromethyl)-5-[6-[(1-methylindazol-4- | |
| | yl)oxymethyl]-3-pyridyl]-1,3,4-oxadiazole | |
| 57 | 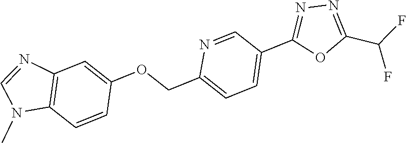 [SMILES: Cn1cnc2cc(OCc3ccc(-c4nnc(C(F)F)o4)cn3)ccc21] | ¹H NMR (400 MHz, METHANOL- d₄) δ = 9.29 (d, J = 1.6 Hz, 1H), 8.54 (dd, J = 2.1, 8.3 Hz, 1H), 8.07 (s, 1H), 7.89 (d, J = 7.8 Hz, 1H), 7.51 (d, J = 8.9 Hz, 1H), 7.40-7.14 (m, 3H), 5.37 (s, 2H), 3.89 (s, 3H). MS (ESI): 358.0 [M + H]+ |
| | 2-(difluoromethyl)-5-[6-[(1-methylbenzimidazol-5- | |
| | yl)oxymethyl]-3-pyridyl]-1,3,4-oxadiazole | |
| 55 | 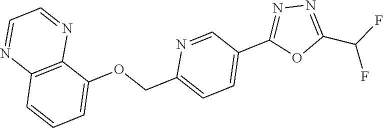 [SMILES: FC(F)c1nnc(-c2ccc(COc3cccc4nccnc34)nc2)o1] 2-(difluoromethyl)-5-[6-(quinoxalin-5-yloxymethyl)- 3-pyridyl]-1,3,4-oxadiazole | ¹H NMR (400 MHz, METHANOL- d₄) δ = 9.31 (d, J = 2.1 Hz, 1H), 8.97 (d, J = 1.9 Hz, 1H), 8.92 (d, J = 1.8 Hz, 1H), 8.55 (dd, J = 2.1, 8.3 Hz, 1H), 8.04 (d, J = 8.5 Hz, 1H), 7.82- 7.74 (m, 2H), 7.43-7.14 (m, 2H), 5.63 (s, 2H). MS (ESI): 356.0 [M + H]+ |
| 38 | 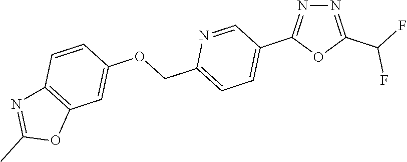 [SMILES: Cc1nc2ccc(OCc3ccc(-c4nnc(C(F)F)o4)cn3)cc2o1] | ¹H NMR (400 MHz, METHANOL- d₄) δ = 9.29 (d, J = 2.0 Hz, 1H), 8.55 (d, J = 2.1, 8.3 Hz, 1H), 7.88 (d, J = 8.4 Hz, 1H), 7.54 (d, J = 8.8 Hz, 1H), 7.40-7.25 (m, 2H), 7.20 (d, J = 8.8 Hz, 1H) 5.38 (s, 2H), 2.61 (s, 3H). MS (ESI): 359.1 [M + H]⁺ |
| | 6-[[5-[5-(difluoromethyl)-1,3,4-oxadiazol-2-yl]-2- | |
| | pyridyl]methoxy]-2-methyl-1,3-benzoxazole | |
| 37 | 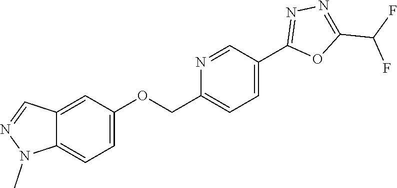 [SMILES: Cn1ncc2cc(OCc3ccc(-c4nnc(C(F)F)o4)cn3)ccc21] | ¹H NMR (400 MHz, METHANOL- d₄) δ = 9.29 (d, J = 1.9 Hz, 1H), 8.55 (d, J = 2.3, 8.3 Hz, 1H), 7.90 (t, J = 4.1 Hz, 2H), 7.55-7.52 (m, 1H), 7.40- 7.27 (m, 3H), 5.36 (s, 2H), 4.06 (s, 3H). MS (ESI): 358.0 [M + H]⁺ |
| | 2-(difluoromethyl)-5-[6-[(1-methylindazol-5- | |
| | yl)oxymethyl]-3-pyridyl]-1,3,4-oxadiazole | |
| 36 | 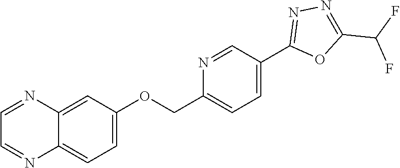 [SMILES: FC(F)c1nnc(-c2ccc(COc3ccc4nccnc4c3)nc2)o1] | ¹H NMR (400 MHz, METHANOL- d₄) δ = 9.33 (d, J = 1.6 Hz, 1H), 8.83 (d, J = 2.0 Hz, 1H), 8.77 (d, J = 1.9 Hz, 1H), 8.57 (d, J = 2.3, 8.3 Hz, 1H), 8.08 (d, J = 9.3 Hz, 1H), 7.93 (d, J = 8.3 Hz, 1H), 7.71 (d, J = 2.8, 9.3 Hz, 1H), 7.56 (d, J = 2.8 Hz, 1H), 7.27 (t, J = 51.6 Hz, 1H), 5.55 (s, 2H). MS (ESI): 356.2 [M + H]⁺ |
| | 2-(difluoromethyl)-5-[6-(quinoxalin-6-yloxymethyl)- | |
| | 3-pyridyl]-1,3,4-oxadiazole | |
| 35 | 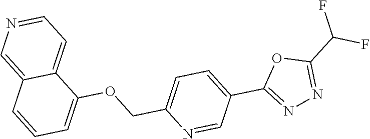 [SMILES: FC(F)c1nnc(-c2ccc(COc3cccc4cnccc34)nc2)o1] | ¹H NMR (400 MHz, METHANOL- d₄) δ = 9.33 (d, J = 2.0 Hz, 1H), 9.24 (s, 1H), 8.59 (d, J = 2.2, 8.2 Hz, 1H), 8.51 (d, J = 5.9 Hz, 1H), 8.28 (d, J = 6.0 Hz, 1H), 8.00 (d, J = 8.3 Hz, 1H), 7.74 (d, J = 8.3 Hz, 1H), 7.64 (t, J = 8.0 Hz, 1H), 7.35 (d, J = 7.8 Hz, 1H), 7.29-7.14 (m, 1H), 5.57 (s, 2H). MS (ESI): 355.1 [M + H]⁺ |
| | 2-(difluoromethyl)-5-[6-(5-isoquinolyloxymethyl)-3- | |
| | pyridyl]-1,3,4-oxadiazole | |
| 34 | 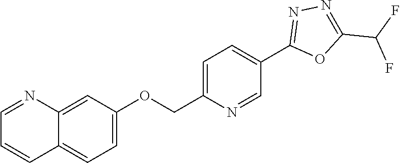 [SMILES: FC(F)c1nnc(-c2ccc(COc3ccc4cccnc4c3)nc2)o1] | ¹H NMR (400 MHz, METHANOL- d₄) δ = 9.32 (d, J = 1.6 Hz, 1H), 8.77 (d, J = 1.7, 4.4 Hz, 1H), 8.56 (d, J = 2.3, 8.3 Hz, 1H), 8.32 (d, J = 1.4, 8.3 Hz, 1H), 7.93 (t, J = 8.4 Hz, 2H), 7.48-7.41 (m, 3H), 7.40-7.14 (m, 1H), 5.52 (s, 2H). MS (ESI): 355.0 [M + H]+ |
| | 2-(difluoromethyl)-5-[6-(7-quinolyloxymethyl)-3- | |
| | pyridyl]-1,3,4-oxadiazole | |
| 33 | 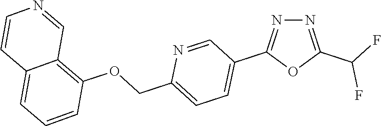 [SMILES: FC(F)c1nnc(-c2ccc(COc3cccc4ccncc34)nc2)o1] | ¹H NMR (400 MHz, METHANOL- d₄) δ = 9.69 (s, 1H), 9.34 (d, J = 1.6 Hz, 1H), 8.59 (d, J = 2.1, 8.3 Hz, 1H), 8.50 (d, J = 5.9 Hz, 1H), 8.00 (d, J = 8.4 Hz, 1H), 7.82 (d, J = 5.4 Hz, 1H), 7.75-7.70 (m, 1H), 7.56 (d, J = 8.1 Hz, 1H), 7.41-7.14 (m, 2H), 5.61 (s, 2H). MS (ESI): 355.0 [M + H]⁺ |
| | 2-(difluoromethyl)-5-[6-(8-isoquinolyloxymethyl)-3- | |
| | pyridyl]-1,3,4-oxadiazole | |
| 32 | 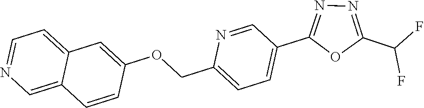 [SMILES: FC(F)c1nnc(-c2ccc(COc3ccc4cnccc4c3)nc2)o1] 2-(difluoromethyl)-5-[6-(6-isoquinolyloxymethyl)-3- pyridyl]-1,3,4-oxadiazole | ¹H NMR (400 MHz, METHANOL- d₄) δ = 9.32 (d, J = 1.8 Hz, 1H), 9.12 (s, 1H), 8.57 (d, J = 2.3, 8.3 Hz, 1H), 8.36 (d, J = 6.0 Hz, 1H), 8.09 (d, J = 9.0 Hz, 1H), 7.92 (d, J = 8.3 Hz, 1H), 7.75 (d, J = 5.9 Hz, 1H), 7.50 (d, J = 2.4, 9.0 Hz, 1H), 7.44 (d, J = 2.4 Hz, 1H), 7.40-7.14 (m, 1H), 5.52 (s, 2H). MS (ESI): 355.0 [M + H]+ |
| 28 | 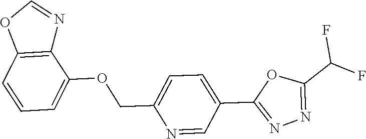 [SMILES: FC(F)c1nnc(-c2ccc(COc3cccc4ocnc34)nc2)o1] | ¹H NMR (400 MHz, METHANOL- d₄) δ = 9.29 (d, J = 1.8 Hz, 1H), 8.56 (d, J = 2.2, 8.3 Hz, 1H), 8.45 (s, 1H), 8.01 (d, J = 8.4 Hz, 1H), 7.43-7.14 (m, 3H), 7.07 (d, J = 7.8 Hz, 1H), 5.63 (s, 2H). MS (ESI): 345.0 [M + H]+ |
| | 4-[[5-[5-(difluoromethyl)-1,3,4-oxadiazol-2-yl]-2- | |
| | pyridyl]methoxy]-1,3-benzoxazole | |
| 53 | 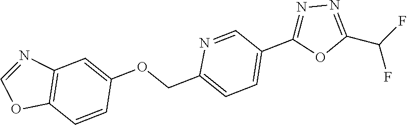 [SMILES: FC(F)c1nnc(-c2ccc(COc3ccc4ocnc4c3)nc2)o1] | ¹H NMR (400 MHz, METHANOL- d₄) δ ppm 9.30 (d, J = 1.75 Hz, 1 H) 8.55 (d, J = 8.32, 2.19 Hz, 1 H) 8.45 (s, 1 H) 7.90 (d, J = 8.63 Hz, 1 H) 7.63 (d, J = 9.01 Hz, 1 H) 7.39-7.41 (m, 1 H) 7.12-7.29 (m, 2 H) 5.40 (s, 2 H) MS (ESI): 345.2 [M + H]⁺ |
| | 5-((5-(5-(difluoromethyl)-1,3,4-oxadiazol-2- | |
| | yl)pyridin-2-yl)methoxy)benzo[d]oxazole | |
| 56 | 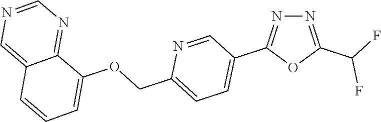 [SMILES: FC(F)c1nnc(-c2ccc(COc3cccc4cncnc34)nc2)o1] | ¹H NMR (400 MHz, METHANOL- d₄) δ = 9.56 (s, 1H), 9.32-9.29 (m, 2H), 8.55 (d, J = 2.1, 8.3 Hz, 1H), 8.05 (d, J = 8.3 Hz, 1H), 7.76-7.69 (m, 2H), 7.59 (d, J = 1.4, 7.4 Hz, 1H), 7.27 (m, 1H), 5.62 (s, 2H) MS (ESI): 356.0 [M + H]⁺ |
| | 2-(difluoromethyl)-5-[6-(quinazolin-8-yloxymethyl)- | |
| | 3-pyridyl]-1,3,4-oxadiazole | |
| 123 | 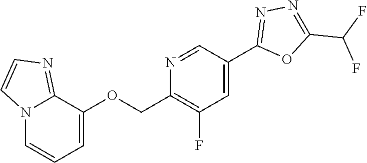 [SMILES: Fc1cc(-c2nnc(C(F)F)o2)cnc1COc1cccn2ccnc12] | ¹H NMR (400 MHz, METHANOL- d₄) δ = 9.03 (s, 1H), 8.26 (d, J = 1.6, 9.5 Hz, 1H), 7.98 (d, J = 6.6 Hz, 1H), 7.72 (d, J = 1.3 Hz, 1H), 7.38 (d, J = 1.3 Hz, 1H), 7.16-7.10 (m, 1H), 6.80- 6.78 (m, 1H), 6.74-6.70 (m, 1H), 5.45 (d, J = 1.9 Hz, 2H) MS (ESI): 362.2 [M + H]⁺ |
| | 2-(difluoromethyl)-5-[6-fluoro-6-(imidazo[1,2- | |
| | a]pyridin-8-yloxymethyl)-3-pyridyl]-1,3,4- | |
| | oxadiazole | |
| 124 | 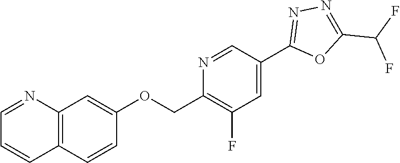 [SMILES: Fc1cc(-c2nnc(C(F)F)o2)cnc1COc1ccc2cccnc2c1] | ¹H NMR (400 MHz, METHANOL- d₄) δ = 9.17 (d, J = 0.6 Hz, 1H), 8.79 (d, J = 1.7, 4.4 Hz, 1H), 8.39 (d, J = 1.8, 9.6 Hz, 1H), 8.32 (d, J = 1.0, 8.3 Hz, 1H), 7.90 (d, J = 9.0 Hz, 1H), 7.58 (d, J = 2.4 Hz, 1H), 7.45-7.15 (m, 3H), 5.57 (d, J = 1.8 Hz, 2H) MS (ESI): 373.0 [M + H]+ |
| | 2-(difluoromethyl)-5-[5-fluoro-6-(7- | |
| | quinolyloxymethyl)-3-pyridyl]-1,3,4-oxadiazole | |
| 125 | 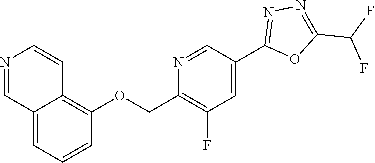 [SMILES: Fc1cc(-c2nnc(C(F)F)o2)cnc1COc1cccc2cnccc12] | ¹H NMR (400 MHz, METHANOL- d₄) δ = 9.20 (s, 1H), 9.17 (s, 1H), 8.43 (d, J = 6.0 Hz, 1H), 8.40 (d, J = 1.7, 9.7 Hz, 1H), 8.10 (d, J = 5.9 Hz, 1H), 7.74-7.70 (m, 1H), 7.66-7.62 (m, 1H), 7.45 (d, J = 7.6 Hz, 1H), 7.28 (s, 1H), 5.63 (d, J = 1.6 Hz, 2H) MS (ESI): 373.0 [M + H]+ |
| | 2-(difluoromethyl)-5-[5-fluoro-6-(5- | |
| | isoquinolyloxymethyl)-3-pyridyl]-1,3,4-oxadiazole | |
| 126 | 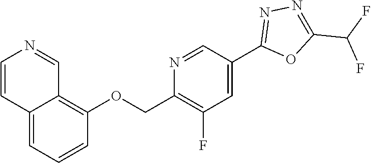 [SMILES: Fc1cc(-c2nnc(C(F)F)o2)cnc1COc1cccc2ccncc12] | ¹H NMR (400 MHz, METHANOL- d₄) δ = 9.52 (s, 1H), 9.19 (s, 1H), 8.46 (d, J = 5.8 Hz, 1H), 8.41 (d, J = 1.7, 9.7 Hz, 1H), 7.81-7.79 (m, 1H), 7.76- 7.72 (m, 1H), 7.55 (d, J = 8.4 Hz, 1H), 7.42-7.16 (m, 2H), 5.67 (d, J = 1.8 Hz, 2H) MS (ESI): 373.0 [M + H]+ |
| | 2-(difluoromethyl)-5-[5-fluoro-6-(8- | |
| | isoquinolyloxymethyl)-3-pyridyl]-1,3,4-oxadiazole | |
| 130 | 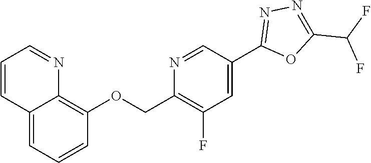 [SMILES: Fc1cc(-c2nnc(C(F)F)o2)cnc1COc1cccc2cccnc12] | ¹H NMR (400 MHz, METHANOL- d₄) δ = 9.15 (s, 1H), 8.80 (d, J = 1.5, 4.3 Hz, 1H), 8.36-8.20 (m, 2H), 7.59- 7.54 (m, 3H), 7.44-7.15 (m, 2H), 5.63 (d, J = 1.4 Hz, 2H) MS (ESI): 373.0 [M + H]+ |
| | 2-(difluoromethyl)-5-[5-fluoro-6-(8- | |
| | quinolyloxymethyl)-3-pyridyl]-1,3,4-oxadiazole | |
| 132 | 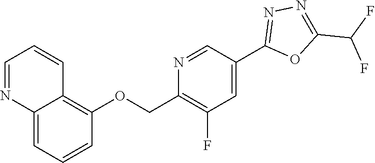 [SMILES: Fc1cc(-c2nnc(C(F)F)o2)cnc1COc1cccc2ncccc12] | ¹H NMR (400 MHz, METHANOL- d₄) δ = 9.18 (s, 1H), 8.85 (d, J = 1.6, 4.3 Hz, 1H), 8.70 (d, J = 0.9, 8.4 Hz, 1H), 8.40 (d, J = 1.7, 9.7 Hz, 1H), 7.75-7.70 (m, 1H), 7.69-7.66 (m, 1H), 7.53 (d, J = 4.4, 8.5 Hz, 1H), 7.42-7.16 (m, 2H), 5.63 (d, J = 1.6 Hz, 2H) MS (ESI): 373.2 [M + H]+ |
| | 2-(difluoromethyl)-5-[5-fluoro-6-(5- | |
| | quinolyloxymethyl)-3-pyridyl]-1,3,4-oxadiazole | |
| 31 | 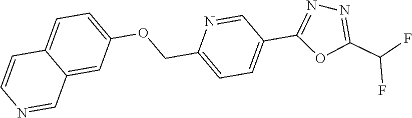 [SMILES: FC(F)c1nnc(-c2ccc(COc3ccc4ccncc4c3)nc2)o1] | ¹H NMR (400 MHz, METHANOL- d₄) δ = 9.32 (d, J = 1.5 Hz, 1H), 9.16 (s, 1H), 8.57 (d, J = 2.2, 8.3 Hz, 1H), 8.35 (d, J = 5.8 Hz, 1H), 7.94 (m, 2H), 7.80 (d, J = 5.8 Hz, 1H), 7.65-7.61 (m, 2H), 7.40-7.14 (m, 1H), 5.51 (s, 2H) MS (ESI): 355.0 [M + H]+ |
| | 2-(difluoromethyl)-5-[6-(7-isoquinolyloxymethyl)-3- | |
| | pyridyl]-1,3,4-oxadiazole | |
| 60 | 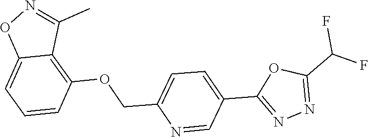 [SMILES: Cc1noc2cccc(OCc3ccc(-c4nnc(C(F)F)o4)cn3)c12] | ¹H NMR (400 MHz, METHANOL- d₄) δ = 9.32 (d, J = 2.1 Hz, 1H), 8.58 (d, J = 2.1, 8.3 Hz, 1H), 7.91 (d, J = 8.3 Hz, 1H), 7.53 (m, 1H), 7.41-7.14 (m, 2H), 6.88 (d, J = 8.0 Hz, 1H), 5.51 (s, 2H), 2.74 (s, 3H) MS (ESI): 359.0 [M + H]⁺ |
| | 4-[[5-[5-(difluoromethyl)-1,3,4-oxadiazol-2-yl]-2- | |
| | pyridyl]methoxy]-3-methyl-1,2-benzoxazole | |
| 95 | 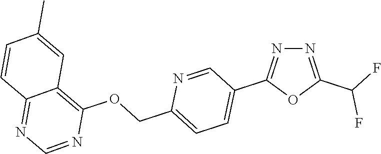 [SMILES: Cc1ccc2ncnc(OCc3ccc(-c4nnc(C(F)F)o4)cn3)c2c1] | ¹H NMR (400 MHz, METHANOL- d₄) δ ppm 9.30 (d, J = 1.6 Hz, 1 H) 8.71 (s, 1 H) 8.56 (d, J = 8.25, 2.25 Hz, 1 H) 8.18 (d, J = 1.00 Hz, 1 H) 7.89 (d, J = 8.25 Hz, 1 H) 7.86 (d, J = 1.13 Hz, 2 H) 7.27 (m, 1 H) 5.91 (s, 2 H) 2.61 (d, J = 0.63 Hz, 3 H) MS (ESI): 370.2 [M + H]⁺ |
| | 2-(difluoromethyl)-5-[6-[(6-methylquinazolin-4- | |
| | yl)oxymethyl]-3-pyridyl]-1,3,4-oxadiazole | |
| 96 | 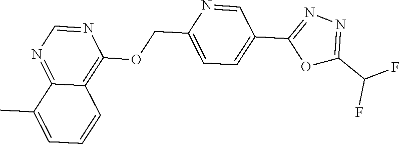 [SMILES: Cc1cccc2c(OCc3ccc(-c4nnc(C(F)F)o4)cn3)ncnc12] | ¹H NMR (400 MHz, METHANOL- d₄) δ ppm 9.30 (d, J = 1.63 Hz, 1 H) 8.78 (s, 1 H) 8.55 (d, J = 8.25, 2.25 Hz, 1 H) 8.24 (d, J = 8.88 Hz, 1 H) 7.88 (d, J = 7.88 Hz, 1 H) 7.83 (d, J = 7.25 Hz, 1 H) 7.58-7.66 (m, 1 H) 7.27 (m, 1 H) 5.90 (s, 2 H) 2.74 (s, 3 H) MS (ESI): 370.0 [M + H]⁺ |
| | 2-(difluoromethyl)-5-[6-[(8-methylquinazolin-4- | |
| | yl)oxymethyl]-3-pyridyl]-1,3,4-oxadiazole | |
| 98 | 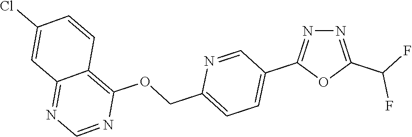 [SMILES: FC(F)c1nnc(-c2ccc(COc3ncnc4cc(Cl)ccc34)nc2)o1] | ¹H NMR (400 MHz, METHANOL- d₄) δ ppm 9.29 (d, J = 1.75 Hz, 1 H) 8.79 (s, 1 H) 8.56 (d, J = 8.32, 2.19 Hz, 1 H) 8.40 (d, J = 8.88 Hz, 1 H) 7.98 (d, J = 1.88 Hz, 1 H) 7.90 (d, J = 8.25 Hz, 1 H) 7.74 (d, J = 8.82, 1.94 Hz, 1 H) 7.27 (t, J = 51.59 Hz, 1 H) 5.92 (s, 2 H) MS (ESI): 390.1 [M + H]⁺ |
| | 2-[6-[(7-chloroquinazolin-4-yl)oxymethyl]-3- | |
| | pyridyl]-5-(difluoromethyl)-1,3,4-oxadiazole | |

### 2-(6-(bromomethyl)-5-fluoropyridin-3-yl)-5-(difluoromethyl)-1,3,4-oxadiazole (Int-K)

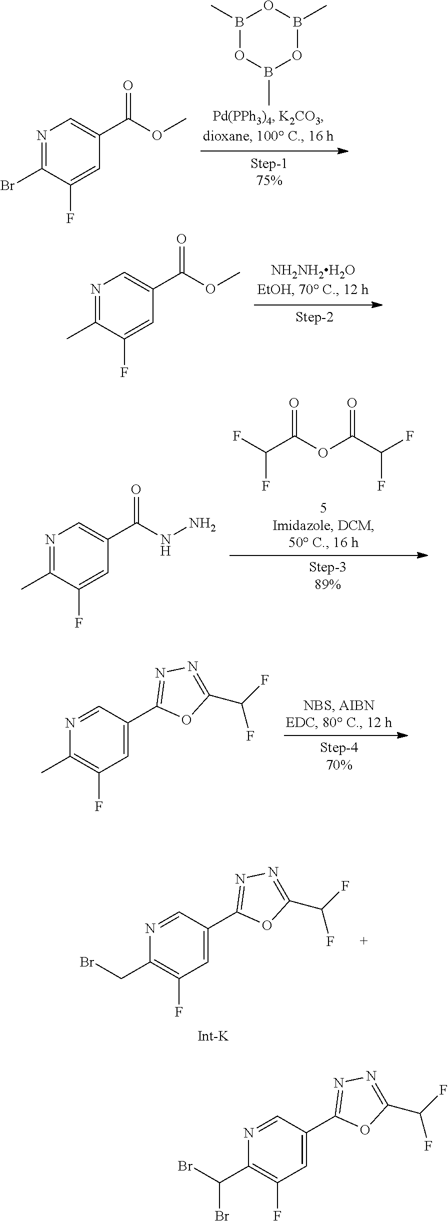

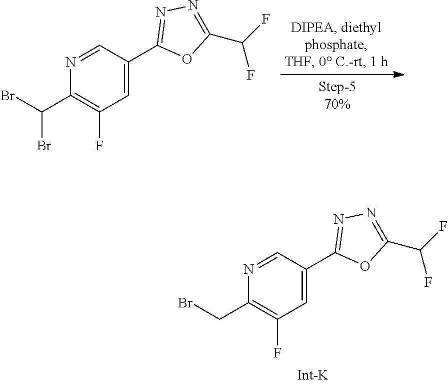 [SMILES: Fc1cc(-c2nnc(C(F)F)o2)cnc1C(Br)Br ; Fc1cc(-c2nnc(C(F)F)o2)cnc1CBr]

Step-1: Synthesis of methyl 5-fluoro-6-methylnicotinate: To a stirred solution of methyl 6-bromo-5-fluoronicotinate (1, 5.0 g, 21.36 mmol, 1.0 equiv.) in 1,4 dioxane (75 mL) was added 2,4,6-trimethyl-1,3,5,2,4,6-trioxatriborinane (13.41 g, 106.8 mmol, 5.0 equiv.) followed by K₂CO₃ (4.429 g, 32.00 mmol, 1.5 equiv.) at room temperature and degassed the reaction mixture with argon for 15 min. To the resulting reaction mixture was added Pd(PPh₃)₄ (2.77 g, 0.235 mmol, 0.11 eq.). The reaction mixture was heated at 100° C. and stirred for 16 h. After complete consumption of the starting material (monitored by TLC), the reaction mixture was diluted with water (100 mL) and extracted with DCM (2×100 mL). The combined organic layer was washed with brine solution (100 mL), dried over anhydrous Na₂SO₄ and concentrated under reduced pressure to obtain crude product. The crude product was purified by CombiFlash column chromatography using ethyl acetate: n-hexane (20%) to afford methyl 5-fluoro-6-methylnicotinate (2.70 g, 75%) as an off white solid.

Step-2: Synthesis of 5-fluoro-6-methylnicotinohydrazide: To a stirred solution of methyl 5-fluoro-6-methylnicotinate (0.740 g, 4.374 mmol, 1.0 equiv.) in EtOH (10 mL) was added hydrazine hydrate (1.402 g, 43.74 mmol, 5.0 equiv.) at room temperature. The reaction mixture was heated at 70° C. and stirred for 12 h. After complete consumption of the starting material (monitored by TLC), the reaction mixture was concentrated under reduced pressure to obtain crude product (0.740 g, crude). The crude product was as such used for next reaction without carried out further purification.

Step-3: Synthesis of 2-(difluoromethyl)-5-(5-fluoro-6-methylpyridin-3-yl)-1,3,4-oxadiazole: To a stirred solution of methyl 5-fluoro-6-methylnicotinohydrazide (0.740 g, 4.374 mmol, 1.0 equiv.) in DCM (50 mL) was added imidazole (0.893 g, 13.12 mmol, 3.0 equiv.) at room temperature for 15 min. To the resulting reaction mixture was added DFAA (2.28 g, 13.12 mmol) at 0° C. temperature. The reaction mixture was heated at 50° C. and stirred for 16 h. After complete consumption of the starting material (monitored by TLC), the reaction mixture was diluted with water (10 mL) and extracted with DCM (3×20 mL). The combined organic layer was washed with brine solution (20 mL), dried over anhydrous Na₂SO₄ and concentrated under reduced pressure to obtain crude product. The crude product was purified by Combi Flash column chromatography using ethyl acetate: n-heptane (30%) to afford 2-(difluoromethyl)-5-(5-fluoro-6-methylpyridin-3-yl)-1,3,4-oxadiazole (1.788 g, 89.4%, from 2 steps) as an off white solid.

Step-4: Synthesis of 2-(6-(bromomethyl)-5-fluoropyridin-3-yl)-5-(difluoromethyl)-1,3,4-oxadiazole (Int-K): To a stirred solution of methyl 5-fluoro-6-methylnicotinohydrazide 2-(difluoromethyl)-5-(5-fluoro-6-methylpyridin-3-yl)-1,3,4-oxadiazole (0.900 g, 3.927 mmol, 1.0 equiv.) in DCE (18 mL) was added NBS (2.10 g, 23.56 mmol, 6.0 equiv.) followed by AIBN (0.322 g, 1.963 mmol, 0.5 equiv.) at room temperature for 15 min. The reaction mixture was heated at 80° C. and stirred for 12 h. After complete consumption of the starting material (monitored by TLC), the reaction mixture was diluted with water (30 mL) and extracted with ethyl acetate (3×20 mL). The combined organic layer was washed with brine solution (30 mL), dried over anhydrous Na₂SO₄ and concentrated under reduced pressure to obtain crude product. The crude product was purified by CombiFlash column chromatography using ethyl acetate: n-heptane (30%) to afford 2-(6-(bromomethyl)-5-fluoropyridin-3-yl)-5-(difluoromethyl)-1,3,4-oxadiazole (Int-K, 0.600 g, 44%, 7, 550 mg, 35%) as an off white solid.

LC-MS: m/z=309.6. ¹H NMR (400 MHz, CDCl₃) δ ppm 9.12 (s, 1H), 8.13 (br. d, J=8.80 Hz, 1H), 6.76-7.10 (m, 1H), 4.66 (s, 3H).

Step-5: Synthesis of 2-(6-(bromomethyl)-5-fluoropyridin-3-yl)-5-(difluoromethyl)-1,3,4-oxadiazole (Int-K): To a stirred solution of 2-(6-(dibromomethyl)-5-fluoropyridin-3-yl)-5-(difluoromethyl)-1,3,4-oxadiazole (0.370 g, 0.953 mmol, 1.0 equiv.) in THF (10 mL) was added DIPEA (0.246 g, 1.907 mmol, 2.0 equiv.) followed by Diethyl phosphate (263.1 g, 1.907 mmol, 1.907 equiv.) at 0° C. temperature. The resulting reaction mixture was allowed to attain room temperature and stirred for 1 h. After complete consumption of the starting material (monitored by TLC), the reaction mixture was diluted with water (10 mL) and extracted with ethyl acetate (3×15 mL). Organic layer was washed with brine solution (10 mL), dried over anhydrous Na₂SO₄ and concentrated under reduced pressure to obtain crude product. The crude product was purified by CombiFlash column chromatography using ethyl acetate: n-heptane (30%) to afford 2-(6-(bromomethyl)-5-fluoropyridin-3-yl)-5-(difluoromethyl)-1,3,4-oxadiazole (Int-K, 0.687 g, 72.3%) as an off white solid. LC-MS: m/z=310.11; ¹H NMR (400 MHz, CDCl₃) δ ppm 9.12 (s, 1H), 8.13 (d, J=8.80 Hz, 1H), 6.76-7.10 (m, 1H), 4.66 (s, 3H).

### 2-(Difluoromethyl)-5-(5-fluoro-6-((naphthalen-2-yloxy) methyl) pyridin-3-yl)-1,3,4-oxadiazole (182)

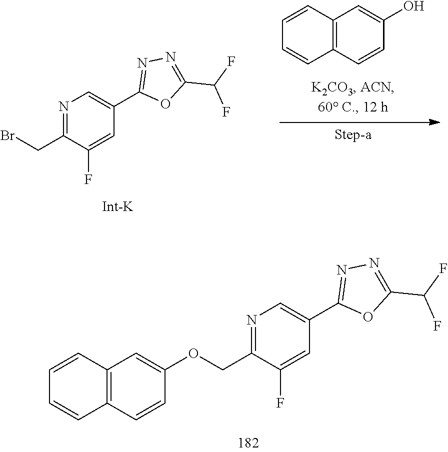 [SMILES: Fc1cc(-c2nnc(C(F)F)o2)cnc1CBr ; Oc1ccc2ccccc2c1 ; Fc1cc(-c2nnc(C(F)F)o2)cnc1COc1ccc2ccccc2c1]

2-(difluoromethyl)-5-(5-fluoro-6-((naphthalen-2-yloxy) methyl) pyridin-3-yl)-1,3,4-oxadiazole (182): To a stirred solution of naphthalen-2-ol (47 mg, 0.324 mmol, 1.0 equiv.) in acetonitrile (5 mL) was added K₂CO₃ (113 mg, 0.817 mmol, 2.5 equiv.) followed by 2-(difluoromethyl)-5-(5-fluoro-6-((naphthalen-2-yloxy)methyl)pyridin-3-yl)-1,3,4-oxadiazole (Int-K, 100 mg, 0.324 mmol, 1.0 equiv.) at room temperature. The reaction mixture was heated at 60° C. and stirred for 12 h. After complete consumption of the starting material (monitored by TLC), the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by CombiFlash column chromatography using ethyl acetate: n-hexane (30%) to afford 2-(difluoromethyl)-5-[5-fluoro-6-(2-naphthyloxymethyl)-3-pyridyl]-1,3,4-oxadiazole (182, 70.0 mg, 40%) as an off white solid. ¹H NMR (400 MHz, DMSO-d₆) δ ppm 9.12 (d, J=1.13 Hz, 1H), 8.45-8.51 (m, 1H), 7.79-7.88 (m, 3H), 7.44-7.73 (m, 3H), 7.34-7.40 (m, 1H), 7.25 (dd, J=8.80, 2.40 Hz, 1H), 5.48 (br. s, 2H). ¹⁹F NMR (400 MHz, DMSO-d₆): δ −120.75; −123.29. LC-MS: m/z=371.9 [M+H]. HPLC: 99.56% at 9.207 min.

### 2-(Difluoromethyl)-5-(5-fluoro-6-((quinazolin-6-yloxy)methyl)pyridin-3-yl)-1,3,4-oxadiazole (181)

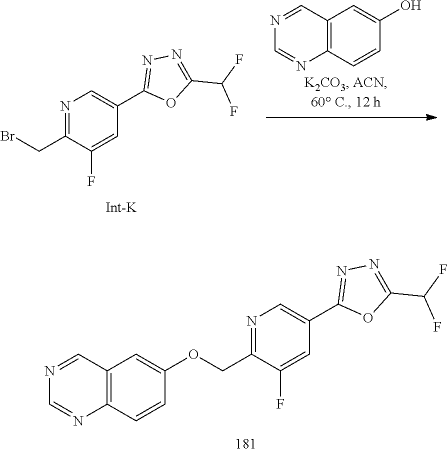

Synthesis of 2-(difluoromethyl)-5-(5-fluoro-6-((quinazolin-6-yloxy)methyl)pyridin-3-yl)-1,3,4-oxadiazole (181): To a stirred solution of quinazolin-6-ol (47 mg, 0.321 mmol, 1.0 equiv.) in acetonitrile (5 mL) was added K₂CO₃ (112 mg, 0.400 mmol, 1.20 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material (monitored by TLC), the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by CombiFlash column chromatography using ethyl acetate: n-hexane (30%) to afford 2-(difluoromethyl)-5-(5-fluoro-6-((quinazolin-6-yloxy)methyl)pyridin-3-yl)-1,3,4-oxadiazole (181, 57 mg, 30%) as an off white solid. ¹H NMR (400 MHz, DMSO-d₆) δ ppm 9.49 (s, 1H), 9.19 (s, 1H), 9.12 (s, 1H), 8.50 (dd, J=10.1, 2.0 Hz, 1H), 7.98 (d, J=9.01 Hz, 1H), 7.46-7.80 (m, 3H), 5.56 (d, J=1.50 Hz, 2H). ¹⁹F NMR (400 MHz, DMSO-d₆): δ −120.75; −123.29. LC-MS: m/z 371.9 [M+H]. HPLC: 99.56% at 9.207 min.

### 2-(difluoromethyl)-5-(5-fluoro-6-((quinazolin-7-yloxy)methyl)pyridin-3-yl)-1,3,4-oxadiazole (180)

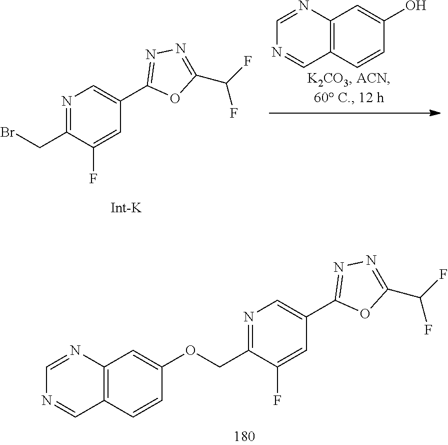

Synthesis of 2-(difluoromethyl)-5-(5-fluoro-6-((quinazolin-7-yloxy)methyl)pyridin-3-yl)-1,3,4-oxadiazole (180): To a stirred solution of quinazolin-7-ol (47 mg, 0.321 mmol, 1.0 equiv.) in acetonitrile (5 mL) was added K₂CO₃ (113 mg, 0.817 mmol, 2.5 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material (monitored by TLC), the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by Combi Flash column chromatography using ethyl acetate: n-hexane (30%) to afford 2-(difluoromethyl)-5-(5-fluoro-6-((quinazolin-7-yloxy)methyl)pyridin-3-yl)-1,3,4-oxadiazole (180, 65 mg, 55%) as an off white solid. ¹H NMR (400 MHz, DMSO-d₆) δ ppm 9.44 (s, 1H), 9.18 (s, 1H), 9.12 (s, 1H), 8.51 (dd, J=9.82, 1.69 Hz, 1H), 8.10 (d, J=9.01 Hz, 1H), 7.55-7.75 (m, 2H), 7.40-7.50 (m, 1H), 5.62 (br. s, 2H). ¹⁹F NMR (400 MHz, DMSO-d₆): δ −120.75; −123.29. LC-MS: m/z 374.4 [M+H]. HPLC: 97.51% at 9.483 min

### 2-(Difluoromethyl)-5-(5-fluoro-6-((quinazolin-5-yloxy)methyl)pyridin-3-yl)-1,3,4-oxadiazole (179)

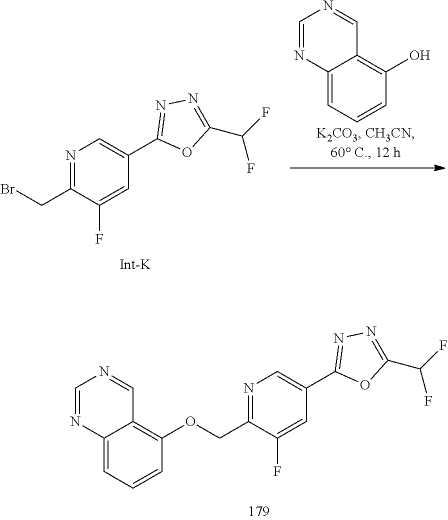 [SMILES: Fc1cc(-c2nnc(C(F)F)o2)cnc1CBr ; Oc1cccc2ncncc12 ; Fc1cc(-c2nnc(C(F)F)o2)cnc1COc1cccc2ncncc12]

Synthesis of 2-(difluoromethyl)-5-(5-fluoro-6-((quinazolin-5-yloxy)methyl)pyridin-3-yl)-1,3,4-oxadiazole (179): To a stirred solution of quinazolin-5-ol (47 mg, 0.321 mmol, 1.0 equiv.) in acetonitrile (5 mL) was added K₂CO₃ (112 mg, 0.817 mmol, 2.5 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material (monitored by TLC), the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by Combi Flash column chromatography using ethyl acetate: n-hexane (30%) to afford 2-(difluoromethyl)-5-(5-fluoro-6-((quinazolin-5-yloxy)methyl)pyridin-3-yl)-1,3,4-oxadiazole (179, 37 mg, 30%) as an off white solid. ¹H NMR (400 MHz, DMSO-d₆) δ ppm 9.67 (s, 1H), 9.30 (s, 1H), 9.11 (s, 1H), 8.52 (t, J=32.6, 1H), 8.52 (dd, J=9.82, 1.69 Hz, 1H), 7.47-7.72 (m, 2H), 7.41 (d, J=7.6 Hz, 1H), 5.69 (br. s, 2H). ¹⁹F NMR (400 MHz, DMSO-d₆): δ −120.76; −123.10. LC-MS: m/z 374.4 [M+H]. HPLC: 98.83% at 5.611 min.

### 2-(Difluoromethyl)-5-(5-fluoro-6-(((2-methyl-2H-indazol-5-yl)oxy)methyl)pyridin-3-yl)-1,3,4-oxadiazole (178)

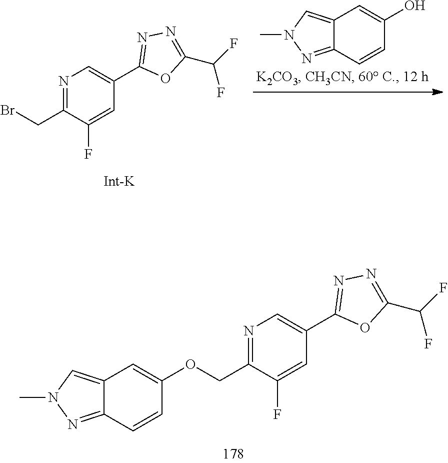

Synthesis of 2-(difluoromethyl)-5-(5-fluoro-6-(((2-methyl-2H-indazol-5-yl)oxy)methyl)pyridin-3-yl)-1,3,4-oxadiazole (178): To a stirred solution of 2-methyl-2H-indazol-5-ol (47.12 mg, 0.317 mmol, 1.0 equiv.) in acetonitrile (10 mL) was added K₂CO₃ (113 mg, 0.817 mmol, 2.5 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material (monitored by TLC), the reaction mixture was cooled to room temperature and quenched with water (10 mL) and extracted with ethyl acetate (3×10 mL). The combined organic layer was washed with brine solution (10 mL), dried over anhydrous Na₂SO₄ and concentrated under reduced pressure to obtain crude product. The crude product was purified by Combi Flash column chromatography using ethyl acetate: n-heptane (30%) to afford 2-(difluoromethyl)-5-(5-fluoro-6-(((2-methyl-2H-indazol-5-yl)oxy)methyl)pyridin-3-yl)-1,3,4-oxadiazole (178, 25 mg, 28%) as an off white solid. ¹H NMR (400 MHz, DMSO-d₆) δ ppm 9.10 (s, 1H), 8.47 (dd, J=1.6 Hz, 11.6 Hz, 1H), 8.17 (s, 1H), 7.47-7.72 (m, 2H), 7.15-7.25 (m, 1H), 6.97 (dd, J=1.0.1 Hz, 1.60 Hz, 1H), 5.69 (br. s, 2H), 4.11 (s, 3H). ¹⁹F NMR (400 MHz, DMSO-d₆): δ −120.75; −123.33. LC-MS: m/z 376.6 [M+H]. HPLC: 96.40% at 6.876 min.

### 6-((5-(5-(difluoromethyl)-1,3,4-oxadiazol-2-yl)-3-fluoropyridin-2-yl)methoxy)-3-methylbenzo[d]isoxazole (137)

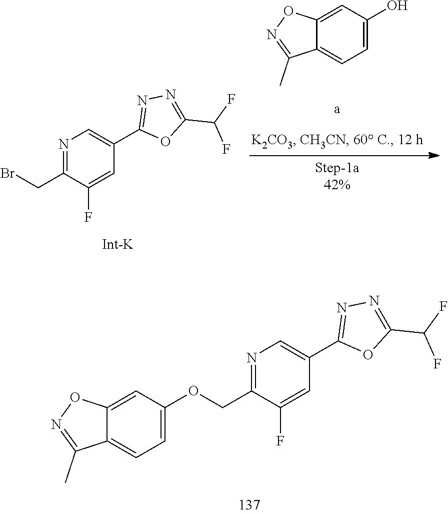 [SMILES: Cc1noc2cc(O)ccc12 ; Fc1cc(-c2nnc(C(F)F)o2)cnc1CBr ; Cc1noc2cc(OCc3ncc(-c4nnc(C(F)F)o4)cc3F)ccc12]

Synthesis of 6-((5-(5-(difluoromethyl)-1,3,4-oxadiazol-2-yl)-3-fluoropyridin-2-yl)methoxy)-3-methylbenzo[d]isoxazole (137): To a stirred solution of 3-methyl-1H-isoindol-6-ol (a, 36.4 mg, 0.244 mmol) in acetonitrile (5 mL) was added K₂CO₃ (101 mg, 0.733 mmol) followed by 2-[6-(bromomethyl)-5-fluoro-3-pyridyl]-5-(difluoromethyl)-1,3,4-oxadiazole (Int-K, 75 mg, 0.244 mmol) at room temperature. The reaction mixture was heated at 60° C. and stirred for 12 h. After complete consumption of the starting material (monitored by TLC), the reaction mixture was cooled to room temperature, quenched with water (10 mL) and extracted with ethyl acetate (15 mL). Organic layer was washed with brine (15 mL), dried over anhydrous Na₂SO₄ and concentrated under reduced pressure to obtain crude product. The crude product was purified by Prep HPLC to afford 6-((5-(5-(difluoromethyl)-1,3,4-oxadiazol-2-yl)-3-fluoropyridin-2-yl)methoxy)-3-methylbenzo[d]isoxazole (137, 38 mg, 42%) as an off white solid. ¹H NMR (400 MHz, CDCl₃) δ ppm 9.18 (s, 1H), 8.19 (dd, J=9.82, 1.69 Hz, 1H), 7.50 (d, J=8.40 Hz, 1H), 7.15 (s, 1H), 6.82-7.07 (d, J=2.13 Hz, 2H), 5.42 (br. s, 2H), 2.53 (s, 3H). LC-MS: m/z: 377.2 [M+H]⁺. HPLC: 99.72% at 8.362 min.

### 2-(difluoromethyl)-5-(5-fluoro-6-(((1-methyl-1H-indazol-5-yl)oxy)methyl)pyridin-3-yl)-1,3,4-oxadiazole (139)

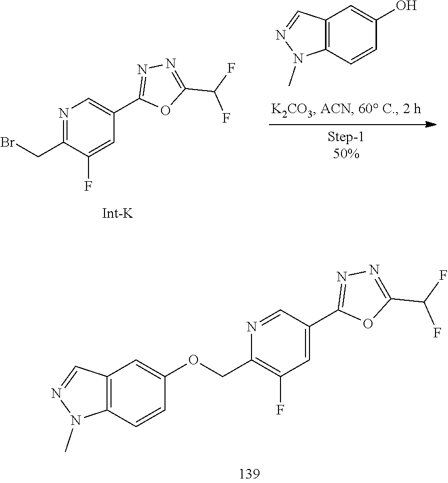 [SMILES: Fc1cc(-c2nnc(C(F)F)o2)cnc1CBr ; Cn1ncc2cc(O)ccc21 ; Cn1ncc2cc(OCc3ncc(-c4nnc(C(F)F)o4)cc3F)ccc21]

Synthesis of 2-(difluoromethyl)-5-(5-fluoro-6-(((1-methyl-1H-indazol-5-yl)oxy)methyl)pyridin-3-yl)-1,3,4-oxadiazole (139): To a stirred solution of 1-methyl-1H-indazol-5-ol (36.19 mg, 0.2442 mmol) in acetonitrile (5 mL) was added K₂CO₃ (101.28 mg, 0.7328 mmol) followed by 2-(6-(bromomethyl)-5-fluoropyridin-3-yl)-5-(difluoromethyl)-1,3,4-oxadiazole (Int-K, 75 mg, 0.2442 mmol) at room temperature. The reaction mixture was heated at 60° C. and stirred for 2 h. After complete consumption of the starting material (monitored by TLC), the reaction mixture was cooled to room temperature and quenched with water (2 mL) and extracted with ethyl acetate (5 mL). Organic layer was washed with brine (5 mL), dried over anhydrous Na2SO4 and concentrated under reduced pressure to obtain crude product. The crude product was purified by Combi Flash column chromatography using ethyl acetate: n-heptane (30%) to afford 2-(difluoromethyl)-5-(5-fluoro-6-(((1-methyl-1H-indazol-5-yl)oxy)methyl)pyridin-3-yl)-1,3,4-oxadiazole (139, 61 mg, 50%) as an off white solid. ¹H NMR (400 MHz, DMSO-d₆) δ ppm 9.10 (s, 1H), 8.45 (dd, J=9.82, 1.69 Hz, 1H), 7.92 (d, J=0.75 Hz, 1H), 7.55-7.60 (m, 2H), 7.35 (d, J=2.13 Hz, 1H), 7.13 (dd, J=9.01, 2.38 Hz, 1H), 5.37 (d, J=1.63 Hz, 2H), 4.01 (s, 3H). LC-MS: m/z: 376.44 [M+H]+. HPLC: 98.23% at 8.170 min.

### 2-(6-(([1,2,4]triazolo[4,3-a]pyridin-7-yloxy)methyl)pyridin-3-yl)-5-(difluoromethyl)-1,3,4-oxadiazole (184)

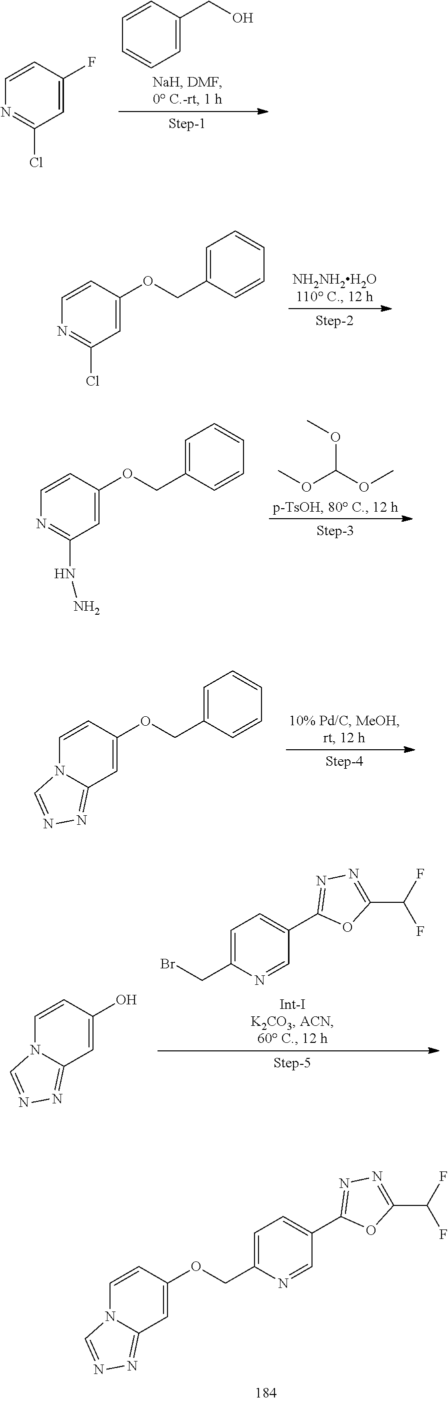

Step-1: Synthesis of 4-(benzyloxy)-2-chloropyridine: To a stirred solution of phenylmethanol (4 g, 40 mmol) in DMF (100 mL, 1290 mmol) was added Sodium hydride (1.3 g, 49 mmol, 1.2 eq) followed by 2-chloro-4-fluoro-pyridine (1, 5 g, 38.011 mmol, 0.95 eq) at 0° C. and stirred for 5 min. The reaction mixture was allowed to attain room temperature and stirred for 30 min. After complete consumption of the starting material (monitored by TLC), the reaction mixture quenched with water (100 mL) and extracted with ethyl acetate (3×50 mL). The combined organic layer was washed with brine solution (50 mL), dried over anhydrous Na₂SO₄ and concentrated under reduced pressure to obtain crude product. The crude product was purified by CombiFlash column chromatography using ethyl acetate: n-heptane (15%) to afford 4-(benzyloxy)-2-chloropyridine (4.7 g, 57%) as an off white solid.

Step-2: Synthesis of 4-(benzyloxy)-2-hydrazineylpyridine: To a stirred solution of 4-benzyloxy-2-chloro-pyridine (500 mg, 2.2761 mmol, 1 eq) in pyridine (5 mL) was added Hydrazine solution (17.7 mg, 11.38 mmol, 5 eq) at room temperature. The reaction mixture was heated at 110° C. for 24 h. After complete consumption of the starting material (monitored by TLC), the reaction mixture was concentrated under reduced pressure to afford (4-benzyloxy-2-pyridyl) hydrazine (400 mg, crude). The crude product as such used for next reaction without carried out further purification.

Step-3: Synthesis of 7-(benzyloxy)-[1,2,4]triazolo[4,3-a]pyridine: To a stirred solution of (4-benzyloxy-2-pyridyl) hydrazine (4, 400 mg, 1.858 mmol, 1 eq) in trimethoxy methane (5, 5 mL) was added P-toluene sulfonic acid (360 mg, 2.102 mmol, 1.1 eq) at room temperature and stirred for 5 min. The reaction mixture was heat 80° C. for 12 h. After complete consumption of the starting material (monitored by TLC), the reaction mixture quenched with water (20 mL) and extracted with ethyl acetate (3×20 mL). The combined organic layer was washed with brine solution (15 mL), dried over anhydrous Na₂SO₄ and concentrated under reduced pressure to afford 7-(benzyloxy)-[1,2,4]triazolo[4,3-a]pyridine (6, 200 mg, crude) as an off white solid. The crude product as such used for next reaction without carried out further purification.

Step-4: Synthesis of [1,2,4]triazolo[4,3-a]pyridin-7-ol: To a stirred solution of 7-benzyloxy-[1,2,4]triazolo[4,3-a]pyridine (400 mg, 1.776 mmol, 1 eq) in methanol (5 mL) were added 10% Pd/C (200 mg, 50% wt) under nitrogen at room temperature and stirred for 12 h under hydrogen pressure at 60 psi. After complete consumption of the starting material (monitored by TLC), the reaction mixture was filtered through a pad of Celite and obtained filtrate was concentrated under reduced pressure to afford [1,2,4]triazolo[4,3-a]pyridin-7-ol (200 mg, crude) as an off white solid. The crude product as such used for next reaction without carried out further purification.

Step-5: Synthesis of 2-(6-(([1,2,4]triazolo[4,3-a]pyridin-7-yloxy)methyl)pyridin-3-yl)-5-(difluoromethyl)-1,3,4-oxadiazole (184): To a stirred solution of [1,2,4]triazolo[4,3-a]pyridin-7-ol (7, 280 mg, 2.0722 mmol, 2 eq) in acetonitrile (5 mL,) were added K₂CO₃ (429 mg, 3.10411 mmol, 3 eq) and 2-[6-(bromomethyl)-3-pyridyl]-5-(difluoromethyl)-1,3,4-oxadiazole (Int-I, 300 mg, 1.0343 mmol, 1 eq) at room temperature. Reaction mixture was heated at 80° C. for 12 h. After complete consumption of the starting material (monitored by TLC), the reaction mixture was cooled to room temperature and concentrated under reduced pressure. The residue obtained was diluted with water (20 mL) and aqueous layer extracted with ethyl acetate (3×20 mL). The combined organic layer was washed with brine solution (20 mL), dried over anhydrous Na₂SO₄ and concentrated under reduced pressure to obtain crude product. The crude product was purified by reverse phase Prep HPLC to afford 2-(6-(([1,2,4]triazolo[4,3-a]pyridin-7-yloxy)methyl)pyridin-3-yl)-5-(difluoromethyl)-1,3,4-oxadiazole (184, 10 mg, 3.0%) as an off white solid. ¹H NMR (400 MHz, DMSO-d₆) δ ppm 9.25 (br. s, 1H), 9.06 (br. s, 1H), 8.47-8.57 (m, 2H), 7.85 (d, J=8.4 Hz, 1H), 7.71 (t, J=52.4 Hz, 1H), 7.21 (br. s, 1H), 6.86 (dd, J=2.4 Hz, 10.0 Hz, 1H), 5.44 (s, 2H). ¹⁹F NMR (400 MHz, DMSO-d₆): δ −120.70. LC-MS: m/z 345.4 [M+H]. HPLC: 97.99% at 5.219 min.

### 2-(Difluoromethyl)-5-(6-((pyrazolo[1,5-a]pyrimidin-6-yloxy)methyl)pyridin-3-yl)-1,3,4-oxadiazole (183)

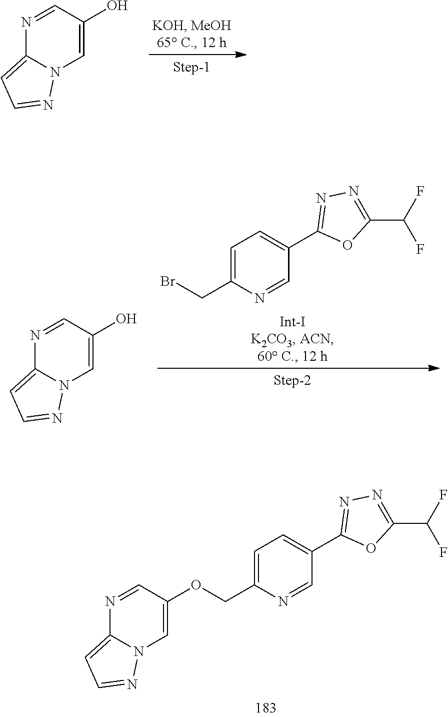 [SMILES: Oc1cnc2ccnn2c1 ; FC(F)c1nnc(-c2ccc(CBr)nc2)o1 ; Oc1cnc2ccnn2c1 ; FC(F)c1nnc(-c2ccc(COc3cnc4ccnn4c3)nc2)o1]

Step-1: Synthesis of pyrazolo[1,5-a]pyrimidin-6-ol: To a stirred solution of 6-bromopyrazolo[1,5-a]pyrimidine (150 mg, 0.7575 mmol, 1.0 equiv.) in methanol (6 mL) was added KOH (259 mg, 4.620 mmol, 6.0 equiv.) at 0° C. temperature. The reaction mixture was heated at 65° C. and stirred for 12 h. After complete consumption of the starting material (monitored by TLC), the reaction mixture was quenched with water (20 mL) and extracted with IPA:DCM (3:1, 3×20 mL). The combined organic layer was washed with brine solution (10 mL), dried over anhydrous Na₂SO₄ and concentrated under reduced pressure to afford pyrazolo[1,5-a]pyrimidin-6-ol (135 mg, crude). The crude product as such used for next reaction without carried out further purification.

Step-2: Synthesis of 2-(difluoromethyl)-5-(6-((pyrazolo[1,5-a]pyrimidin-6-yloxy)methyl)pyridin-3-yl)-1,3,4-oxadiazole (183): To a stirred solution of pyrazolo[1,5-a]pyrimidin-6-ol (2, 14 mg, 0.1034 mmol, 1.0 equiv.) in acetonitrile (3 mL) was added K₂CO₃ (42 mg, 0.3102 mmol, 3.0 equiv.) followed by 2-(6-(bromomethyl)pyridin-3-yl)-5-(difluoromethyl)-1,3,4-oxadiazole (Int-I, 30 mg, 0.1034 mmol, 1.0 equiv.) at room temperature. The reaction mixture was heated at 80° C. and stirred for 12 h. After complete consumption of the starting material (monitored by TLC), the reaction mixture was cooled to room temperature and quenched with water (10 mL) and extracted with ethyl acetate (3×10 mL). The combined organic layer was washed with brine (10 mL), dried over anhydrous Na₂SO₄ and concentrated under reduced pressure to obtain crude product. The crude product was purified by reverse phase Prep HPLC to afford 2-(difluoromethyl)-5-(6-((pyrazolo[1,5-a]pyrimidin-6-yloxy)methyl)pyridin-3-yl)-1,3,4-oxadiazole (183, 20 mg, 56%) as an off white solid. ¹H NMR (400 MHz, DMSO-d₆) δ ppm 9.30-9.40 (m, 1H), 8.49-8.59 (m, 2H), 8.30-8.40 (m, 1H), 8.4 (s, 1H), 7.79 (dd, J=0.4 Hz, 8.8 Hz, 1H), 7.08 (t, J=51.6 Hz, 1H), 6.69 (dd, J=0.8 Hz, 2.4 Hz, 1H), 5.32 (s, 2H). ¹⁹F NMR (400 MHz, DMSO-d₆): δ −120.667, −120.804. LC-MS: m/z 345.3 [M+H]. HPLC: 96.29% at 6.997 min.

### 2-(difluoromethyl)-5-(6-((imidazo[1,2-a]pyrazin-8-yloxy)methyl)pyridin-3-yl)-1,3,4-oxadiazole (86)

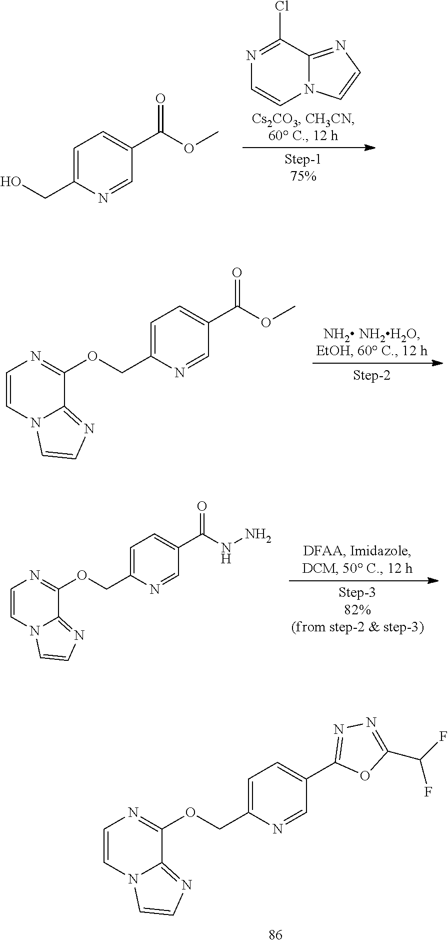 [SMILES: COC(=O)c1ccc(CO)nc1 ; Clc1nccn2ccnc12 ; COC(=O)c1ccc(COc2nccn3ccnc23)nc1 ; NNC(=O)c1ccc(COc2nccn3ccnc23)nc1 ; FC(F)c1nnc(-c2ccc(COc3nccn4ccnc34)nc2)o1]

Step-1: Synthesis of methyl 6-((imidazo[1,2-a]pyrazin-8-yloxy)methyl)nicotinate: To a solution of (methyl 6-(hydroxymethyl)pyridine-3-carboxylate (5.0 g, 29.91 mmol) in acetonitrile (100 mL) in were added Cs₂CO₃ (17.0 g, 52.17 mmol) and 8-chloroimidazo[1,2-a]pyrazine (4.1 g, 27 mmol) at room temperature and stirred for 5 min. The reaction mixture was heated at 60° C. temperature and stirred for 12 h. Progress of reaction was monitored by TLC. After complete consumption of the starting material, the reaction mixture was cooled to room temperature and concentrated under reduced pressure. The residue obtained was dissolved in water (100 mL) and extracted with ethyl acetate (2×250 mL). Organic layer was washed with brine (100 mL), dried over anhydrous Na₂SO₄ and concentrated under reduced pressure to obtain crude product. The crude product was purified by Combi Flash column chromatography using methanol in DCM (0-10%) to afford methyl 5-fluoro-6-methylnicotinate (2.70 g, 75%) as an off-white solid. LC-MS: m/z 285.4 [M+H].

Step-2: Synthesis of 6-((imidazo[1,2-a]pyrazin-8-yloxy)methyl)nicotinohydrazide: To a solution of methyl 6-(imidazo[1,2-a]pyrazin-8-yloxymethyl)pyridine-3-carboxylate (4.2 g, 15 mmol) in ethanol (100 mL) were added hydrazine hydride (3.5 g, 110 mmol) at room temperature and stirred for 5 min. The reaction mixture was heated at 70° C. temperature and stirred for 12 h. Progress of reaction was monitored by TLC. After complete consumption of the starting material, the reaction mixture was cooled to room temperature and concentrated under reduced pressure to afford 6-((imidazo[1,2-a]pyrazin-8-yloxy)methyl)nicotinohydrazide (3.6 g, crude). The crude was as such used for next reaction without carried out further purification. LC-MS: m/z 286.0 [M+H].

Step-3: Synthesis of 2-(difluoromethyl)-5-(6-((imidazo[1,2-a]pyrazin-8-yloxy)methyl)pyridin-3-yl)-1,3,4-oxadiazole (86): To a solution of (6-(imidazo[1,2-a]pyrazin-8-yloxymethyl)pyridine-3-carbohydrazide (6.05 g, 21.3 mmol) in DCM (200 mL) were added imidazole (4.34 g, 62.5 mmol) and difluoroacetic anhydride (11.12 g, 54.31 mmol) at 0° C. temperature and stirred for 5 min. The resulting reaction mixture was heated at 50° C. temperature and stirred for 4 h. Progress of reaction was monitored by TLC. After complete consumption of the starting material, the reaction mixture was cooled to room temperature and concentrated under reduced pressure. The residue obtained was dissolved in water (100 mL) and extracted with DCM (2×250 mL). Organic layer was washed with brine (100 mL), dried over anhydrous Na₂SO₄ and concentrated under reduced pressure to obtain crude product. The crude product was purified by Combi Flash column chromatography using methanol in DCM (0-5%) to afford methyl 5-fluoro-6-methylnicotinate (86, 6.0 g, 82%, From 2 steps) as an off-white solid. LC-MS: m/z 344.93 [M+H]. ¹H NMR (400 MHz, DMSO-d₆) δ ppm 9.22 (d, J=1.47 Hz, 1H), 8.48 (dd, J=8.25, 2.14 Hz, 1H), 8.27 (d, J=4.65 Hz, 1H), 8.12 (d, J=0.98 Hz, 1H), 7.69-7.77 (m, 2H), 7.37-7.59 (m, 2H), 5.74 (s, 2H). HPLC=97.54%, RT=6.815 min.

The following compounds were prepared in a manner analogous to that used for preparing compound (86).

| Compound | Structure/Name | Characterization |
|---|---|---|
| 89 | 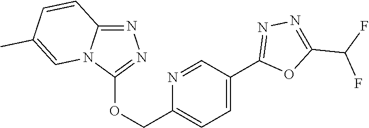 [SMILES: Cc1ccc2nnc(OCc3ccc(-c4nnc(C(F)F)o4)cn3)n2c1] | ¹HNMR (400 MHz, METHANOL-d4) δ = 9.30 (d, J = 1.6 Hz, 1H), 8.57 (dd, J = 2.3, 8.3 Hz, 1H), 7.98 (d, J = 1.3 Hz, 1H), 7.94 (d, J = 8.1 Hz, 1H), 7.46 (d, J = 9.6 Hz, 1H), 7.41-7.14 (m, 2H), 5.86 (s, 2H), 2.36 (d, J = 1.0 Hz, 3H) ¹⁹F NMR (377 MHz, METHANOL-d4) δ = −122.41 (s, 2F) |
| 87 | 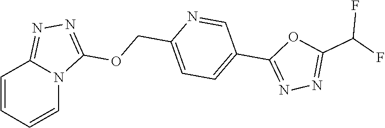 [SMILES: FC(F)c1nnc(-c2ccc(COc3nnc4ccccn34)nc2)o1] | ¹HNMR (400 MHz, DMSO-d6) δ ppm 9.15 (s, 1H), 8.42 (dd, J = 2.4 Hz, J = 10.4 Hz, 1H), 7.91 (d, J = 7.90 Hz, 1H), 7.44-7.70 (m, 2H), 7.24 (m, 2H), 6.65 (t, 1H), 5.35 (s, 2H) LC-MS: m/z 345.12 [M + H]. |

### Example 1B. Synthesis of Additional Compounds of Example 1

### General Experimental Condition for the Chemical Reactions of Example 1B:

### NMR:

¹H NMR spectrum was recorded on Bruker 400 MHz and Varian 400 MHz instruments internally referenced to a tetramethylsilane (TMS) signal. Chemical shifts (6) were expressed in parts per million.

### LCMS:

### Instrument: Agilent 1290 Infinity LAB LC/MSD.

Method-A: Column: Poroshell 120 EC-18 (50 mm*3.0, 1.9 μm); 0.05% TFA in Water, Mobile Phase B: 0.05% TFA in Acetonitrile; Flow rate: 0.7 mL/min; Column temperature: 45° C.

Gradient Program (Time/B %): 0.01/2, 0.3/2, 2/98, 4.5/98, 4.8/2, 5.0/2 HPLC:

Instrument: Waters H-class plus.

Method-A: Column: X-SELECT CSH C18 (150 mm*4.6, 5 μm); Mobile Phase A: 5 mM Ammonium bicarbonate in Water, Mobile Phase B: 100% Acetonitrile; Flow Rate: 1.0 mL/min Gradient (Time/% B): 0.0/05, 1.0/05, 8.0/100, 12.0/100, 14.0/5, 18.0/5. Diluent: ACN: Water (80:20)+DMSO; Column temperature: 45° C.

Instrument: Waters H-class plus.

### Step-1: Synthesis of methyl 5-fluoro-6-methylnicotinate (3)

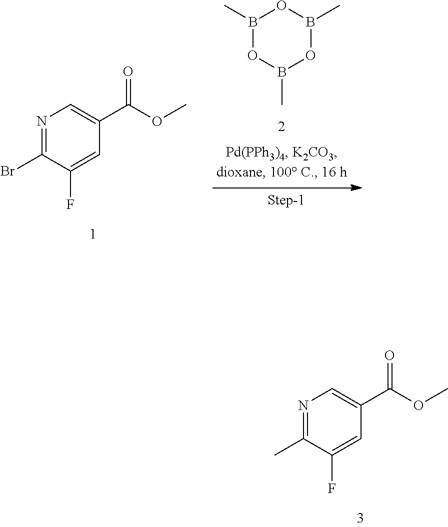

To a stirred solution of methyl 6-bromo-5-fluoronicotinate (1) (5.0 g, 21.36 mmol, 1.0 equiv.) in 1,4 dioxane (75 mL) was added 2,4,6-trimethyl-1,3,5,2,4,6-trioxatriborinane 2 (13.41 g, 106.8 mmol, 5.0 equiv.) followed by K₂CO₃ (4.429 g, 32.00 mmol, 1.5 equiv.) at room temperature and degassed the reaction mixture with argon for 15 min. To the resulting reaction mixture was added Pd(PPh₃)₄ (2.77 g, 0.235 mmol, 0.11 eq.). The reaction mixture was heated at 100° C. and stirred for 16 h. After complete consumption of the starting material, the reaction mixture was diluted with water (100 mL) and extracted with DCM (200 mL). Organic layer was washed with water, brine, dried over anhydrous Na₂SO₄ and concentrated under reduced pressure to obtain crude product. Crude product was purified by Combi Flash column chromatography using ethyl acetate: n-hexane (20%) to afford methyl 5-fluoro-6-methylnicotinate (2.70 g, 75%) as an off white solid. LC-MS: m/z 170.15 [M+H].

### Step-2: Synthesis of 5-fluoro-6-methylnicotinohydrazide (4)

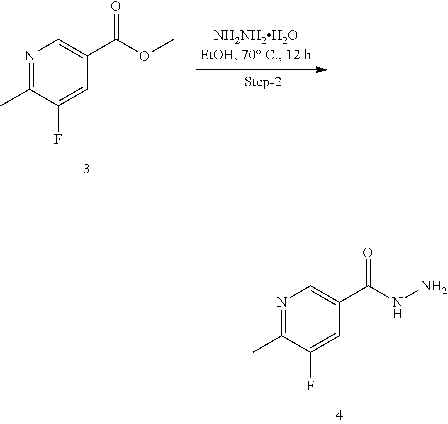

To a stirred solution of methyl 5-fluoro-6-methylnicotinate (3) (0.740 g, 4.374 mmol, 1.0 equiv.) in EtOH (10 mL) was added hydrazine hydrate (1.402 g, 43.74 mmol, 5.0 equiv.) at room temperature. The reaction mixture was heated at 70° C. and stirred for 12 h. After complete consumption of the starting material, the reaction mixture was concentrated under reduced pressure to obtain crude product (4, 0.740 g, crude). The crude was as such used for next reaction without carried out further purification. LC-MS: m/z 170.17 [M+H].

### Step-3: Synthesis of 2-(difluoromethyl)-5-(5-fluoro-6-methylpyridin-3-yl)-1,3,4-oxadiazole (6)

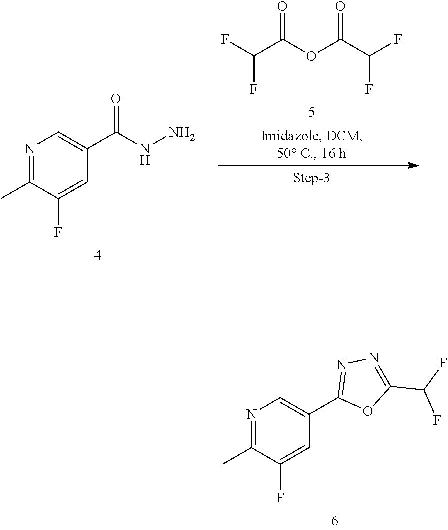 [SMILES: O=C(OC(=O)C(F)F)C(F)F ; Cc1ncc(C(=O)NN)cc1F ; Cc1ncc(-c2nnc(C(F)F)o2)cc1F]

To a stirred solution of methyl 5-fluoro-6-methylnicotinohydrazide (3) (0.740 g, 4.374 mmol, 1.0 equiv.) in DCM (50 mL) was added imidazole (0.893 g, 13.12 mmol, 3.0 equiv.) at room temperature for 15 min. To the resulting reaction mixture was added DFAA (2.28 g, 13.12 mmol) at 0° C. temperature. The reaction mixture was heated at 50° C. and stirred for 16 h. After complete consumption of the starting material (monitored by TLC), the reaction mixture was diluted with water (10 mL) and extracted with DCM (20 mL). Organic layer was washed with water, brine, dried over anhydrous Na₂SO₄ and concentrated under reduced pressure to obtain crude product. Crude product was purified by Combi Flash column chromatography using ethyl acetate: n-heptane (30%) to afford 2-(difluoromethyl)-5-(5-fluoro-6-methylpyridin-3-yl)-1,3,4-oxadiazole (6, 1.788 g, 89.4%) as an off white solid. LC-MS: m/z 230.20 [M+H].

### Step-4: Synthesis of 2-(6-(bromomethyl)-5-fluoropyridin-3-yl)-5-(difluoromethyl)-1,3,4-oxadiazole (Int-K)

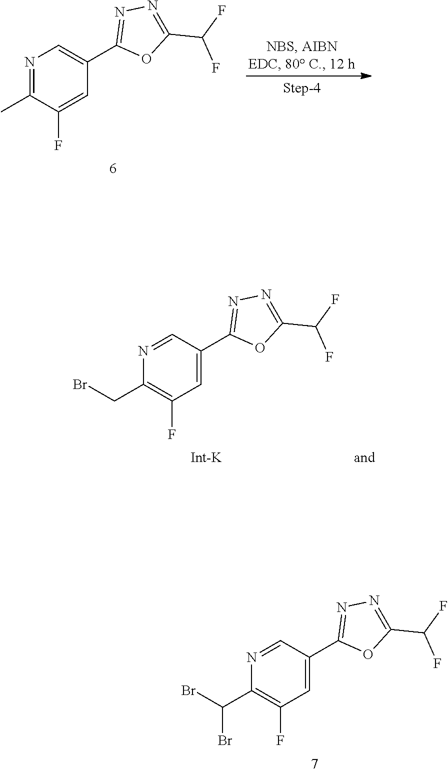 [SMILES: Cc1ncc(-c2nnc(C(F)F)o2)cc1F ; Fc1cc(-c2nnc(C(F)F)o2)cnc1CBr ; Fc1cc(-c2nnc(C(F)F)o2)cnc1C(Br)Br]

To a stirred solution of methyl 5-fluoro-6-methylnicotinohydrazide 2-(difluoromethyl)-5-(5-fluoro-6-methylpyridin-3-yl)-1,3,4-oxadiazole (6) (0.900 g, 3.927 mmol, 1.0 equiv.) in DCE (18 mL) was added NBS (2.10 g, 23.56 mmol, 6.0 equiv.) followed by AIBN (0.322 g, 1.963 mmol, 0.5 equiv.) at room temperature for 15 min. The reaction mixture was heated at 80° C. and stirred for 12 h. After complete consumption of the starting material (monitored by TLC), the reaction mixture was diluted with water (30 mL) and extracted with ethyl acetate (50 mL). Organic layer was washed with water, brine, dried over anhydrous Na₂SO₄ and concentrated under reduced pressure to obtain crude product. Crude product was purified by Combi Flash column chromatography using ethyl acetate: n-heptane (30%) to afford 2-(6-(bromomethyl)-5-fluoropyridin-3-yl)-5-(difluoromethyl)-1,3,4-oxadiazole (Int-K, 0.600 g, 44%, 7, 550 mg, 35%) as an off white solid. 1H NMR (400 MHz, CHLOROFORM-d) δ ppm 9.12 (s, 1H), 8.13 (br. d, J=8.80 Hz, 1H), 6.76-7.10 (m, 1H), 4.66 (s, 3H). LC-MS: m/z=309.6.

### Step-5: Synthesis of 2-(6-(bromomethyl)-5-fluoropyridin-3-yl)-5-(difluoromethyl)-1,3,4-oxadiazole (Int-K)

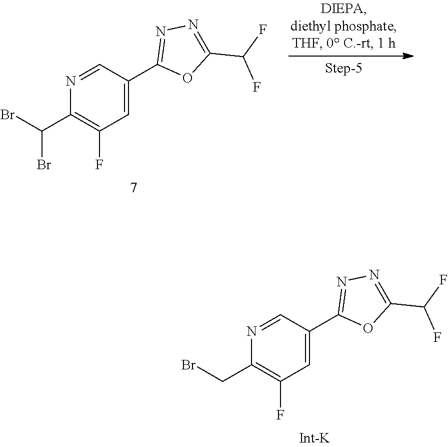 [SMILES: Fc1cc(-c2nnc(C(F)F)o2)cnc1C(Br)Br ; Fc1cc(-c2nnc(C(F)F)o2)cnc1CBr]

To a stirred solution of 2-(6-(dibromomethyl)-5-fluoropyridin-3-yl)-5-(difluoromethyl)-1,3,4-oxadiazole (0.370 g, 0.953 mmol, 1.0 equiv.) in THF (10 mL) was added DIPEA (0.246 g, 1.907 mmol, 2.0 equiv.) followed by Diethyl phosphate (263.1 g, 1.907 mmol, 1.907 equiv.) at 0° C. temperature. The resulting reaction mixture was allowed to attain room temperature and stirred for 1 h. After complete consumption of the starting material, the reaction mixture was diluted with water (5 mL) and extracted with ethyl acetate (10 mL). Organic layer was washed with water, brine, dried over anhydrous Na₂SO₄ and concentrated under reduced pressure to obtain crude product. Crude product was purified by Combi Flash column chromatography using ethyl acetate: n-heptane (30%) to afford 2-(6-(bromomethyl)-5-fluoropyridin-3-yl)-5-(difluoromethyl)-1,3,4-oxadiazole (Int-K, 0.687 g, 72.3%) as an off white solid. 1H NMR (400 MHz, CHLOROFORM-d) δ ppm 9.12 (s, 1H), 8.13 (br. d, J=8.80 Hz, 1H), 6.76-7.10 (m, 1H), 4.66 (s, 3H). LC-MS: m/z=310.11.

### 2-(difluoromethyl)-5-[2-(quinoxalin-6-yloxymethyl)pyrimidin-5-yl]-1,3,4-oxadiazole (Compound 150)

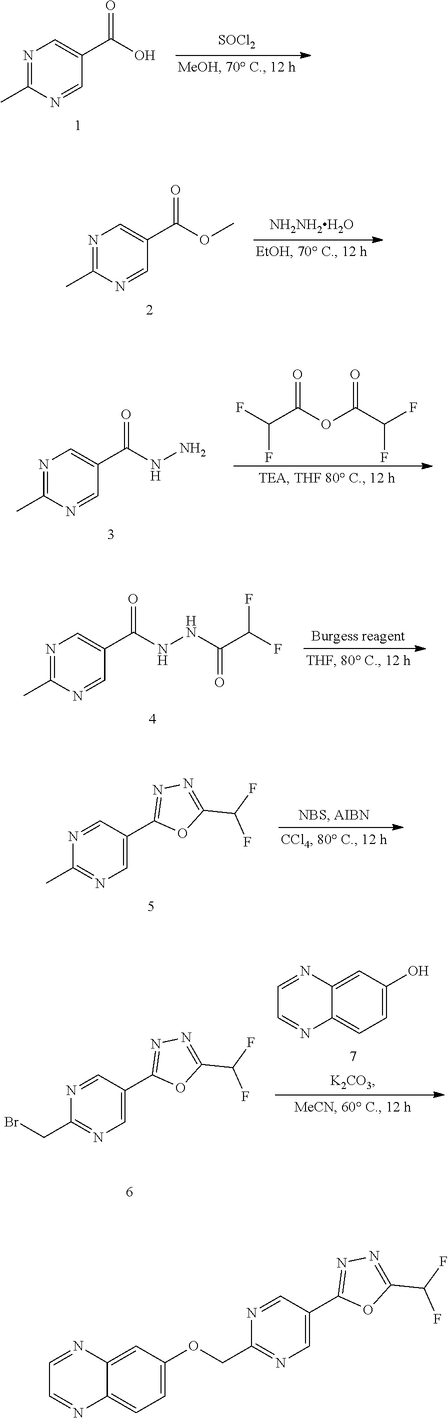

### Step 1: methyl 2-methylpyrimidine-5-carboxylate

To a solution of 2-methylpyrimidine-5-carboxylic acid (50 g, 361.99 mmol, 1 eq) in MeOH (600 mL) was degassed and purged with N₂ for 3 times at 25° C., and then SOCl₂ (129.20 g, 1.09 mol, 3 eq) was added dropwise at 25° C. over 30 min, and then the mixture was stirred at 70° C. for 3 hr under N₂ atmosphere. LCMS showed the reaction was completed. The reaction mixture was concentrated under reduced pressure. The residue was quenched with NaHCO₃ (aq 800 mL) at 0° C., and extracted with DCM (500 mL*2). The combined organic phase were washed with brine (1 L*2), dried over Na₂SO₄, filtered and concentrated under reduced pressure to obtain methyl 2-methylpyrimidine-5-carboxylate (26 g, crude) as an off-white solid. MS (ESI): 153.0 [M+H]⁺

### Step 2: 2-methylpyrimidine-5-carbohydrazide

To a solution of ethyl 2-methylpyrimidine-5-carboxylate (26 g, 156.46 mmol, 1 eq) in EtOH (400 mL) was added NH₂NH₂·H₂O (31.97 g, 625.84 mmol, 98% purity, 4 eq). The mixture was stirred at 70° C. under N₂ atmosphere for 12 hr. LCMS showed the reaction was completed. The reaction mixture was concentrated under reduced pressure. The residue was purified by re-crystallization from ethyl acetate (300 mL) to obtain 2-methylpyrimidine-5-carbohydrazide (21 g, 126.98 mmol, 81.16% yield, 92% purity) as a white solid. ¹H NMR (400 MHz, DMSO-d₆) δ=9.02 (s, 2H), 2.67 (s, 3H), 1.75 (s, 3H). MS(ESI): 153.0 [M+H]⁺

### Step 3: N′-(2,2-difluoroacetyl)-2-methyl-pyrimidine-5-carbohydrazide

A mixture of 2-methylpyrimidine-5-carbohydrazide (20 g, 131.45 mmol, 1 eq) and TEA (26.60 g, 262.89 mmol, 36.59 mL, 2 eq) in THF (200 mL) was degassed and purged with N₂ for 3 times at 25° C., and then 2,2-difluoroacetic anhydride (45.76 g, 262.89 mmol, 2 eq) was added dropwise at 25° C. The mixture was stirred at 80° C. for 12 hr under N₂ atmosphere. LCMS showed the reaction was completed. The reaction mixture was concentrated under reduced pressure. The residue was diluted with H₂O (3 mL) and extracted with DCM (200 mL*3). The combined organic phase was washed with H₂O (150 mL*2), dried over Na₂SO₄, filtered and concentrated under reduced pressure. The residue was purified by column chromatography (80 g Silica Flash Column, Eluent of 0-10% Petroleum ether:Ethyl acetate @80 mL/min) to obtain N′-(2,2-difluoroacetyl)-2-methyl-pyrimidine-5-carbohydrazide (20 g, 77.33 mmol, 58.83% yield, 89% purity) as a white solid. MS (ESI): 231.0 [M+H]⁺

### Step 4: 2-(difluoromethyl)-5-(2-methylpyrimidin-5-yl)-1,3,4-oxadiazole

A mixture of N′-(2,2-difluoroacetyl)-2-methyl-pyrimidine-5-carbohydrazide (20 g, 86.89 mmol, 1 eq) and methoxycarbonyl-(triethylammonio)sulfonyl-azanide (41.41 g, 173.78 mmol, 2 eq) in THF (200 mL) was degassed and purged with N₂ for 3 times, and then the mixture was stirred at 80° C. for 12 hr under N₂ atmosphere. LCMS showed the reaction was completed. The reaction mixture was concentrated under reduced pressure and purified by column chromatography (80 g Silica Flash Column, Eluent of 0-30% Petroleum ether:Ethyl acetate @80 mL/min) to obtain 2-(difluoromethyl)-5-(2-methylpyrimidin-5-yl)-1,3,4-oxadiazole (6.6 g, 30.49 mmol, 35.09% yield, 98% purity) as a white solid. MS (ESI): 213.0 [M+H]⁺

### Step 5: 2-[2-(bromomethyl)pyrimidin-5-yl]-5-(difluoromethyl)-1,3,4-oxadiazole

To a solution of 2-(difluoromethyl)-5-(2-methylpyrimidin-5-yl)-1,3,4-oxadiazole (8.8 g, 41.48 mmol, 1 eq) in CCl₄ (90 mL) was added AIBN (681.13 mg, 4.15 mmol, 0.1 eq) and NBS (29.53 g, 165.92 mmol, 4 eq). The mixture was stirred at 80° C. for 12 hr. LCMS showed 20% of desired compound was detected. The mixture was concentrated under reduced pressure. The residue was diluted with H₂O (30 mL) and extracted with DCM (120 mL*2). The combined organic phase were washed with H₂O (80 mL*2), dried over Na₂SO₄, filtered and concentrated under reduced pressure. The residue was purified by column chromatography (80 g Silica Flash Column, Eluent of 0-10% Petroleum ether:Ethyl acetate @80 mL/min) to obtain 2-[2-(bromomethyl)pyrimidin-5-yl]-5-(difluoromethyl)-1,3,4-oxadiazole (1.2 g, 3.71 mmol, 8.94% yield, 90% purity) as an off-white solid. ¹H NMR (400 MHz, METHANOL-d₄) δ=9.45 (s, 2H), 7.30-7.28 (m, 1H), 4.74 (s, 2H). MS (ESI): 292.0 [M+H]⁺

### Step 6: 2-(difluoromethyl)-5-[2-(quinoxalin-6-yloxymethyl)pyrimidin-5-yl]-1,3,4-oxadiazole (Compound 150)

To a solution of 2-[2-(bromomethyl)pyrimidin-5-yl]-5-(difluoromethyl)-1,3,4-oxadiazole (100 mg, 343.58 umol, 1 eq) and K₂CO₃ (94.97 mg, 687.16 umol, 2 eq) in MeCN (5 mL) was added quinoxalin-6-ol (50.21 mg, 343.58 umol, 1 eq). The mixture was stirred at 60° C. for 12 hr. LCMS showed the reaction was completed. The mixture was concentrated under reduced pressure. The residue was diluted with H₂O (3 ml) and extracted with DCM (5 mL*3). The combined organic phase was washed with H₂O (5 mL*2), dried over Na₂SO₄, filtered and concentrated under reduced pressure. The residue was purified by prep-HPLC (column: Phenomenex Luna C18 150*25 mm*10 um; mobile phase: [water (NH₄HCO₃)-ACN]; B %: 22%-52%, 8 min) to obtain 2-(difluoromethyl)-5-[2-(quinoxalin-6-yloxymethyl)pyrimidin-5-yl]-1,3,4-oxadiazole (20.91 mg, 56.52 umol, 16.45% yield, 96.3% purity) as a brown solid. ¹H NMR (400 MHz, METHANOL-d₄) δ=9.49 (s, 2H), 8.80 (d, J=1.9 Hz, 1H), 8.75 (d, J=2.0 Hz, 1H), 8.06 (d, J=9.3 Hz, 1H), 7.71-7.68 (m, 1H), 7.50 (d, J=2.9 Hz, 1H), 7.42-7.15 (m, 1H), 5.67 (s, 2H). MS (ESI): 357.0 [M+H]⁺

### Synthesis of Compound 151: 2-(difluoromethyl)-5-(5-fluoro-6-((quinoxalin-6-yloxy) methyl) pyridin-3-yl)-1,3,4-oxadiazole

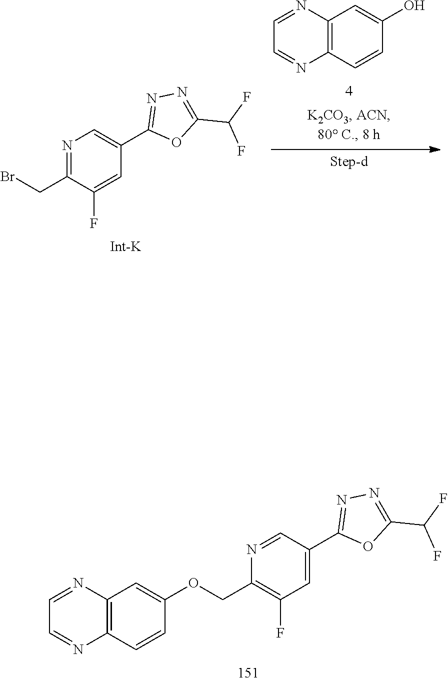 [SMILES: Oc1ccc2nccnc2c1 ; Fc1cc(-c2nnc(C(F)F)o2)cnc1CBr ; Fc1cc(-c2nnc(C(F)F)o2)cnc1COc1ccc2nccnc2c1]

### Step-d: Synthesis of 2-(difluoromethyl)-5-(5-fluoro-6-((quinoxalin-6-yloxy methyl) pyridin-3-yl)-1,3,4-oxadiazole (Compound 151)

To a stirred solution of quinoxalin-6-ol (4, 100 mg, 0.480 mmol, 1.0 equiv.) in acetonitrile (10 mL) was added K₂CO₃ (199 mg, 1.43 mmol, 3.0 equiv.) followed by 2-(6-(bromomethyl)-5-fluoropyridin-3-yl)-5-(difluoromethyl)-1,3,4-oxadiazole (Int-K, 100 mg, 0.480 mmol, 1.0 equiv.) at room temperature. The reaction mixture was heated at 80° C. and stirred for 3 h. After complete consumption of the starting material, the reaction mixture was cooled to room temperature and quenched with water (8 mL) and extracted with ethyl acetate (15 mL). Organic layer was washed with brine (5 mL), dried over anhydrous Na₂SO₄ and concentrated under reduced pressure to obtain crude product. Crude product was purified by Combi Flash column chromatography using ethyl acetate: n-heptane (30%) to afford 2-(difluoromethyl)-5-(5-fluoro-6-((quinoxalin-6-yloxy) methyl) pyridin-3-yl)-1,3,4-oxadiazole (EKZ-0002651, 25 mg, 13%) as an off white solid. 1H NMR (400 MHz, DMSO-d₆) δ ppm 9.12 (s, 1H), 8.95 (d, J=1.75 Hz, 1H), 8.89 (d, J=1.75 Hz, 1H), 8.50 (dd, J=9.76, 1.63 Hz, 1H), 7.76-7.82 (m, 1H), 7.67-7.74 (m, 1H), 7.60 (s, 1H), 7.46-7.52 (m, 1H), 5.60 (d, J=1.50 Hz, 2H). LC-MS: m/z=373.88 [M+H]⁺ HPLC: 99.39% at 7.140 min.

### Synthesis of Compound 155: 2-(difluoro methyl)-5-(5-fluoro-6-((isoquinolin-6-yloxy) methyl) pyridin-3-yl)-1,3,4-oxadiazole

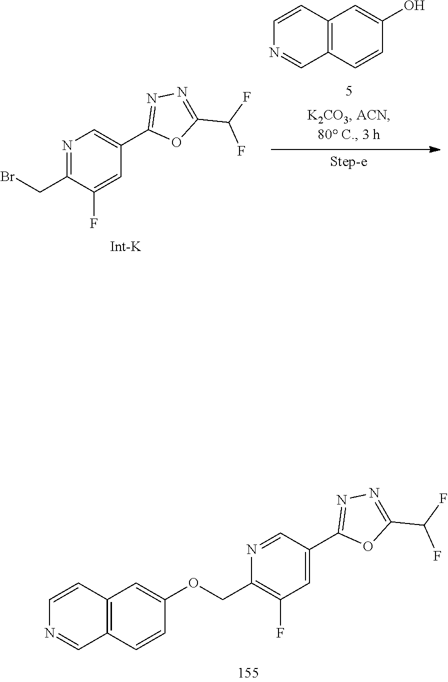 [SMILES: Oc1ccc2cnccc2c1 ; Fc1cc(-c2nnc(C(F)F)o2)cnc1CBr ; Fc1cc(-c2nnc(C(F)F)o2)cnc1COc1ccc2cnccc2c1]

### Step-e: Synthesis of 2-(difluoro methyl)-5-(5-fluoro-6-((isoquinolin-6-yloxy) methyl) pyridin-3-yl)-1,3,4-oxadiazole (Compound 155)

To a stirred solution of isoquinolin-6-ol (5, 47.12 mg, 0.324 mmol, 1.0 equiv.) in acetonitrile (10 mL) was added K₂CO₃ (134.6 mg, 0.97 mmol, 3.0 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 80° C. and stirred for 3 h. After complete consumption of the starting material, the reaction mixture was cooled to room temperature and quenched with water (5 mL) and extracted with ethyl acetate (10 mL). Organic layer was washed with brine (5 mL), dried over anhydrous Na₂SO₄ and concentrated under reduced pressure to obtain crude product. Crude product was purified by Combi Flash column chromatography using ethyl acetate: n-heptane (30%) to afford 2-(difluoro methyl)-5-(5-fluoro-6-((isoquinolin-6-yloxy) methyl) pyridin-3-yl)-1,3,4-oxadiazole (Compound 155, 28 mg, 23%) as an off white solid. 1H NMR (400 MHz, DMSO-d₆) δ ppm 9.08-9.21 (m, 2H), 8.36-8.53 (m, 2H), 8.06 (d, J=9.01 Hz, 1H), 7.68-7.75 (m, 1H), 7.50-7.61 (m, 2H), 7.31-7.47 (m, 1H), 5.49-5.59 (m, 2H). LC-MS: m/z=373.2 [M+H]⁺ HPLC: 99.66% at 7.853 min.

### Synthesis of Compound 157: 2-(difluoromethyl)-5-(5-fluoro-6-((isoquinolin-7-yloxy)methyl)pyridin-3-yl)-1,3,4-oxadiazole

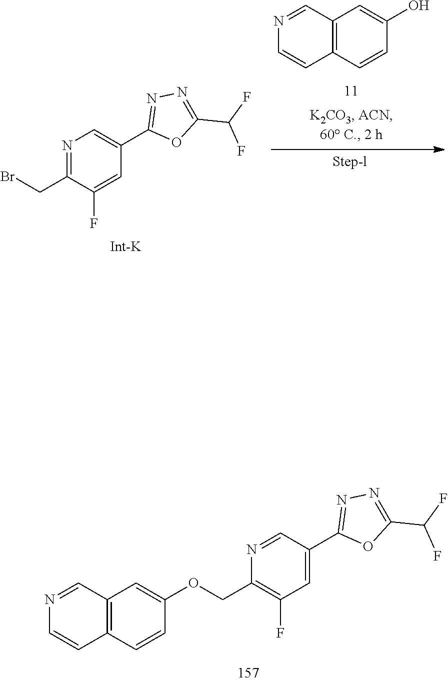 [SMILES: Oc1ccc2ccncc2c1 ; Fc1cc(-c2nnc(C(F)F)o2)cnc1CBr ; Fc1cc(-c2nnc(C(F)F)o2)cnc1COc1ccc2ccncc2c1]

### Step-1: Synthesis of 2-(difluoromethyl)-5-(5-fluoro-6-((isoquinolin-7-yloxy)methyl)pyridin-3-yl)-1,3,4-oxadiazole (Compound 157)

To a stirred solution of isoquinolin-7-ol (11, 35 mg, 0.244 mmol, 1.0 equiv.) in acetonitrile (7.5 mL) was added K₂CO₃ (101.28 mg, 0.7328 mmol, 3.0 equiv.) followed by 2-(6-(bromomethyl)-5-fluoropyridin-3-yl)-5-(difluoromethyl)-1,3,4-oxadiazole (Int-K, 75 mg, 0.2442 mmol, 1.0 equiv.) at room temperature. The reaction mixture was heated at 60° C. and stirred for 2 h. After complete consumption of the starting material, the reaction mixture was cooled to room temperature and quenched with water (2 mL) and extracted with ethyl acetate (5 mL). Organic layer was washed with brine (5 mL), dried over anhydrous Na₂SO₄ and concentrated under reduced pressure to obtain crude product. Crude product was purified by Combi Flash column chromatography using ethyl acetate: n-heptane (30%) to afford 2-(difluoromethyl)-5-(5-fluoro-6-((isoquinolin-7-yloxy)methyl)pyridin-3-yl)-1,3,4-oxadiazole (Compound 157, 25.43 mg, 28.2%) as an off white solid. 1H NMR (400 MHz, DMSO-d₆) δ ppm 9.21-9.21 (m, 1H), 9.12 (s, 1H), 8.49 (dd, J=9.82, 1.69 Hz, 1H), 8.39 (d, J=5.63 Hz, 1H), 7.93 (d, J=9.01 Hz, 1H), 7.77 (d, J=5.63 Hz, 1H), 7.69-7.72 (m, 1H), 7.60 (s, 1H), 7.46-7.54 (m, 1H), 5.54 (d, J=1.50 Hz, 2H). LC-MS: m/z: 373.2 [M+H]⁺ HPLC: 99.81% at 2.595 min.

### Synthesis of Compound B: 2-(difluoromethyl)-5-(5-fluoro-6-((quinazolin-6-yloxy)methyl)pyridin-3-yl)-1,3,4-oxadiazole

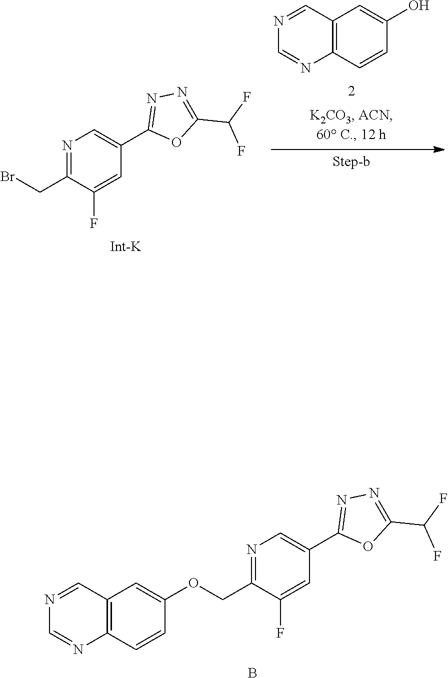

### Step-b: Synthesis of 2-(difluoromethyl)-5-(5-fluoro-6-((quinazolin-6-yloxy)methyl)pyridin-3-yl)-1,3,4-oxadiazole (Compound B)

To a stirred solution of quinazolin-6-ol (2, 47 mg, 0.321 mmol, 1.0 equiv.) in acetonitrile (5 mL) was added K₂CO₃ (112 mg, 0.400 mmol, 1.20 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material, the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by CombiFlash column chromatography using ethyl acetate: n-hexane (30%) to afford 2-(difluoromethyl)-5-(5-fluoro-6-((quinazolin-6-yloxy)methyl)pyridin-3-yl)-1,3,4-oxadiazole (Compound B, 57 mg, 30%) as an off white solid.

### Synthesis of Compound C: 2-(difluoromethyl)-5-(5-fluoro-6-((quinazolin-7-yloxy)methyl)pyridin-3-yl)-1,3,4-oxadiazole

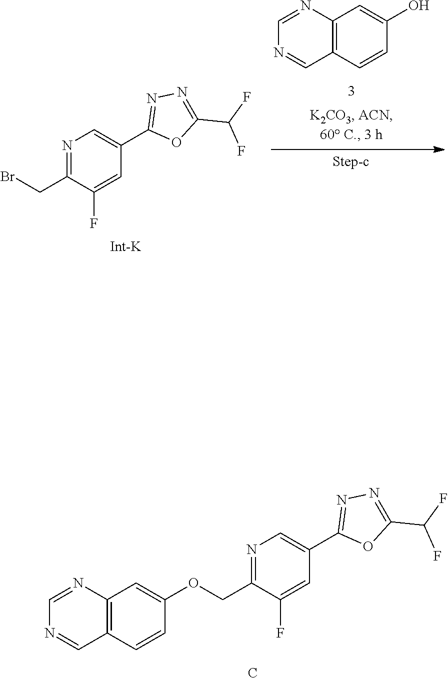

### Step-c: Synthesis of 2-(difluoromethyl)-5-(5-fluoro-6-((quinazolin-7-yloxy)methyl)pyridin-3-yl)-1,3,4-oxadiazole (Compound C)

To a stirred solution of quinazolin-7-ol (3, 47 mg, 0.321 mmol, 1.0 equiv.) in acetonitrile (5 mL) was added K₂CO₃ (113 mg, 0.817 mmol, 2.5 equiv.) followed by 2-(6-(bromomethyl)-5-fluoropyridin-3-yl)-5-(difluoromethyl)-1,3,4-oxadiazole (Int-K, 100 mg, 0.324 mmol, 1.0 equiv.) at room temperature. The reaction mixture was heated at 60° C. and stirred for 12 h. After complete consumption of the starting material, the reaction mixture was cooled to room temperature and concentrated under reduced pressure. The residue obtained was diluted with water (10 mL) and aqueous layer extracted with ethyl acetate (3×10 mL). The combined organic layer was washed with brine solution (10 mL), dried over anhydrous Na₂SO₄ and concentrated under reduced pressure to obtain crude product. The crude product was purified by Combi Flash column chromatography using ethyl acetate: n-hexane (30%) to afford 2-(difluoromethyl)-5-(5-fluoro-6-((quinazolin-7-yloxy)methyl)pyridin-3-yl)-1,3,4-oxadiazole (Compound C, 65 mg, 55%) as an off white solid.

### Synthesis of Compound D: 2-(6-(((6-bromoimidazo[1,2-a]pyridin-8-yl)oxy)methyl)-5-fluoropyridin-3-yl)-5-(difluoro-methyl)-1,3,4-oxadiazole

### Step-1: Synthesis of 6-bromoimidazo[1,2-a]pyridin-8-ol

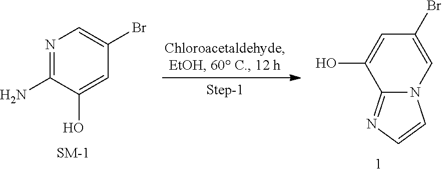 [SMILES: Nc1ncc(Br)cc1O ; Oc1cc(Br)cn2ccnc12]

To a stirred solution of 2-amino-5-bromopyridin-3-ol (5 g, 26.45 mmol, 1 eq) in ethanol (50 mL) was added chloroacetaldehyde in water (50%) (8.31 g, 52.91 mmol, 2 eq) at 0° C., then reaction mixture was heated to 60° C. for 12 h. The progress of the reaction was monitored by TLC and LCMS. After completion, the reaction mixture was concentrated under reduced pressure to obtain brown solid which was washed with acetone (100 mL) and dried to obtain 6-bromoimidazo[1,2-a]pyridin-8-ol (5.9 g, 28.0 mmol, 100% yield) as pale brown solid. ¹H NMR (400 MHz, DMSO-d₆) δ=8.74 (s, 1H), 8.24 (s, 1H), 8.13 (s, 1H), 7.38 (s, 1H) ppm (OH-peak was not observed). LCMS (Method-D): 96.74%; 213.0 [M+H]⁺

### Step-2: Synthesis of 2-(6-(((6-bromoimidazo[1,2-a]pyridin-8-yl)oxy)methyl)-5-fluoropyridin-3-yl)-5-(difluoromethyl)-1,3,4-oxadiazole (Compound D)

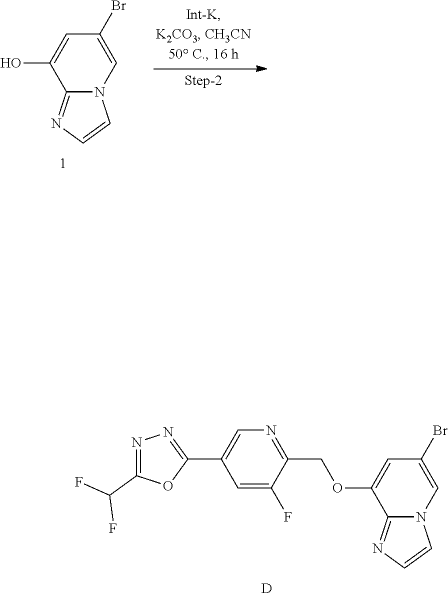

To a stirred solution of 6-bromoimidazo[1,2-a]pyridin-8-ol (1 g, 4.69 mmol, 1 eq) in acetonitrile (10 mL) was added potassium carbonate (1.95 g, 14.08 mmol, 3 eq) followed by the addition of 2-[6-(bromomethyl)-5-fluoro-3-pyridyl]-5-(difluoromethyl)-1,3,4-oxadiazole (Int-K, 1.59 g, 5.16 mmol, 1.1 eq). The resulting reaction mixture was stirred at 50° C. for 16 h. After completion, the reaction mixture was concentrated under reduced pressure and water (50 mL) was added, extracted with ethyl acetate (50 mL×3), combined organics were dried over sodium sulfate, filtered and concentrated under reduced pressure. The crude was purified by combi-flash column chromatography (230-400 mesh silica gel, gradient: 0-5% MeOH in DCM) to obtain 2-(6-(((6-bromoimidazo[1,2-a]pyridin-8-yl)oxy)methyl)-5-fluoropyridin-3-yl)-5-(difluoro-methyl)-1,3,4-oxadiazole (468 mg, 1.063 mmol, 22.7% yield) as an off white solid. 1H NMR (400 MHz, DMSO-d₆) δ=9.12 (t, J=2.4 Hz, 1H), 8.56-8.49 (m, 2H), 7.91 (d, J=0.8 Hz, 1H), 7.60 (t, J=51.2 Hz, 1H, CHF₂), 7.49 (d, J=1.2 Hz, 1H), 7.02 (d, J=1.6 Hz, 1H), 5.59 (d, J=1.6 Hz, 2H) ppm. LCMS (Method-E): 98.87%; 400.0 [M+H]⁺ HPLC-A (Method-A): 99.15% purity.

### Synthesis of Compound E: 2-(difluoromethyl)-5-(5-fluoro-6-(((6-(4-fluorophenyl)imidazo[1,2-a]pyridin-8-yl)oxy)methyl)pyridin-3-yl)-1,3,4-oxadiazole

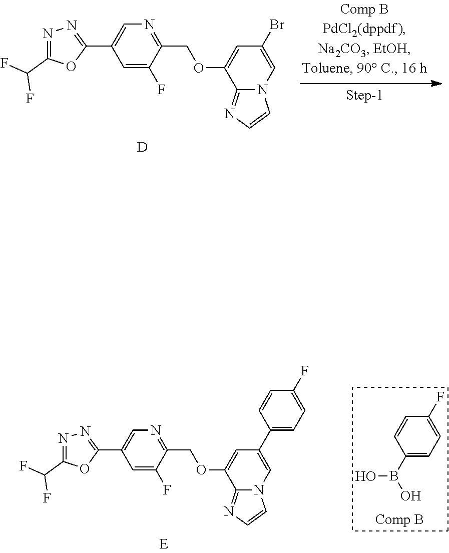

### Step-1: Synthesis of 2-(difluoromethyl)-5-(5-fluoro-6-(((6-(4-fluorophenyl)imidazo[1,2-a]pyridin-8-yl)oxy)methyl)pyridin-3-yl)-1,3,4-oxadiazole

To a stirred solution of 2-(6-(((6-bromoimidazo[1,2-a]pyridin-8-yl)oxy)methyl)-5-fluoropyridin-3-yl)-5-(difluoromethyl)-1,3,4-oxadiazole (100 mg, 0.23 mmol, 1 eq) and (4-fluorophenyl)boronic acid (47.7 mg, 0.34 mmol, 1.5 eq) in ethanol (0.4 mL) and toluene (1 mL) was added sodium carbonate (36.12 mg, 0.34 mmol, 1.5 eq), reaction mixture was degassed under argon for 15 min followed by addition of [1,1′-bis(diphenylphosphino)ferrocene]dichloropalladium(II) (8.5 mg, 0.01 mmol, 0.05 eq). The resulting reaction mixture was heated to 90° C. for 16 h. After completion, the reaction mixture was concentrated under reduced pressure and water (30 mL) added and extracted with ethyl acetate (30 mL×3), combined organics were dried over sodium sulfate, filtered and concentrated under reduced pressure. The crude was purified by combi-flash column chromatography (230-400 mesh silica gel, gradient: 0-5% MeOH in DCM) to obtain 2-(difluoromethyl)-5-(5-fluoro-6-(((6-(4-fluorophenyl)imidazo[1,2-a]pyridin-8-yl)oxy)methyl)pyridin-3-yl)-1,3,4-oxadiazole (Compound E) (35 mg, 0.08 mmol, 33.83% yield) as brown solid. 1H NMR (400 MHz, DMSO-d₆) δ=9.14 (s, 1H), 8.57 (d, J=1.2 Hz, 1H), 8.51 (dd, J=10.0, 1.6 Hz, 1H), 7.95 (d, J=0.8 Hz, 1H), 7.80-7.76 (m, 2H), 7.73-7.47 (m, 2H), 7.37-7.32 (m, 2H), 7.17 (d, J=1.2 Hz, 1H), 5.68 (d, J=1.2 Hz, 2H) ppm. LCMS (Method-A): 98.30%; 456.0 [M+H]⁺ HPLC-A (Method-A): 97.12% purity.

### Synthesis of Compound F: 2-(difluoromethyl)-5-(5-fluoro-6-(((6-(3-fluorophenyl)imidazo[1,2-a]pyridin-8-yl)oxy)methyl)pyridin-3-yl)-1,3,4-oxadiazole

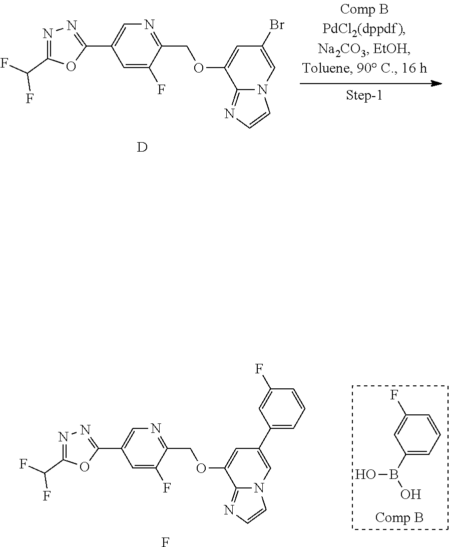

### Step-1: Synthesis of 2-(difluoromethyl)-5-(5-fluoro-6-(((6-(3-fluorophenyl)imidazo[1,2-a]pyridin-8-yl)oxy)methyl)pyridin-3-yl)-1,3,4-oxadiazole

To a stirred solution of 2-(6-(((6-bromoimidazo[1,2-a]pyridin-8-yl)oxy)methyl)-5-fluoropyridin-3-yl)-5-(difluoromethyl)-1,3,4-oxadiazole (100 mg, 0.23 mmol, 1 eq) and (3-fluorophenyl)boronic acid (47.7 mg, 0.34 mmol, 1.5 eq) in ethanol (0.4 mL) and toluene (1.0 mL) was added sodium carbonate (36.12 mg, 0.34 mmol, 1.5 eq), reaction mixture was de gassed under argon for 15 min followed by addition of [1,1′-bis(diphenylphosphino)ferrocene]dichloropalladium(II) (8.5 mg, 0.01 mmol, 0.05 eq). The resulting reaction mixture was heated to 90° C. for 16 h. After completion, the reaction mixture was concentrated under reduced pressure, added water (30 mL) and extracted with ethyl acetate (30 mL×3). The combined organics were dried over anhydrous Na₂SO₄, filtered and concentrated under reduced pressure. The crude was purified by combi-flash column chromatography (230-400 mesh silica gel, gradient: 0-5% MeOH in DCM) to obtain 2-(difluoromethyl)-5-(5-fluoro-6-(((6-(3-fluorophenyl)imidazo[1,2-a]pyridin-8-yl)oxy)methyl)pyridin-3-yl)-1,3,4-oxadiazole (Compound F) (30 mg, 0.06 mmol, 29.0% yield) as brown solid. ¹H NMR (400 MHz, CDCl₃) δ=9.18 (s, 1H), 8.17 (dd, J=9.2, 2.0 Hz, 1H), 8.02 (d, J=1.6 Hz, 1H), 7.63-7.62 (m, 2H), 7.45-7.42 (m, 1H), 7.35-7.33 (m, 1H), 7.24-7.22 (m, 1H), 7.10-6.82 (m, 3H), 5.68 (d, J=1.6 Hz, 2H) ppm. LCMS (Method-A): 98.8%; 456.0 [M+H]⁺ HPLC-A (Method-A): 96.7% purity.

### Synthesis of Compound G: 2-(difluoromethyl)-5-(5-fluoro-6-(((6-(2-fluorophenyl)imidazo[1,2-a]pyridin-8-yl)oxy)methyl)pyridin-3-yl)-1,3,4-oxadiazole

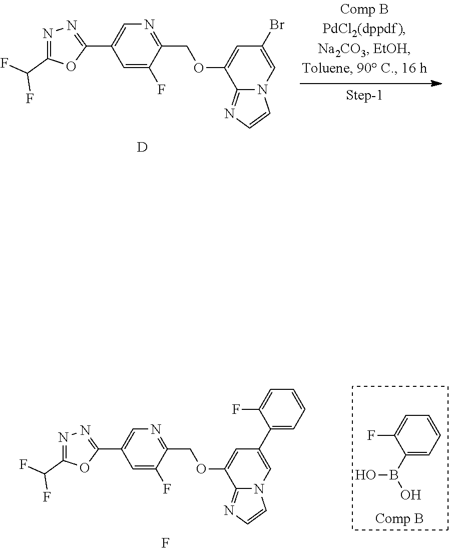

### Step-1: Synthesis of 2-(difluoromethyl)-5-(5-fluoro-6-(((6-(2-fluorophenyl)imidazo[1,2-a]pyridin-8-yl)oxy)methyl)pyridin-3-yl)-1,3,4-oxadiazole

To a stirred solution of 2-(6-(((6-bromoimidazo[1,2-a]pyridin-8-yl)oxy)methyl)-5-fluoropyridin-3-yl)-5-(difluoromethyl)-1,3,4-oxadiazole (100 mg, 0.23 mmol, 1 eq) and (2-fluorophenyl)boronic acid (47.7 mg, 0.34 mmol, 1.5 eq) in ethanol (0.4 mL) and toluene (1.0 mL) was added sodium carbonate (36.12 mg, 0.34 mmol, 1.5 eq), reaction mixture was degassed under argon for 15 min followed by addition of [1,1′-bis(diphenylphosphino)ferrocene]dichloropalladium (II) (8.5 mg, 0.01 mmol, 0.05 eq). The resulting reaction mixture was heated to 90° C. for 16 h. After completion, the reaction mixture was concentrated under reduced pressure and water (30 mL) added and extracted with ethyl acetate (30 mL×3), combined organics were dried over anhydrous Na₂SO₄, filtered and concentrated under reduced pressure. The crude was purified by combi-flash column chromatography (230-400 mesh silica gel, gradient: 0-5% MeOH in DCM) to obtain 2-(difluoromethyl)-5-(5-fluoro-6-(((6-(2-fluorophenyl)imidazo[1,2-a]pyridin-8-yl)oxy)methyl)pyridin-3-yl)-1,3,4-oxadiazole (Compound G) (56 mg, 0.12 mmol, 54.13% yield) as brown solid. ¹H NMR (400 MHz, DMSO-d₆) δ=9.13 (d, J=1.2 Hz, 1H), 8.52-8.48 (m, 2H), 8.01 (d, J=1.2 Hz, 1H), 7.73-7.45 (m, 4H), 7.39-7.33 (m, 2H), 7.02 (s, 1H), 5.64 (d, J=1.2 Hz, 2H) ppm. LCMS (Method-A): 99.28%; 456.0 [M+H]⁺ HPLC-A (Method-A): 99.01% purity.

### Synthesis of Compound H: 2-(difluoromethyl)-5-(5-fluoro-6-(((6-(pyrazin-2-yl)imidazo[1,2-a]pyridin-8-yl)oxy)methyl)pyridin-3-yl)-1,3,4-oxadiazole

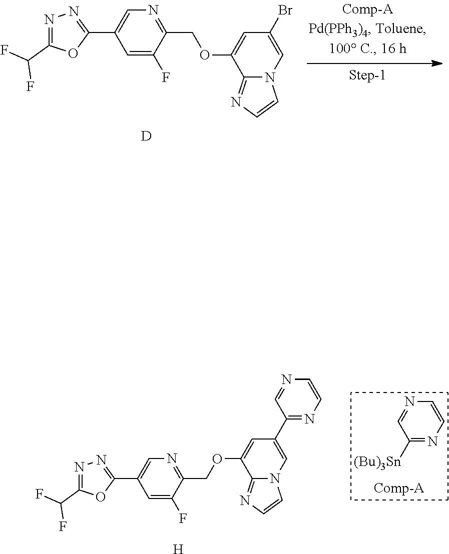

### Step-1: Synthesis of 2-(difluoromethyl)-5-(5-fluoro-6-(((6-(pyrazin-2-yl)imidazo[1,2-a]pyridin-8-yl)oxy)methyl)pyridin-3-yl)-1,3,4-oxadiazole

To a stirred solution of 2-(6-(((6-bromoimidazo[1,2-a]pyridin-8-yl)oxy)methyl)-5-fluoropyridin-3-yl)-5-(difluoromethyl)-1,3,4-oxadiazole (100 mg, 0.23 mmol, 1 eq) in toluene (1.0 mL) was added tetrakis(triphenylphosphine)palladium(0) (27.1 mg, 0.02 mmol, 0.1 eq) followed by addition of 2-(tributylstannyl)pyrazine (105 mg, 0.27 mmol, 1.2 eq). The resulting reaction mixture was heated to 100° C. for 16 h. After completion, the reaction mixture was concentrated under reduced pressure and water (30 mL) added and extracted with ethyl acetate (30 mL×3), combined organics were dried over anhydrous Na₂SO₄, filtered and concentrated under reduced pressure. The crude was purified by combi-flash column chromatography (230-400 mesh silica gel, gradient: 0-5% MeOH in DCM) to obtain 2-(difluoromethyl)-5-(5-fluoro-6-(((6-(pyrazin-2-yl)imidazo[1,2-a]pyridin-8-yl)oxy)methyl)pyridin-3-yl)-1,3,4-oxadiazole (Compound H) (12 mg, 0.03 mmol, 12% yield) as a white solid. 1H NMR (400 MHz, DMSO-d₆) δ=9.31 (d, J=1.6 Hz, 1H), 9.13 (d, J=1.6 Hz, 2H), 8.75-8.74 (m, 1H), 8.64 (d, J=2.4 Hz, 1H), 8.52 (dd, J=9.6, 1.6 Hz, 1H), 8.06 (d, J=1.2 Hz, 1H), 7.23-7.47 (m, 3H), 5.68 (d, J=1.2 Hz, 2H) ppm. LCMS (Method-A): 98.86%; 440.0 [M+H]⁺ HPLC-A (Method-A): 98.90% purity.

| Compound | Structure | Characterization |
|---|---|---|
| 36 | 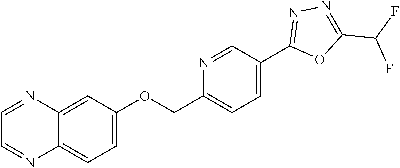 [SMILES: FC(F)c1nnc(-c2ccc(COc3ccc4nccnc4c3)nc2)o1] | ¹HNMR δ = 9.33 (d, J = 1.6 Hz, 1H), 8.83 (d, J = 2.0 Hz, 1H), 8.77 (d, J = 1.9 Hz, 1H), 8.57 (d, J = 2.3, 8.3 Hz, 1H), 8.08 (d, J = 9.3 Hz, 1H), 7.93 (d, J = 8.3 Hz, 1H), 7.71 (d, J = 2.8, 9.3 Hz, 1H), 7.56 (d, J = 2.8 Hz, 1H), 7.27 (t, J = 5.6 Hz, 1H), 5.55 (s, 2H); LCMS (m/z) 356.2 [M + H]+ |
| 56 | 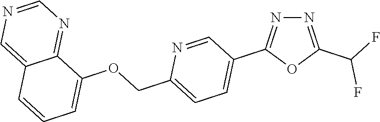 [SMILES: FC(F)c1nnc(-c2ccc(COc3cccc4cncnc34)nc2)o1] | ¹HNMR δ = 9.56 (s, 1H), 9.32-9.29 (m, 2H), 8.55 (d, J = 2.1, 8.3 Hz, 1H), 8.05 (d, J = 8.3 Hz, 1H), 7.76-7.69 (m, 2H), 7.59 (d, J = 1.4, 7.4 Hz, 1H), 7.27 (m, 1H), 5.62 (s, 2H) LCMS (m/z) 359.0 [M + H]+ |
| 86 (A) | 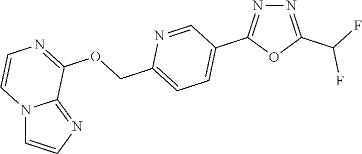 [SMILES: FC(F)c1nnc(-c2ccc(COc3nccn4ccnc34)nc2)o1] | ¹HNMR δ = 9.21 (s, 1H), 8.49-8.47 (m, 1H), 8.26 (m, 1H), 8.11 (s, 1H), 7.76-7.38 (m, 4H), 5.75 (s, 2H) LCMS (m/z) 354.2 [M + H]+ |
| 123 | 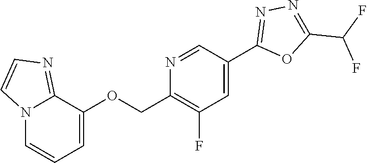 [SMILES: Fc1cc(-c2nnc(C(F)F)o2)cnc1COc1cccn2ccnc12] | ¹HNMR δ = 9.03 (s, 1H), 8.26 (d, J = 1.6, 9.5 Hz, 1H), 7.98 (d, J = 6.6 Hz, 1H), 7.72 (d, J = 1.3 Hz, 1H), 7.38 (d, J = 1.3 Hz, 1H), 7.16-7.10 (m, 1H), 6.80-6.78 (m, 1H), 6.74-6.70 (m, 1H), 5.45 (d, J = 1.9 Hz, 2H) LCMS (m/z) 362.2 [M + H]+ |
| 144 | 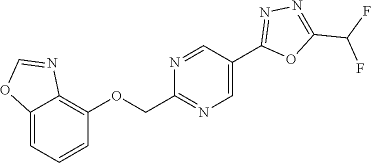 [SMILES: FC(F)c1nnc(-c2cnc(COc3cccc4ocnc34)nc2)o1] | ¹HNMR δ = 9.45 (s, 2H), 8.36 (s, 1H), 7.41-7.15 (m, 3H), 7.00 (d, J = 7.9 Hz, 1H), 5.78 (s, 2H) LCMS (m/z) 346.2 [M + H]+ |
| 150 | 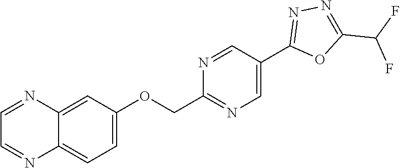 [SMILES: FC(F)c1nnc(-c2cnc(COc3ccc4nccnc4c3)nc2)o1] | ¹HNMR δ = 9.49 (s, 2H), 8.80 (d, J = 1.9 Hz, 1H), 8.75 (d, J = 2.0 Hz, 1H), 8.06 (d, J = 9.3 Hz, 1H), 7.71-7.68 (m, 1H), 7.50 (d, J = 2.9 Hz, 1H), 7.42- 7.15 (m, 1H), 5.67 (s, 2H) LCMS (m/z) 357.2 [M + H]+ |
| 151 | 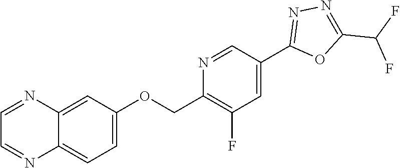 [SMILES: Fc1cc(-c2nnc(C(F)F)o2)cnc1COc1ccc2nccnc2c1] | ¹HNMR 9.12 (s, 1H), 8.95 (d, J = 1.75 Hz, 1H), 8.89 (d, J = 1.75 Hz, 1H), 8.50 (dd, J = 9.76, 1.63 Hz, 1H), 7.76- 7.82 (m, 1H), 7.67-7.74 (m, 1H), 7.60 (s, 1H), 7.46-7.52 (m, 1H), 5.60 (d, J = 1.50 Hz, 2H) ppm LCMS (m/z) 373.9 [M + H]+ |
| 155 | 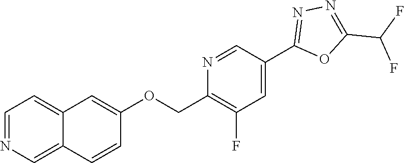 [SMILES: Fc1cc(-c2nnc(C(F)F)o2)cnc1COc1ccc2cnccc2c1] | ¹HNMR 9.21 (m, 2H), 8.36-8.53 (m, 2H), 8.06 (d, J = 9.01 Hz, 1H), 7.68- 7.75 (m, 1H), 7.50-7.61 (m, 2H), 7.31- 7.47 (m, 1H), 5.49-5.59 (m, 2H) ppm LCMS (m/z) 373.2 [M + H]+ |
| 157 | 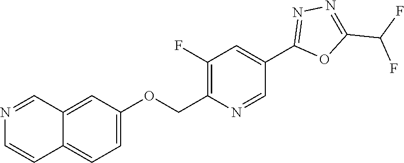 [SMILES: Fc1cc(-c2nnc(C(F)F)o2)cnc1COc1ccc2ccncc2c1] | ¹H NMR 9.21-9.21 (m, 1H), 9.12 (s, 1H), 8.49 (dd, J = 9.82, 1.69 Hz, 1H), 8.39 (d, J = 5.63 Hz, 1H), 7.93 (d, J = 9.01 Hz, 1H), 7.77 (d, J = 5.63 Hz, 1H), 7.69-7.72 (m, 1H), 7.60 (s, 1H), 7.46-7.54 (m, 1H), 5.54 (d, J = 1.50 Hz, 2H) ppm; LCMS 373.2 [M + H]+ |
| 158 | 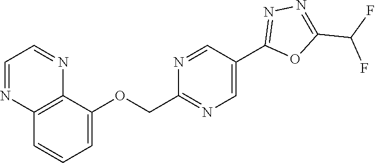 [SMILES: FC(F)c1nnc(-c2cnc(COc3cccc4nccnc34)nc2)o1] | ¹HNMR δ = 9.46 (s, 2H), 8.95 (d, J = 1.3 Hz, 1H), 8.89 (s, 1H), 7.76-7.71 (m, 2H), 7.41-7.15 (m, 2H), 5.76 (s, 2H) LCMS (m/z) 357.2 [M + H]+ |
| 160 | 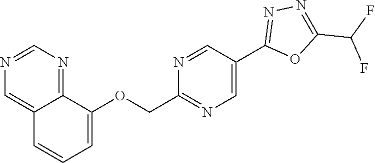 [SMILES: FC(F)c1nnc(-c2cnc(COc3cccc4cncnc34)nc2)o1] | ¹HNMR δ = 9.55 (s, 1H), 9.45 (s, 2H), 9.27 (s, 1H), 7.75-7.72 (m, 1H), 7.69- 7.64 (m, 1H), 7.52 (d, J = 7.8 Hz, 1H), 7.29-7.27 (m, 1H), 5.77 (s, 2H) LCMS (m/z) 357.2 [M + H]+ |
| B | 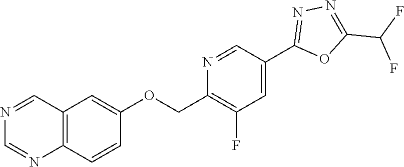 [SMILES: Fc1cc(-c2nnc(C(F)F)o2)cnc1COc1ccc2ncncc2c1] | ¹HNMR δ = 9.49 (s, 1H), 9.20 (s, 1H), 9.12 (s, 1H), 8.51-8.48 (m, 1H), 7.97 (d, 1H), 7.78-7.42 (m, 3H), 5.56 (s, 2H) LCMS (m/z) 373.8 [M + H]+ |
| C | 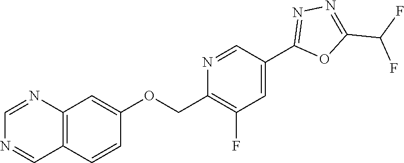 [SMILES: Fc1cc(-c2nnc(C(F)F)o2)cnc1COc1ccc2cncnc2c1] | ¹HNMR δ = 9.44 (s, 1H), 9.19 (s, 1H), 9.12 (s, 1H), 8.51-8.48 (m, 1H), 7.72-7.46 (m, 3H), 5.62 (s, 2H) LCMS (m/z) 374.4 [M + H]+ |
| D | 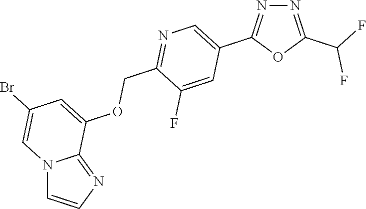 [SMILES: Fc1cc(-c2nnc(C(F)F)o2)cnc1COc1cc(Br)cn2ccnc12] | ¹HNMR δ = 9.12 (t, J = 2.4 Hz, 1H), 8.56-8.49 (m, 2H), 7.91 (d, J = 0.8 Hz, 1H), 7.60 (t, J = 51.2 Hz, 1H, CHF2), 7.49 (d, J = 1.2 Hz, 1H), 7.02 (d, J = 1.6 Hz, 1H), 5.59 (d, J = 1.6 Hz, 2H) ppm LCMS (m/z) 400.0 [M + H]+ |
| E | 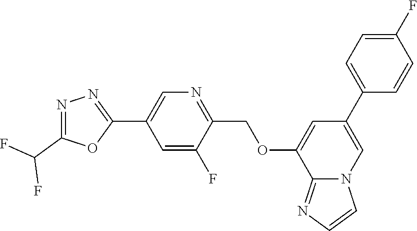 [SMILES: Fc1ccc(-c2cc(OCc3ncc(-c4nnc(C(F)F)o4)cc3F)c3nccn3c2)cc1] | ¹HNMR δ = 9.14 (s, 1H), 8.57 (d, J = 1.2 Hz, 1H), 8.51 (dd, J = 10.0, 1.6 Hz, 1H), 7.95 (d, J = 0.8 Hz, 1H), 7.80- 7.76 (m, 2H), 7.73-7.47 (m, 2H), 7.37- 7.32 (m, 2H), 7.17 (d, J = 1.2 Hz, 1H), 5.68 (d, J = 1.2 Hz, 2H) ppm LCMS (m/z) 456.0 [M + H]+ |
| F | 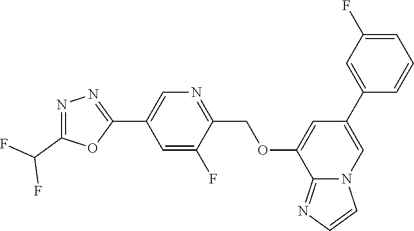 [SMILES: Fc1cccc(-c2cc(OCc3ncc(-c4nnc(C(F)F)o4)cc3F)c3nccn3c2)c1] | ¹HNMR δ = 9.18 (s, 1H), 8.17 (dd, J = 9.2, 2.0 Hz, 1H), 8.02 (d, J = 1.6 Hz, 1H), 7.63-7.62 (m, 2H), 7.45-7.42 (m, 1H), 7.35-7.33 (m, 1H), 7.24-7.22 (m, 1H), 7.10-6.82 (m, 3H), 5.68 (d, J = 1.6 Hz, 2H) ppm LCMS (m/z) 456.0 [M + H]+ |
| G | 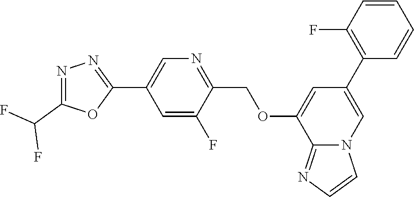 [SMILES: Fc1ccccc1-c1cc(OCc2ncc(-c3nnc(C(F)F)o3)cc2F)c2nccn2c1] | ¹HNMR δ = 9.13 (d, J = 1.2 Hz, 1H), 8.52-8.48 (m, 2H), 8.01 (d, J = 1.2 Hz, 1H), 7.73-7.45 (m, 4H), 7.39-7.33 (m, 2H), 7.02 (s, 1H), 5.64 (d, J = 1.2 Hz, 2H) ppm LCMS (m/z) 456.0 [M + H]+ |
| H | 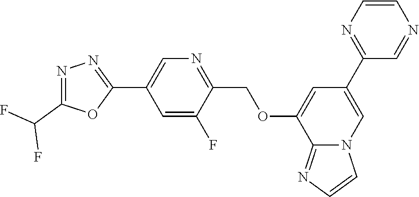 [SMILES: Fc1cc(-c2nnc(C(F)F)o2)cnc1COc1cc(-c2cnccn2)cn2ccnc12] | ¹HNMR δ = 9.31 (d, J = 1.6 Hz, 1H), 9.13 (d, J = 1.6 Hz, 2H), 8.75-8.74 (m, 1H), 8.64 (d, J = 2.4 Hz, 1H), 8.52 (dd, J = 9.6, 1.6 Hz, 1H), 8.06 (d, J = 1.2 Hz, 1H), 7.23-7.47 (m, 3H), 5.68 (d, J = 1.2 Hz, 2H) ppm LCMS (m/z) 440.0 [M + H]+ |

### Synthesis of 2-(difluoromethyl)-5-[6-(quinoxalin-6-yloxymethyl)-3-pyridyl]-1,3,4-oxadiazole (Compound 36)

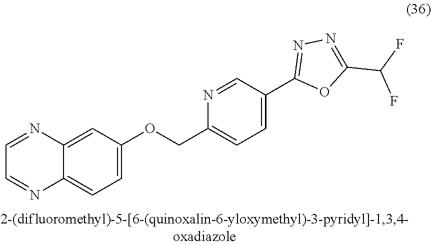 [SMILES: FC(F)c1nnc(-c2ccc(COc3ccc4nccnc4c3)nc2)o1]

Starting from 3-pyridinecarboxylic acid, prepare the hydrazide using hydrazine hydrate. React the hydrazide with difluoroacetic anhydride to form the 1,3,4-oxadiazole ring. Introduce a hydroxymethyl group ortho to the nitrogen to allow further substitution, by directed lithiation or halogenation at position 6 of the pyridine ring followed by formylation and reduction (e.g., halogenate at position 6 then formylate then reduce to hydroxymethyl), or use a pre-functionalized pyridine derivative (e.g. 6-(hydroxymethyl)-3-pyridinecarboxylic acid or related derivatives).

Synthesize the 6-(quinoxalin-6-yloxymethyl)-3-pyridyl intermediate separately: (1) synthesize 6-hydroxyquinoxaline (from o-phenylenediamine+2,3-diketone to form quinoxaline, followed by hydroxylation at position 6 or start with 6-hydroxyquinoxaline) and (2) React 6-hydroxyquinoxaline with 6-(chloromethyl)-3-pyridyl derivative (prepared from hydroxymethyl via chlorination using SOCl₂ or PPh₃/CCl₄) under basic conditions (e.g., K₂CO₃ in DMF) to form the ether.

Incorporate the substituted 3-pyridyl moiety prepared above into the 1,3,4-oxadiazole core by (a) introducing the substituted pyridine before cyclization (early stage), or (b) etherification post-oxadiazole formation (this is preferable for regioselectivity), the oxadiazole ring with the 3-pyridyl group is already prepared (late stage).

¹HNMR δ=9.33 (d, J=1.6 Hz, 1H), 8.83 (d, J=2.0 Hz, 1H), 8.77 (d, J=1.9 Hz, 1H), 8.57 (d, J=2.3, 8.3 Hz, 1H), 8.08 (d, J=9.3 Hz, 1H), 7.93 (d, J=8.3 Hz, 1H), 7.71 (d, J=2.8, 9.3 Hz, 1H), 7.56 (d, J=2.8 Hz, 1H), 7.27 (t, J=51.6 Hz, 1H), 5.55 (s, 2H);

LCMS (m/z) 356.2 [M+H]⁺

### Example 2. Human HDAC Potency and Selectivity

Compound A is a small molecule HDAC6 inhibitor that demonstrated similar in vitro inhibitory potency against human and mouse HDAC6, and in vitro selectivity against other HDAC paralogs.

Compound A is potent (<100 nM) and selective (>1000-fold) for HDAC6 over all other HDACs. Compound A demonstrates target engagement in the CNS and has favorable bioavailability. Compound A shows no dose-limiting toxicities or histological findings in a 14-day non-glp exploratory rat toxicology study.

HDAC6 potency experiments were performed to determine the potency of Compound A for human recombinant HDAC6 enzyme. The data is shown in Table 2A. The data shows that Compound A has low nanomolar potency (24 nM) for HDAC6.

TABLE 2A
HDAC6 potency

| Compound | IC50 (nM) HDAC6 |
|---|---|
| Compound A | 24 |
| Tubastatin A | 6.6 |
| Ricolinostat (ACY-1215) | 3.5 |
| Tubacin | 1.3 |

Experiments were performed to determine the selectivity of Compound A for human recombinant HDAC6 enzyme compared to all other human recombinant HDAC enzymes with Zn²⁺ dependent binding, specifically those paralogs comprising Class I, II and, IV (HDACs 1,2,3,4,5,7,8,9,10,11). The data shows that Compound A is >1699 fold selective for HDAC6 over all other HDAC paralogs. Compound A does not inhibit class I HDACs (HDACs 1, 2, 3, 8). Inhibition of class I HDACs was observed for comparators, demonstrating the high selectivity of Compound A for HDAC6. Data is shown in Table 2B. Selectivity ranges (ratio of HDAC-X IC₅₀/HDAC6 IC₅₀): I: 0.1-1; II: >1-10; III: >10-100; IV: >100-1000; V: >1000.

TABLE 2B
HDAC Selectivity

| | HDAC paralogs (HDAC-_) Fold Selectivity | | | | | | | | | |
|---|---|---|---|---|---|---|---|---|---|---|
| Cmpd | 1 | 2 | 3 | 4 | 5 | 7 | 8 | 9 | 10 | 11 |
| A | V | V | V | V | V | V | V | V | V | V |
| Tubastatin A | IV | V | IV | IV | IV | IV | IV | IV | V | V |
| Ricolinostat | III | IV | II | V | V | V | IV | V | III | V |
| (ACY-1215) | | | | | | | | | | |
| Tubacin | IV | IV | IV | V | V | V | V | V | IV | V |

These experiments were performed to determine the potency of Compound A to inhibit recombinant human HDAC6 enzyme from deacetylating a synthetic acetylated peptide substrate. The data show that Compound A has low nanomolar potency (24 nM) to inhibit HDAC6. This data demonstrates that Compound A was able to block the deacetylase function of HDAC6 at low nanomolar levels. These experiments were performed to determine the selectivity of Compound A for human HDAC6 enzyme compared to all other human HDAC enzymes with Zn2+ dependent binding, specifically those paralogs comprising Class I, II and, IV (HDACs 1,2,3,4,5,7,8,9,10,11). The data show that Compound A is >1699 fold selective for HDAC6 over all other HDAC paralogs (recombinant human HDAC enzymes tested against individually optimized acetylated peptide substrates). This data demonstrates that Compound A, which has an oxadiazole scaffold, has high selectivity for HDAC6. The level of selectivity is superior to hydroxamic acid comparators. Critically, Compound A does not inhibit class I HDAC paralogs (HDACs 1, 2, 3, 8). Inhibition of class I HDACs is linked to dose-limiting toxicity in the clinic and the high selectivity of Compound A for HDAC6 supports a good safety profile.

Compound A is a potent, selective inhibitor of HDAC6, demonstrates target engagement in the CNS, and has favorable bioavailability (Table 2C).

TABLE 2C

| Parameter | Compound A | |
|---|---|---|
| Potency | <100 | nM |
| Biochemical human HDAC6 IC₅₀ | | |
| Selectivity | >1000 | X |

| | |
|---|---|
| Human HDAC6 IC₅₀ vs other* HDACs | |
| Brain Penetration | 0.6 |
| K_{p, uu, brain} in SOD1^{G93A} mice | |
| PK Target Coverage in Brain (30 mpk, p.o.) | 48 |
| C_{avg, u}/mouse HDAC6 IC₅₀ in SOD1^{G93A} mice | |
| PD Target Coverage in Brain (30 mpk, p.o.) | 26% increase |
| Tub-Ac/Tub** at 2 h post dose in SOD1^{G93A} mice | |
| Bioavailability | 121% |
| F % in mouse | |

*Human HDACs 1, 2, 3, 8 (class I), 4, 5, 7, 9 (class IIa), 10 (class IIb), and 11 (class IV)
**Alpha tubulin acetylation on lysine 40 (Tub-Ac), relative to total alpha tubulin (Tub) levels, was evaluated using Homogenous Time Resolved Fluorescence (HTRF)
K_{p, uu, brain} = unbound brain-to-plasma ratio
C_{avg, u} = average unbound concentration over 24 hours
F % = bioavailability

In addition, Compound A exhibits a clean in vitro safety profile and shows no dose-limiting toxicities or histological findings in a 14-day exploratory rat toxicology study (Table 2D).

TABLE 2D

| Parameter | Compound A | |
|---|---|---|
| Cardiovascular Liabilities | >30 | μM |
| hERG, hCav1.2, hNav1.5 IC₅₀ | | |
| CYP Inhibition Liabilities | >50 | μM |
| CYP1A2, CYP3A4 IC₅₀ | | |
| CYP Induction Liabilities | >100/100/30 | μM |

| | |
|---|---|
| CYP1A2, CYP2B6, CYP3A4 EC₅₀ | |
| Off-target Screening | <50% at 10 μM for all 87 targets |
| SafetyScreen87 panel | |
| Genotoxicity Liabilities | Negative |
| In vitro Ames (5 strains) | |
| and micronucleus, +/−S9 | |
| Exploratory Non-GLP 14-Day | No mortality, No test-item-related |
| DRF in Rat | clinical signs, No test-item-related |
| Summary findings, highest | change in organ weight, No |
| tested dose 300 mpk | histopathological findings*, |
| | Exposure verified by toxicokinetics |

*Brain, heart, kidneys, liver, spleen, stomach, testes evaluated

### Materials and Methods

Compound A was screened in vitro in fluorogenic HDAC activity assays (IC₅₀). A 12-point concentration curve of each small molecule inhibitor was used (in 3-fold serial dilutions with 10 μM as the top concentration for HDAC6 and 100 μM as the top concentration for all other HDAC paralogs (HDAC 1,2,3,4,5,7,8,9,10 and 11).

Purified recombinant human HDAC enzyme substrate, and cofactor(s) were obtained from BPS Biosciences (SanDiego, CA, USA). Each enzyme was pre-incubated in a microtiter plate with dilutions of test compounds or control/comparator small molecule inhibitor compounds for three hours to maximize engagement of inhibitors with slow-on rates. Acetylated peptide substrate and cofactor(s) were then added, followed by incubation with reaction monitoring for 1 hour at 25° C. The final reaction mixture comprised: 100 mM HEPES, pH 7.5; 0.01% BSA; 0.01% Triton X-100; 25 mM KCl; 1% DMSO (from small molecule inhibitors, solubilized as 10 mM stocks in 100% DMSO); 1.25 μM Fluorogenic HDAC substrate 3 (LGK(Ac)-AMC, BPS Catalog #50037) for HDAC1,2,3,6,10 or Fluorogenic HDAC class 2a substrate (LGK(Tfa), BPS Catalog #50040) for HDAC4,5,7,8,9,11. At the end of the incubation, the reaction was quenched by the addition of a developer buffer comprising Trichostatin A and trypsin which reacts with the deacetylated substrate to release fluorophore. Plates were read using a BioTek Synergy Neo2 and quantitated by fluorescence intensity.

These assays demonstrate that Compound A displayed high potency (24 nM) to inhibit HDAC6 and high selectivity (>1699-fold for HDAC8 and >1924-fold versus all other HDAC paralogs).

### Example 3. HDAC Enzyme Activity Assay

### Caliper Endpoint Assay for HDAC Enzymatic Activity

Enzymatic activity of compounds of the disclosure were determined in one of the two biochemical assays described below. Both follow the same protocol. The first relies on a caliper chip readout, while the second relies on a fluorogenic readout.

HDAC reactions were assembled in 384 well plates (Greiner) in a total volume of 20 μL as follows: HDAC proteins (and their regulatory subunit, if applicable) were pre-diluted in the assay buffer comprising: 100 mM HEPES, pH 7.5, 0.1% BSA, 0.01% Triton X-100, 25 mM KCl and dispensed into a 384 well plate (10 μL per well). An example of enzyme concentrations used in each assay is listed in the table below.

| | | | | | Substrate | |
|---|---|---|---|---|---|---|
| | | Regulatory | [Enzyme] | Substrate | Conc | Incubation |
| Assay | Expression Construct | subunit | nM | Peptide | (μM) | Time (hr) |
| HDAC6 | Full length Human HDAC6 | None | 6 | FAM- | 1 | 5 |
| | with C-terminal FLAG-tag, | | | RHKK(Ac)- | | |
| | expressed in baculovirus | | | NH2 | | |
| | expression system. | | | | | |
| HDAC8 | Full length Human HDAC8 | None | 5 | FAM- | 1 | 3 |
| | with N-terminal HIS-tag, | | | RHKK(TFAc)- | | |
| | expressed in baculovirus | | | NH2 | | |
| | expression system. | | | | | |

Test compounds were serially pre-diluted in 100% DMSO using 3-fold dilution steps and added to the protein samples by acoustic dispensing (Labcyte Echo). Concentration of DMSO was equalized to 1% in all samples. Final compound concentration in assays typically ranged from 100 μM to 0.00056 μM for a 12-point concentration-response format.

Control samples (0%-inhibition in the absence of inhibitor, DMSO only) and 100%-inhibition (in the absence of enzyme) were assembled in replicates of four (for each caliper sipper) and used to calculate the %-inhibition in the presence of compounds. At this step compounds were pre-incubated with enzyme for 30 minutes at room temperature (20-23° C.). The reactions were initiated by addition of 10 μL of the FAM-labeled substrate peptide (see table above) pre-diluted in the same assay buffer. Final concentration of substrate peptide was 1 μM. The reactions were allowed to proceed at room temperature (20-23° C.). Typical incubation times for each HDAC, based on pre-determined enzyme progress curves, vary and are listed in table above.

Following incubation, the reactions were quenched by addition of 50 μL of termination buffer (100 mM HEPES, pH7.5, 0.01% Triton X-100, 0.05% SDS). Terminated plates were analyzed on a microfluidic electrophoresis instrument (Caliper LabChip® 3000, Caliper Life Sciences/Perkin Elmer) which enables electrophoretic separation of deacetylated product from acetylated substrate. A change in the relative intensity of the peptide substrate and product is the parameter measured. Activity in each test sample was determined as the product to sum ratio (PSR): P/(S+P), where P is the peak height of the product, and S is the peak height of the substrate. Percent inhibition (Pᵢₙₕ) is determined using the following equation:

Pᵢₙₕ=(PSR_{0% inh}−PSR_{compound})/(PSR_{0% inh}−PSR_{100% inh})*100,

in which: PSR_{compound} is the product/sum ratio in the presence of compound, PSR_{0% inh} is the product/sum ratio in the absence of compound and the PSR_{100% inh} is the product/sum ratio in the absence of the enzyme. To determine the IC₅₀ of compounds (50%-inhibition) the %-inh data (Pᵢₙₕ versus compound concentration) were fitted by a 4 parameter sigmoid dose-response model using XLfit software (IDBS).

### Nanosyn Biochemical HDAC6 Assay (Fluorogenic Format)

HDAC protein composition and respective substrate peptides are summarized in table below:

| Assay | | Regulatory | Substrate |
|---|---|---|---|
| name | Expression Construct | subunit | peptide |
| hHDAC6 | Full length Human HDAC6 with C-terminal | None | LGK(Ac)-AMC |
| | FLAG-tag, expressed in baculovirus expression | | |
| | system. | | |
| mHDAC6 | Full length Mouse HDAC6, a.a. 2-1149 (end) with | None | LGK(Ac)-AMC |
| | N-terminal GST-Tag, expressed in baculovirus | | |
| | expression system. | | |

The biochemical HDAC6 assay was performed using fluorescence detection (Fluor-De-Lys assay). In this assay, deacetylation of the Lysine residue in the LGK(Ac)-AMC peptide substrate, results in a cleavable bond between the fluorescent moiety of amino-methyl coumarin (AMC) and lysine. The AMC is released by the auxiliary treatment with trypsin, which is added to the termination buffer. The cleaved AMC generates strong fluorescent signal which is being detected (360 nm excitation and 460 nm emission).

HDAC reactions are assembled in black low binding 384 well plates (Corning) in a total volume of 20 mL as following:

HDAC6 protein is pre-diluted in the assay buffer comprising of: 100 mM HEPES, pH 7.5, 0.01% BSA, 0.01% Triton X-100, 25 mM KCl and dispensed into 384 well plate (10 uL per well).

Test compounds are serially pre-diluted in DMSO and added to the protein samples by acoustic dispensing (Labcyte Echo). Concentration of DMSO is equalized to 1% in all samples.

Control samples (0%-inhibition in the absence of inhibitor, DMSO only) and 100%-inhibition (in the absence of enzyme) are assembled in the same plate and used to calculate the %-inhibition in the presence of compounds.

At this step compounds are pre-incubated with enzyme for 30 min.

The reactions are initiated by addition of 10 uL of the LGK(Ac)-AMC substrate peptide pre-diluted in the same assay buffer.

Final concentration of hHDAC6 enzyme is 0.5 nM. Final concentration of mHDAC6 enzyme is 1 nM. Final concentration of substrate peptide is 1.25 uM.

The reactions are allowed to proceed at room temperature for 1 h.

Following incubation, the reactions are quenched by addition of 20 mL of termination buffer comprising of Trichostatin A (included as a termination agent) and trypsin (BPS catalog #50030). Terminated plates are read on Synergy Neo2 luminometer. A change in the relative luminescence intensity (RLUs) is the parameter measured. Percent inhibition (Pᵢₙₕ) is determined using the following equation:

Pᵢₙₕ=(RLU_{0% inh}−RLU_{compound})/(RLU_{0% inh}−RLU_{100% inh})*100,

in which: RLU_{compound} is the sample luminescence in the presence of compound, RLU_{0% inh} is the sample luminescence in the absence of compound and the RLU_{100% inh} is the sample luminescence in the absence of the enzyme. To determine IC50 of compounds (50%-inhibition) the %-inh data (Pᵢₙₕ versus compound concentration) are fitted by a 4 parameter sigmoid dose-response model using XLfit software (IDBS)

Exemplary compounds were evaluated for inhibitory activity of a panel of HDAC paralogs. The results in Table 1 demonstrate that compounds of the disclosure have potent activity against HDAC6, and many compounds selectively inhibit HDAC6 over the Class I HDAC paralog HDAC8.

IC₅₀ ranges: A: 0.001-0.1 μM; B: >0.1-1 μM; C: >1-10 M; D: >10-100 μM; E: >100 μM.

Selectivity ranges (ratio of HDAC8 IC₅₀/HDAC6 IC₅₀): I: 0.1-1; II: >1-10; III: >10-100; IV: >100-1000; V: >1000

TABLE 1
In Vitro Enzymatic IC₅₀ values for exemplary compounds

| | | | Selectivity | | |
|---|---|---|---|---|---|
| | HDAC6 | HDAC8 | (6 v 8) | | HDAC6 |
| Compound | IC50 | IC50 | (fold) | Compound | IC50 |
| 10 | A | E | V | 123 | A |
| 14 | B | | | 124 | A |
| 28 | A | E | V | 125 | A |
| 31 | A | C | IV | 126 | A |
| 32 | A | D | V | 130 | A |
| 33 | A | | | 132 | A |
| 34 | A | E | V | 134 | A |
| 35 | A | E | V | 136 | A |
| 36 | A | E | V | 137 | A |
| 37 | A | D | V | 138 | A |
| 38 | A | | | 139 | A |
| 53 | A | E | V | 140 | A |
| 55 | A | E | V | 141 | A |
| 56 | A | E | V | 144 | A |
| 58 | A | E | V | 146 | A |
| 59 | A | E | V | 147 | A |
| 60 | A | D | V | 150 | A |
| 61 | A | D | IV | 151 | A |
| 62 | A | D | V | 152 | A |
| 63 | A | E | V | 153 | A |
| 82 | A | E | V | 155 | A |
| 86 | A | | | 156 | A |
| 87 | A | | | 157 | A |
| 88 | A | D | V | 158 | A |
| 89 | A | | | 159 | A |
| 95 | A | | | 160 | A |
| 96 | A | | | 161 | A |
| 97 | A | E | V | 178 | A |
| 98 | A | | | 179 | A |
| 184 | B | E | IV | 180 | A |
| 202 | B | E | IV | 181 | A |
| 203 | C | E | III | 182 | A |
| 204 | A | E | V | 183 | A |
| 209 | C | E | III | 208 | A |
| 210 | C | E | III | 213 | A |
| 211 | B | E | IV | | |
| 212 | B | E | IV | | |
| trichostatin A | A | B | IV | | |
| (TSA) | | | | | |

### Example 4. Pharmacokinetic Profiles and Brain Penetration of Compound A

### Example 4A

These experiments were performed to determine the pharmacokinetic profile of Compound A in mouse across multiple doses (3, 10, 30 mg/kg) after a single oral administration.

FIG. 1A is a graph showing the plasma, brain and spinal cord concentrations of Compound A (ng/mL) in mice within 8 hours after the oral administration of Compound A at a dose of 3 mg/kg. FIG. 1B is a graph showing the plasma, brain and spinal cord concentrations of Compound A (ng/mL) in mice within 8 hours after the oral administration of Compound A at a dose of 10 mg/kg. FIG. 1C is a graph showing the brain and spinal cord concentrations of Compound A (ng/mL) in mice within 8 hours, and the plasma concentrations of Compound A (ng/mL) in mice within 24 hours, after the oral administration of Compound A at a dose of 30 mg/kg.

The data (Table 4A and FIGS. 1A, 1B and 1C) show that Compound A has a Cₘₐₓ (ng/mL) of 1,469 at 3 mg/kg, 5,002 at 10 mg/kg, and 11,335 at 30 mg/kg. Compound A has a T max (h) of 0.5 at 3 mg/kg, 0.5 at 10 mg/kg, and 2.0 at 30 mg/kg. Compound A has an AUCₗₐₛₜ (ng*h/mL) of 4,571 at 3 mg/kg, 16,939 at 10 mg/kg, and 80,797 at 30 mg/kg. This data demonstrates that the exposure of Compound A increases with increasing dose in a more than dose proportional manner and exhibits sustained target coverage relative to the biochemical human HDAC6 IC50 for at least 8 h following a single oral dose at 3, 10, and 30 mg/kg.

TABLE 4A
Mouse PK data following single oral dose of Compound A.

| Dose | Cₘₐₓ | Tₘₐₓ | AUCₗₐₛₜ | Brain Kₚ | | Brain Kₚᵤᵤ | Spinal Cord Kp | |
|---|---|---|---|---|---|---|---|---|
| (mg/kg) | (ng/mL) | (h) | (ng h · mL) | Cₘₐₓ | AUC | AUC | Cₘₐₓ | AUC |
| 3 | 1,469 | 0.50 | 4,571 | 0.49 | 0.47 | 0.47 | 0.36 | 0.34 |
| 10 | 5,002 | 0.50 | 16,939 | 0.45 | 0.46 | 0.46 | 0.31 | 0.34 |
| 30 | 11,335 | 2.00 | 80,797 | 0.49 | 0.34 | 0.34 | 0.37 | 0.27 |

### Example 4B

Experiments were performed to determine the brain to plasma ratio (Kp) and brain to plasma ratio corrected for unbound compound concentrations (Kpuu) across multiple doses (3, 10, 30 mg/kg) in mouse after single oral administration. The data (Table 4A) show that Compound A has a Kp range of 0.34-0.47 and a Kpuu range of 0.34-0.47 in mouse. This data demonstrates that Compound A penetrates the brain with a high unbound concentration across doses. The comparator ricolinostat (ACY-1215) does not get into the brain. Ricolinostat Kp could not be quantified as compound concentration in mouse brain was below level of quantification after a single oral administration at 30 mg/kg. Extrapolation from literature suggests that ricolinostat has a Kp of 0.03 in mouse brain at Cmax (Ma et al., 2018; 6: 103).

An experiment was performed to determine the brain to plasma ratio (Kp) at 1,000 mg/kg in rat. The data (Table 4B) shows that Compound A has a Kp (0.45) in rat. This data demonstrates that Compound A penetrates into the brain.

### Materials and Methods

### Brain Protein Binding Protocol

Compound concentration: 5 μM; Matrix: Mice brain; Incubation temperature: 37° C.; Incubation time: 4 hours; fractional DMSO in final reaction: 0.5%; Replicates: 3. Phosphate buffered saline pack (0.1 M sodium phosphate and 0.15 M sodium chloride, pH 7.2) was dissolved in 500 mL of deionized water and pH was adjusted to 7.4 with 0.5 N NaOH. Brain tissue homogenate samples were prepared by diluting one volume of whole brain tissue with three volumes of dialysis buffer (phosphate buffered saline pH 7.4-0.1 M sodium phosphate and 0.15 M sodium chloride) to yield 4 times diluted homogenate. 1 mM stock solutions of test compounds were prepared in DMSO and diluted 200-fold in mouse brain homogenate to yield concentration of 5 μM. The final DMSO concentration was 0.5%.

Rapid equilibrium dialysis was performed with a rapid equilibrium dialysis (RED) device containing dialysis membrane with a molecular weight cut-off of 8,000 Daltons. Each dialysis insert contains two chambers. The red chamber was for brain homogenate while the white chamber for buffer. A 200 μL aliquot of test compound and positive controls in brain homogenate (in triplicate) was added to the red chamber of dialysis inserts. A 350 μL aliquot of dialysis buffer was added to the buffer chamber of the inserts. After sealing the RED device with an adhesive film, dialysis was done at 37° C. with shaking at 100 RPM for 4 hours.

Recovery and stability: A 50 μL aliquot of test compound and positive controls were separately added to 0.5 mL eppendorf tubes. Two aliquots were frozen immediately (0-minute sample). Two other aliquots were incubated at 37° C. for 4 hours along with the RED device.

Following dialysis, an aliquot of 50 μL was removed from each well (brain homogenate and buffer) and diluted with equal volume of opposite matrix to nullify the matrix effect. Similarly, buffer was added to recovery and stability samples. An aliquot of 100 μL was submitted for LC-MS/MS analysis.

A 25 μL aliquot of the positive control and test compound were crashed with 100 μL of acetonitrile containing internal standards (glipizide) and vortexed for 5 min. The samples were centrifuged at 4000 RPM at 4° C. for 10 min and 100 μL of supernatant was submitted for LC-MS/MS analysis. Samples were monitored for parent compound in MRM mode using LC-MS/MS.

The percentage of the test compound bound is calculated based on the following formula:

$$\% Free = ( Concentration buffer chamber / Concentration plasma chamber ) \times 100 \%$$

$$\% Bound = 100 \% - \% Free$$

$$\% Recovery = \{ ( Vdialysate \times Cfree + Vplasma \times Ctotal ) / ( Vplasma \times Czero ) ] \times 100 \%$$

The unbound concentration of Compound A as determined in this assay for mouse brain homogenate was 0.34 (34% unbound); this represents a moderate free fraction in mouse brain and was substantially higher than the free fraction of Ricolinostat (ACY-1215) at 0.062 (6.2% unbound).

### Plasma Protein Binding Protocol

A 10 mM stock of compound was prepared in DMSO. From the stock solution, a 2.5 mM working solution of compound was prepared in DMSO and spiked in neat plasma (from desired species, mice—C57BL/6; Rat—Sprague Dawley Rat Plasma (K2EDTA), Human—Matrix: Sodium citrate Plasma-Blank, to achieve the final concentration of 10 μM in Incubation mixture. Subsequently, 50 μL of sample from this mixture was added into 50 μL of DPBS and immediately vortexed with 400 μL of precipitation buffer (cold 90/10 Methanol/Acetonitrile with 0.1% formic acid) containing internal standard—Carbamazepine (60 ng/mL). This resulting sample (T=0 min) was incubated on ice for 30 minutes and then centrifuged at 3200×g and 4° C. for 30 minutes.

The RED device was placed onto the base plate following which 200 μL of the incubation mixture is added in the chamber of the RED device which is encircled by a red ring. In the other chamber 350 μL of buffer (DPBS, pH 7.4) was added and sealed (with a sealer) to avoid any evaporation or spilling of the samples from the chamber. The base plate was then put on an orbital shaker at 300 rpm for 5 hours at 37° C. After this incubation period, the base plate was taken out and subsequently, 50 μL of post dialysis samples from both chambers were collected in separate wells of the 96 well deep well plates.

Following this, 50 μL of pertinent plasma was added to the collected buffer sample and 50 μL of buffer was added to the collected plasma sample. Precipitation buffer (500 μL; cold 90/10 Methanol/Acetonitrile with 0.1% formic acid) containing internal standard—Carbamazepine (60 ng/mL) was added to precipitate the protein and to release the compound. The samples were vortexed and incubated on ice for 30 minutes. Following the incubation, the samples were centrifuged at 3200×g and 4° C. for 30 minutes. Supernatants from all the samples were collected in LCMS vials and analyzed in duplicates using LCMS/MS. The concentration of test compound was determined in the buffer and plasma chamber from the peak areas obtained (LCMS/MS analysis).

The percentage of the test compound bound is calculated based on the following formula:

$$\% Free = ( Concentration buffer chamber / Concentration plasma chamber ) \times 100 \%$$

$$\% Bound = 100 \% - \% Free$$

$$\% Recovery = \{ ( Vdialysate \times Cfree + Vplasma \times Ctotal ) / ( Vplasma \times Czero ) ] \times 100 \%$$

The unbound concentration of Compound A as determined in this assay for mouse plasma was 0.267 and 0.338 (26.7 and 33.8% unbound, respectively); and in human plasma 0.34 (34% unbound); this represents a moderate free fraction in mouse and human plasma and was substantially higher than the free fraction of Ricolinostat (ACY-1215) in mouse and human plasma at 0.0265 and 0.087 respectively (2.6% and 8.7% unbound, respectively.

### Brain to Plasma Partition Coefficient

The brain to plasma partition coefficient (Kp) is a calculated value based on the ratio of the brain compound concentrations at Cmax or AUC (ng*h/mL) and plasma Cmax or AUC (ng*h/mL) obtained as part of a single PK experiment. Refinements for unbound compound concentrations present in brain and plasma (Kpuu) also take into account data from two additional experiments wherein protein binding is determined for the small molecule inhibitor in homogenized mouse brain or in mouse plasma. Brain protein binding or plasma protein binding fraction unbound (fu,brain or fu, plasma) are expressed as separate values, each between 0.0 (fully bound to brain or plasma proteins) or 1.0 (fully unbound to brain or plasma proteins).

Kp values>1.0 indicate a compound has a higher amount in the brain compartment than the plasma compartment. Kpuu values (Kp*fu,brain/fu, plasma) may be highly similar to Kp values when the unbound fraction in brain and in plasma is very high (close to 1.0).

Compound A Kp and Kpuu was calculated in: mouse=0.34-0.47, 0.34-0.47

Compound A Kp was calculated as 0.45 in rat.

The data in mice are presented as a range given that each of the experiments underlying Kp and Kpuu were repeated across dose and demonstrated similar but distinct values.

A Kp and Kpuu of 0.34-0.47 represents excellent brain partitioning with robust fractions of available compound.

### Example 5. Selective In Vitro Target Engagement of Compound A

Experiments were performed to confirm Compound A target engagement in vitro, as measured by an increase in alpha-tubulin acetylation levels, which is the dominant substrate for HDAC6. The data shows a dose-dependent increase in alpha-tubulin acetylation levels beginning after ˜0.1 micromolar with a 310% increase observed at 1 micromolar and a >600% increase observed at 10 micromolar and 30 micromolar in human SH-SY5Y cells. This data demonstrates that Compound A is cell permeable and engages the HDAC6 target. Experiments were performed to confirm that Compound A does not engage the off-target class I HDACs in vitro, as measured by an increase in histone H3 lysine 9 acetylation levels. The data shows that at 10 micromolar and 30 micromolar Compound A does not increase histone acetylation in human SH-SY5Y cells, while the broad-acting HDAC inhibitor SAHA increases histone acetylation by ˜600%. This data demonstrates that Compound A is selective for HDAC6.

FIG. 2 is a graph showing target engagement data obtained for the HDAC6 substrate acetyl alpha tubulin relative to total alpha tubulin (Ac-Tubulin/Tubulin) normalized to the 0.1% DMSO condition vs concentration (micromolar) of Compound A in human SH-SY5Y cells.

FIG. 3A is a bar graph comparing the specific target engagement via the HDAC6 substrate acetyl alpha tubulin relative to total alpha tubulin (Ac-Tub/Total Tub) normalized to the 0.1% DMSO condition for Compound A and SAHA at 10 micromolar and 30 micromolar in human SH-SY5Y cells.

FIG. 3B is a bar graph comparing off-target engagement via the class I HDAC substrate acetyl histone H3 lysine 9 relative to total histone H3 (Ac-H3K9/Total H3) normalized to the 0.1% DMSO condition for Compound A and SAHA at 10 micromolar and 30 micromolar in human SH-SY5Y cells.

### Materials and Methods for In Vitro Target Engagement Via Acetylated Tubulin

SH-SY5Y cells were plated at a density of 35,000 cells per well in a 96 well plate (Corning #3904) with 90 μL complete media (DMEM:F12 with 10% FBS) per well. Plates were incubated at 37° C., 5% CO₂ overnight. Eleven doses of Compound A (100, 33, 11, 3.7, 1.2, 0.41, 0.14, 0.046, 0.015, 0.0051, 0.0017 μM) and two doses of SAHA (33, 11 μM) were prepared at 10× their final concentration in 1% DMSO and complete media. To each well of the cell plate, 10 μL of the corresponding dose or vehicle control (DMSO) was added and incubated at 37° C. and 5% CO₂ for 4 hrs.

Homogeneous Time Resolved Fluorescence (HTRF) was used to analyze the ratio of acetyl-α-tubulin (63ADK072PEH; Revvity, Waltham, MA, USA) to total α-tubulin (63ADK073PEH; Revvity, Waltham, MA, USA). Cells were washed with PBS and lysed using 100 μL of 1× Cisbio lysis buffer (64KL1FDF; Revvity, Waltham, MA, USA) supplemented with protease (11836170001; Sigma Aldrich, St. Louis, MO, USA) and phosphatase inhibitors (04906837001; Roche, Basel, Switzerland) on ice for 25 min. Following a further 1:5 dilution of samples in 1× Cisbio lysis buffer, 16 μL of each lysate was added to duplicate wells of a white opaque 384 well low volume plate (6008280; Revvity, Waltham, MA, USA) for both the acetyl-α-tubulin and total α-tubulin assays. A D2 and Cryptate mixture was added to each well. Buffer blank was included as a negative control along with the kit specific positive control. The plate was incubated for 2 hours at room temperature and then read on an Envision plate reader at 665 nm and 620 nm. The 665 nm/620 nm ratio was used to calculate the acetyl-α-tubulin and total α-tubulin specific HTRF ratios. The ratio of acetyl-α-tubulin to total α-tubulin was then calculated for each sample and normalized to the vehicle (DMSO) control. Compound A treated SH-SY5Y cells showed a dose dependent increase in acetyl-α-tubulin with an ˜8-fold increase over the vehicle (DMSO) control at the highest tested dose (100 μM). SAHA, a pan HDAC inhibitor, increased acetyl-α-tubulin with an ˜8-9-fold increase over the vehicle (DMSO) control at 10 and 30 μM. This data demonstrates that Compound A engages the HDAC6 target.

### Materials and Methods for Off Target Class I HDAC Engagement Via Histone Acetylation

SH-SY5Y cells were plated at a density of 35,000 cells per well in a 96 well plate (Corning #3904) with 90 μL complete media (DMEM:F12 with 10% FBS) per well. Plates were incubated at 37° C., 5% CO₂ overnight. Eleven doses of Compound A (100, 33, 11, 3.7, 1.2, 0.41, 0.14, 0.046, 0.015, 0.0051, 0.0017 μM) and two doses of SAHA (33, 11 μM) were prepared at 10× their final concentration in 1% DMSO and complete media. To each well of the cell plate, 10 μL of the corresponding dose or vehicle control (DMSO) was added and incubated at 37° C. and 5% CO₂ for 4 hrs. Homogeneous Time Resolved Fluorescence (HTRF) was used to analyze the ratio of acetyl-α-tubulin (63ADK072PEH; Revvity, Waltham, MA, USA) to total α-tubulin (63ADK073PEH; Revvity, Waltham, MA, USA). Cells were washed with PBS and lysed using 100 μL of 1× Cisbio lysis buffer (64KL1FDF; Revvity, Waltham, MA, USA) supplemented with protease (11836170001; Sigma Aldrich, St. Louis, MO, USA) and phosphatase inhibitors (04906837001; Roche, Basel, Switzerland) on ice for 25 min.

AlphaLISA immunoassay was used for the detection of acetyl-histone H3 Lysine 9 (H3K9ac) (AL714C; Revvity, Waltham, MA, USA) and unmodified histone H3 Lysine 4 (H3K4) (AL719C; Revvity, Waltham, MA, USA). Lysates were diluted in histone lysis buffer (AL009C1; Revvity, Waltham, MA, USA) and added to the PerkinElmer 96-well ½ AreaPlate (6005560; Revvity, Waltham, MA, USA). Cell-Histone extraction buffer (AL009C2; Revvity, Waltham, MA, USA) was added to each well and incubated for 10 min at room temperature. Acceptor antibody was diluted 1:50 in 1× histone detection buffer (AL009C3; Revvity, Waltham, MA, USA) and then biotinylated anti-Histone H3 antibody was added. The acceptor-biotinylated antibody mixture was added to each well of the plate, which was then covered with foil seal (232699; Thermo Fisher Scientific, Waltham, MA, USA) and incubated at room temperature for 60 min. Donor antibody was diluted 1:50 in 1× histone detection buffer and then added to each well of the plate, which was then covered with foil seal and incubated at room temperature for 30 min. The plate was then read on an Envision plate reader 96-well HTS Alpha Screen aperture. The blank well OD value was subtracted from each of the assay wells to determine the amount of H3K9ac and unmodified H3K4 per samples. H3K9ac was normalized to unmodified H3K4 for each sample and the fold change was calculated by comparing normalized H3K9ac levels in the vehicle (DMSO) to that of the samples. Compound A treated SH-SY5Y cells did not show an increase in acetyl-H3K9 over the vehicle (DMSO) control at the highest tested dose (30 PM). SAHA, a pan HDAC inhibitor, increased acetyl-H3K9 levels with a ˜6-7-fold increase over the vehicle (DMSO) control at 10 and 30 μM. This data demonstrates that Compound A is selective in cells and does not engage class I HDACs, which are off-target.

### Example 6. Pharmacokinetics and Tissue (Brain and Spinal Cord) Distribution of Compound A in Male C57BL/6 Mice Following a Single Oral Administration

The objective of this study was to investigate the plasma pharmacokinetics and tissue (brain and spinal cord) distribution of Compound A in male C57BL/6 mice following a single oral administration at doses of 3, 10, and 30 mg/kg. A total of thirty-nine male mice were included in the study and were divided into four groups as Group 1, Group 2, and Group 3 (n=12/group) and Group 4 (n=3; untreated control group) with 3 mice/time point design. Animals in Group 1 and Group 2 were administered orally with solution formulation and Group 3 were administered with suspension formulation of Compound A at 3, 10, and 30 mg/kg dose, respectively. The formulation vehicle was 7.5% v/v NMP, 5% v/v Solutol HS-15, 30% v/v PEG-400 and 57.5% v/v Citric acid (10 mM).

Blood samples (approximately 60 μL) were collected under light isoflurane anesthesia (Surgivet®) from retro orbital plexus from a set of three mice at 0.5, 2, 8, and 24 hrs post dose. Immediately after blood collection, plasma was harvested by centrifugation at 10000 rpm, 10 min at 4° C. and samples were stored at −70±10° C. until bioanalysis. Following blood collection, immediately animals were sacrificed followed by abdominal vena-cava was cut open and whole body was perfused from heart using 25 mL of normal saline. Brain, spinal cord and spleen samples were collected from set of three mice at 0.5, 2, 8, and 24 hrs post dose. After isolation, brain, spinal cord and spleen samples were rinsed three times in ice cold normal saline (for 5-10 seconds/rinsed using ˜5-10 mL normal saline in disposable petri dish for each rinse) and dried on blotting paper. Brain samples were sagittally bisected, weighed and half portion was homogenized using ice-cold phosphate buffer saline (pH-7.4) and used for PK analysis. Total homogenate volume was three times the tissue weight. All homogenates were stored below −70±10° C. until bioanalysis. Remaining half portion was separated into three portions (cortex, hippocampus and remaining brain). The spinal cord was bisected along rostro-caudal axis. The rostro spinal cord (half towards nose side) was used for PK analysis. These brain regions, caudal spinal cord (half towards tail side) and spleen samples obtained from treatment group and untreated control (n=3 mice; at 0 h) were frozen on dry ice and stored at −70±10° C.

Brain and spinal cord samples were diluted (1-part of tissue: 2-part of buffer) and homogenized. The homogenate was submitted for bioanalysis and the concentrations (ng/mL) received were corrected with dilution factor (3×) and the final reported concentrations were represented in ng/g.

All samples (plasma, half brain, and rostral spinal cord) were processed for analysis by protein precipitation method and analyzed with fit-for-purpose LC-MS/MS method (LLOQ=2.02 ng/mL for plasma, brain and spinal cord). The pharmacokinetic parameters were estimated using non-compartmental analysis tool of Phoenix® WinNonlin software (Ver 8.3) and parameters are summarized below:

TABLE A

| | | Dose | Tₘₐₓ | Cₘₐₓ | AUCₗₐₛₜ | Cₘₐₓ | AUCₗₐₛₜ | | |
|---|---|---|---|---|---|---|---|---|---|
| Matrix | Route | (mg/kg) | (h) | (ng/mL) | (h*ng/mL) | Ratio | Ratio | F (%) | — |
| Plasma | PO | 3 | 0.50 | 1469 | 4571 | — | — | | — |
| | | 10 | 0.50 | 5002 | 16939 | 3.41 | 3.71 | | — |
| | | 30 | 2.00 | 11335 | 80797 | 7.72 | 17.7 | | — |

| | | Dose | Tₘₐₓ | Cₘₐₓ | AUCₗₐₛₜ | Cₘₐₓ | AUCₗₐₛₜ | Brain-Kp | Brain-Kp |
|---|---|---|---|---|---|---|---|---|---|
| Matrix | Route | (mg/kg) | (h) | (ng/g) | (h*ng/g) | Ratio | Ratio | (Cₘₐₓ) | (AUCₗₐₛₜ) |
| Brain^{#} | PO | 3 | 0.50 | 717 | 2155 | — | — | 0.49 | 0.47 |
| | | 10 | 0.50 | 2240 | 7747 | 3.12 | 3.60 | 0.45 | 0.46 |
| | | 30 | 2.00 | 5568 | 27698 | 7.76 | 12.9 | 0.49 | 0.34 |
| | | Dose | Tₘₐₓ | Cₘₐₓ | AUCₗₐₛₜ | Cₘₐₓ | AUCₗₐₛₜ | Brain-Kp | Brain-Kp |
| Matrix | Route | (mg/kg) | (h) | (ng/g) | (h*ng/g) | Ratio | Ratio | (Cₘₐₓ) | (AUCₗₐₛₜ) |
| Spinal | PO | 3 | 0.50 | 535 | 1565 | — | — | 0.36 | 0.34 |
| Cord^{#} | | 10 | 0.50 | 1567 | 5812 | 2.93 | 3.71 | 0.31 | 0.34 |
| | | 30 | 2.00 | 4210 | 22170 | 7.87 | 14.2 | 0.37 | 0.27 |

^{#}Brain and spinal cord, Cmax and AUC are expressed as ng/g and h* ng/g, respectively, Cₘₐₓ and AUCₗₐₛₜ ratio were calculated from lowest dose administered.

Following a single oral administration of Compound A in male C57BL/6 mice at 3, 10, and 30 mg/kg, the peak plasma concentrations (Cₘₐₓ) were observed at 0.5, 0.5, and 2 h post dose, respectively, suggesting rapid rate of absorption.

Brain concentrations were quantifiable up to 8 h post dose following oral administration at 3, 10, and 30 mg/kg dose with brain exposure-Kp of 0.47, 0.46 and 0.34 respectively.

Spinal cord concentrations were quantifiable up to 8 h following oral administration at 3, 10, and 30 mg/kg dose with spinal cord exposure-Kp of 0.34, 0.34, and 0.27 respectively.

Following single oral administration of Compound A at 3, 10, and 30 mg/kg, more than dose proportional increase in exposure with increasing doses was observed in plasma, brain, and spinal cord.

Following a single oral administration of Compound A in male C57BL/6 mice at 3, 10, and 30 mg/kg dose, the peak plasma concentrations (Cₘₐₓ) were observed at 0.5, 0.5, and 2 hrs post dose, respectively, suggesting rapid rate of absorption.

Brain concentrations were quantifiable up to 8 h following oral administration at 3, 10, and 30 mg/kg with brain exposure-Kp of 0.47, 0.46, and 0.34, respectively.

Spinal cord concentrations were quantifiable up to 8 h following oral administration at 3, 10, and 30 mg/kg with spinal cord exposure-Kp of 0.34, 0.34, and 0.27, respectively.

Following single oral administration of Compound A at 3, 10, and 30 mg/kg, more than dose proportional increase in exposure with increasing doses was observed in plasma, brain and spinal cord.

TABLE B
Mean PK parameters of Compound A following a single oral administration
in male C57BL/6 mice (Dose: 3, 10 and 30 mg/kg)

| | | Dose | Tₘₐₓ | Cₘₐₓ | AUCₗₐₛₜ | Cₘₐₓ | AUCₗₐₛₜ | | |
|---|---|---|---|---|---|---|---|---|---|
| Matrix | Route | (mg/kg) | (h) | (ng/mL) | (h*ng/mL) | Ratio | Ratio | F (%) | — |
| Plasma | PO | 3 | 0.50 | 1469 | 4571 | — | — | | — |
| | | 10 | 0.50 | 5002 | 16939 | 3.41 | 3.71 | | — |
| | | 30 | 2.00 | 11335 | 80797 | 7.72 | 17.7 | | — |

| | | Dose | Tₘₐₓ | Cₘₐₓ | AUCₗₐₛₜ | Cₘₐₓ | AUCₗₐₛₜ | Brain-Kp | Brain-Kp |
|---|---|---|---|---|---|---|---|---|---|
| Matrix | Route | (mg/kg) | (h) | (ng/g) | (h*ng/g) | Ratio | Ratio | (Cₘₐₓ) | (AUCₗₐₛₜ) |
| Brain^{#} | PO | 3 | 0.50 | 717 | 2155 | — | — | 0.49 | 0.47 |
| | | 10 | 0.50 | 2240 | 7747 | 3.12 | 3.60 | 0.45 | 0.46 |
| | | 30 | 2.00 | 5568 | 27698 | 7.76 | 12.9 | 0.49 | 0.34 |
| | | Dose | Tₘₐₓ | Cₘₐₓ | AUCₗₐₛₜ | Cₘₐₓ | AUCₗₐₛₜ | Brain-Kp | Brain-Kp |
| Matrix | Route | (mg/kg) | (h) | (ng/g) | (h*ng/g) | Ratio | Ratio | (Cₘₐₓ) | (AUCₗₐₛₜ) |
| Spinal | PO | 3 | 0.50 | 535 | 1565 | — | — | 0.36 | 0.34 |
| Cord^{#} | | 10 | 0.50 | 1567 | 5812 | 2.93 | 3.71 | 0.31 | 0.34 |
| | | 30 | 2.00 | 4210 | 22170 | 7.87 | 14.2 | 0.37 | 0.27 |

^{#}Brain and spinal cord, Cmax and AUC are expressed as ng/g and h* ng/g, respectively, Cₘₐₓ and AUCₗₐₛₜ ratio were calculated from lowest dose administered.

TABLE C
Mean brain-to-plasma concentration ratio of Compound
A in male C57BL/6 mice following a single oral
administration (Dose: 3, 10, and 30 mg/kg)

| | | | Mean plasma | Mean brain | Brain/ |
|---|---|---|---|---|---|
| | Dose | Time | Concentration | Concentration | Plasma |
| Route | (mg/kg) | (h) | (ng/mL) | (ng/g) | ratio |
| PO | 3 | 0.5 | 1468.74 | 717.36 | 0.49 |
| | | 2 | 760.36 | 361.64 | 0.48 |
| | | 8 | 83.61 | 27.03 | 0.32 |
| | | 24 | BLQ | BLQ | NA |
| | 10 | 0.5 | 5001.75 | 2239.75 | 0.45 |
| | | 2 | 3091.06 | 1429.40 | 0.46 |
| | | 8 | 115.20 | 49.07 | 0.43 |
| | | 24 | BLQ | BLQ | NA |
| | 30 | 0.5 | 10736.75 | 4374.34 | 0.41 |
| | | 2 | 11335.05 | 5567.99 | 0.49 |
| | | 8 | 2502.64 | 814.52 | 0.33 |
| | | 24 | 3.10 | BLQ | NA |

LLOQ = 2.02 ng/mL for plasma and brain,
BLQ—Below Level of Quantitation,
NA—Not Applicable

TABLE D
Mean spinal cord-to-plasma concentration ratio of
Compound A in male C57BL/6 mice following a single
oral administration (Dose: 3, 10, and 30 mg/kg)

| | | | Mean plasma | Mean spinal cord | Spinal |
|---|---|---|---|---|---|
| | Dose | Time | Concentration | Concentration | cord/Plasma |
| Route | (mg/kg) | (h) | (ng/mL) | (ng/g) | ratio |
| PO | 3 | 0.5 | 1468.74 | 534.71 | 0.36 |
| | | 2 | 760.36 | 260.25 | 0.34 |
| | | 8 | 83.61 | 18.27 | 0.22 |
| | | 24 | BLQ | BLQ | NA |
| | 10 | 0.5 | 5001.75 | 1566.87 | 0.31 |
| | | 2 | 3091.06 | 1114.39 | 0.36 |
| | | 8 | 115.20 | 21.98 | 0.19 |
| | | 24 | BLQ | BLQ | NA |
| | 30 | 0.5 | 10736.75 | 3460.81 | 0.32 |
| | | 2 | 11335.05 | 4209.60 | 0.37 |
| | | 8 | 2502.64 | 974.54 | 0.39 |
| | | 24 | 3.10 | BLQ | NA |

LLOQ = 2.02 ng/mL for plasma and spinal cord,
BLQ—Below Level of Quantitation,
NA—Not Applicable

FIGS. 4A and 4B are graphs of a linear plot (FIG. 4A) and Semi-log plot (FIG. 4B) of the plasma, brain and spinal cord concentrations-time profiles (mean+SD) of Compound A following a single oral administration in male C57BL/6 mice (Dose: 3 mg/kg).

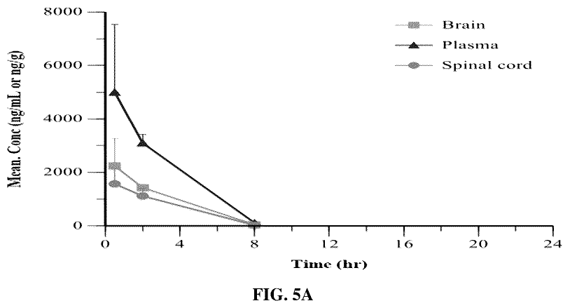

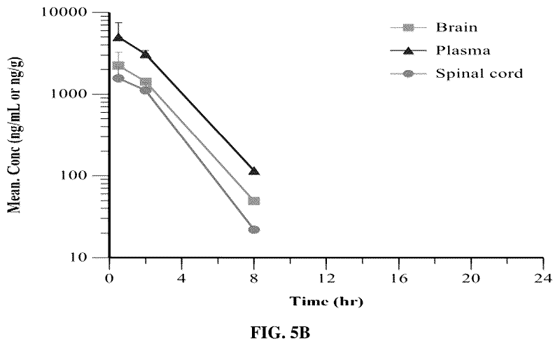

FIGS. 5A and 5B are graphs of a linear plot (FIG. 5A) and semi-log plot (FIG. 5B) of the plasma, brain and spinal cord concentrations-time profiles (mean+SD) of Compound A following a single oral administration in male C57BL/6 mice (Dose: 10 mg/kg).

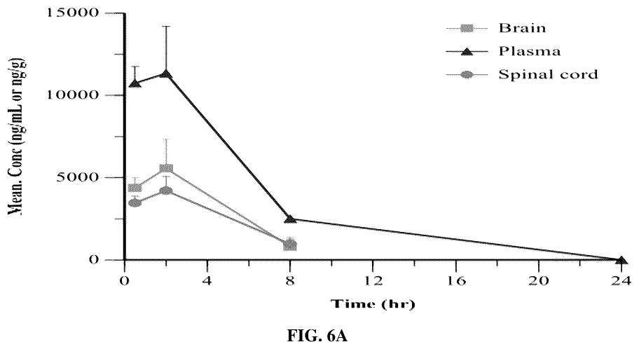

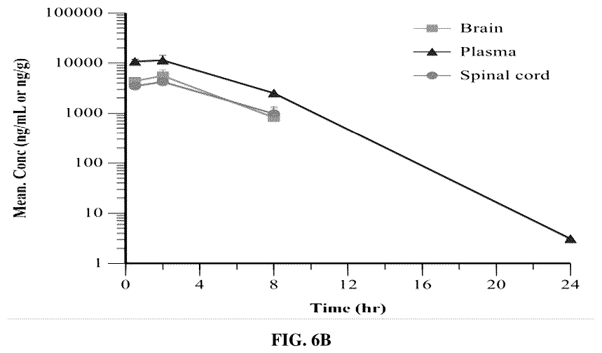

FIGS. 6A and 6B are graphs of a linear plot (FIG. 6A) and semi-log plot (FIG. 6B) of the plasma, brain, and spinal cord concentrations-time profiles (mean+SD) of Compound A following a single oral administration in male C57BL/6 mice (Dose: 30 mg/kg).

Following single oral administration of Compound A at 3, 10, and 30 mg/kg, more than dose proportional increase in exposure with increasing doses was observed in plasma, brain and spinal cord.

### Example 7. Homogeneous Time Resolved Fluorescence (HTRF) Analysis of Acetyl-a-Tubulin (K40) and a-Tubulin in SH-SY5Y Cells Following Treatment with HDAC6 Inhibitors

SH-SY5Y cells (ATCC; CRL2266) were treated with an 11-point dose (1.7 nM-100 M) response of test compound or SAHA and incubated at 37° C. and 5% CO2 for 4 hrs. Cells were then lysed using 1× Cisbio lysis buffer and Homogeneous Time Resolved Fluorescence (HTRF) analysis was performed following manufacturer instructions (Revvity; 63ADK072PEH, 63ADK073PEH) with a cell density of approximately 30,000 to 40,000 cells per well and volume of cell lysate of approximately 15-20 μL) Cell treatment and analysis was repeated for a total of three biological replicates. Acetylated-tubulin(K40) HTRF ratios were normalized to total-tubulin HTRF ratios for each sample. Acetylated-tubulin(K40)/total-tubulin ratios were normalized to vehicle (DMSO)-treated cells. Statistics were performed using one-way ANOVA followed by FDR multiple comparisons correction with each dose compared to DMSO-treated cells.

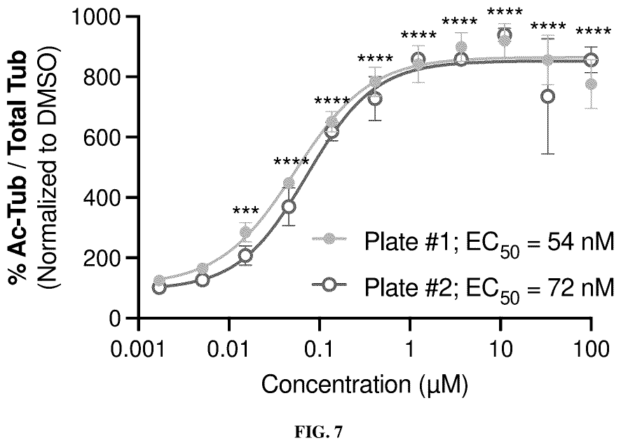

FIG. 7 is a graph showing the dose-response of acetyl-a-tubulin (K40)/total a-tubulin in SH-SY5Y cells following treatment with SAHA. Mean±SEM, N=3. ***, q-value<0.001; ****, q-value<0.0001.

HTRF measures levels of acetylated α-tubulin (K40) relative to total α-tubulin. An increase in acetyl-K40 signal following HDAC6 inhibitor treatment indicates that the inhibitor is biologically active and successfully blocking HDAC6 function. The table below provides the ratio of HDAC6 IC50 for each compound relative to Compound A, and the percent acetylated tubulin over total tubulin at 1 micromolar [DMSO normalized] according to the HTRF analysis of Example 5.

Numerous compounds were identified that are characterized by both a greater HDAC6 IC₅₀ potency and a greater ratio of acetylated to total tubulin relative to Compound A (86) in the HTRF assay of Example 5.

| | HDAC6 IC50/ | Ac-Tub/Total Tub@ 1 mM (%) |
|---|---|---|
| Compound | HDAC6 IC50 (Cmpd A) | [DMSO Normalized] |
| A (86) | 1.00 | 152 |
| 36 | 0.70 | 330 |
| 56 | 1.26 | 258 |
| 123 | 0.88 | 264 |
| 144 | 0.67 | 222 |
| 150 | 0.46 | 213 |
| 151 | 0.22 | 276 |
| 155 | 0.15 | 338 |
| 157 | 0.20 | 283 |
| 158 | 0.81 | 191 |
| 160 | 1.57 | 159 |
| B | 0.18 | 223 |
| C | 0.10 | 238 |
| D | 0.20 | 307 |
| E | 0.29 | 196 |
| F | 0.15 | 217 |
| G | 0.21 | 181 |
| H | — | 258 |

### Example 8. Pharmacokinetic (PK) Study of Compounds of Formula (I) in Rats Test System

Healthy Male Sprague Dawley Rats (8-12 weeks old) weighing between 250±50 g were procured from Hylasco, India. Three rats were housed in each cage. Temperature and humidity were maintained at 22±3° C. and 30-70%, respectively and illumination was controlled to give a sequence of 12 h light and 12 h dark cycle. Temperature and humidity were recorded by auto-controlled data logger system. All the animals were provided laboratory rodent diet (Altromin). Reverse osmosis water treated with ultraviolet light was provided ad libitum.

### Study Design

A total of six male rats were included into study and divided into two groups as Group-1/IV1 (n=3) and Group-2/IV2 (n=3) with serial sampling design and all the animals were administered intravenously as slow bolus injection through tail vein, with solution formulation of compounds at 0.5 mg/kg dose of each compound. The formulation vehicle used was 10% v/v DMSO, 10% v/v Tween 80 and 80% v/v Normal saline. The dosing volume for intravenous administration was 2 mL/kg. The assignment of animals was shown in the table below:

| | | Dose | | |
|---|---|---|---|---|
| Group | Route | (mg/kg) | Matrix | Animal ID |
| 1 | IV1 | 0.5 each compound | Plasma | R6212 to R6214 |
| 2 | IV2 | 0.5 each compound | Plasma & | R6215 to R6217 |
| | | | Brain | |

### Formulation Preparation

IV (0.25 mg/mL for each compound): Each compound was weighed separately as required for 10.0 mg/mL DMSO stock preparation and taken in a vial, which were corrected for purity. Calculated volume of DMSO was added to each vial separately and vortexed thoroughly to obtain a clear solution of 10.0 mg/mL DMSO stock. 0.125 mL of DMSO stock from each vial was transferred to the labelled bottle, and to this 0.500 mL of Tween-80 and 4.00 mL of normal saline was added and vortexed to get clear solution. The amount weighed and calculation details are as below:

### Data Analysis

The Non-Compartmental-Analysis tool of Phoenix WinNonlin® (Version 8.3) was used to assess the pharmacokinetic parameters. The areas under the concentration time curve (AUCₗₐₛₜ and AUC_{inf}) were calculated by linear trapezoidal rule. The terminal elimination rate constant, ke was determined by regression analysis of the linear terminal portion of the log plasma concentration-time curve. Clearance was estimated as Dose/AUC_{inf} and Vss as CL*MRT. Also, tissue to plasma concentration ratios (tissue KP) were calculated and reported in Microsoft excel format.

### Results

| Molecule | PK | | Dose | C0 | T½ | AUClast | AUCinf | CL | V_{SS} |
|---|---|---|---|---|---|---|---|---|---|
| Name | Species | Route | (mg/kg) | (μM) | (hr) | (h*μM) | (h*μM) | (mL/min/kg) | (L/kg) |
| Compound A | Rat SD | iv | 0.5 | 4.3 | 1.6 | 7.4 | 7.7 | 3.2 | 0.5 |
| Compound 36 | Rat SD | iv | 0.5 | 1.3 | 0.3 | 0.7 | 0.7 | 33.4 | 0.6 |
| Compound 123 | Rat SD | iv | 0.5 | 6.2 | 3.3 | 10.2 | 11.8 | 2.0 | 0.4 |
| Compound 150 | Rat SD | iv | 0.5 | 5.5 | 0.3 | 1.8 | 1.8 | 13.5 | 0.3 |
| Compound 151 | Rat SD | iv | 0.5 | 4.1 | 0.9 | 2.7 | 2.9 | 8.6 | 0.6 |
| Compound 155 | Rat SD | iv | 0.5 | 2.1 | 0.2 | 0.3 | 0.3 | 74.5 | 0.9 |
| Compound D | Rat SD | iv | 0.5 | 4.4 | 1.7 | 10.8 | 10.9 | 1.7 | 0.3 |
| Compound E | Rat SD | iv | 0.5 | 16.0 | 18.4 | 151.0 | 249.0 | 0.1 | 0.1 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method treating a neurodegenerative disease by selectively inhibiting HDAC6 over HDAC8 while inhibiting alpha tubulin deacetylation (K40), the method comprising administering to a subject in need thereof, a therapeutically effective amount of a compound of Formula (I), 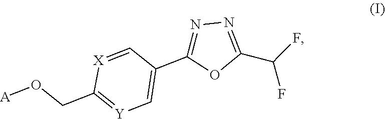
or a pharmaceutically acceptable salt thereof,
wherein:
X is N or CR¹, and Y is CR¹ or N, provided that at least one of X and Y is N;
R¹ is hydrogen or halogen;
A is a 6,6-bicyclic aryl ring system, a 5,6-bicyclic heteroaryl ring system or a 6,6-bicyclic heteroaryl ring system, wherein each heteroaryl ring system comprising one or more nitrogen or oxygen heteroatoms, and A is optionally substituted with one or more independent substituents R²; and
each occurrence of R² is independently halogen, amido, cyano, amino, acyl, C₁₋₄ alkyl, C₁₋₄ alkenyl, C₁₋₄ alkynyl, 3-6 member heteroaliphatic, C₃₋₆ carbocyclyl, 3-6-member heterocyclyl, C₆₋₁₄ aryl, or 6-10 member heteroaryl; wherein each amido, amino, acyl, alkyl, alkenyl, alkynyl, heteroaliphatic is optionally substituted with one or more halogen, amido, cyano, amino, acyl, C₁₋₄ alkyl, C₁₋₄ alkenyl, C₁₋₄ alkynyl, 3-6 member heteroaliphatic, C₃₋₆ carbocyclyl, 3-6-member heterocyclyl, C₆₋₁₄ aryl, or 6-10 member heteroaryl; and each carbocyclyl, heterocyclyl, aryl, or heteroaryl is optionally substituted with one or more halogen, amido, cyano, amino, acyl, C₁₋₄ alkyl, C₁₋₄ alkenyl, or C₁₋₄ alkynyl; or
any two occurrences of R² are joined with their intervening atoms to form substituted or unsubstituted 3-6 member carbocyclyl, substituted or unsubstituted 3-6 member heterocyclyl, substituted or unsubstituted C₆₋₁₄ aryl, or substituted or unsubstituted 6-10 member heteroaryl; each carbocyclyl, heterocyclyl, aryl, or heteroaryl is optionally substituted with one or more halogen, amido, cyano, amino, acyl, C₁₋₄ alkyl, C₁₋₄ alkenyl, or C₁₋₄ alkynyl.

2. The method of claim 1, wherein R¹ is hydrogen or F.

3. The method of claim 2, wherein A is a 5,6-bicyclic heteroaryl group or a 6,6-bicyclic heteroaryl group comprising one or more nitrogen atoms.

4. The method of claim 3, wherein A is unsubstituted or A is substituted with 1-3 R² and each R² is independently halogen, amino, C₁₋₄ alkyl, C₁₋₄ alkoxy, C₃₋₆ cycloalkyl, C₁₋₄ haloalkyl, acyl, or amide; wherein each amino is optionally substituted with one or more C₁₋₄ alkyl, and each alkyl or alkoxy is optionally substituted with one or more amino or 3-6 member heterocycloalkyl or C₃₋₆ cycloalkyl each optionally substituted with one or more C₁₋₄ alkyl.

5. The method of claim 3, wherein A is quinazoline, quinoxaline, isoquinoline, 2,3-dihydroimidazo[1,2-a]pyrazine, imidazo[1,2-a]pyridine, or benzo[d]oxazole, optionally substituted with 1-3 R².

6. The method of claim 5, wherein R² is halogen, C₁₋₄ alkyl, phenyl optionally substituted with halogen or 6-member heteroaryl comprising one or more nitrogen heteroatom.

7. The method of claim 6, wherein R² is fluorine, bromine, chlorine, methyl, ethyl, methoxy, pyridine, pyrimidine or phenyl optionally substituted with fluorine, bromine, chlorine, methyl, ethyl, or methoxy.

8. The method of claim 7, wherein A is unsubstituted, or A is substituted with one R².

9. The method of claim 2, wherein A is

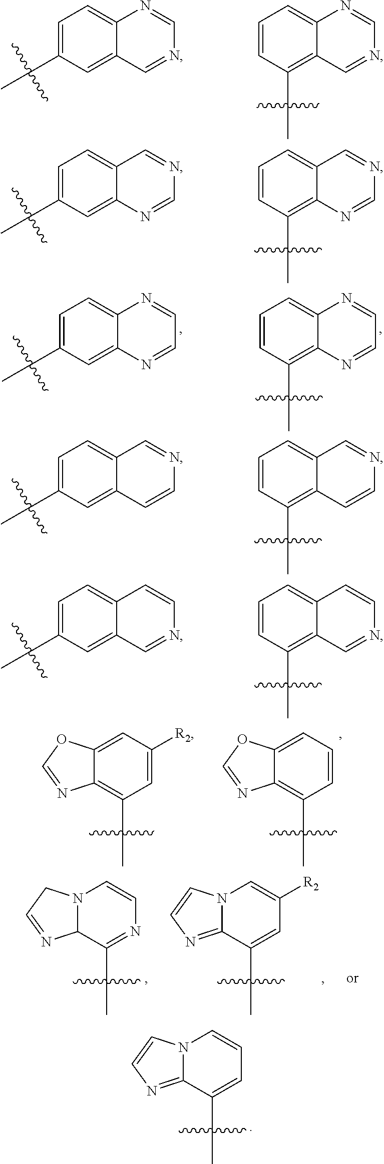

10. The compound of claim 9, wherein R² is halogen, phenyl optionally substituted with fluoro, or 6-member heteroaryl comprising one or more nitrogen heteroatoms.

11. The method of claim 1, wherein X is N, and Y is CH or CF.

12. The method of claim 11, wherein A is an unsubstituted 5,6-bicyclic heteroaryl group or an unsubstituted 6,6-bicyclic heteroaryl group comprising one or more nitrogen atoms.

13. The method of claim 11, wherein A is a 5,6-bicyclic heteroaryl group or a 6,6-bicyclic heteroaryl group comprising one or more nitrogen atoms, wherein A is substituted with a single halogen, pyridine, pyrimidine, or phenyl optionally substituted with one or more halogen.

14. The method of claim 1, wherein the compound is 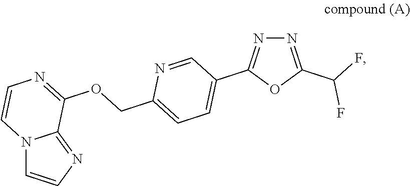 [SMILES: FC(F)c1nnc(-c2ccc(COc3nccn4ccnc34)nc2)o1]
or a pharmaceutically acceptable salt thereof.

15. The method of claim 1, wherein the compound is 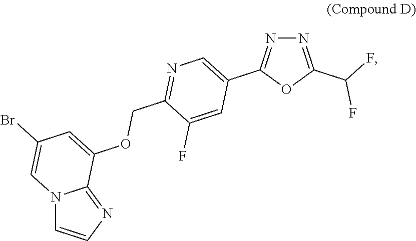 [SMILES: Fc1cc(-c2nnc(C(F)F)o2)cnc1COc1cc(Br)cn2ccnc12]
or a pharmaceutically acceptable salt thereof.

16. The method of claim 1, wherein the compound is 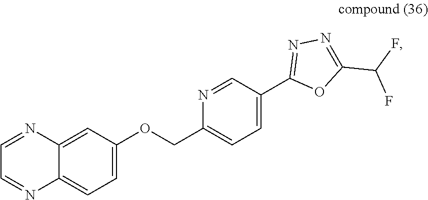 [SMILES: FC(F)c1nnc(-c2ccc(COc3ccc4nccnc4c3)nc2)o1]
or a pharmaceutically acceptable salt thereof.

17. The method of claim 1, wherein the compound is 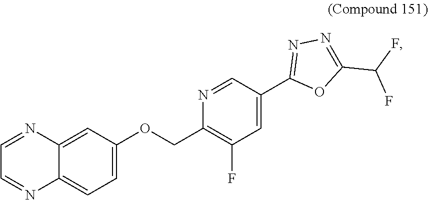 [SMILES: Fc1cc(-c2nnc(C(F)F)o2)cnc1COc1ccc2nccnc2c1]
or a pharmaceutically acceptable salt thereof.

18. The method of claim 1, wherein the compound is 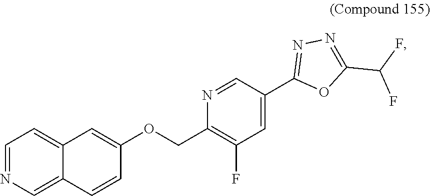 [SMILES: Fc1cc(-c2nnc(C(F)F)o2)cnc1COc1ccc2cnccc2c1]
or a pharmaceutically acceptable salt thereof.

19. The method of claim 1, wherein the compound is 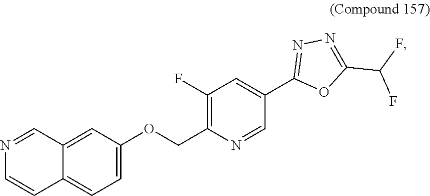 [SMILES: Fc1cc(-c2nnc(C(F)F)o2)cnc1COc1ccc2ccncc2c1]
or a pharmaceutically acceptable salt thereof.

20. A compound selected from the group consisting of: 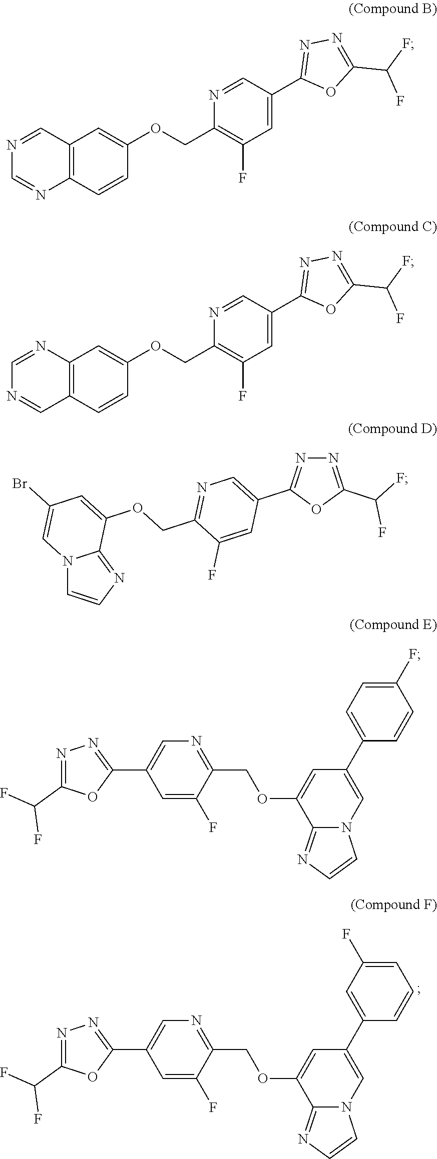 [SMILES: Fc1cc(-c2nnc(C(F)F)o2)cnc1COc1ccc2ncncc2c1 ; Fc1cc(-c2nnc(C(F)F)o2)cnc1COc1ccc2cncnc2c1 ; Fc1cc(-c2nnc(C(F)F)o2)cnc1COc1cc(Br)cn2ccnc12 ; Fc1ccc(-c2cc(OCc3ncc(-c4nnc(C(F)F)o4)cc3F)c3nccn3c2)cc1 ; Fc1cccc(-c2cc(OCc3ncc(-c4nnc(C(F)F)o4)cc3F)c3nccn3c2)c1] 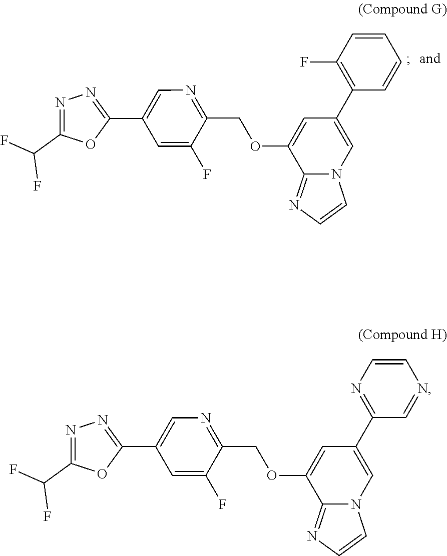 [SMILES: Fc1ccccc1-c1cc(OCc2ncc(-c3nnc(C(F)F)o3)cc2F)c2nccn2c1 ; Fc1cc(-c2nnc(C(F)F)o2)cnc1COc1cc(-c2cnccn2)cn2ccnc12]
or a pharmaceutically acceptable salt thereof.
